(19) United States
(12) Patent Application Publication
(10) Pub. No.: US 2024/0156972 A1
(43) Pub. Date: May 16, 2024

(54) ANTIBODY RECRUITMENT MOLECULES AND METHODS OF TREATING CANCER USING SAME

(71) Applicant: McMaster University, Hamilton (CA)

(72) Inventors: Anthony RULLO, Hamilton (CA); Yonghong WAN, Ancaster (CA); Harrison McCann, Whitby (CA); Benjamin LAKE, Ancaster (CA)

(73) Assignee: McMaster University, Hamilton, ON (CA)

(21) Appl. No.: 18/547,714

(22) PCT Filed: Feb. 24, 2022
(86) PCT No.: PCT/CA2022/050267

§ 371 Date: Aug. 24, 2023

Related U.S. Application Data
(60) Provisional application No. 63/278,183, filed on Nov. 11, 2021.
(60) Provisional application No. 63/153,500, filed on Feb. 25, 2021.

(51) Int. Cl. A61K 47/64; A61K 35/763; A61K 47/54; A61K 47/60; A61P 35/00; C07K 14/005

(52) U.S. Cl. CPC A61K 47/64; A61K 35/763; A61K 47/545; A61K 47/60; A61P 35/00; C07K 14/005; C12N 2710/16622

(57) ABSTRACT

The present disclosure relates generally to the field of cancer immunotherapy. More particularly, the present disclosure provides antibody recruitment molecules, pharmaceutical compositions comprising same and kits comprising same. The present disclosure also provides methods of treating cancer using the antibody recruitment molecules in combination with oncolytic virus therapy, and methods for enhancing the efficacy and/or reducing the toxicity of the oncolytic virus therapy.

### CROSS REFERENCE TO PRIOR APPLICATIONS

This application claims priority under the Paris Convention to U.S. Provisional Patent Application No. 63/153,500 filed on Feb. 25, 2021 and U.S. Provisional Patent Application No. 63/278,183 filed on Nov. 11, 2021, which are incorporated herein by reference as if set forth in their entirety.

### FIELD OF THE DISCLOSURE

The present disclosure relates generally to the field of cancer immunotherapy. More particularly, the present disclosure relates to compounds and methods for recruiting endogenous antibodies to tumor cells, and for enhancing the efficacy and/or reducing the toxicity of oncolytic virus (OV) therapy.

### BACKGROUND OF THE DISCLOSURE

OV therapy exploits the abilities of oncolytic viruses to infect tumor cells and mediate their destruction by direct tumor cell lysis and/or by stimulating host immune responses against the tumor. OV therapy has demonstrated tremendous promise in affecting tumor regression in pre-clinical studies and clinical trials. In 2015, Talimogene Laherparepvec (“T-VEC”, marketed under the trade name IMLYGIC™ and also referred to as AMG-678, OncoVEX or OncoVEX^{GM-CSF}) became the first OV therapy to be approved for clinical use in the United States and the European Union. T-VEC comprises an attenuated, genetically modified Herpes Simplex Virus Type 1 (HSV-1).

The anti-tumor efficacy of OV therapy can become compromised by intrinsic OV immunogenicity. Upon initial or repeated OV administration, endogenous pre-existing and/or OV-induced serum antibodies may neutralize and clear the OV, thereby diminishing the anti-tumor efficacy of the treatment. In particular, a number of HSV-specific serum antibodies have been identified that might play a role in viral sequestration prior to or following administration of T-VEC. These serum antibodies appear to recognize epitopes on the surface of HSV-1 and may also cross-react with epitopes on the surface of HSV-2. One such epitope that binds to these serum antibodies is the N-terminal domain of the HSV viral surface protein, glycoprotein D (gD). Thus, endogenous HSV-specific antibodies may decrease the therapeutic efficacy of T-VEC.

There is a need for new therapeutics and treatment modalities in order to enhance the efficacy and/or reduce the toxicity of OV therapy in cancer patients.

Antibody Recruitment Molecules (ARMs), also known as Antibody Engagers (AEs) are bi-functional small molecules capable of delivering antibodies to disease-causing entities, such as tumor cells. Previously, ARMs have been developed to recruit endogenous serum antibodies against tumor cells, such as anti-dinitrophenyl (DNP) and total immunoglobulin G (pan-IgG) antibodies, to elicit anti-tumor immune responses mediated by the fragment crystallizable (Fc) regions of the serum antibodies.

However, some features of the earlier ARMs limit their efficacy in particular modes of cancer immunotherapy. For example, the predominant anti-DNP antibody isotype in human serum is immunoglobulin M (IgM), which is incapable of activating endogenous natural killer (NK) cells against the tumor. On the other hand, limitations of pan-IgG recruitment against a tumor include (i) recruitment of inhibitory IgG2b antibodies, which are part of the pan-IgG repertoire; and (ii) formation of autoinhibitory complexes promoted by the high concentration of pan-IgG, where both the tumor cell surface and immune cell surface are saturated with the bi-functional ARM molecules bound to serum pan-IgG. A potential limitation of ARM-mediated antibody recruitment as a stand-alone cancer immunotherapy is that the Fc-mediated immune response may not be sufficiently robust to eliminate solid tumors and prevent tumor relapse.

There is a need for improved ARM compounds and antibody recruitment strategies for cancer immunotherapy.

### SUMMARY OF THE DISCLOSURE

The inventors have invented ARM compounds, pharmaceutical compositions comprising the ARM compounds, kits comprising the ARM compounds, and methods for treating cancer and for enhancing the efficacy and/or reducing the toxicity of an OV therapy in a subject using the ARM compounds.

In a first aspect of the present disclosure, a compound of Formula (I) or a pharmaceutically acceptable salt or solvate thereof is provided,

(TBT)ₙ-(L)m-(ABT)ₚ (I)

wherein:
TBT is a target binding terminus comprising at least one moiety that binds to at least one target protein;
L is an optional linker;
ABT is an antibody binding terminus comprising at least one epitope or epitope mimetic of a Herpes Simplex Virus (HSV) surface protein;
each of n and p is independently 1 or any integer greater than 1; and
m is 0 or any integer greater than 0.

In a second aspect of the present disclosure, a compound is provided. The compound comprises:
a. at least one target binding terminus (TBT) comprising one or more moieties that bind to one or more target proteins;
b. at least one antibody binding terminus (ABT) comprising one or more epitopes or epitope mimetics of an HSV surface protein; and
c. optionally, at least one linker connecting the at least one TBT with the at least one ABT, or a pharmaceutically acceptable salt or solvate thereof.

In certain embodiments of the compounds provided herein, the HSV surface protein is a glycoprotein.

In certain embodiments of the compounds provided herein, the HSV surface protein is gD.

In certain embodiments of the compounds provided herein, the epitope or epitope mimetic comprises the amino acid sequence set forth in any one of SEQ ID NOs: 1-9, or a variant thereof.

In certain embodiments of the compounds provided herein, the epitope or epitope mimetic consists of the amino acid sequence set forth in any one of SEQ ID NOs: 1-9, or a variant thereof.

In certain embodiments of the compounds provided herein, the compound further comprises one or more reactive groups that mediate covalent conjugation of the compound with an HSV-specific antibody and/or the target protein.

In certain embodiments of the compounds provided herein, the HSV-specific antibody is a serum antibody.

In certain embodiments of the compounds provided herein, the reactive group comprises an electrophilic functional group that reacts with an amino acid nucleophile in a nucleophilic substitution reaction.

In certain embodiments of the compounds provided herein, the reactive group comprises an acyl imidazole group having the following structure:

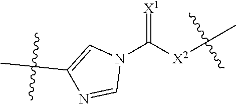

wherein:
X¹ is S, O or NR¹;
X² is O or NR²; and
R¹ and R² are independently H or C₁₋₄ alkyl.

In certain embodiments of the compounds provided herein, the reactive group comprises a fluorosulfate-I-tyrosine (FSY) group or an aryl-sulfonyl fluoride (ASF) group.

In certain embodiments of the compounds provided herein, the target protein is expressed on the surface of a cancer cell.

In certain embodiments of the compounds provided herein, the target protein is urokinase receptor (uPAR), prostate-specific membrane antigen (PSMA), human epidermal growth factor receptor 2 (HER2), or folate receptor.

In certain embodiments of the compounds provided herein, the target protein is PSMA and the TBT has the following structure:

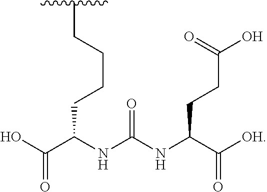 [SMILES: *CCCC[C@H](NC(=O)N[C@@H](CCC(=O)O)C(=O)O)C(=O)O]

In certain embodiments of the compounds provided herein, the compound is:

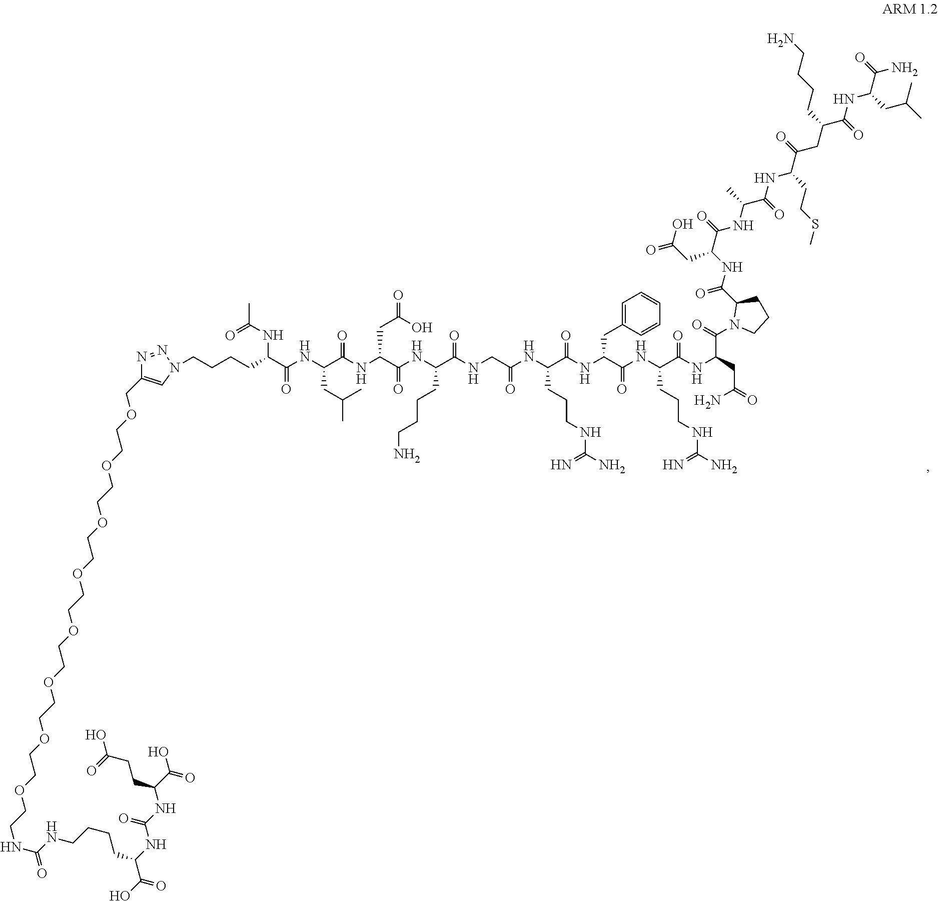 [SMILES: CSCC[C@H](NC(=O)[C@@H](C)NC(=O)[C@@H](CC(=O)O)NC(=O)[C@H]1CCCN1C(=O)[C@@H](CC(N)=O)NC(=O)[C@H](CCCNC(=N)N)NC(=O)[C@@H](Cc1ccccc1)NC(=O)[C@H](CCCNC(=N)N)NC(=O)CNC(=O)[C@H](CCCCN)NC(=O)[C@@H](CC(=O)O)NC(=O)[C@H](CC(C)C)NC(=O)[C@H](CCCCn1cc(COCCOCCOCCOCCOCCOCCOCCOCCNC(=O)NCCCC[C@H](NC(=O)N[C@@H](CCC(=O)O)C(=O)O)C(=O)O)nn1)NC(C)=O)C(=O)C[C@H](CCCCN)C(=O)N[C@@H](CC(C)C)C(N)=O]

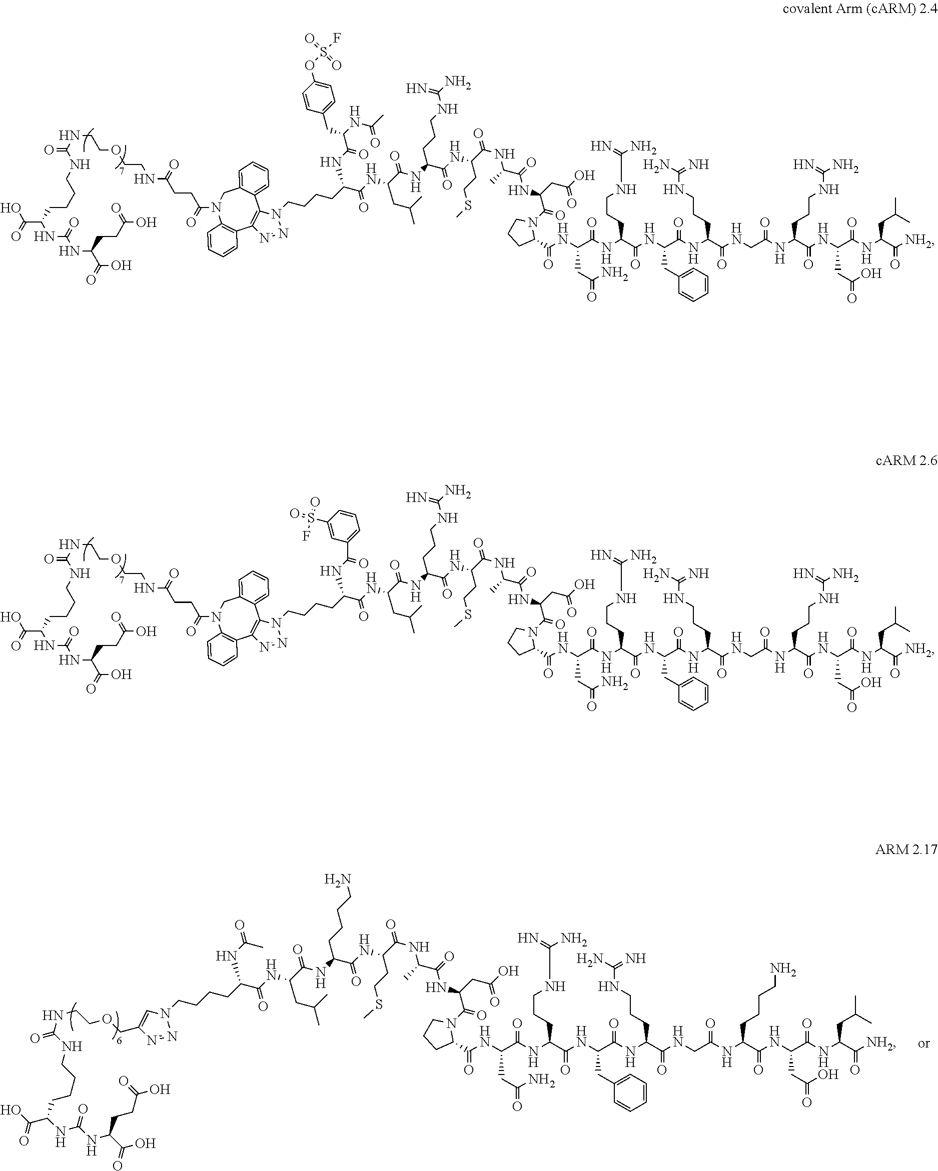

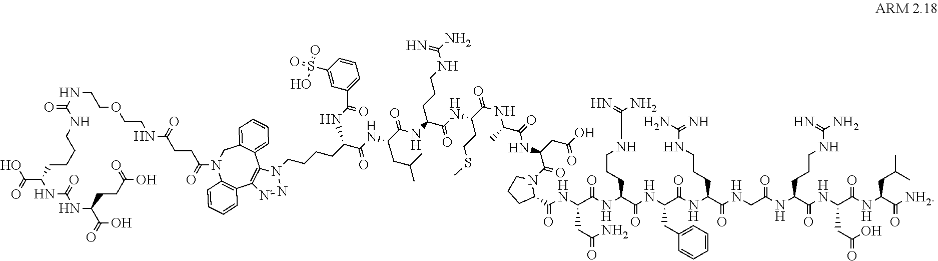 [SMILES: CSCC[C@H](NC(=O)[C@H](CCCNC(=N)N)NC(=O)[C@H](CC(C)C)NC(=O)[C@H](CCCCn1nnc2c1-c1ccccc1CN(C(=O)CCC(=O)NCCOCCNC(=O)NCCCC[C@H](NC(=O)N[C@@H](CCC(=O)O)C(=O)O)C(=O)O)c1ccccc1-2)NC(=O)c1cccc(S(=O)(=O)O)c1)C(=O)N[C@@H](C)C(=O)N[C@@H](CC(=O)O)C(=O)N1CCC[C@H]1C(=O)N[C@@H](CC(N)=O)C(=O)N[C@@H](CCCNC(=N)N)C(=O)N[C@@H](Cc1ccccc1)C(=O)N[C@@H](CCCNC(=N)N)C(=O)NCC(=O)N[C@@H](CCCNC(=N)N)C(=O)N[C@@H](CC(=O)O)C(=O)N[C@@H](CC(C)C)C(N)=O]

In certain embodiments of the compounds provided herein, the target protein is uPAR and the TBT has the following structure:

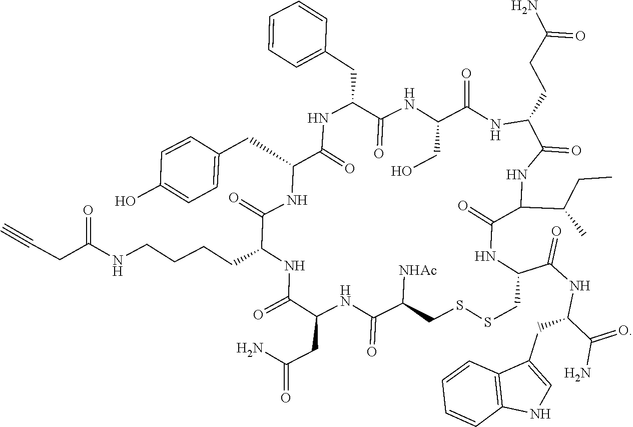

In certain embodiments of the compounds provided herein, the target protein is HER2 and the TBT has the following structure:

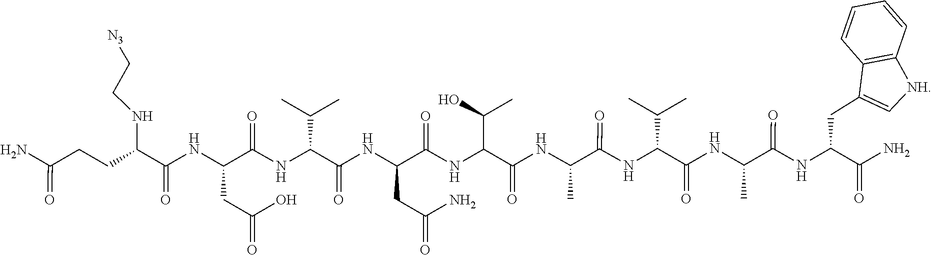 [SMILES: CC(C)[C@@H](NC(=O)[C@H](C)NC(=O)C(NC(=O)[C@@H](CC(N)=O)NC(=O)[C@H](NC(=O)[C@H](CC(=O)O)NC(=O)[C@H](CCC(N)=O)NCCN=[N+]=[N-])C(C)C)[C@H](C)O)C(=O)N[C@@H](C)C(=O)N[C@H](Cc1c[nH]c2ccccc12)C(N)=O]

In certain embodiments of the compounds provided herein, the target protein is folate receptor and the TBT comprises methotrexate or folate.

In certain embodiments of the compounds provided herein, the target protein is expressed on the surface of a pathogen or a cell infected with a pathogen.

In certain embodiments of the compounds provided herein, the target protein is expressed on the surface of a pathogen or a cell infected with a pathogen, and the pathogen comprises a virus, bacterium, fungus or parasite.

In certain embodiments of the compounds provided herein, the TBT is biotin or a derivative thereof.

In certain embodiments of the compounds provided herein, the TBT has the following structure:

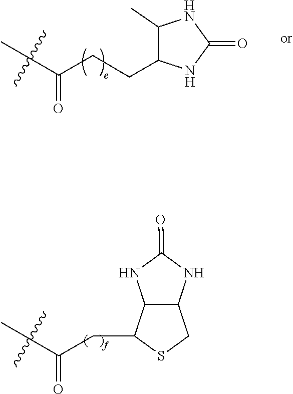

wherein e and f are, independently, an integer from 0 to 15.

In certain embodiments of the compounds provided herein, the compound is:

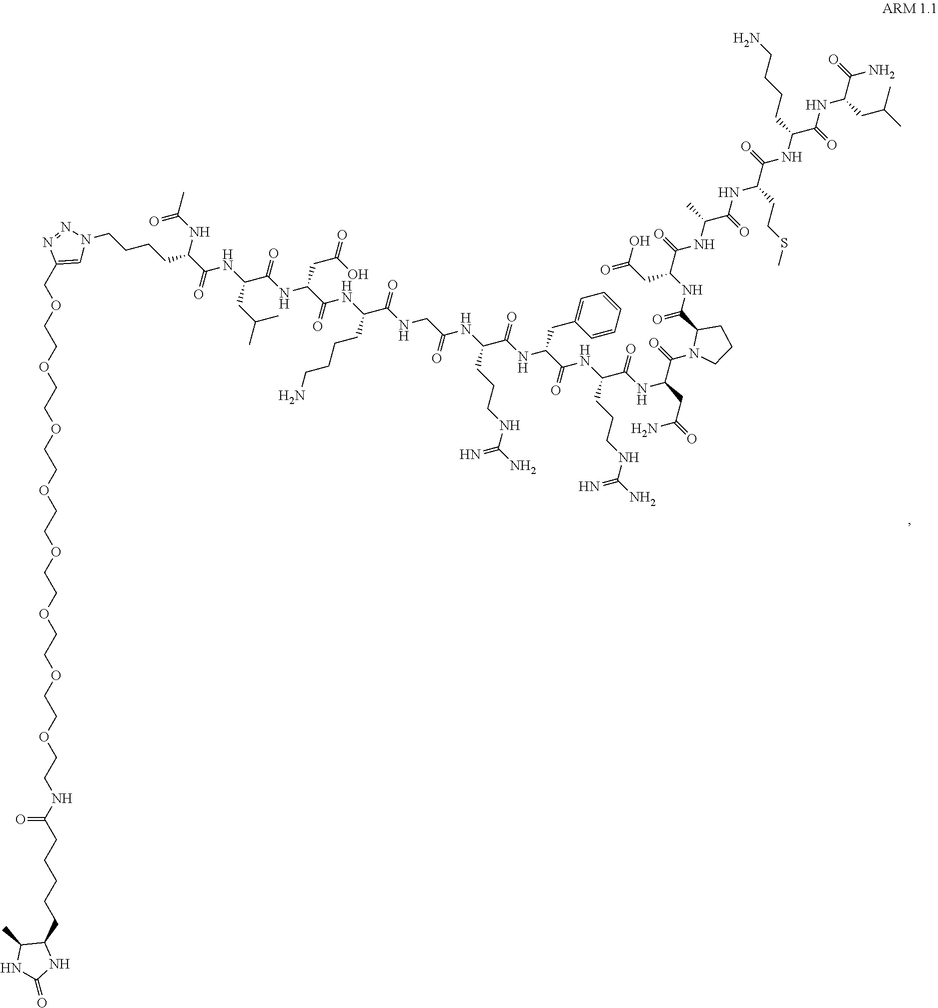 [SMILES: CSCC[C@H](NC(=O)[C@@H](C)NC(=O)[C@@H](CC(=O)O)NC(=O)[C@H]1CCCN1C(=O)[C@@H](CC(N)=O)NC(=O)[C@H](CCCNC(=N)N)NC(=O)[C@@H](Cc1ccccc1)NC(=O)[C@H](CCCNC(=N)N)NC(=O)CNC(=O)[C@H](CCCCN)NC(=O)[C@@H](CC(=O)O)NC(=O)[C@H](CC(C)C)NC(=O)[C@H](CCCCn1cc(COCCOCCOCCOCCOCCOCCOCCOCCNC(=O)CCCCC[C@H]2NC(=O)N[C@H]2C)nn1)NC(C)=O)C(=O)N[C@H](CCCCN)C(=O)N[C@@H](CC(C)C)C(N)=O]

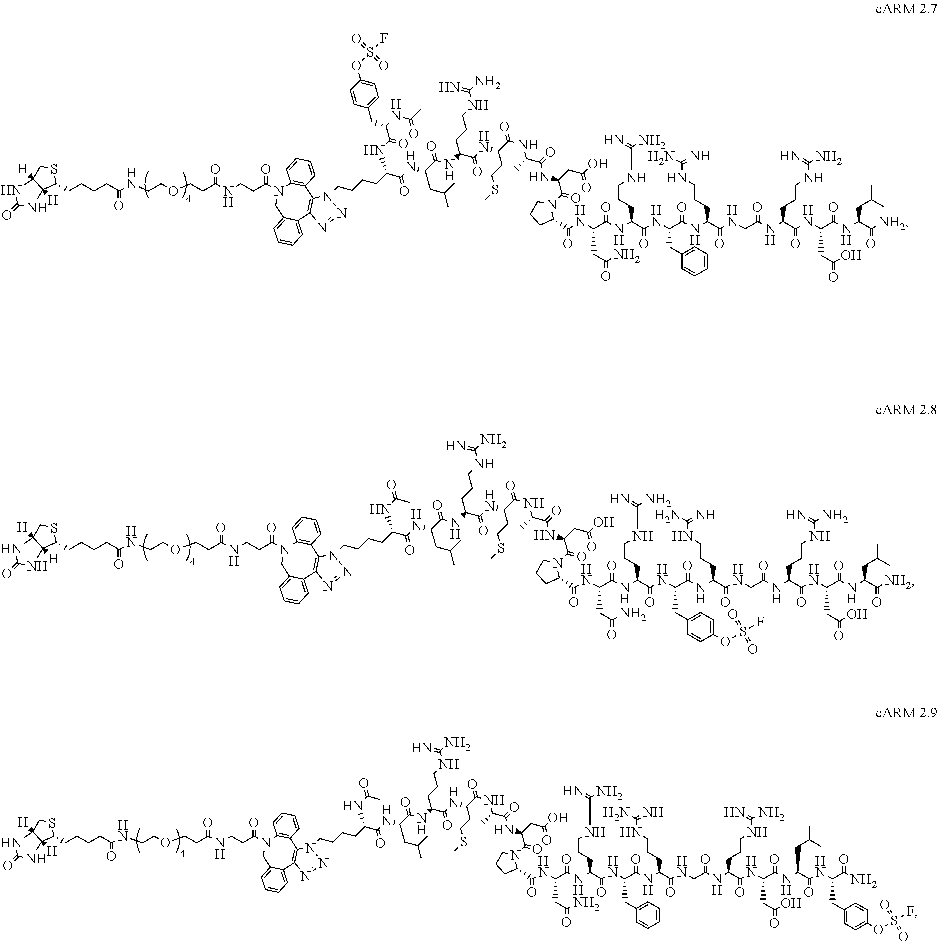

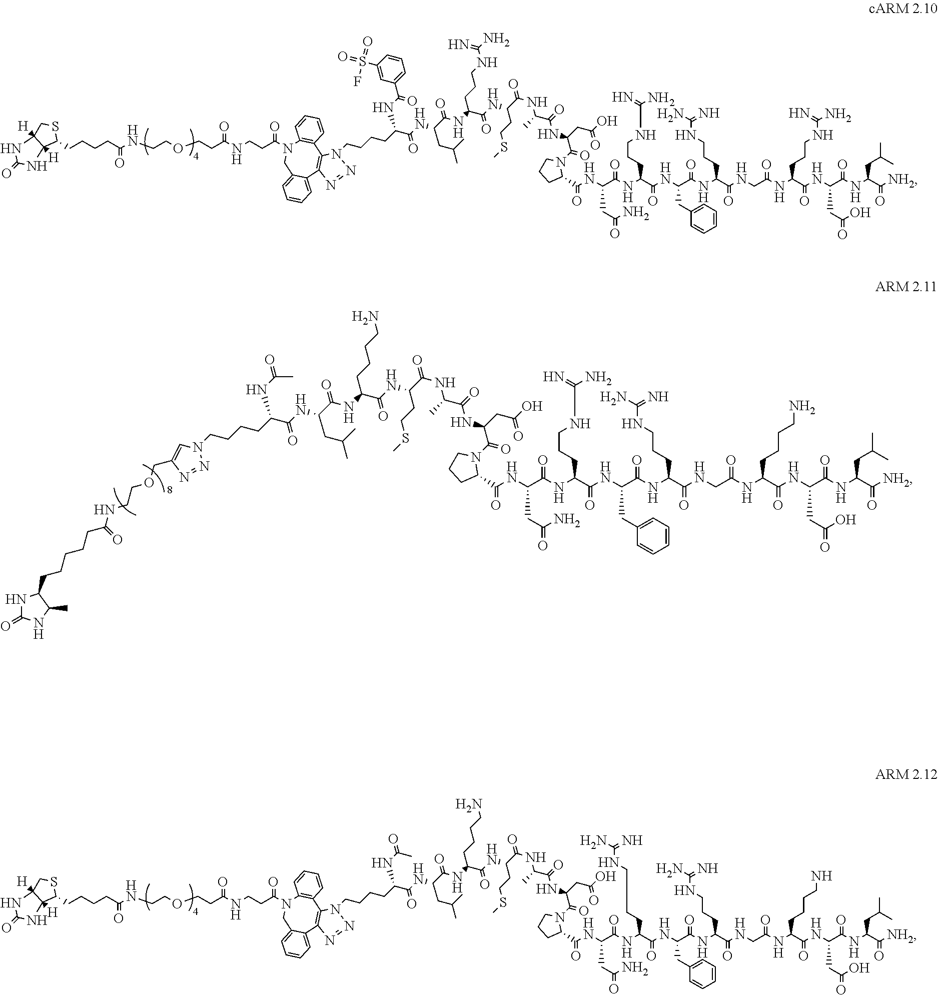

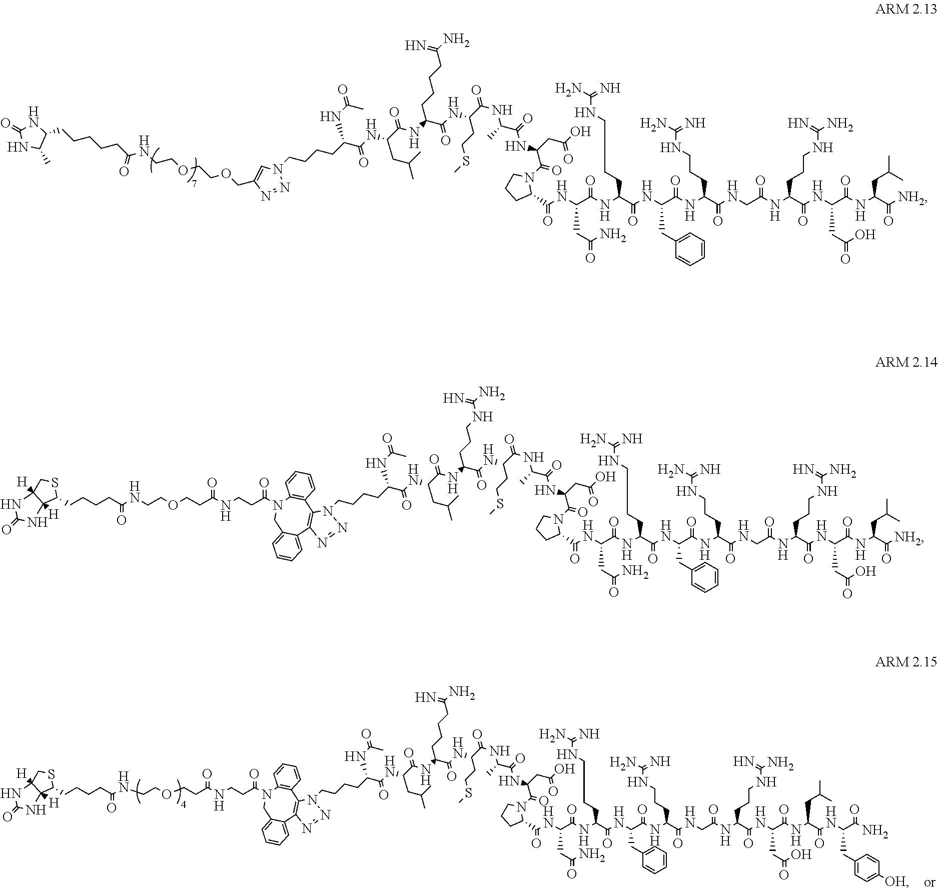

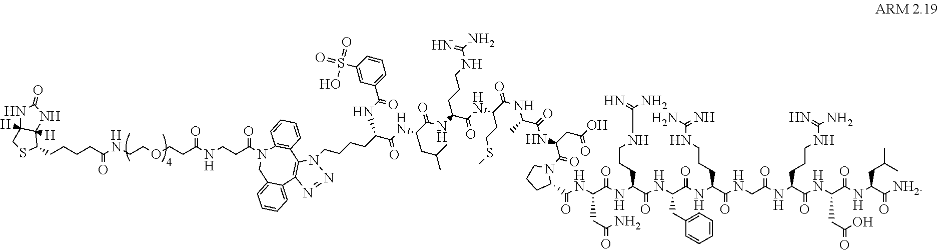

In certain embodiments of the compounds provided herein, the TBT comprises a fluorescent reporter.

In certain embodiments of the compounds provided herein, the TBT has the following structure:

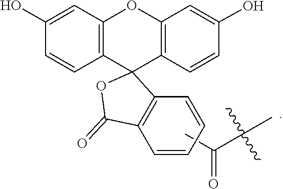

In certain embodiments of the compounds provided herein, the compound is:

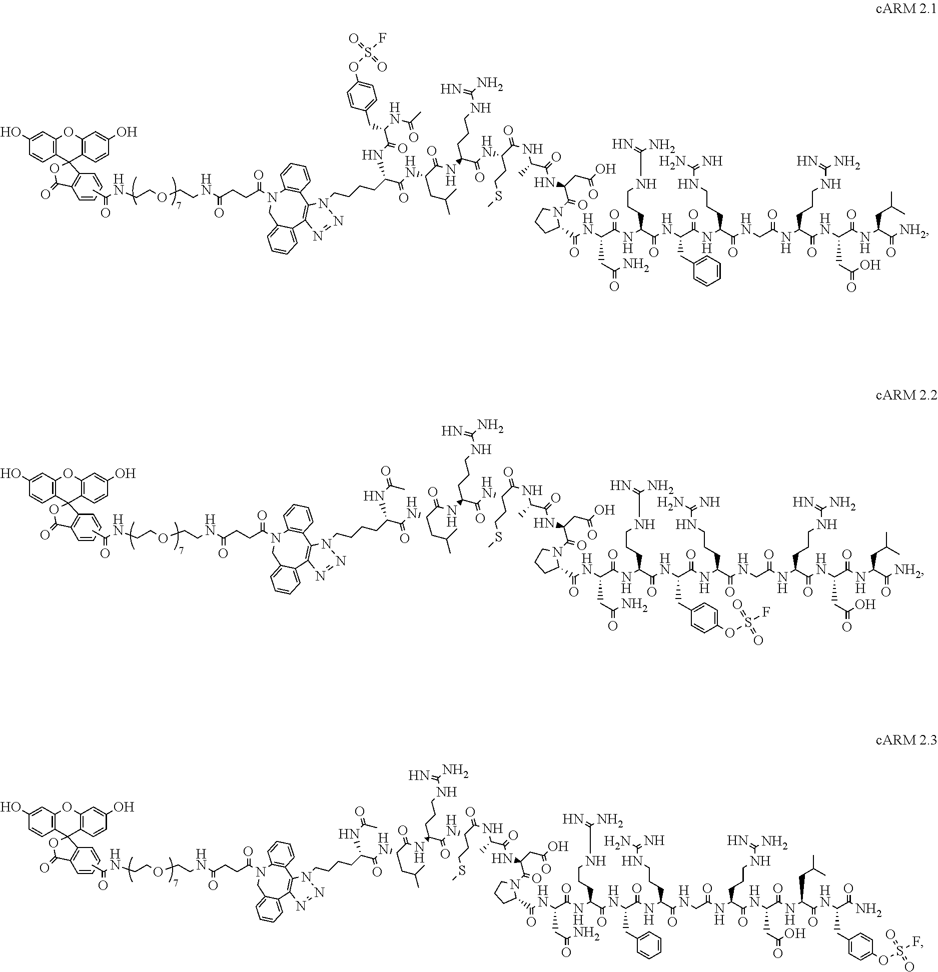

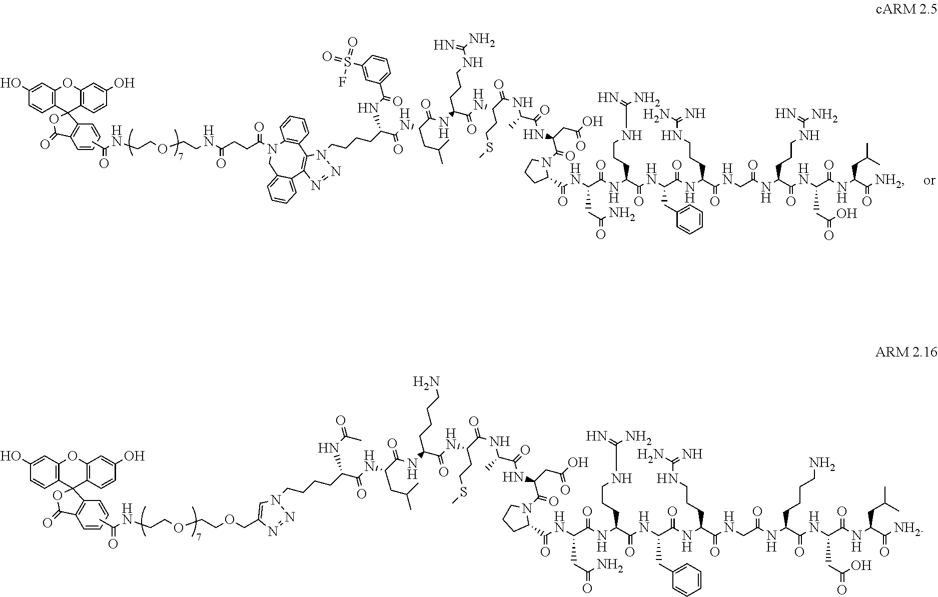

In an embodiment of the compounds provided herein, the compound is:

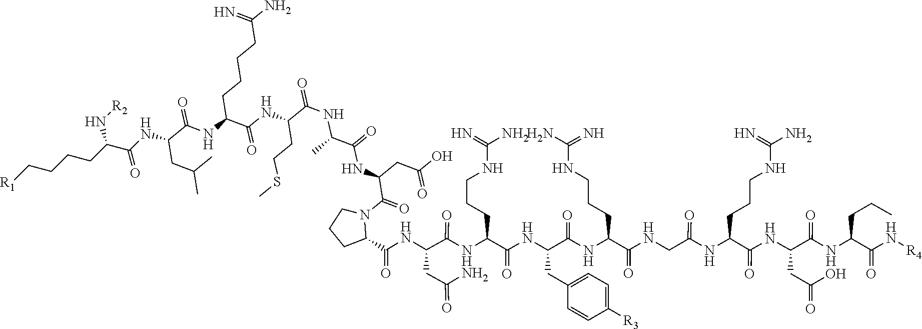

wherein R, is:

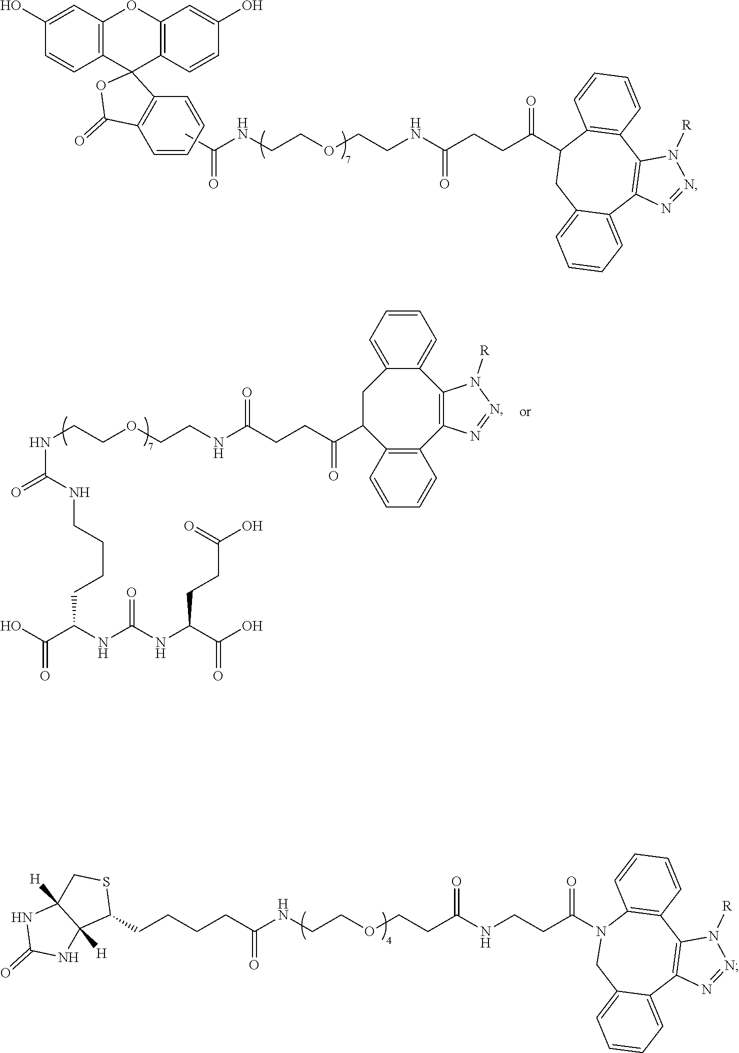

### R₂ is:

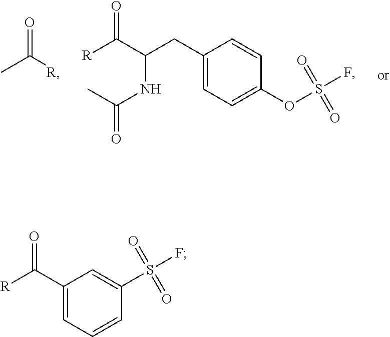

### R₃ is:

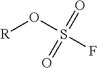

or hydrogen;

### R₄ is:

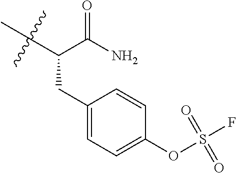 [SMILES: *[C@@H](Cc1ccc(OS(=O)(=O)F)cc1)C(N)=O]

or hydrogen, and R is the point of covalent attachment of R₁, R₂, R₃ and R₄ to the compound.

In a third aspect of the present disclosure, a composition is provided. The composition comprises the compound of the first aspect or the second aspect, or a pharmaceutically acceptable salt or solvate thereof, and at least one pharmaceutically acceptable carrier, diluent or excipient.

In a fourth aspect of the present disclosure, a kit is provided. The kit comprises the compound of the first aspect or the second aspect, or a pharmaceutically acceptable salt or solvate thereof, and an OV.

In an embodiment of the kit provided herein, the compound and the OV are formulated together.

In an embodiment of the kit provided herein, the compound and the OV are formulated separately.

In an embodiment of the kit provided herein, the OV comprises an oncolytic HSV.

In an embodiment of the kit provided herein, the OV is T-VEC.

In a fifth aspect of the present disclosure, a method of recruiting an HSV-specific antibody to a cancer cell in a subject is provided. The method comprises administering the compound of the first aspect or the second aspect, or a pharmaceutically acceptable salt or solvate thereof, or the composition of the third aspect of the invention to the subject.

In a sixth aspect of the present disclosure, a method of recruiting an HSV-specific antibody to a pathogen or a cell infected with a pathogen in a subject is provided. The method comprises administering the compound of the first aspect or the second aspect, or a pharmaceutically acceptable salt or solvate thereof, or the composition of the third aspect of the invention to the subject.

In certain embodiments of the methods of recruiting an HSV-specific antibody to a cancer cell, a pathogen or a cell infected with a pathogen in a subject provided herein, the HSV-specific antibody is a serum antibody.

In a seventh aspect of the present disclosure, a method of treating cancer in a subject is provided. The method comprises administering an effective amount of a compound comprising
a. at least one TBT comprising one or more moieties that bind to one or more target proteins on the cancer;
b. at least one ABT comprising one or more epitopes or epitope mimetics of a Herpes Simplex Virus (HSV) surface protein; and
c. optionally, at least one linker connecting the at least one TBT with the at least one ABT,
   or a pharmaceutically acceptable salt or solvate thereof, and an oncolytic virus (OV) therapy to the subject.

In an eighth aspect of the present disclosure, a method for enhancing the efficacy and/or reducing the toxicity of an oncolytic virus (OV) therapy in a subject with cancer is provided. The method comprises administering an effective amount of a compound comprising
a. at least one target binding terminus (TBT) comprising one or more moieties that bind to one or more target proteins on the cancer;
b. at least one antibody binding terminus (ABT) comprising one or more epitopes or epitope mimetics of a Herpes Simplex Virus (HSV) surface protein; and
c. optionally, at least one linker connecting the at least one TBT with the at least one ABT,
   or a pharmaceutically acceptable salt or solvate thereof to the subject.

In certain embodiments of the methods provided herein, the OV therapy comprises an oncolytic HSV.

In certain embodiments of the methods provided herein, the OV therapy is T-VEC.

In certain embodiments of the methods provided herein, the HSV surface protein is a glycoprotein.

In certain embodiments of the methods provided herein, the HSV surface protein is gD.

In certain embodiments of the methods provided herein, the epitope or epitope mimetic comprises the amino acid sequence set forth in any one of SEQ ID NOs: 1-9, or a variant thereof.

In certain embodiments of the methods provided herein, the epitope or epitope mimetic consists of the amino acid sequence set forth in any one of SEQ ID NOs: 1-9, or a variant thereof.

In certain embodiments of the methods provided herein, the compound further comprises one or more reactive groups that mediate covalent conjugation of the compound with an HSV-specific antibody and/or the target protein.

In certain embodiments of the methods provided herein, the HSV-specific antibody is a serum antibody.

In certain embodiments of the methods provided herein, the reactive group comprises an electrophilic functional group that reacts with an amino acid nucleophile in a nucleophilic substitution reaction.

In certain embodiments of the methods provided herein, the reactive group comprises an acyl imidazole group having the following structure:

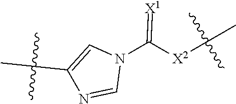

wherein:
X¹ is S, O or NR¹;
X² is O or NR²; and
R¹ and R² are independently H or C₁₋₄ alkyl.

In certain embodiments of the methods provided herein, the reactive group comprises a fluorosulfate-I-tyrosine (FSY) group or an aryl-sulfonyl fluoride (ASF) group.

In certain embodiments of the methods provided herein, the cancer is prostate cancer and the target protein is PSMA.

In certain embodiments of the methods provided herein, the TBT has the following structure:

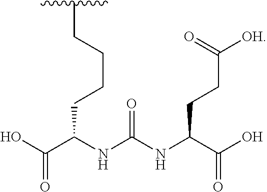 [SMILES: *CCCC[C@H](NC(=O)N[C@@H](CCC(=O)O)C(=O)O)C(=O)O]

In certain embodiments of the methods provided herein, the compound is

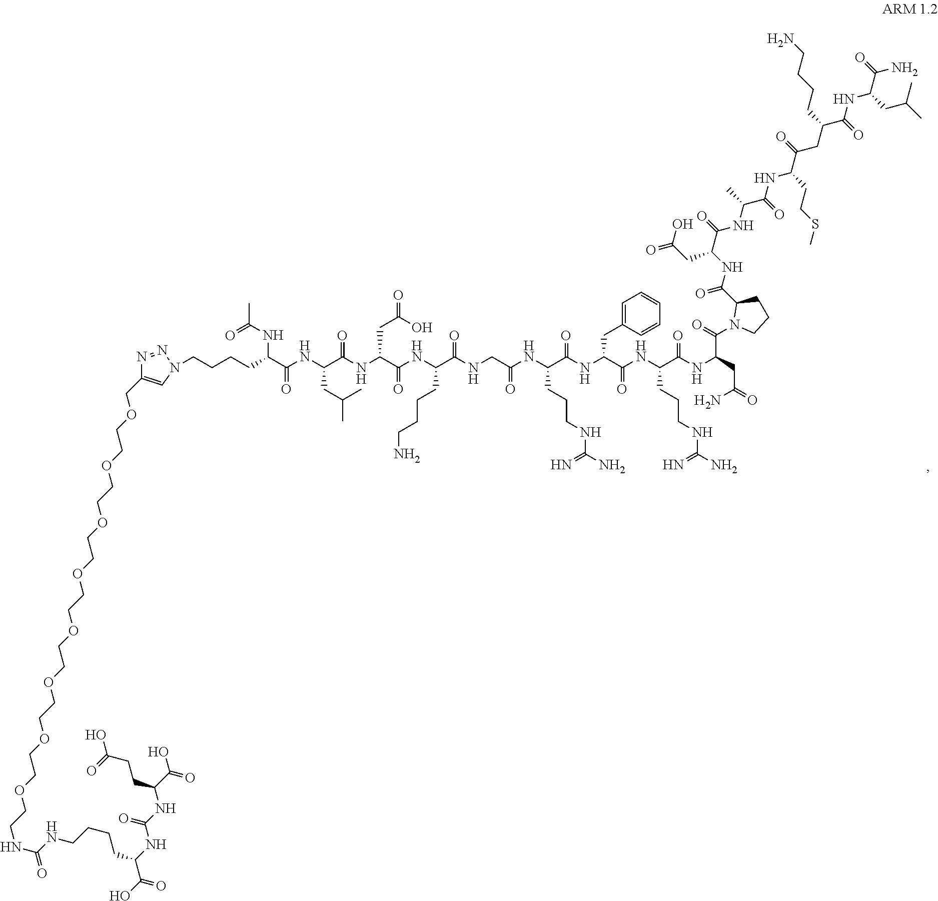 [SMILES: CSCC[C@H](NC(=O)[C@@H](C)NC(=O)[C@@H](CC(=O)O)NC(=O)[C@H]1CCCN1C(=O)[C@@H](CC(N)=O)NC(=O)[C@H](CCCNC(=N)N)NC(=O)[C@@H](Cc1ccccc1)NC(=O)[C@H](CCCNC(=N)N)NC(=O)CNC(=O)[C@H](CCCCN)NC(=O)[C@@H](CC(=O)O)NC(=O)[C@H](CC(C)C)NC(=O)[C@H](CCCCn1cc(COCCOCCOCCOCCOCCOCCOCCOCCNC(=O)NCCCC[C@H](NC(=O)N[C@@H](CCC(=O)O)C(=O)O)C(=O)O)nn1)NC(C)=O)C(=O)C[C@H](CCCCN)C(=O)N[C@@H](CC(C)C)C(N)=O]

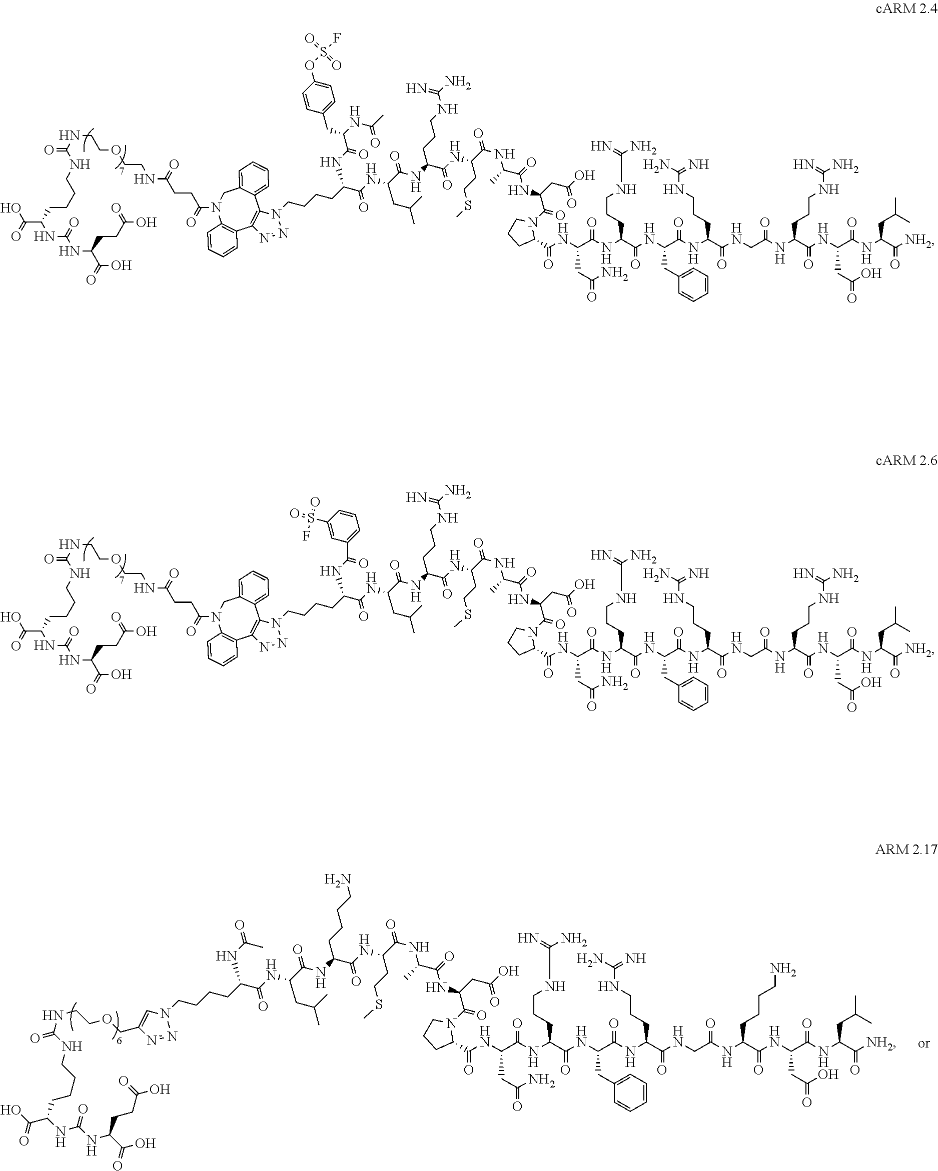

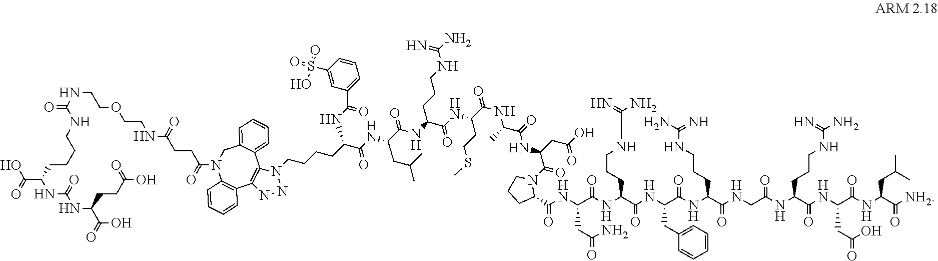 [SMILES: CSCC[C@H](NC(=O)[C@H](CCCNC(=N)N)NC(=O)[C@H](CC(C)C)NC(=O)[C@H](CCCCn1nnc2c1-c1ccccc1CN(C(=O)CCC(=O)NCCOCCNC(=O)NCCCC[C@H](NC(=O)N[C@@H](CCC(=O)O)C(=O)O)C(=O)O)c1ccccc1-2)NC(=O)c1cccc(S(=O)(=O)O)c1)C(=O)N[C@@H](C)C(=O)N[C@@H](CC(=O)O)C(=O)N1CCC[C@H]1C(=O)N[C@@H](CC(N)=O)C(=O)N[C@@H](CCCNC(=N)N)C(=O)N[C@@H](Cc1ccccc1)C(=O)N[C@@H](CCCNC(=N)N)C(=O)NCC(=O)N[C@@H](CCCNC(=N)N)C(=O)N[C@@H](CC(=O)O)C(=O)N[C@@H](CC(C)C)C(N)=O]

In certain embodiments of the methods provided herein, the cancer is glioblastoma and the target protein is uPAR.

In certain embodiments of the methods provided herein, the cancer is glioblastoma, the target protein is uPAR and the TBT has the following structure:

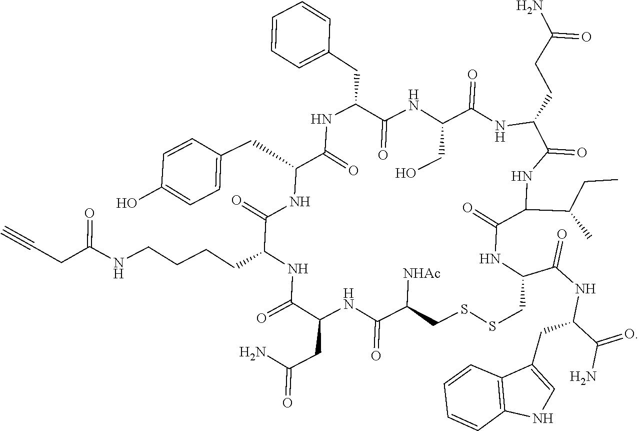 [SMILES: C#CCC(=O)NCCCC[C@H]1NC(=O)[C@H](CC(N)=O)NC(=O)[C@@H](NC(C)=O)CSSC[C@@H](C(=O)N[C@@H](Cc2c[nH]c3ccccc23)C(N)=O)NC(=O)C([C@@H](C)CC)NC(=O)[C@@H](CCC(N)=O)NC(=O)[C@H](CO)NC(=O)[C@@H](Cc2ccccc2)NC(=O)[C@@H](Cc2ccc(O)cc2)NC1=O]

In certain embodiments of the methods provided herein, the cancer is breast or ovarian cancer, and the target protein is HER2.

In certain embodiments of the methods provided herein, the cancer is breast or ovarian cancer, the target protein is HER2 and the TBT has the following structure:

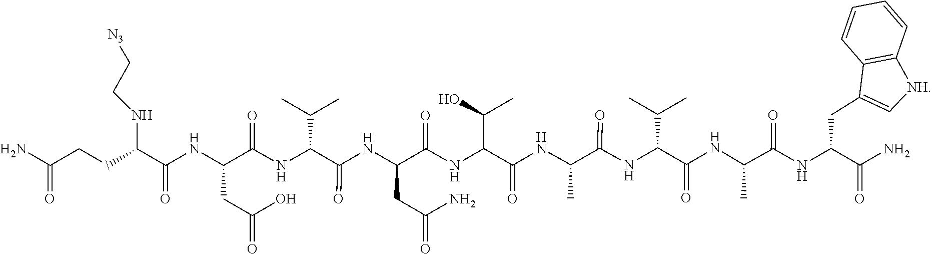

In certain embodiments of the methods provided herein, the cancer is ovarian cancer and the target protein is folate receptor.

In certain embodiments of the methods provided herein, the cancer is ovarian cancer, the target protein is folate receptor and the TBT comprises methotrexate or folate.

In certain embodiments of the methods provided herein, the compound is administered to the subject before, concurrently with, and/or after administration of the OV therapy.

In an embodiment of the methods provided herein, the compound is:

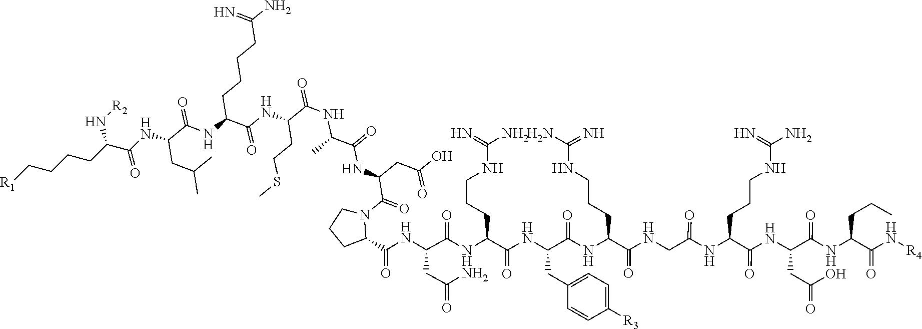

wherein R₁ is:

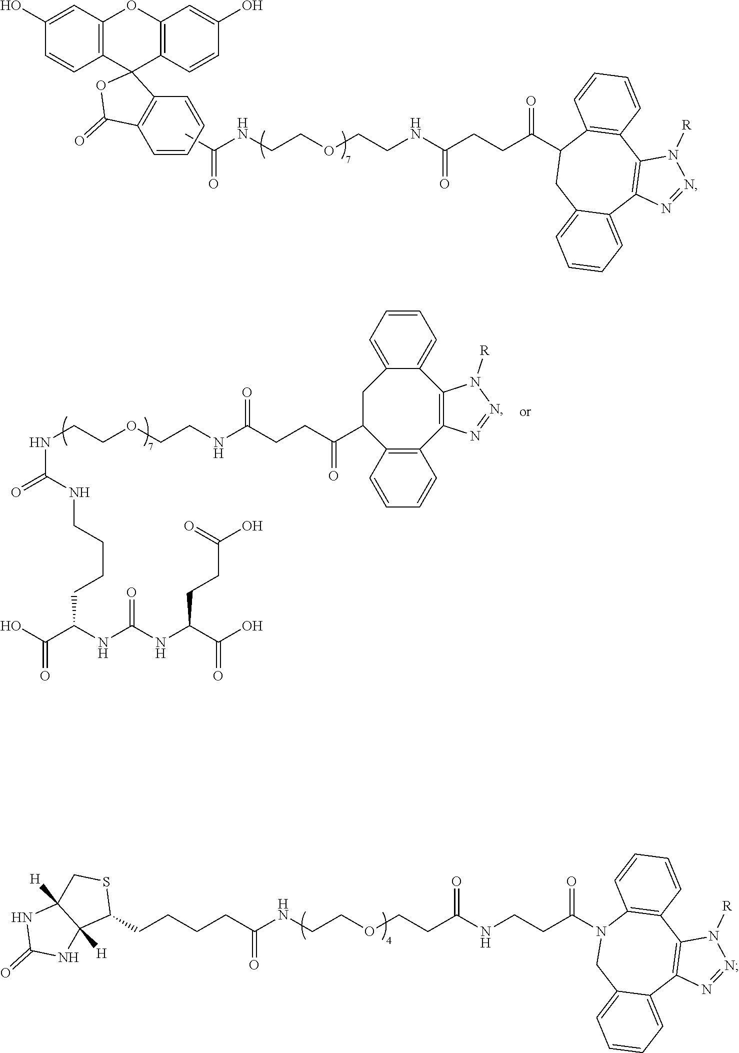

### R₂ is:

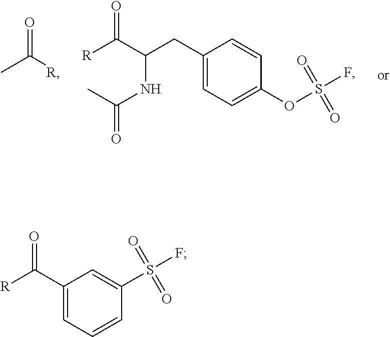

### R₃ is:

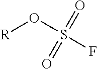

or hydrogen;

### R₄ is:

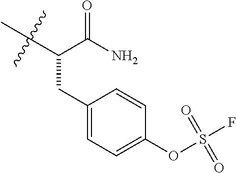

or hydrogen, and R is the point of covalent attachment of R₁, R₂, R₃ and R₄ to the compound.

### BRIEF DESCRIPTION OF THE DRAWINGS

In order that the subject matter of the present disclosure may be readily understood, embodiments are illustrated by way of the accompanying drawings.

FIG. 1 is a schematic illustration of a ternary complex formed between a serum antibody, a bifunctional ARM and a target tumor antigen; and a quaternary complex formed between a serum antibody, a bifunctional ARM, a target tumor antigen and an immune cell.

FIG. 2A is a total ion current (TIC) trace (top) and a UV chromatogram (bottom) from a liquid chromatography-mass spectrometry (LCMS) analysis of post-column purified targeting fragment 1A, which consists of desthiobiotin conjugated to a click chemistry-compatible polyethylene glycol (PEG8) linker having the following formula:

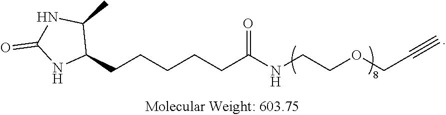

FIG. 2B is a mass spectrum of targeting fragment 1A.

FIG. 2C is a proton nuclear magnetic resonance (¹H NMR) spectrum of targeting fragment 1A.

FIG. 3A is a TIC trace (top) and a UV chromatogram (bottom) from a LCMS analysis of post-column purified antibody-binding fragment 1B having the following formula:

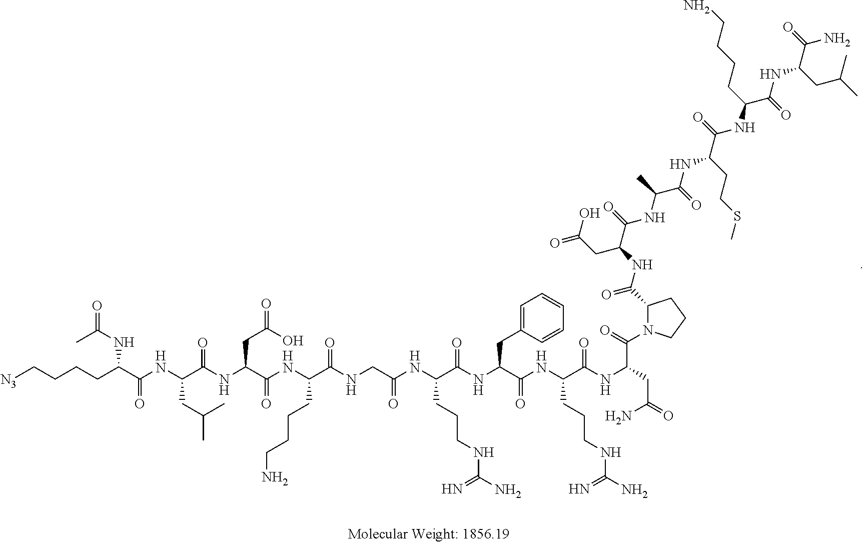 [SMILES: CSCC[C@H](NC(=O)[C@H](C)NC(=O)[C@H](CC(=O)O)NC(=O)[C@@H]1CCCN1C(=O)[C@H](CC(N)=O)NC(=O)[C@H](CCCNC(=N)N)NC(=O)[C@H](Cc1ccccc1)NC(=O)[C@H](CCCNC(=N)N)NC(=O)CNC(=O)[C@H](CCCCN)NC(=O)[C@H](CC(=O)O)NC(=O)[C@H](CC(C)C)NC(=O)[C@H](CCCCN=[N+]=[N-])NC(C)=O)C(=O)N[C@@H](CCCCN)C(=O)N[C@@H](CC(C)C)C(N)=O]

FIG. 3B is a mass spectrum of antibody-binding fragment 1B.

FIG. 4A is a TIC trace (top) and a UV chromatogram (bottom) from a LCMS analysis of post-column purified ARM 1.1, which comprises targeting fragment 1A and antibody-binding fragment 1B connected by a PEG8 linker having the following formula:

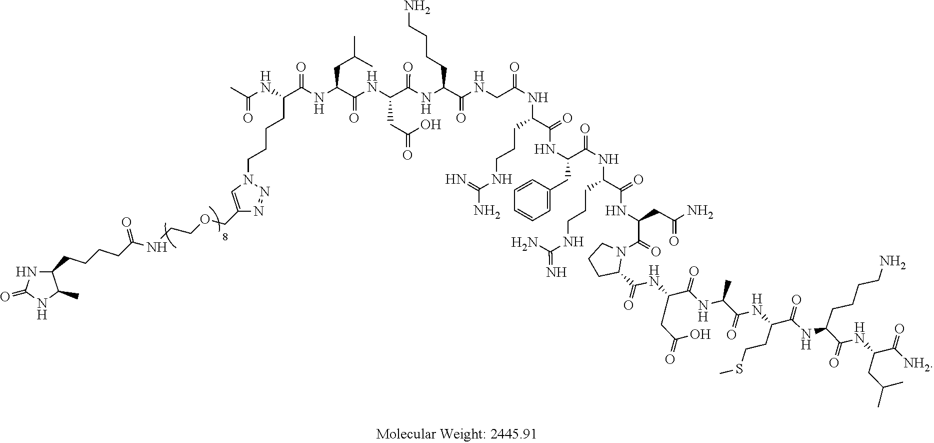

FIG. 4B is a mass spectrum of ARM 1.1.

FIG. 5A is a TIC trace (top) and a UV chromatogram (bottom) from a LCMS analysis of post-column purified, click chemistry-compatible linker fragment 1C having the following formula:

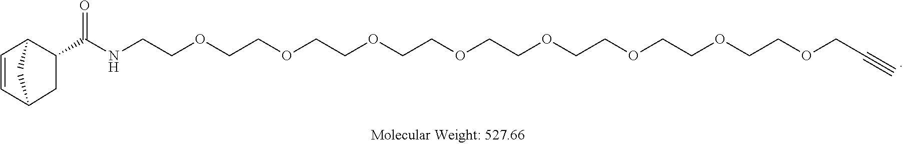 [SMILES: C#CCOCCOCCOCCOCCOCCOCCOCCOCCNC(=O)[C@@H]1C[C@H]2C=C[C@@H]1C2]

FIG. 5B is a mass spectrum of linker fragment 1C.

FIG. 5C is a ¹H NMR spectrum of linker fragment 1C.

FIG. 6A is a TIC trace (top) and a UV chromatogram (bottom) from a LCMS analysis of post-column purified, fluorescent reporter-conjugated, click chemistry-compatible linker fragment 1D having the following formula:

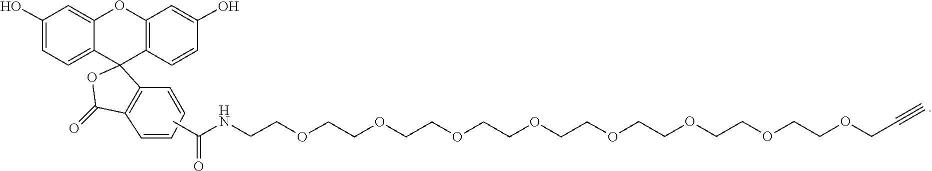

FIG. 6B is a mass spectrum of linker fragment 1D.

FIG. 6C is a ¹H NMR spectrum of linker fragment 1D.

FIG. 7A is a TIC trace (top) and a UV chromatogram (bottom) from a LCMS analysis of post-column purified targeting fragment 2A, which consists of a PSMA-binding glutamate urea ligand having the following formula:

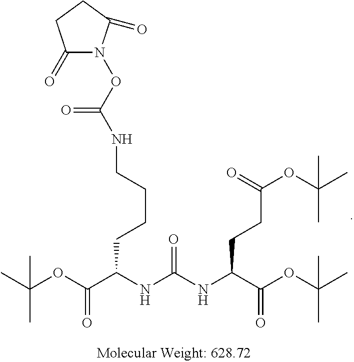 [SMILES: CC(C)(C)OC(=O)CC[C@H](NC(=O)N[C@@H](CCCCNC(=O)ON1C(=O)CCC1=O)C(=O)OC(C)(C)C)C(=O)OC(C)(C)C]

FIG. 7B is a mass spectrum of targeting fragment 2A.

FIG. 7C is a ¹H NMR spectrum of targeting fragment 2A.

FIG. 8A is a TIC trace (top) and a UV chromatogram (bottom) from a LCMS analysis of post-column purified targeting fragment 2A conjugated to a click chemistry-compatible linker fragment 2B having the following formula:

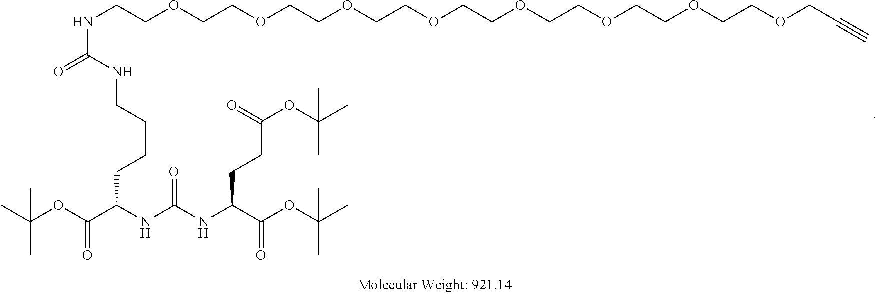 [SMILES: C#CCOCCOCCOCCOCCOCCOCCOCCOCCNC(=O)NCCCC[C@H](NC(=O)N[C@@H](CCC(=O)OC(C)(C)C)C(=O)OC(C)(C)C)C(=O)OC(C)(C)C]

FIG. 8B is a mass spectrum of targeting fragment 2A conjugated to linker fragment 2B.

FIG. 8C is a ¹H NMR spectrum of targeting fragment 2A conjugated to linker fragment 2B.

FIG. 9A is a TIC trace (top) and a UV chromatogram (bottom) from a LCMS analysis of post-column purified ARM 1.2, which comprises targeting fragment 2A and antibody-binding fragment 1B connected by a PEG8 linker having the following formula:

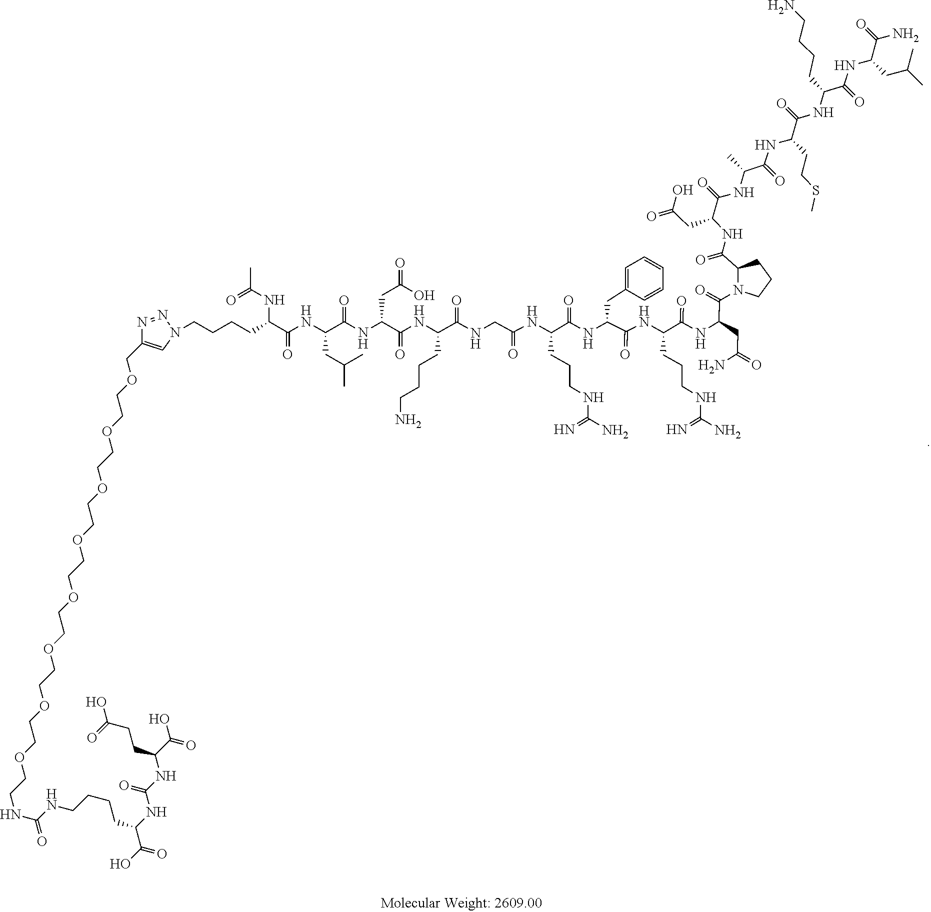 [SMILES: CSCC[C@H](NC(=O)[C@@H](C)NC(=O)[C@@H](CC(=O)O)NC(=O)[C@H]1CCCN1C(=O)[C@@H](CC(N)=O)NC(=O)[C@H](CCCNC(=N)N)NC(=O)[C@@H](Cc1ccccc1)NC(=O)[C@H](CCCNC(=N)N)NC(=O)CNC(=O)[C@H](CCCCN)NC(=O)[C@@H](CC(=O)O)NC(=O)[C@H](CC(C)C)NC(=O)[C@H](CCCCn1cc(COCCOCCOCCOCCOCCOCCOCCOCCNC(=O)NCCCC[C@H](NC(=O)N[C@@H](CCC(=O)O)C(=O)O)C(=O)O)nn1)NC(C)=O)C(=O)N[C@H](CCCCN)C(=O)N[C@@H](CC(C)C)C(N)=O]

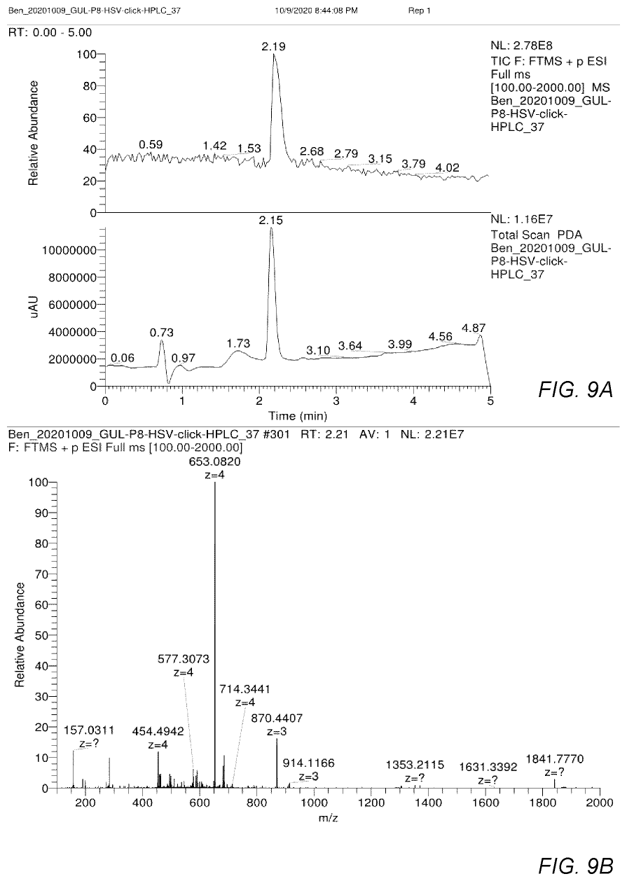

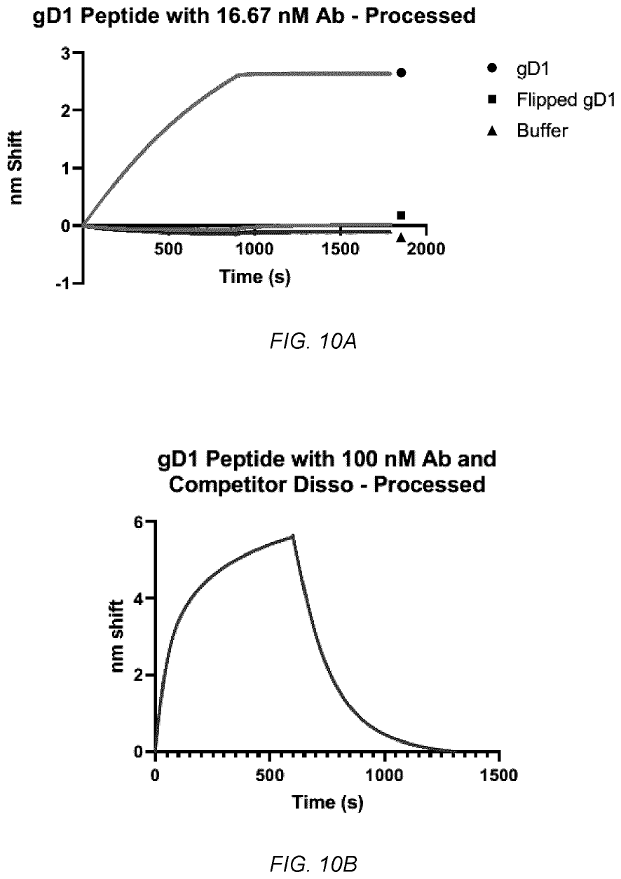

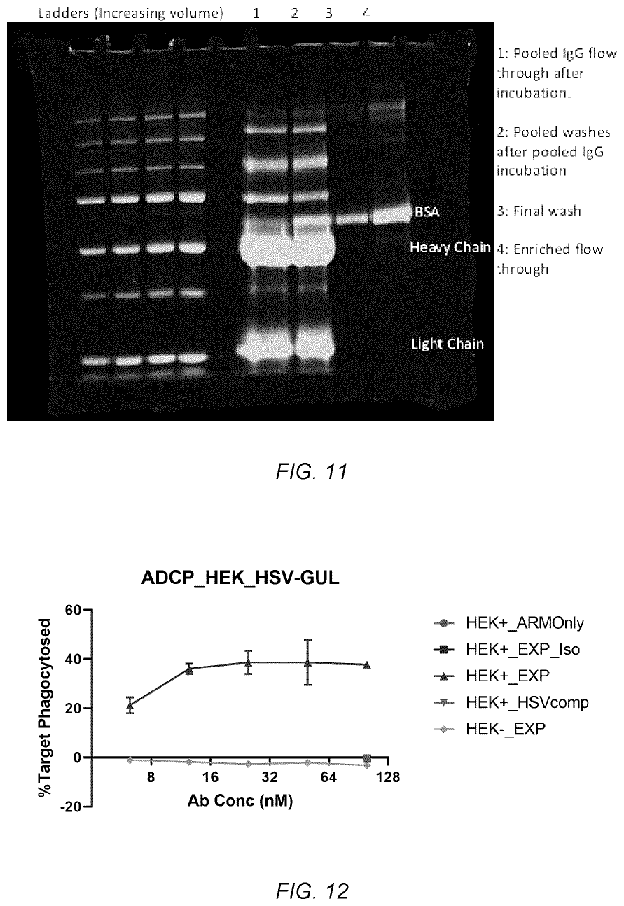

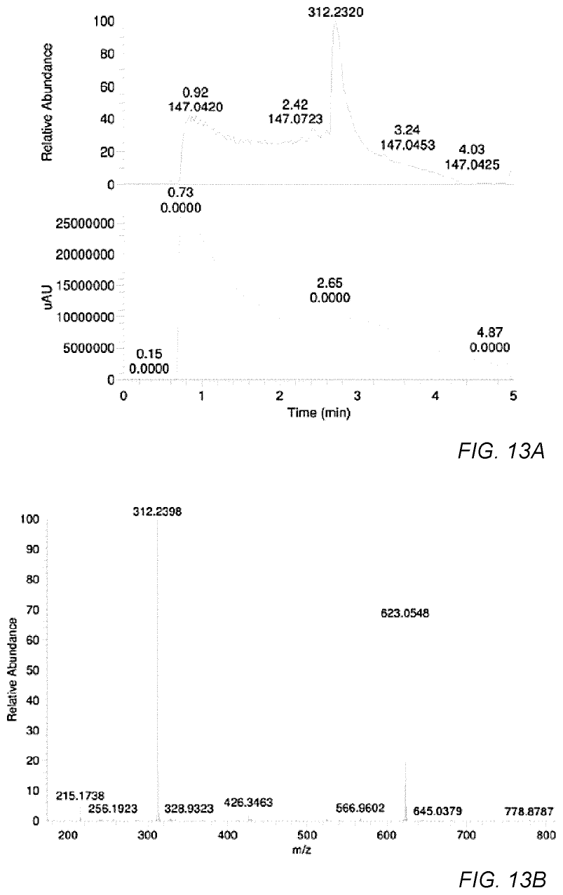

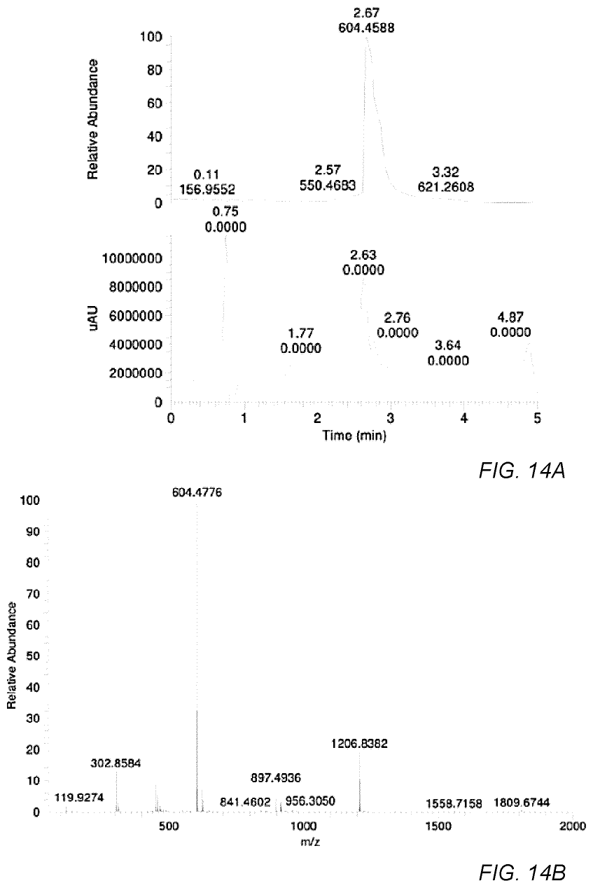

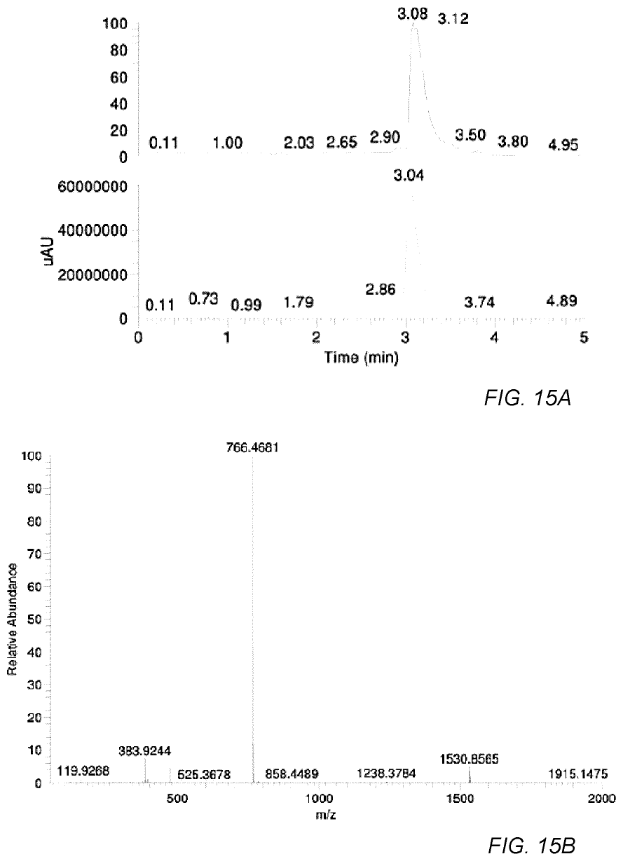

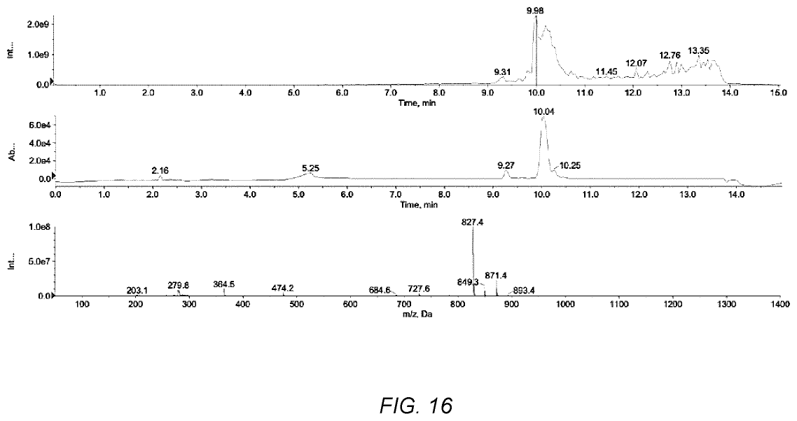

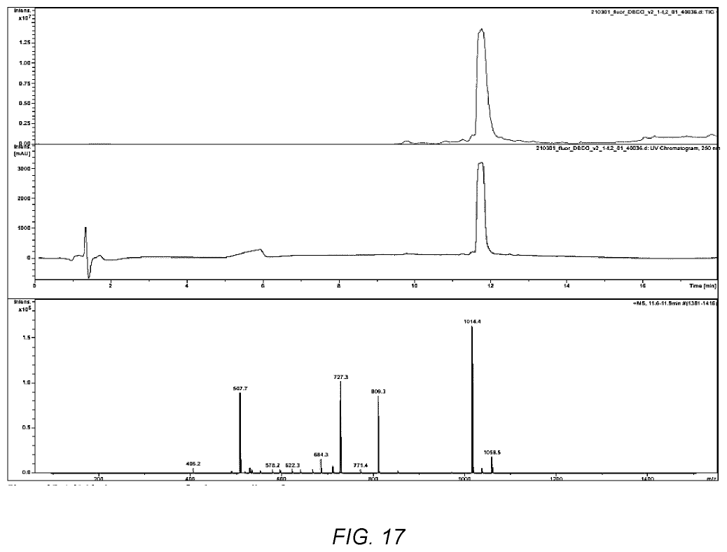

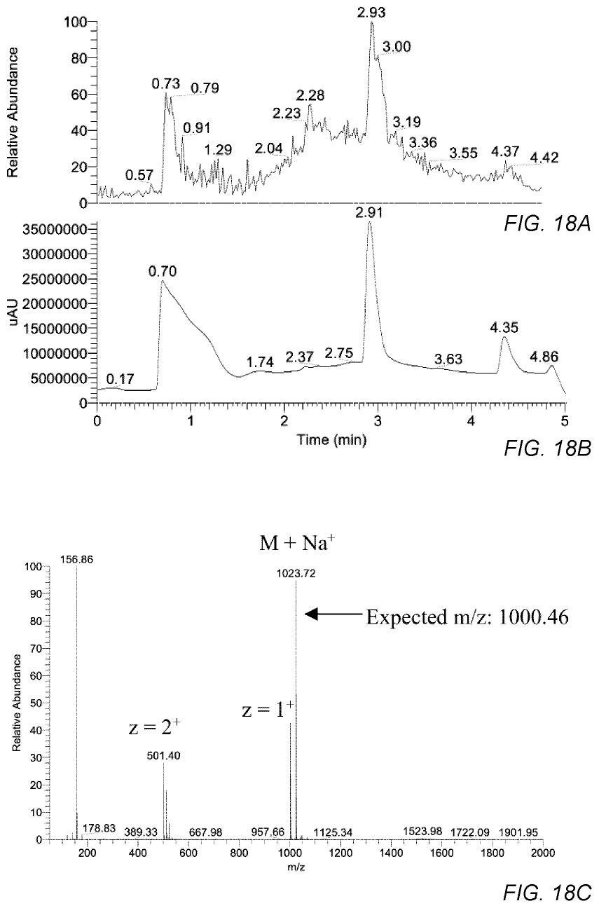

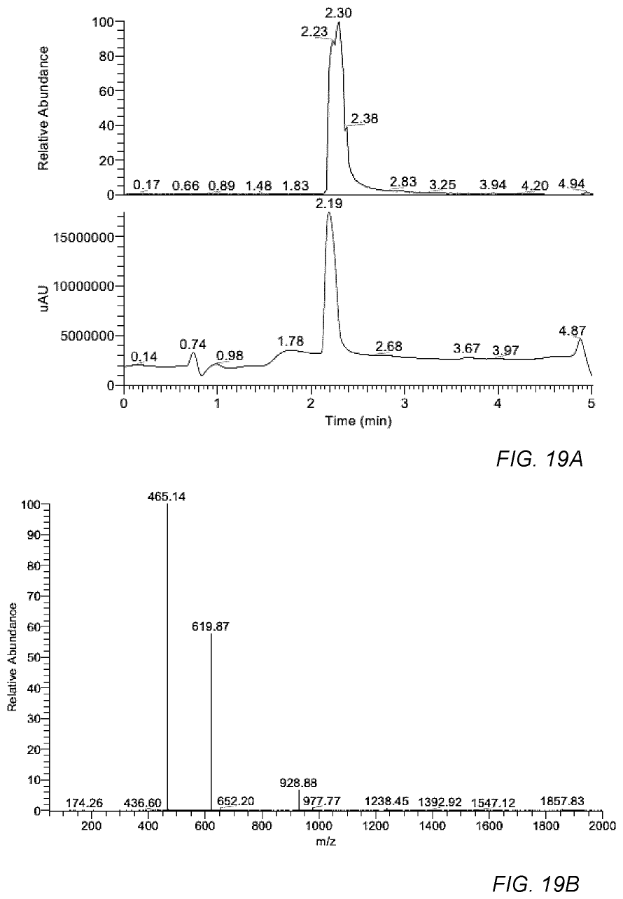

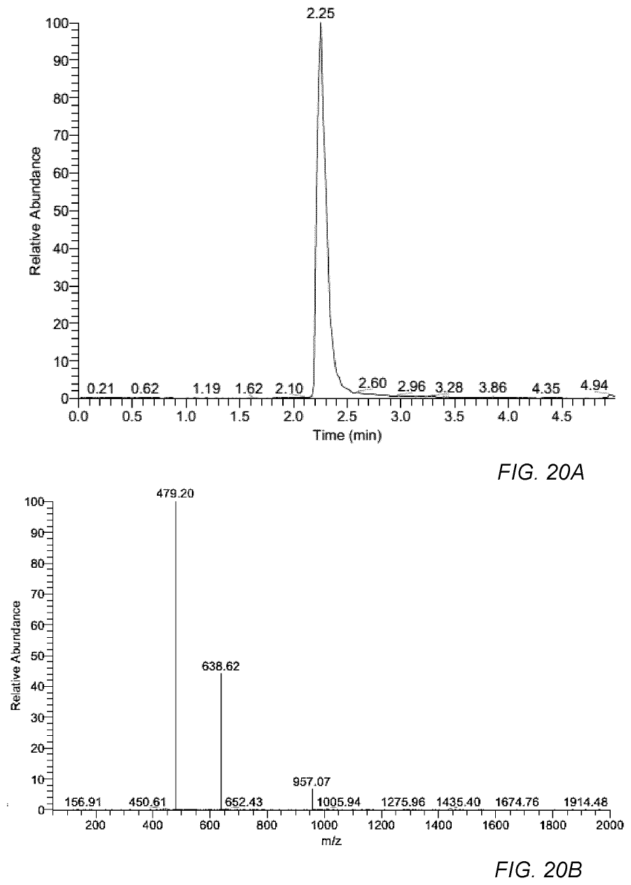

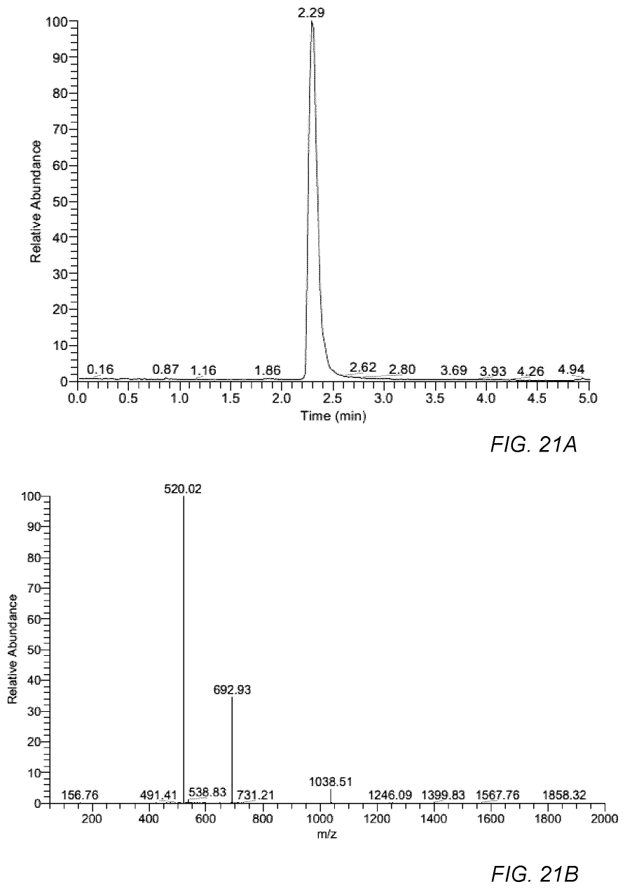

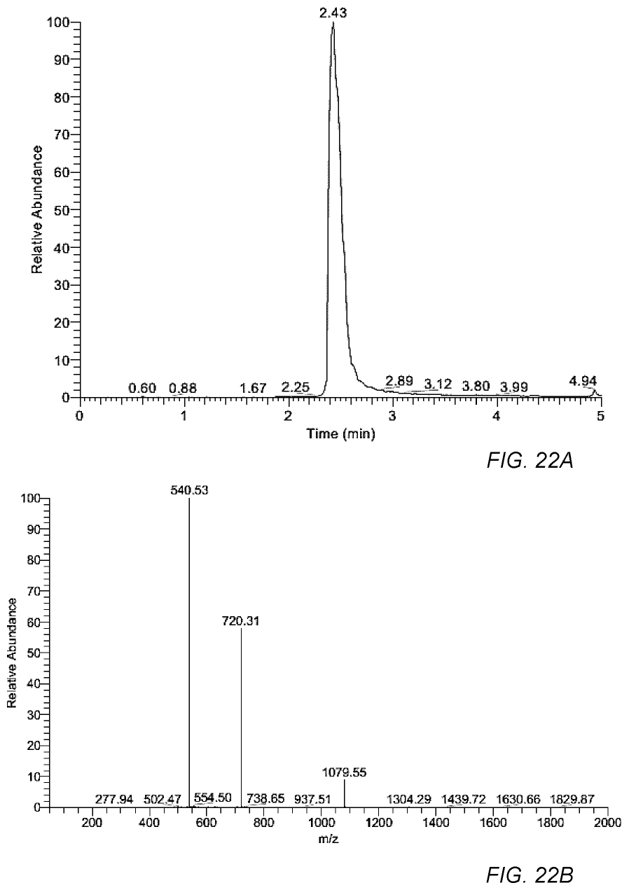

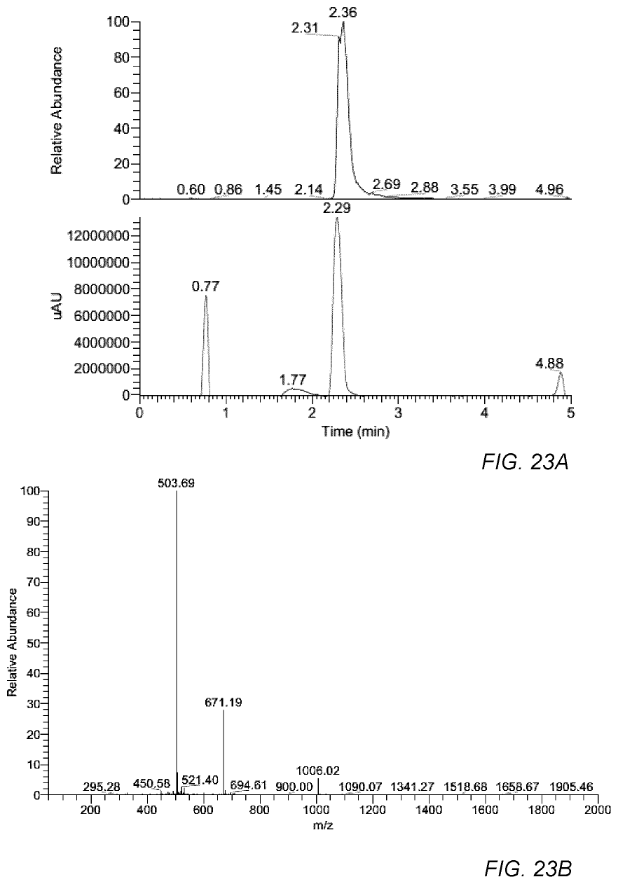

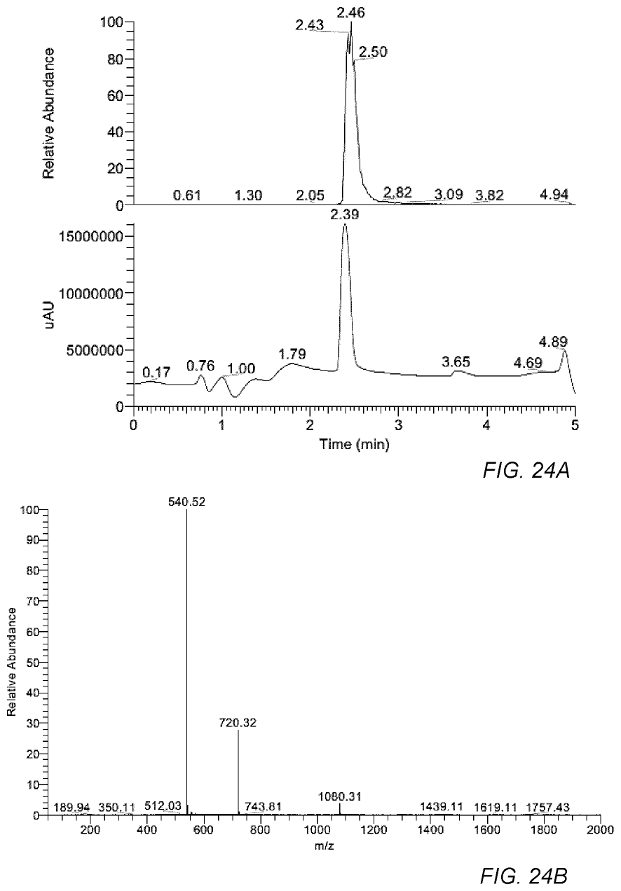

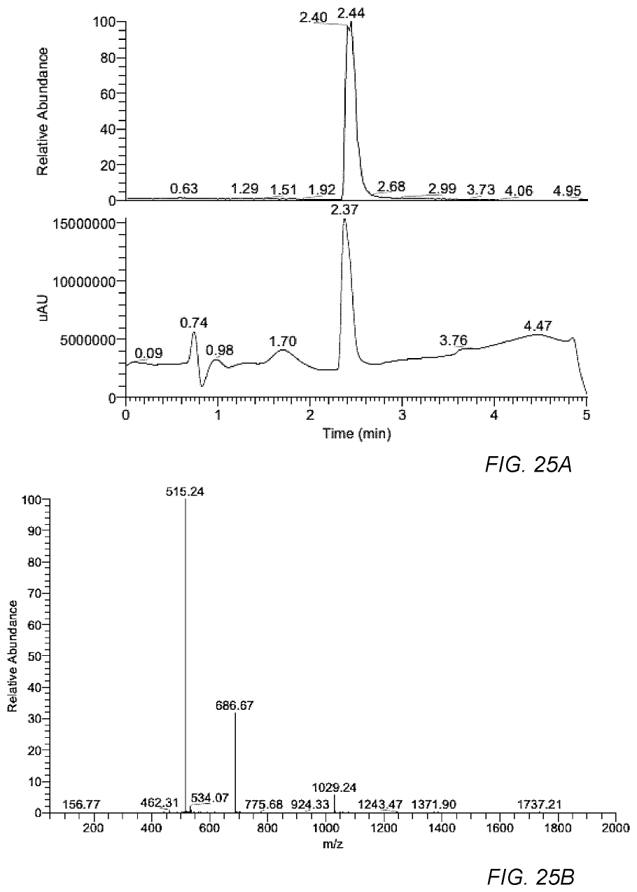

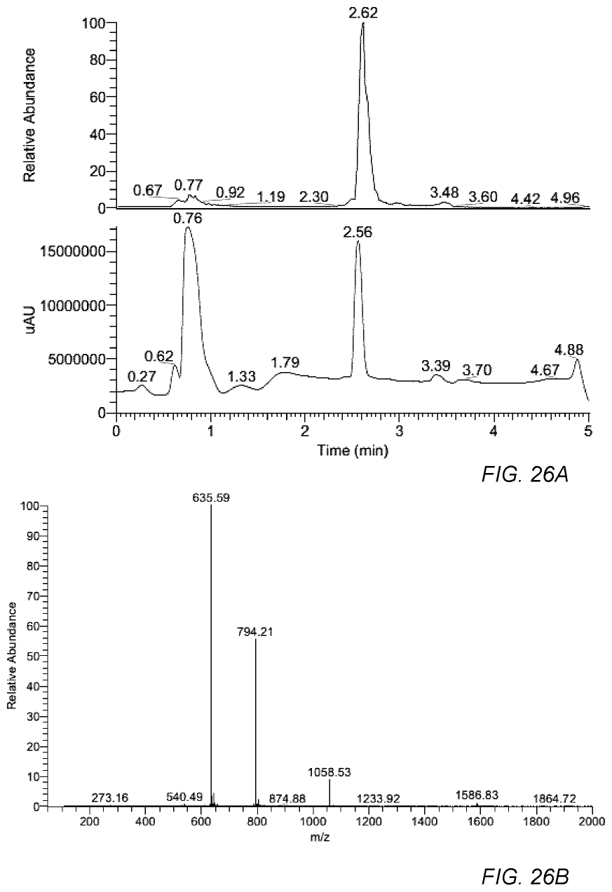

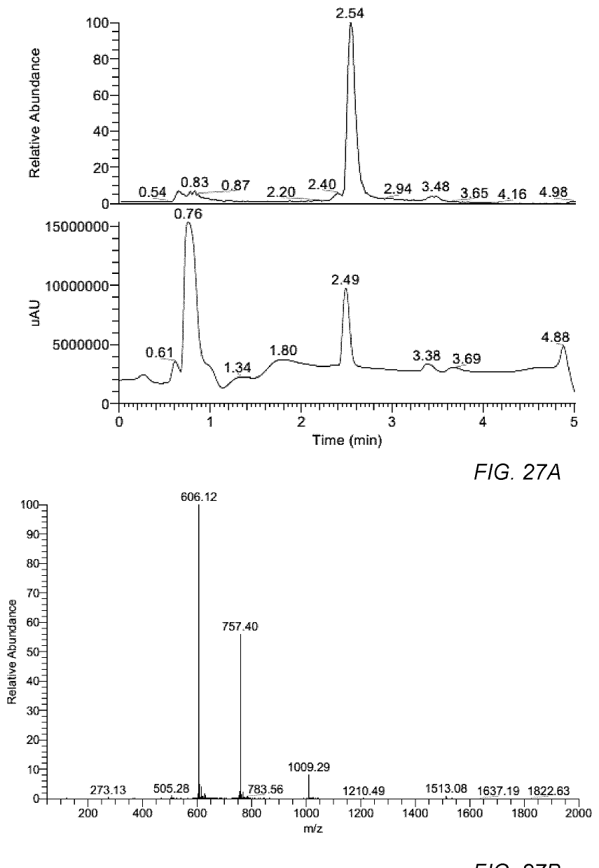

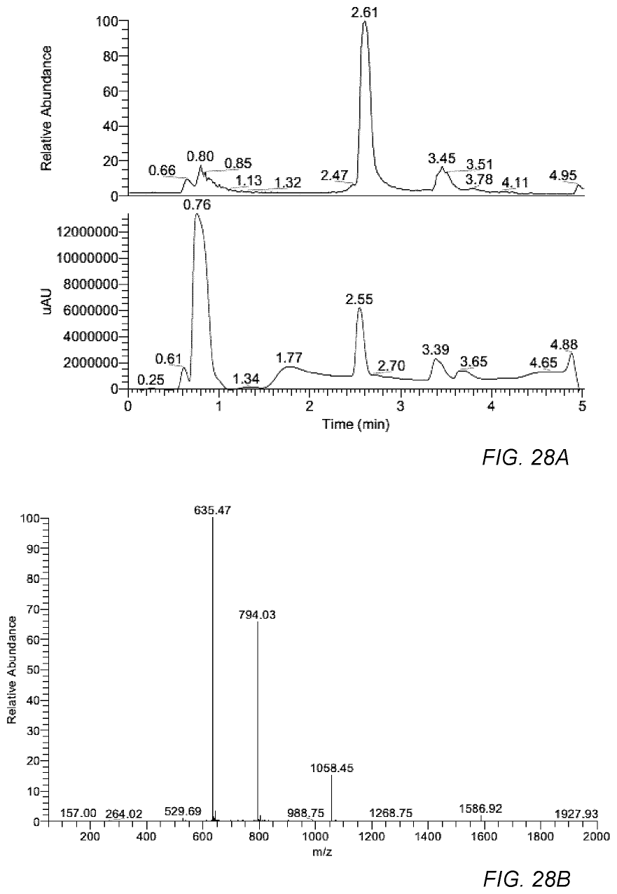

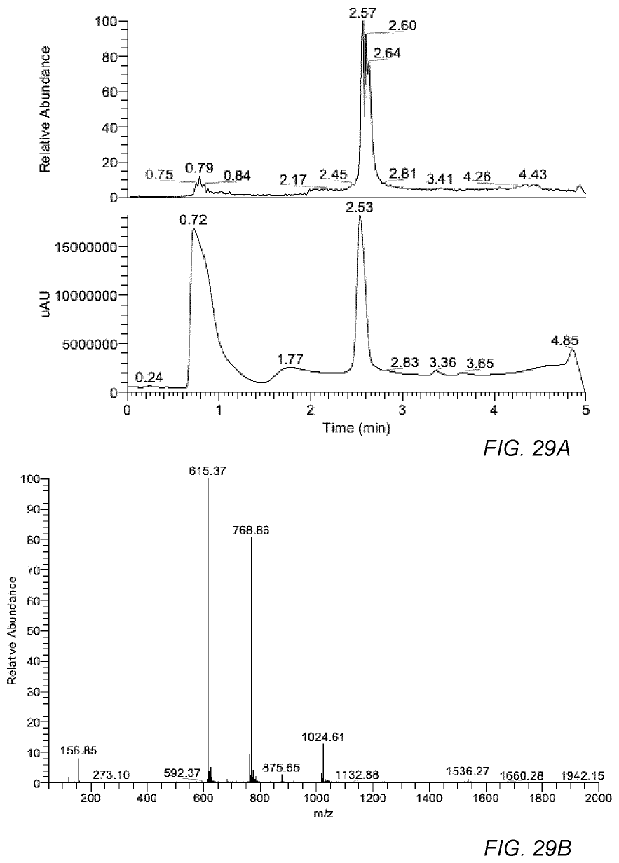

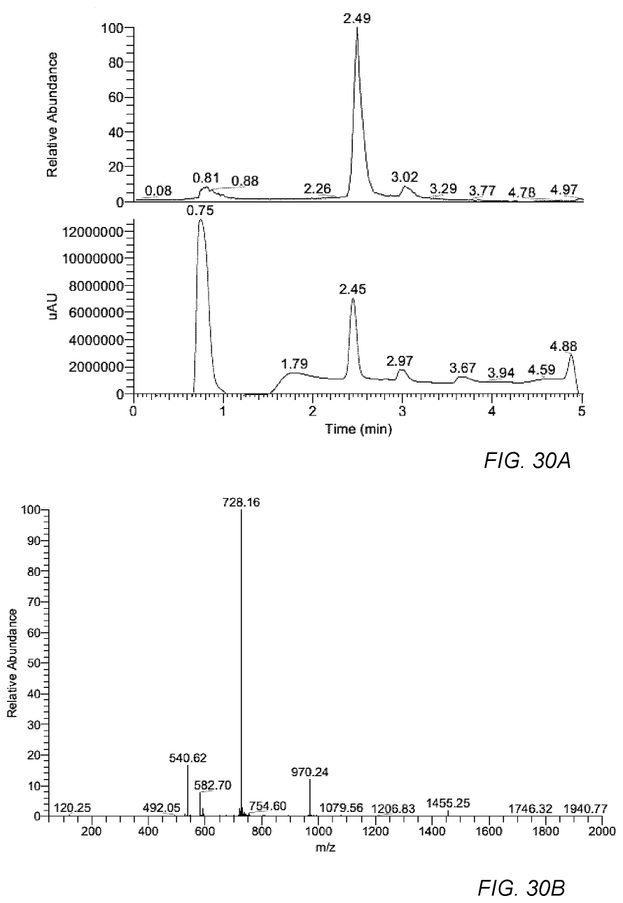

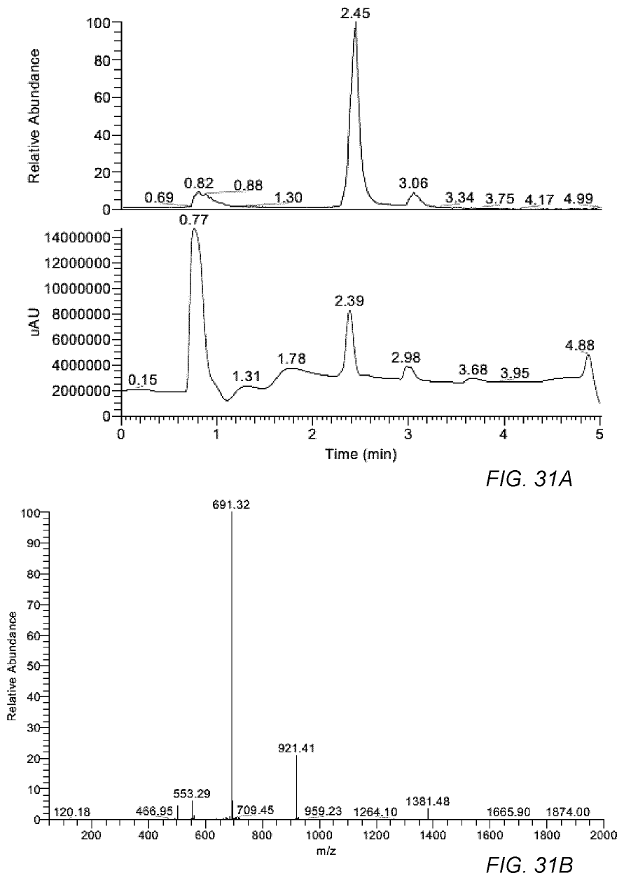

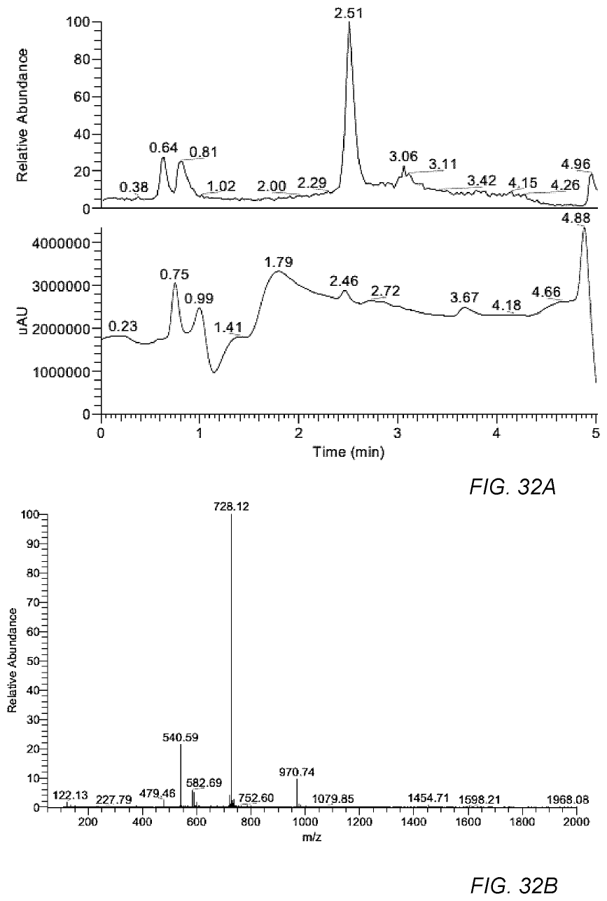

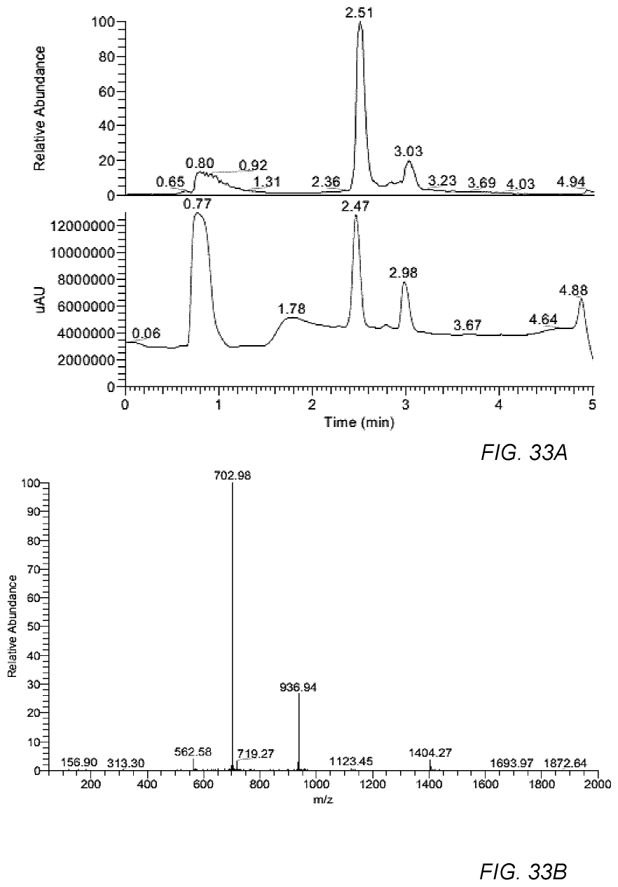

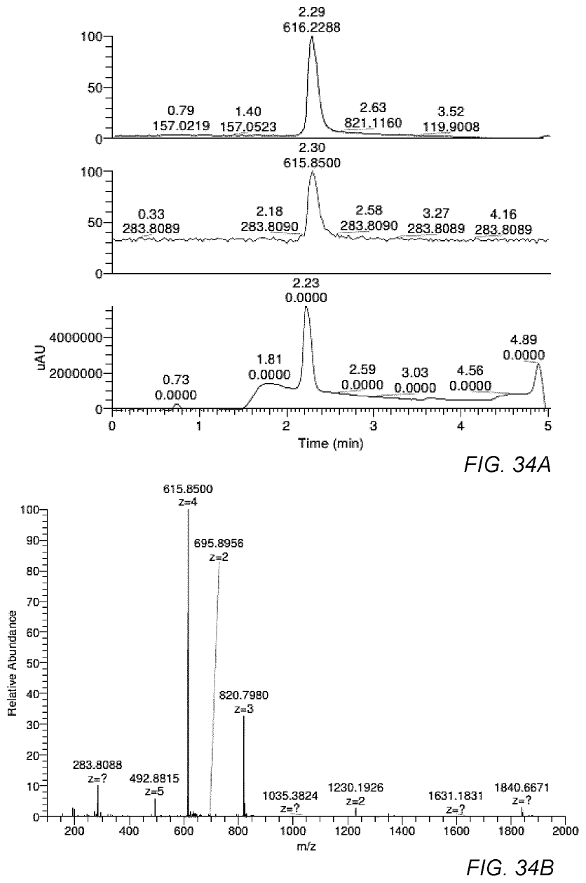

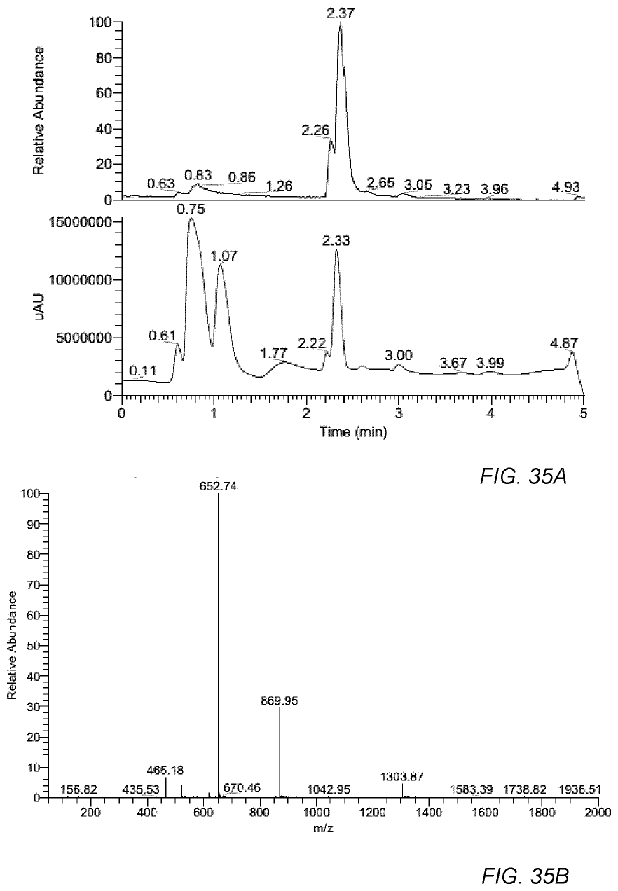

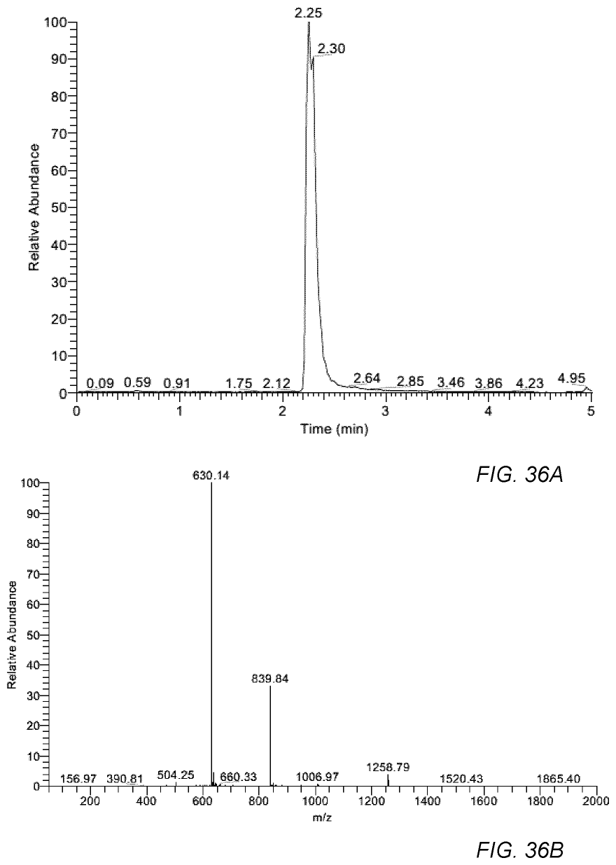

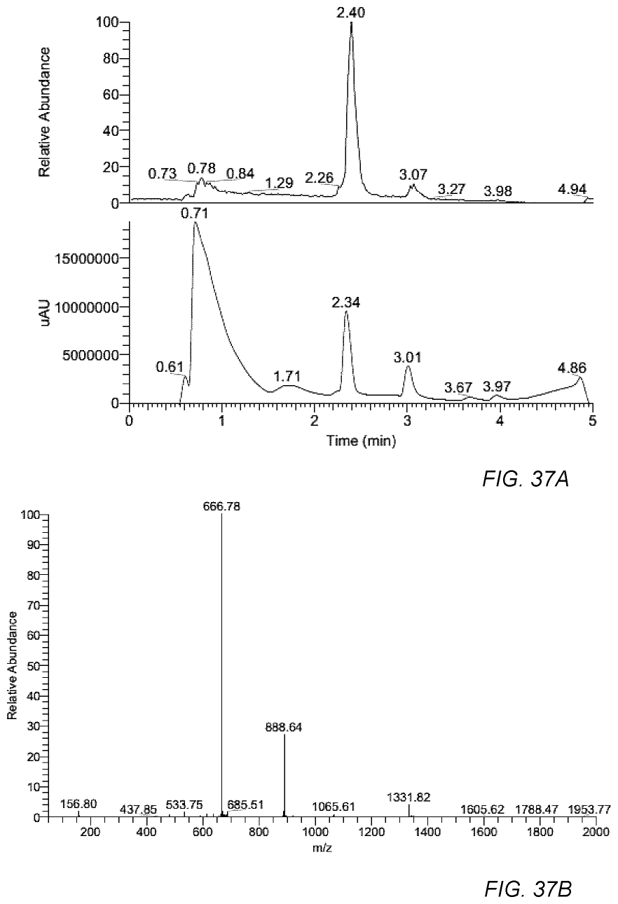

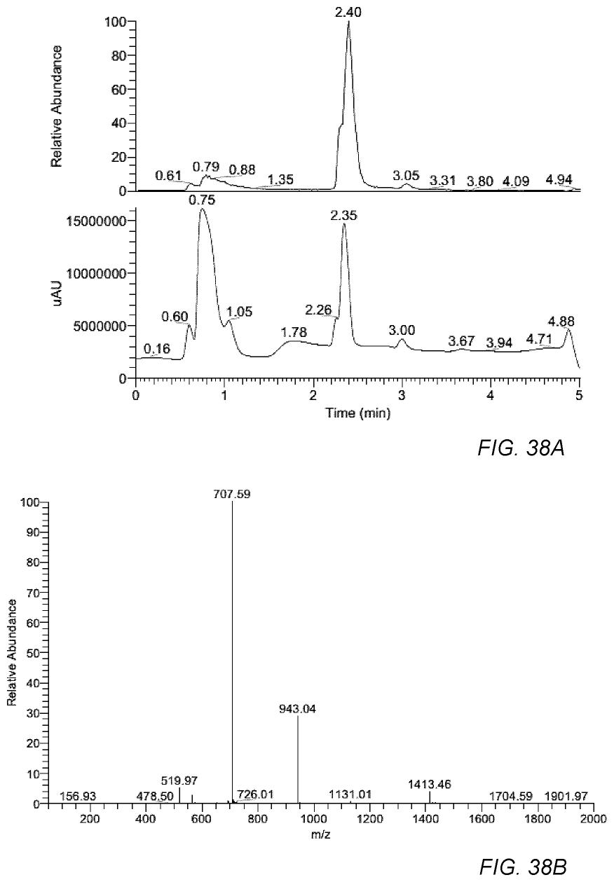

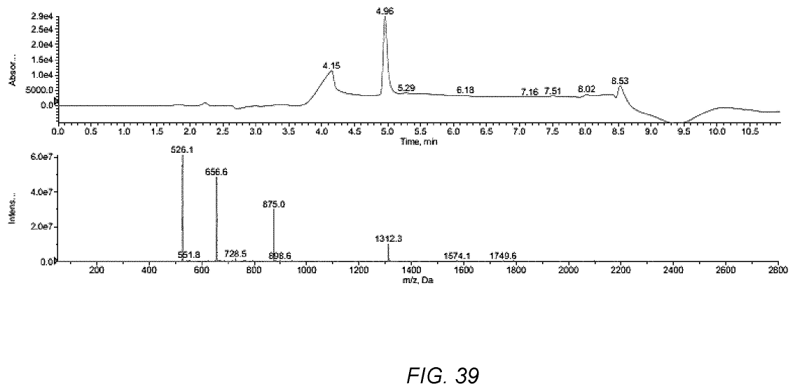

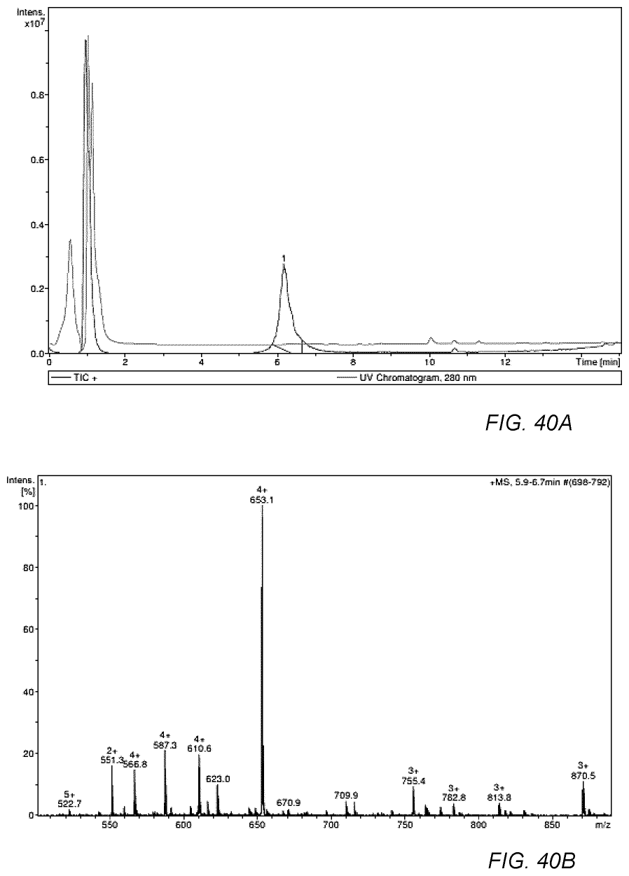

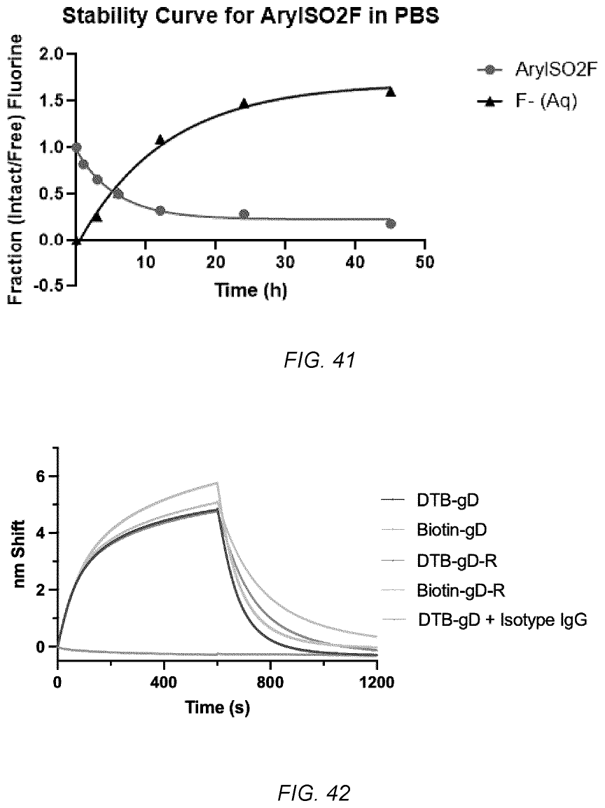

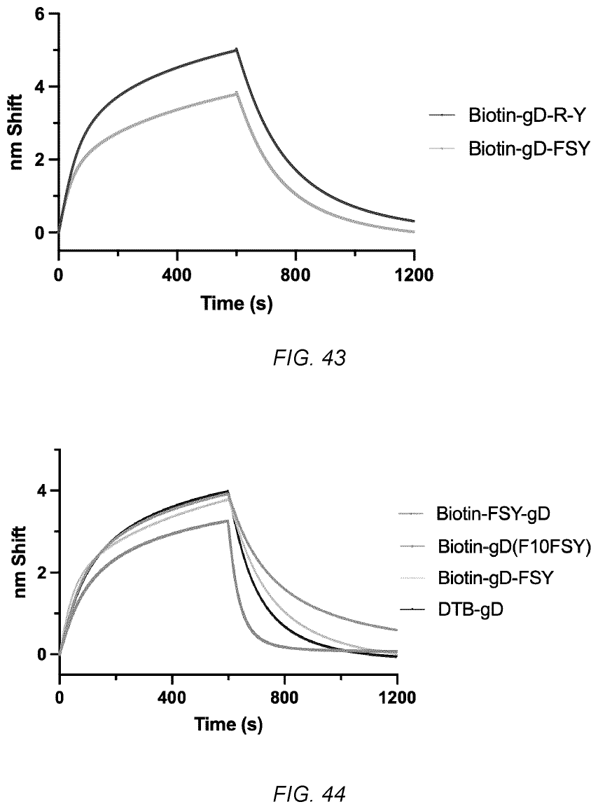

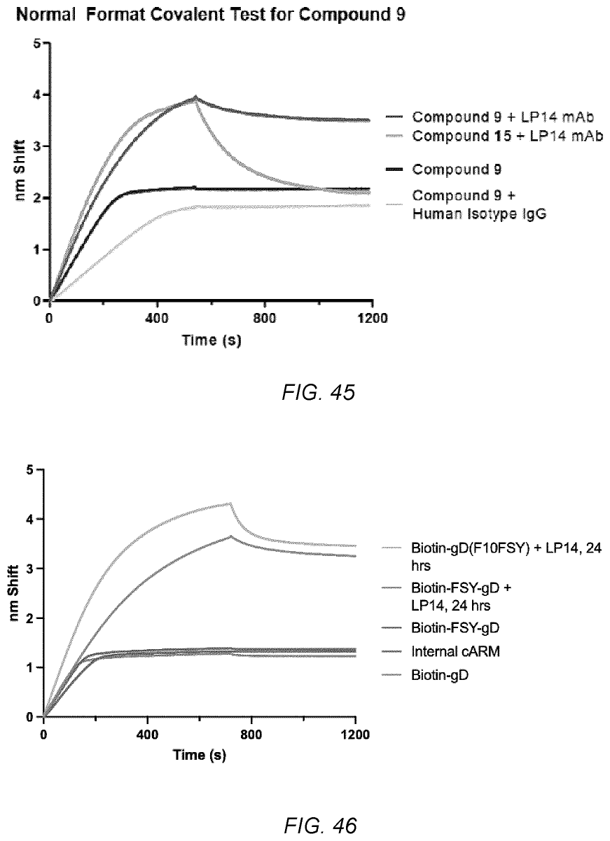

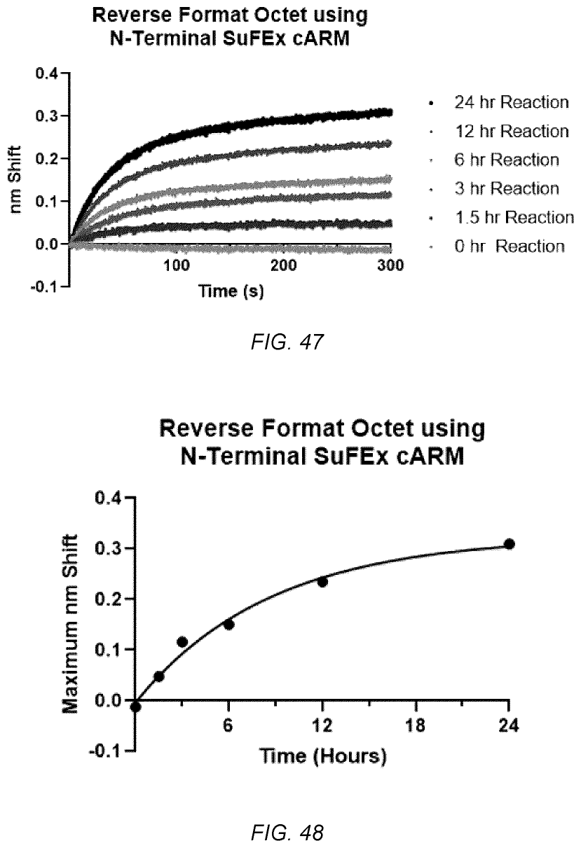

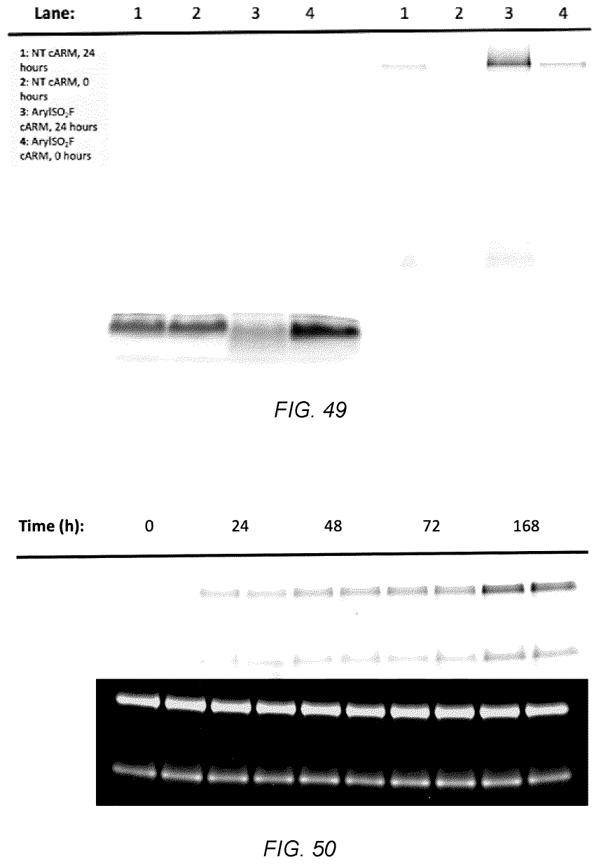

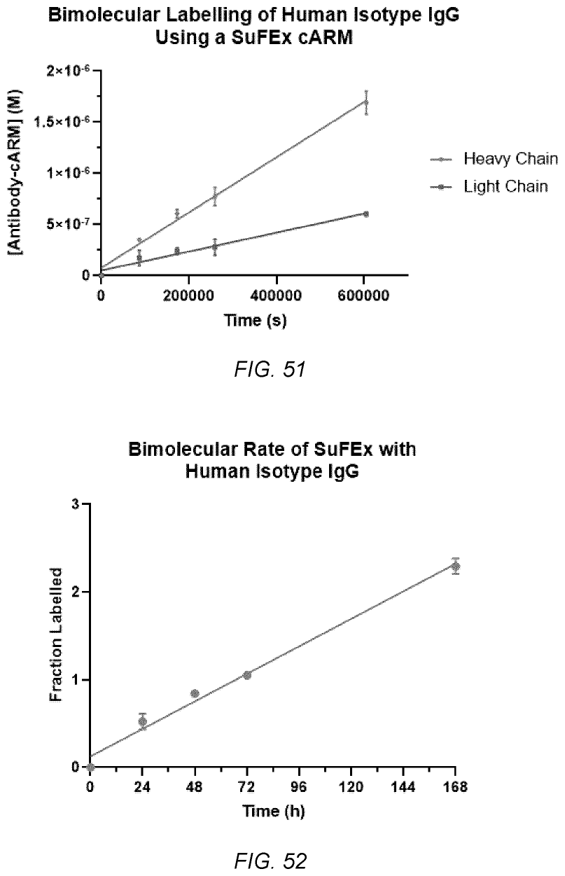

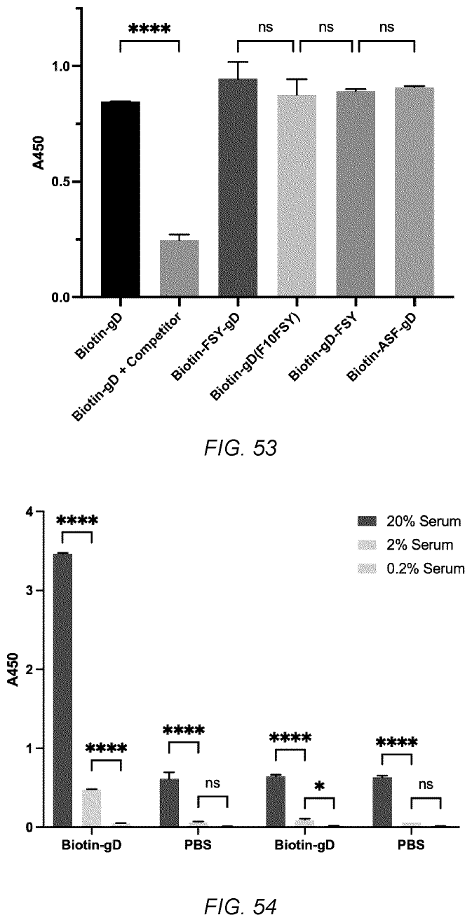

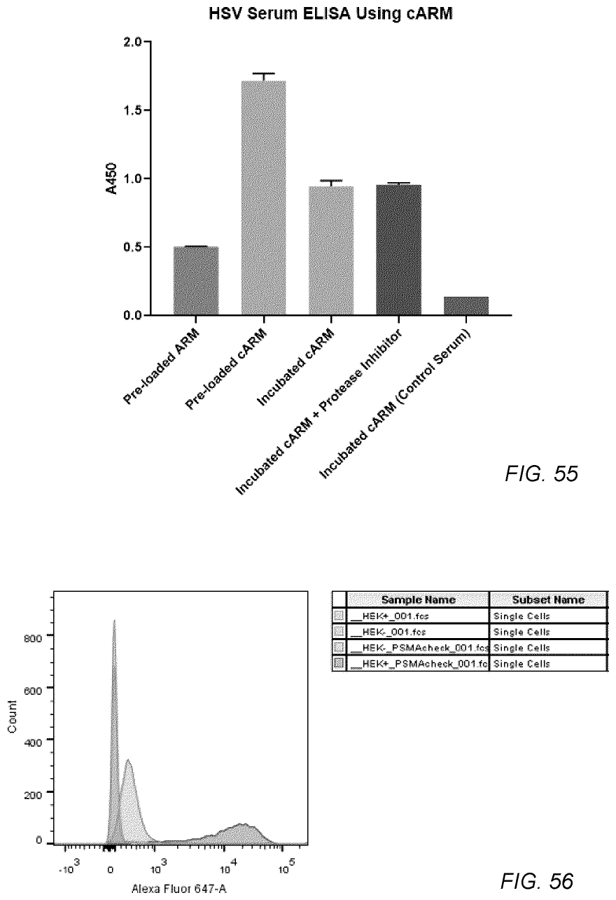

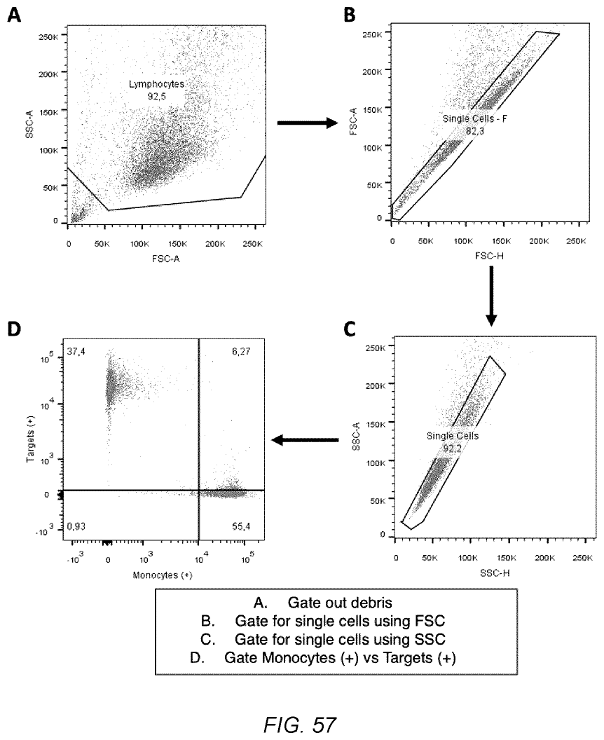

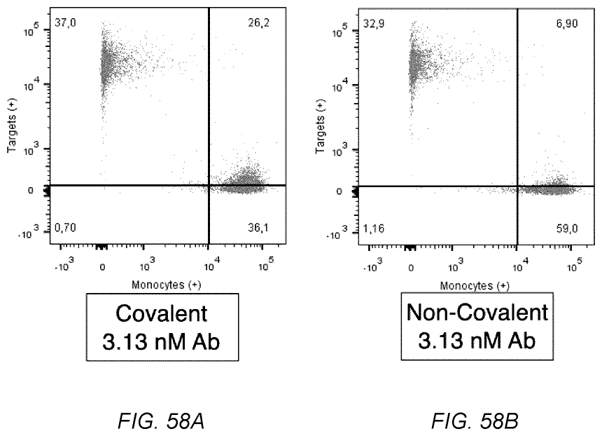

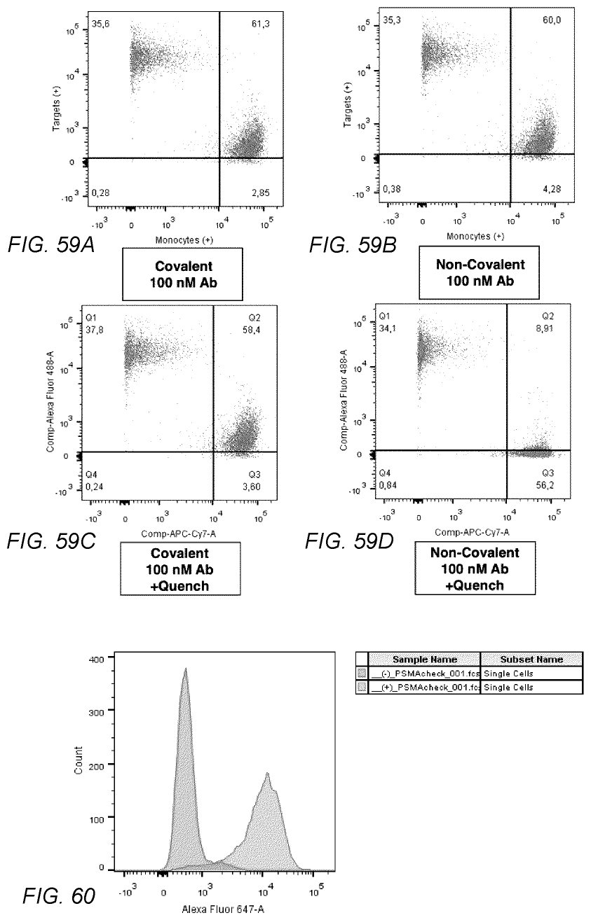

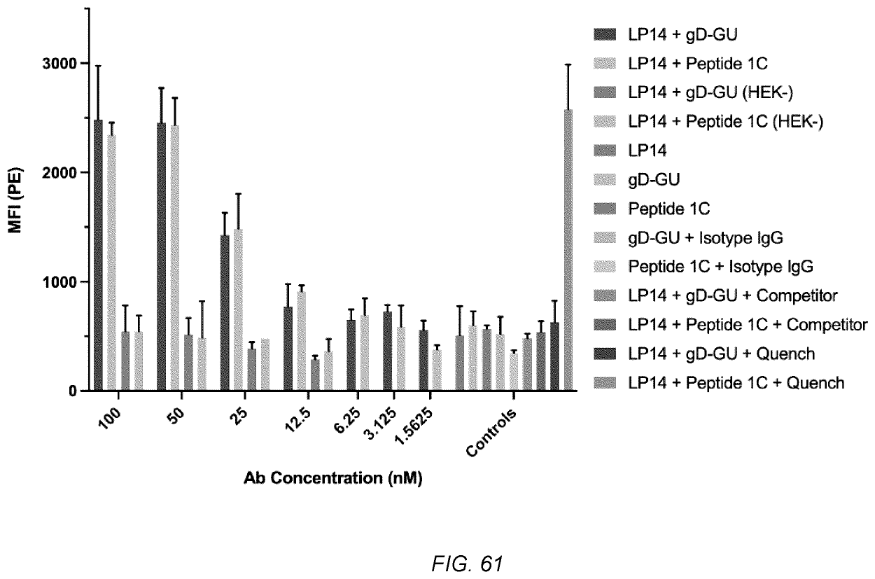

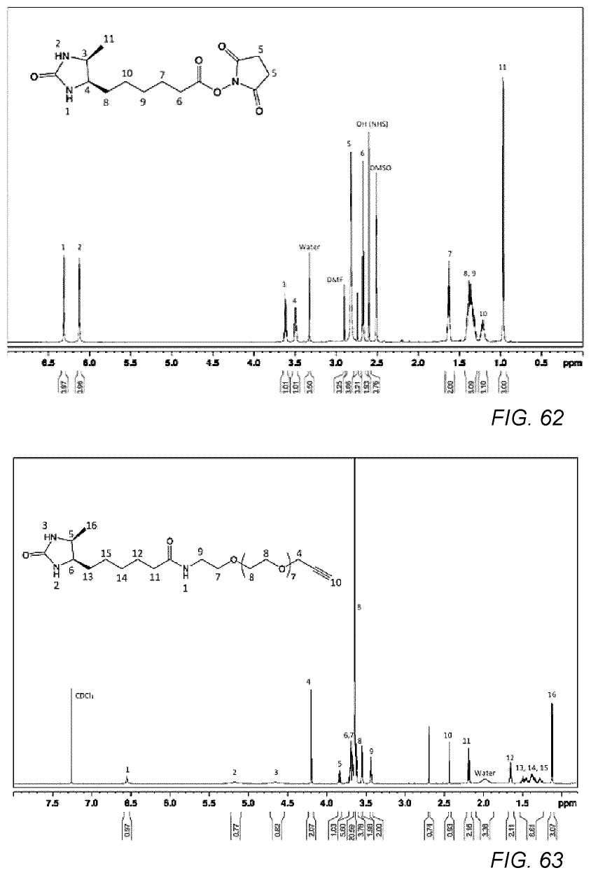

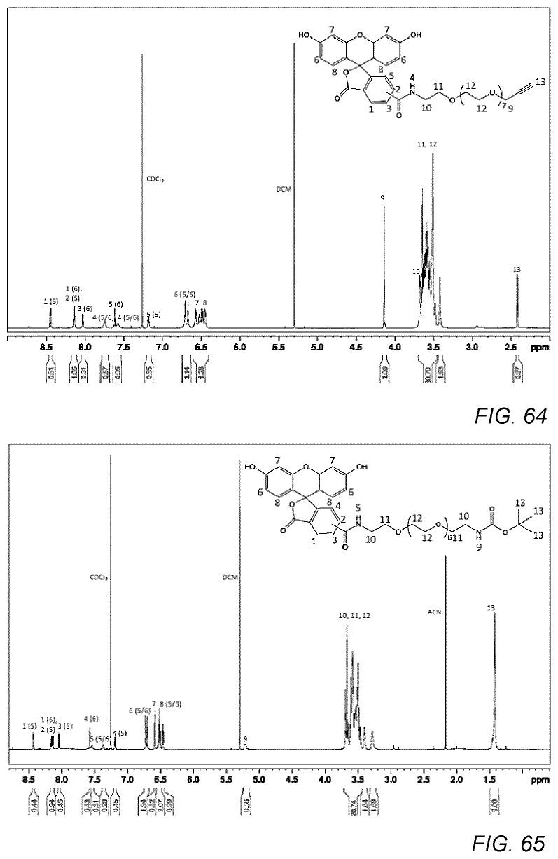

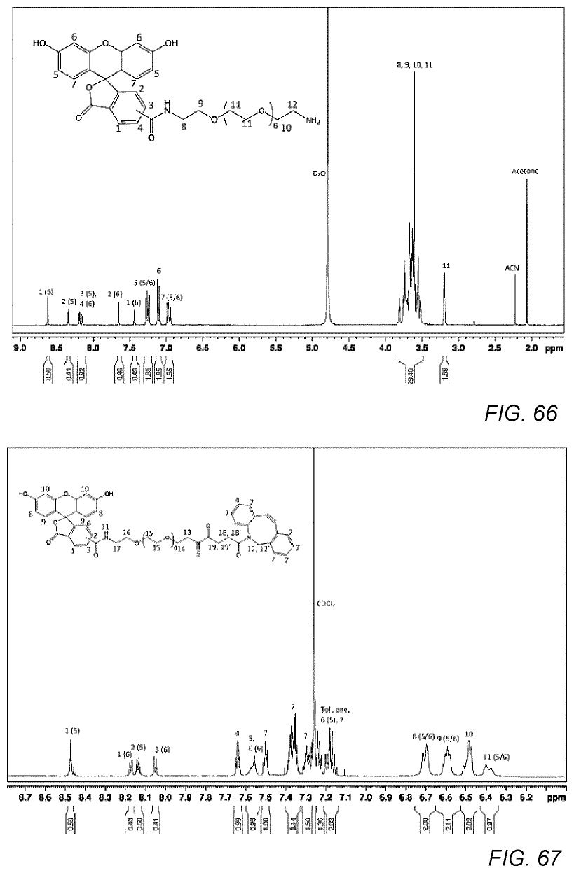

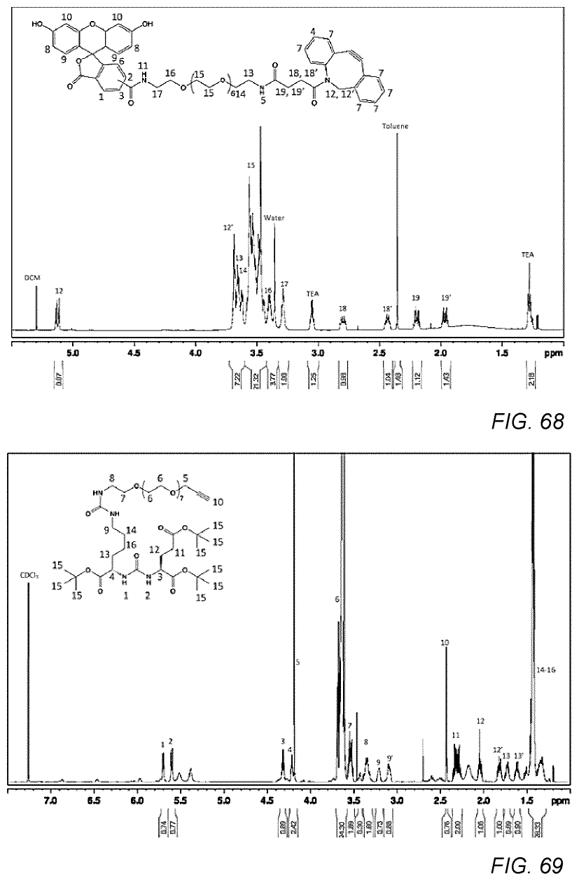

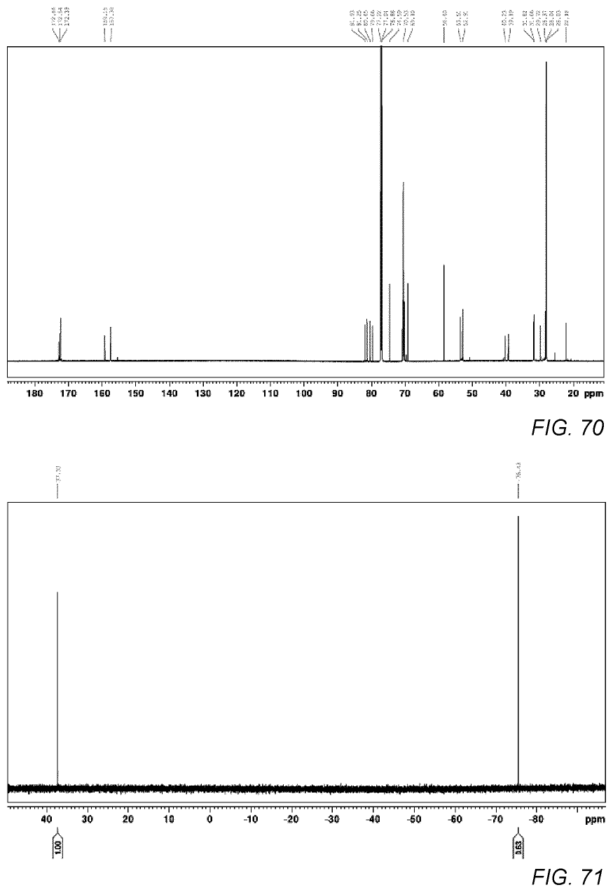

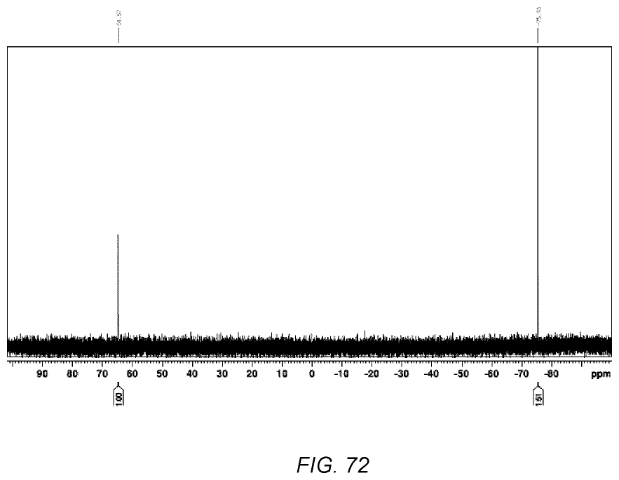

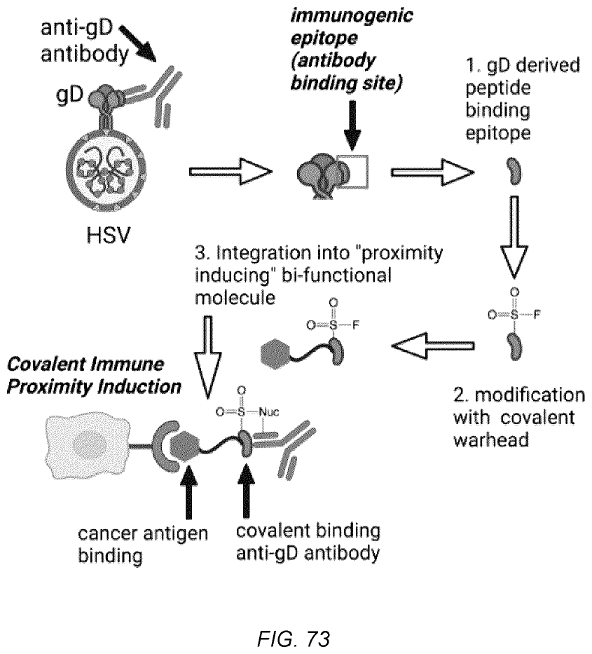

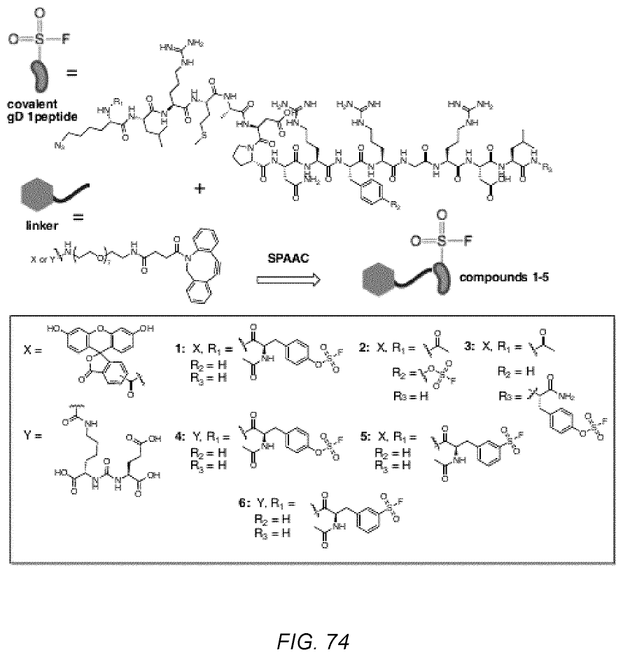

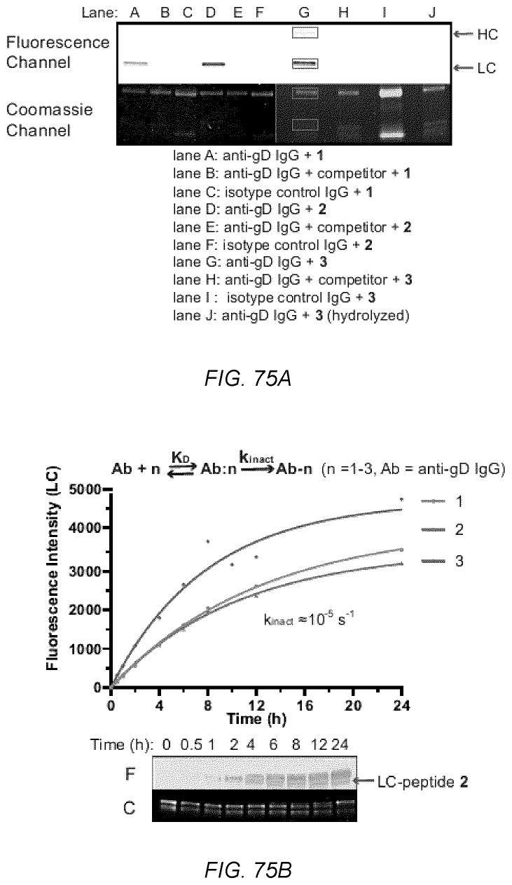

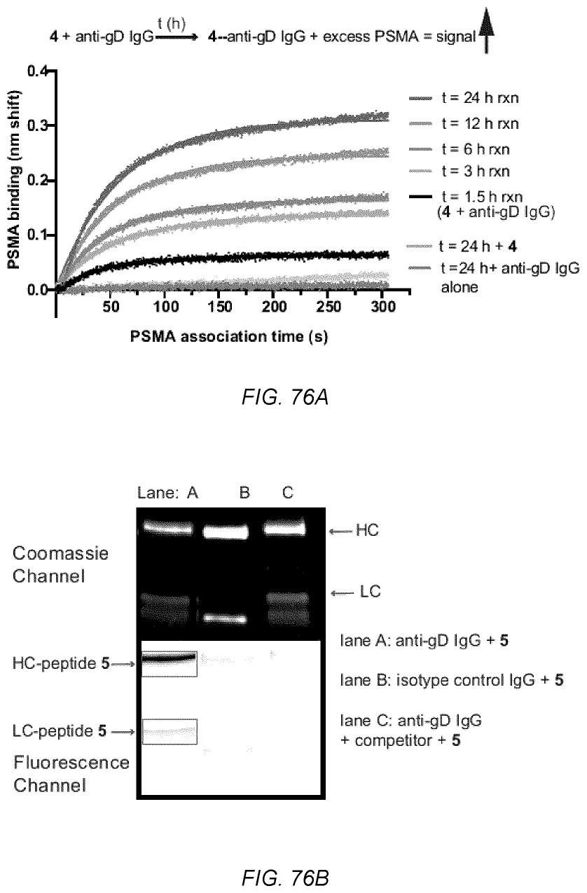

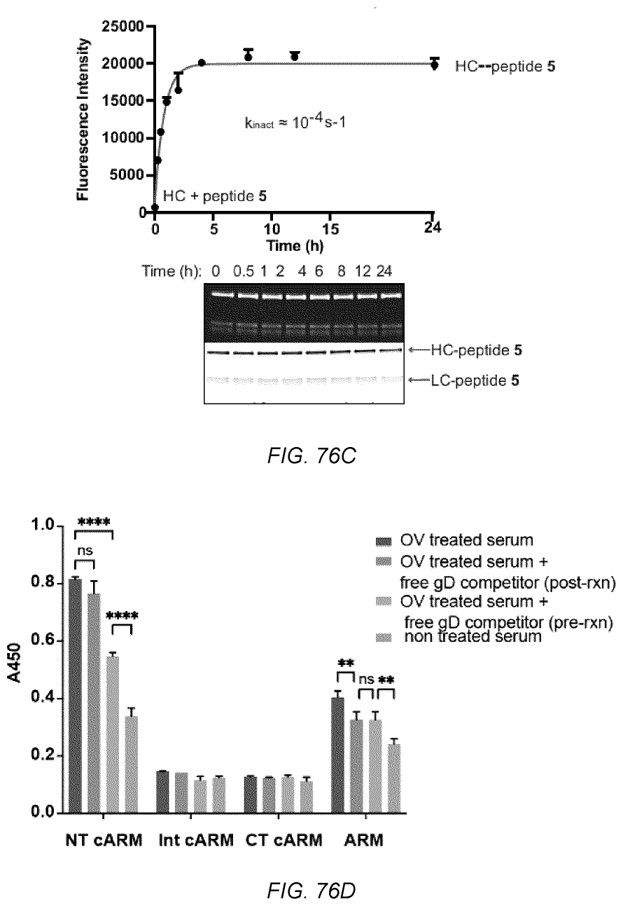

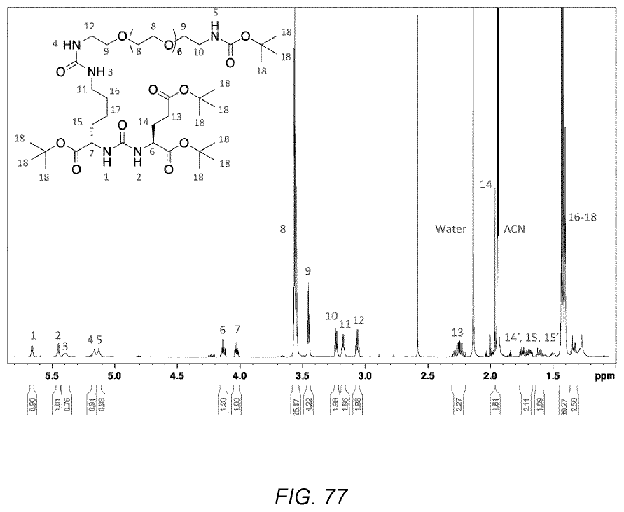

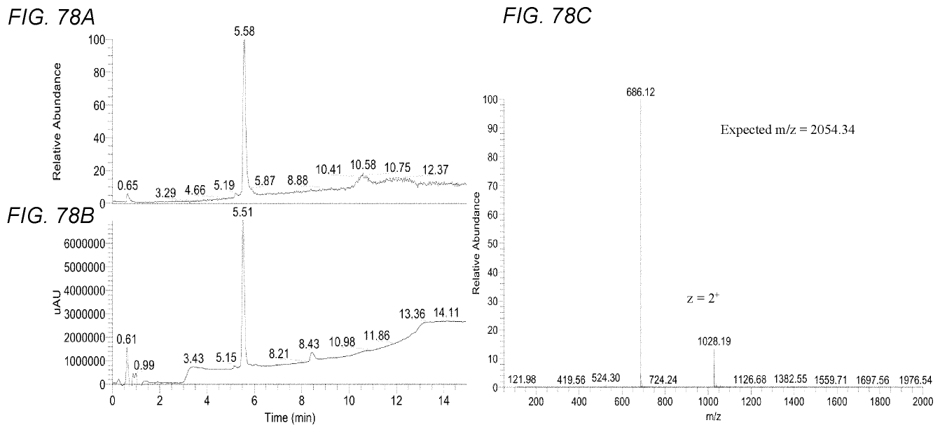

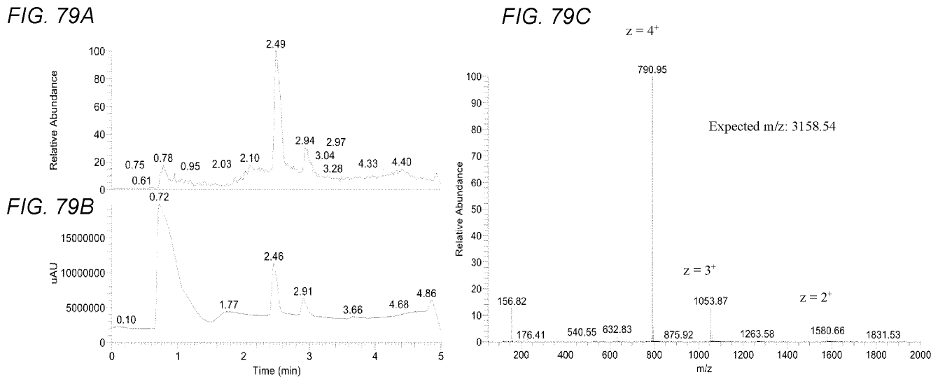

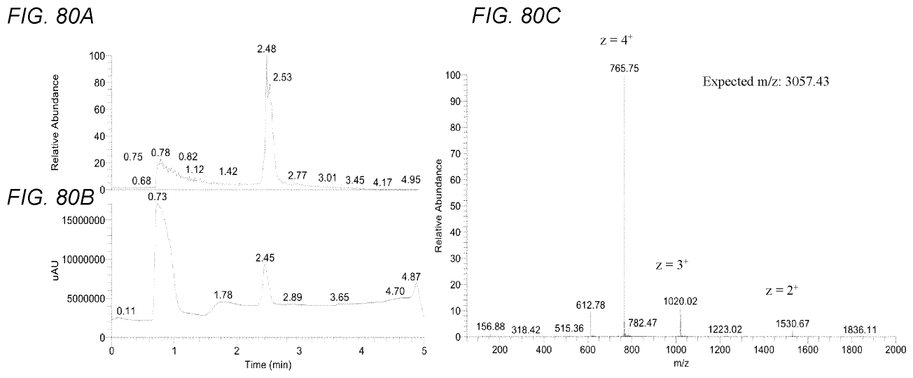

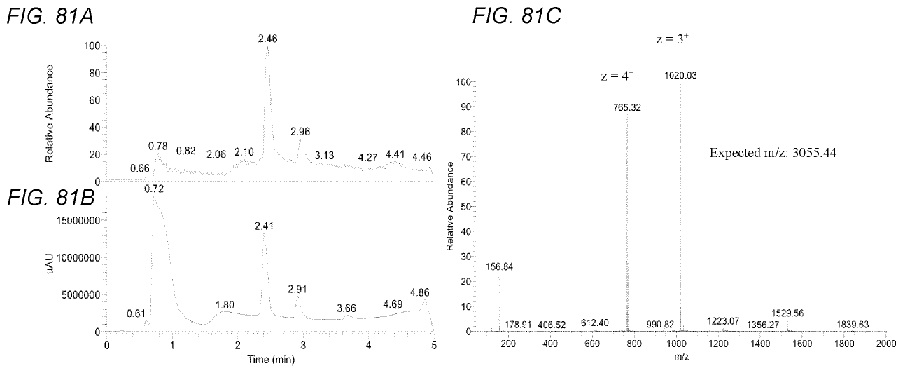

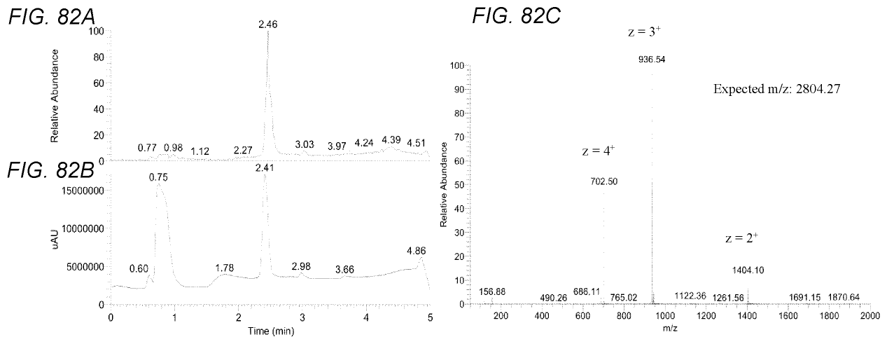

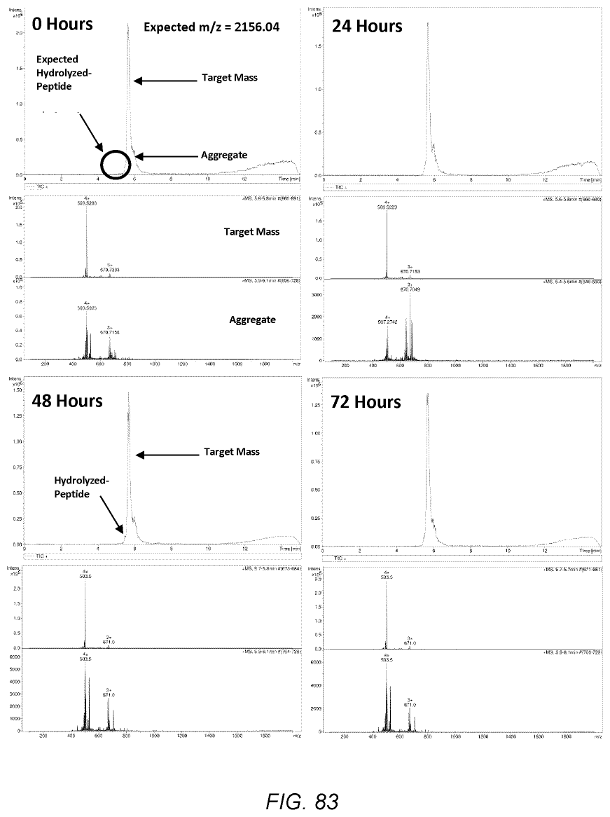

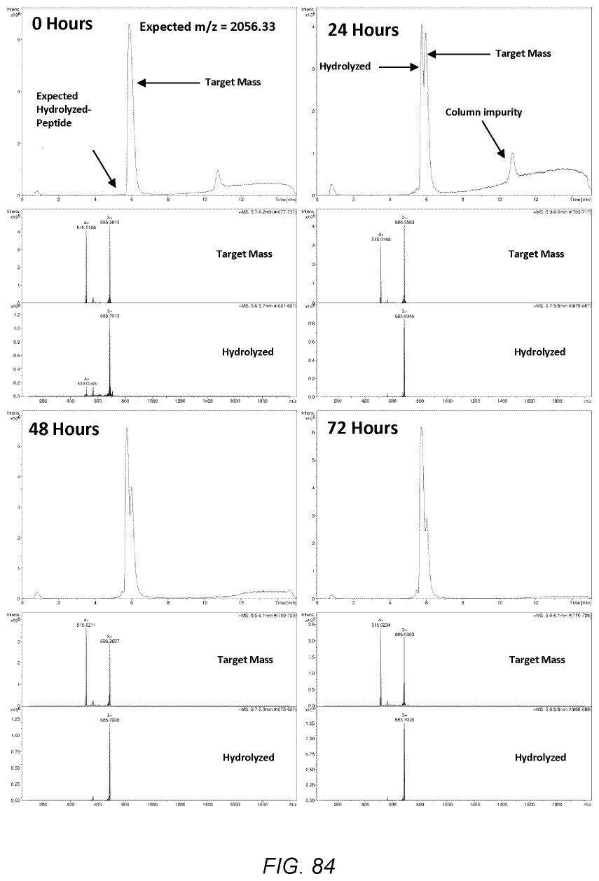

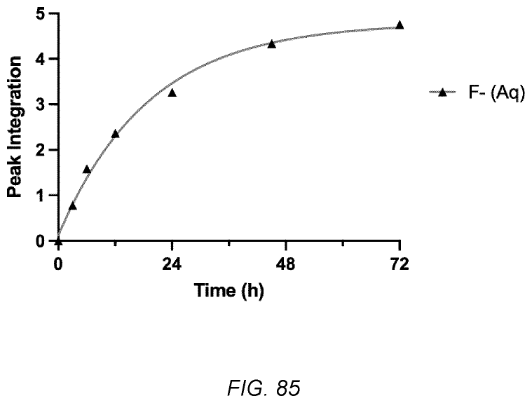

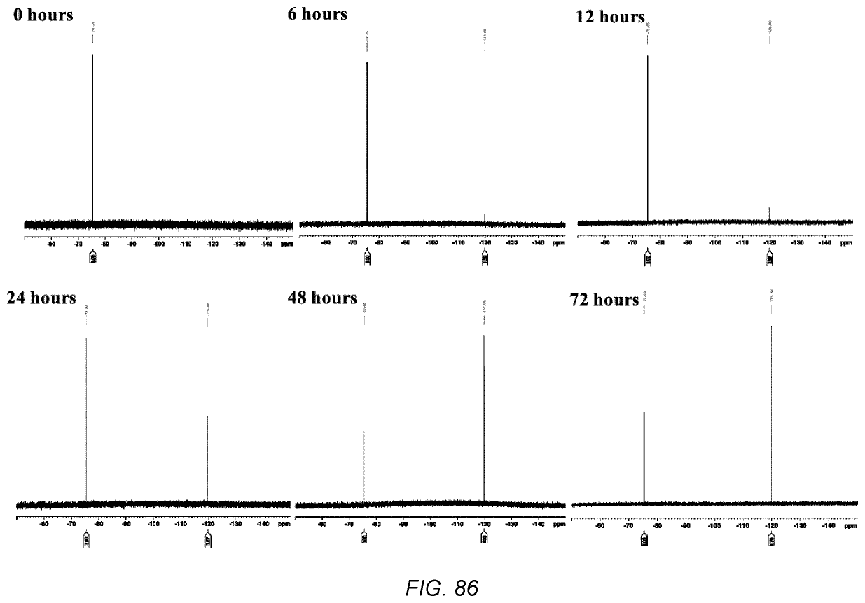

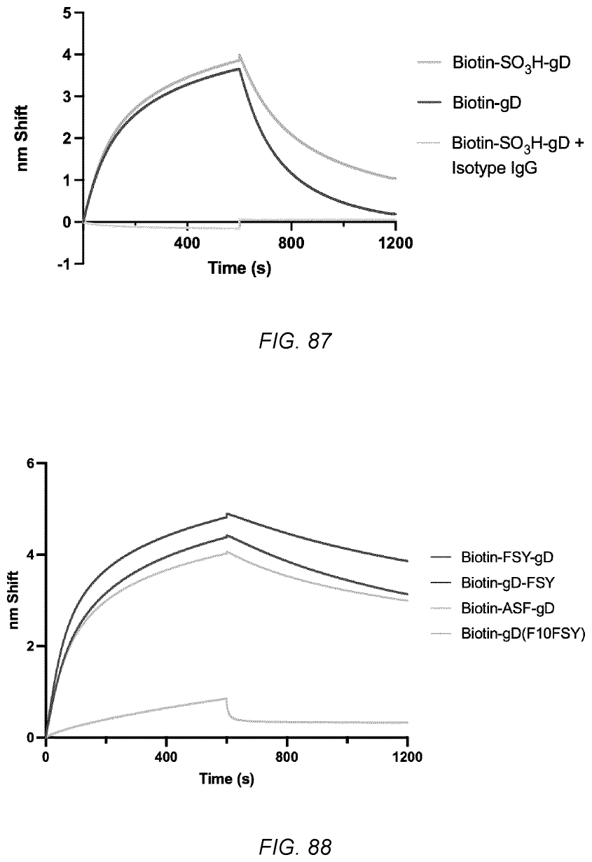

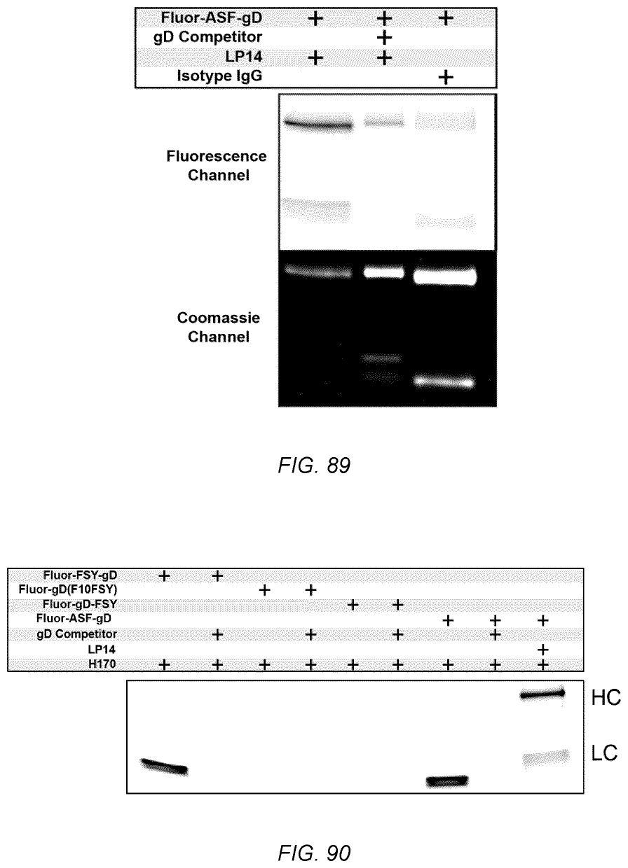

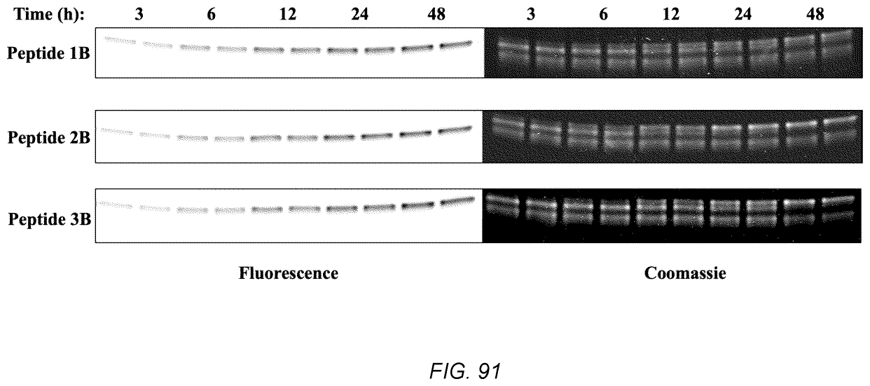

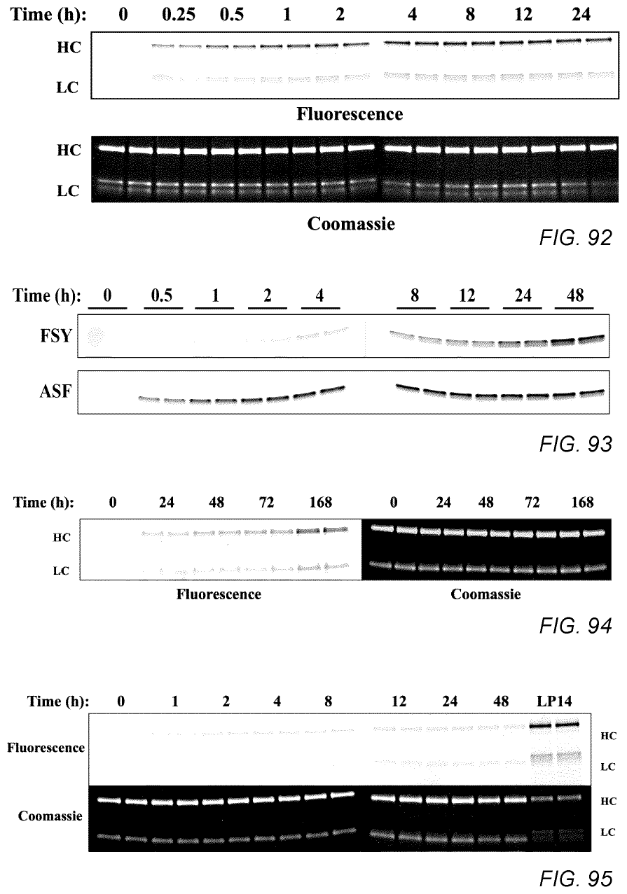

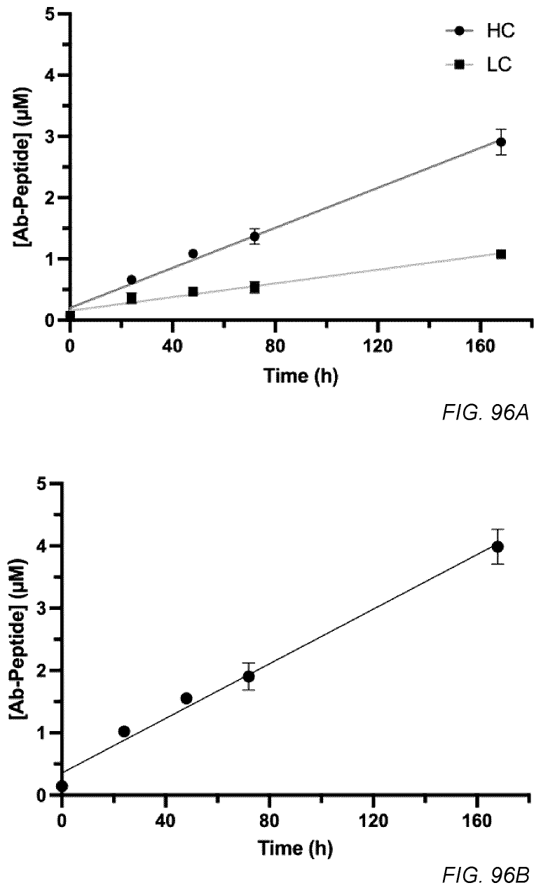

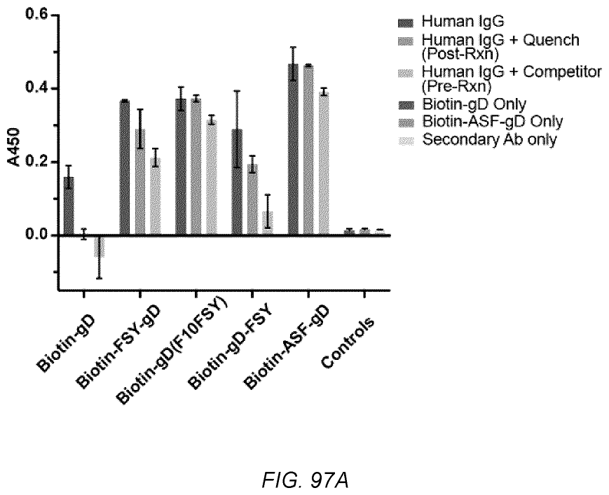

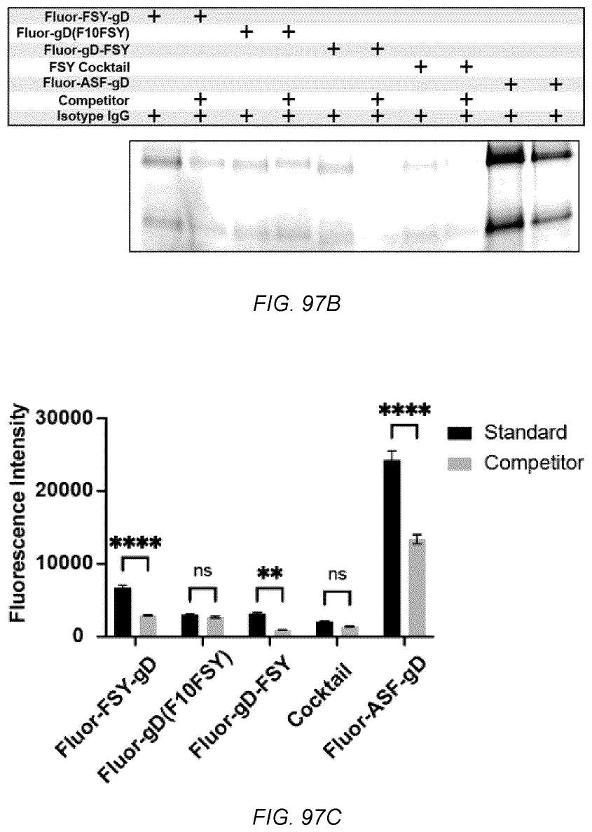

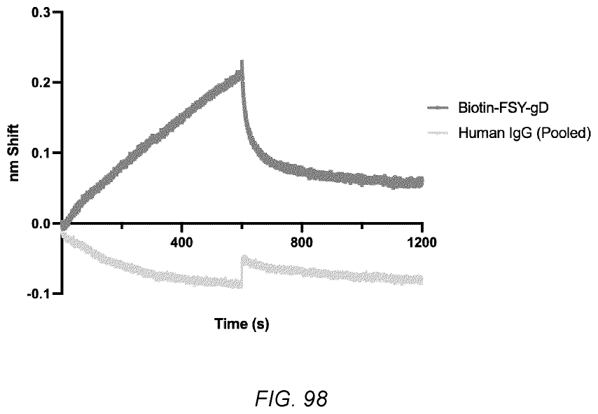

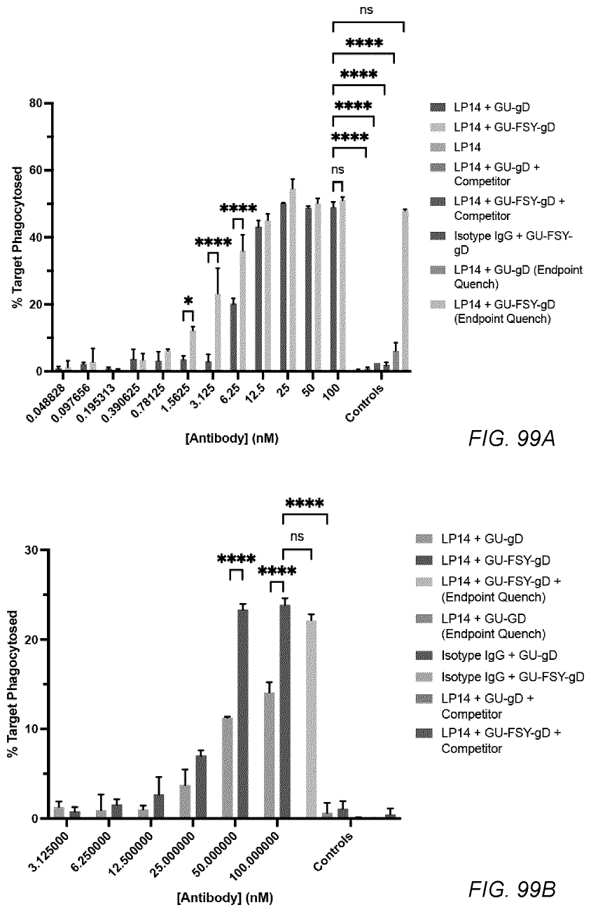

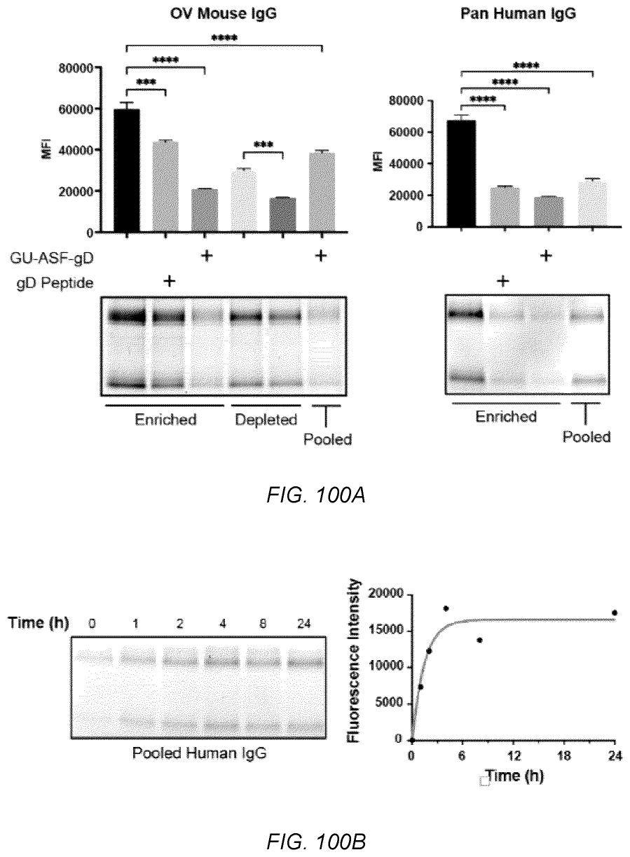

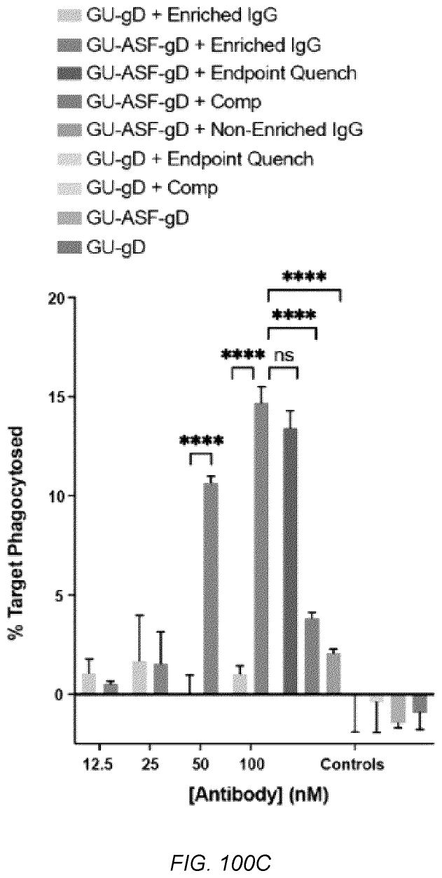

FIG. 9B is a mass spectrum of ARM 1.2.

FIG. 10A is a plot of the wavelength shift over time as measured by bio-layer interferometry (BLI) analysis of the recruitment of an anti-HSV gD1 monoclonal antibody to streptavidin-immobilized ARM 1.1. Buffer alone served as a negative control. “Flipped” gD1 peptide, which served as a control for selective binding of the anti-HSV antibody to the ARM, contains the same amino acids as the “correct” gD1 peptide, but has an incorrect amino acid sequence. nm: nanometer

FIG. 10B is a plot of the wavelength shift overtime as measured by BLI analysis of competitive dissociation of an anti-HSV gD1 monoclonal antibody from streptavidin-immobilized ARM 1.1 using free gD1 peptide. nm: nanometer

FIG. 11 is a Coomassie-stained SDS-PAGE gel of ARM 1.1-mediated isolation of anti-HSV gD1 polyclonal antibodies (pAb) from pooled human serum IgG. Lanes 1 and 2 contain serum IgG that does not retain on an affinity resin loaded with ARM 1.1 during buffer wash steps and is either non-specific IgG or weak affinity anti-HSV gD1 IgG. Lane 3 contains eluate from an additional wash step. Lane 4 contains specific anti-HSV gD1 IgG that was affinity-isolated using beads coated with ARM 1.1. BSA: bovine serum albumin

FIG. 12 is a graph of ARM 1.2-mediated antibody-dependent cellular phagocytosis (ADCP) of PSMA-expressing human embryonic kidney (HEK) cells by U937 human monocytes, as determined by 2-color flow cytometry. Samples:
HEK+_EXP (experimental sample): includes PSMA-expressing HEK cells, anti-HSV antibody and ARM 1.2;
HEK+_ARMOnly (control sample): same as HEK+_EXP, but excluding anti-HSV antibody;
HEK+_EXP_Iso (control sample): same as HEK+_EXP, but using an IgG antibody that does not bind the HSV gD1 peptide on the ARM but does activate human monocytes if recruited to the target cell surface;
HEK+_HSVcomp (control sample): same as HEK+_EXP, but further including a free competitor molecule that prevents binding of the anti-HSV antibody to ARM 1.2;
HEK−_EXP (control sample): same as HEK+_EXP, but using isogenic control HEK cells that do not express PSMA.

FIG. 13A is a TIC trace (top) and a UV chromatogram (bottom) from a LCMS analysis of intermediate 1.

FIG. 13B is a mass spectrum of intermediate 1.

FIG. 14A is a TIC trace (top) and a UV chromatogram (bottom) from a LCMS analysis of intermediate 2.

FIG. 14B is a mass spectrum of intermediate 2.

FIG. 15A is a TIC trace (top) and a UV chromatogram (bottom) from a LCMS analysis of intermediate 3.

FIG. 15B is a mass spectrum of intermediate 3.

FIG. 16 is a TIC trace (top), a UV chromatogram (middle) and a mass spectrum (bottom) from a LCMS analysis of intermediate 4.

FIG. 17 is a TIC trace (top), a UV chromatogram (middle) and a mass spectrum (bottom) from a LCMS analysis of intermediate 6.

FIG. 18A is a TIC trace from a LCMS analysis of intermediate 10.

FIG. 18B is a UV chromatogram from the LCMS analysis of intermediate 10. A peak appearing at 0.70 minutes corresponds to PBS/acetonitrile.

FIG. 18C is a mass spectrum of ions detected at 2.93 minutes in the LCMS analysis of intermediate 10.

FIG. 19A is a TIC trace (top) and a UV chromatogram (bottom) from a LCMS analysis of peptide 1.

FIG. 19B is a mass spectrum of peptide 1.

FIG. 20A is a TIC trace (top) and a UV chromatogram (bottom) from a LCMS analysis of peptide 2.

FIG. 20B is a mass spectrum of peptide 2.

FIG. 21A is a TIC trace (top) and a UV chromatogram (bottom) from a LCMS analysis of peptide 3.

FIG. 21B is a mass spectrum of peptide 3.

FIG. 22A is a TIC trace (top) and a UV chromatogram (bottom) from a LCMS analysis of peptide 4.

FIG. 22B is a mass spectrum of peptide 4.

FIG. 23A is a TIC trace (top) and a UV chromatogram (bottom) from a LCMS analysis of peptide 5.

FIG. 23B is a mass spectrum of peptide 5.

FIG. 24A is a TIC trace (top) and a UV chromatogram (bottom) from a LCMS analysis of peptide 6.

FIG. 24B is a mass spectrum of peptide 6.

FIG. 25A is a TIC trace (top) and a UV chromatogram (bottom) from a LCMS analysis of peptide 7.

FIG. 25B is a mass spectrum of ions detected at 2.44 minutes for peptide 7.

FIG. 26A is a TIC trace (top) and a UV chromatogram (bottom) from a LCMS analysis of cARM 2.1. A sharp UV peak at 0.76 minutes is from DMSO used to dissolve fluorescein-PEG8-DBCO.

FIG. 26B is a mass spectrum of ions detected at 2.62 minutes for cARM 2.1.

FIG. 27A is a TIC trace (top) and a UV chromatogram (bottom) from a LCMS analysis of cARM 2.2. A sharp peak at 0.76 minutes corresponds to DMSO used to dissolve intermediate 6.

FIG. 27B is a mass spectrum of ions detected at 2.54 minutes for cARM 2.2.

FIG. 28A is a TIC trace (top) and a UV chromatogram (bottom) from a LCMS analysis of cARM 2.3.

FIG. 28B is a mass spectrum of cARM 2.3.

FIG. 29A is a TIC trace (top) and a UV chromatogram (bottom) from a LCMS analysis of cARM 2.5. A sharp peak at 0.72 minutes corresponds to DMSO used to dissolve intermediate 6.

FIG. 29B is a mass spectrum of ions detected at 2.57 minutes for cARM 2.5.

FIG. 30A is a TIC trace (top) and a UV chromatogram (bottom) from a LCMS analysis of cARM 2.7. A sharp peak at 0.75 minutes corresponds to DMSO used to dissolve biotin-PEG4-DBCO.

FIG. 30B is a mass spectrum of ions detected at 2.49 minutes for cARM 2.7.

FIG. 31A is a TIC trace (top) and a UV chromatogram (bottom) from a LCMS analysis of cARM 2.8. A sharp peak at 0.76 minutes corresponds to DMSO used to dissolve biotin-PEG4-DBCO.

FIG. 31B is a mass spectrum of ions detected at 2.45 minutes for cARM 2.8.

FIG. 32A is a TIC trace (top) and a UV chromatogram (bottom) from a LCMS analysis of cARM 2.9.

FIG. 32B is a mass spectrum of cARM 2.9.

FIG. 33A is a TIC trace (top) and a UV chromatogram (bottom) from a LCMS analysis of cARM 2.10. A sharp peak at 0.77 minutes corresponds to DMSO used to dissolve biotin-PEG4-DBCO. A UV peak at 2.98 minutes corresponds to unreacted biotin-PEG4-DBCO.

FIG. 33B is a mass spectrum of ions detected at 2.51 minutes for cARM 2.10.

FIG. 34A is a TIC trace (top) and a UV chromatogram (bottom) from a LCMS analysis of ARM 2.11.

FIG. 34B is a mass spectrum of ARM 2.11.

FIG. 35A is a TIC trace (top) and a UV chromatogram (bottom) from a LCMS analysis of ARM 2.12.

FIG. 35B is a mass spectrum of ARM 2.12.

FIG. 36A is a TIC trace (top) and a UV chromatogram (bottom) from a LCMS analysis of ARM 2.13.

FIG. 36B is a mass spectrum of ARM 2.13.

FIG. 37A is a TIC trace (top) and a UV chromatogram (bottom) from a LCMS analysis of ARM 2.14.

FIG. 37B is a mass spectrum of ARM 2.14.

FIG. 38A is a TIC trace (top) and a UV chromatogram (bottom) from a LCMS analysis of ARM 2.15.

FIG. 38B is a mass spectrum of ARM 2.15.

FIG. 39 is a UV chromatogram (top) and a mass spectrum (bottom) of ARM 2.16.

FIG. 40A is a TIC trace and a UV chromatogram (overlaid) from a LCMS analysis of ARM 2.17.

FIG. 40B is a mass spectrum of ARM 2.17.

FIG. 41 is a graph of the hydrolysis decay of Aryl-SO₂F, monitored by ¹⁹F NMR. Peptide 6 was dissolved in 90% PBS and 10% D₂O and monitored by ¹⁹F NMR for F-generation as a function of time.

FIG. 42 is a graph of the binding of modified gD peptides to LP14 mAb analyzed by BLI. Analysis was performed using biotinylated non-covalent ARMs (200 μM) which were immobilized onto streptavidin-coated probes before association with anti-gD LP14 mAb (100 μM). A human IgG isotype control was used to control for non-specific binding to DTB-gD. DTB-gD=ARM 2.11; Biotin-gD=ARM 2.12; DTB-gDR=ARM 2.13; and Biotin-gDR=ARM 2.14.

FIG. 43 is a graph of the binding of C-terminal modified gDR-biotin conjugates to LP14 mAb, analyzed by BLI as above. Biotin-gDR-Y=ARM 2.15; Biotin-gDR-FSY=cARM 2.9.

FIG. 44 is a graph showing how changing the location of the fluorosulfate-I-tyrosine (FSY) covalent reactive group affects k_{off} in BLI. Each of the N-terminal (cARM 2.7: Biotin-FSY-gDR), Internal (cARM 2.8: Biotin-gDR(F10FSY)), and C-terminal (cARM 2.9: Biotin-gDR-FSY) SuFEx variants were compared with the original peptide (ARM 2.11: DTB-gD) as a positive control for differences in binding to anti-HSV gD LP14 mAb.

FIG. 45 is a graph showing that LP14 mAb pre-incubated with cARM demonstrated a covalent reaction in BLI. 2 μM of ARM 2.15 (Biotin-gDR-Y) or cARM 2.9 (Biotin gDR-FSY) was pre-incubated with 1 μM antibody over a 24-hour period before diluting 10-fold. This was then directly loaded onto a probe until near saturation, where dissociation was monitored in the presence of free competitor peptide.

FIG. 46 is a graph showing proximity-induced covalent labeling of LP14 mAb with cARM 2.7 (Biotin-FSY-gDR) or cARM 2.8 (Biotin-gDR(F10FSY)), analyzed by BLI. Conditions were kept identical to those used to assess the covalent reaction between cARM 2.9 and LP14 mAb in FIG. 45.

FIG. 47 is a graph showing the results of a reverse format BLI experiment for in-solution proximity labelling kinetics of covalent cARM 2.4 and LP14 mAb. LP14 mAb was labelled with 10× excess cARM 2.4 for 24 hours prior to detection. Fc-capture probes immobilized 75 nM antibody-cARM 2.4, before a 20-minute competition phase with 100 μM competitor peptide 1 ensured no residual non-covalent interactions between immobilized antibody and cARM 2.4. 500 nM PSMA was used to perform an association readout, where nm shift is correlated to the [Ab:cARM].

FIG. 48 is a graph showing the results of covalent labelling of LP14 mAb by cARM 2.4 monitored by reverse format BLI. The average 50 points of the processed PSMA association readout was plotted as a function of reaction time.

FIG. 49 is an image showing the results of a selectivity experiment between each SuFEx chemistry and PSMA. PSMA and cARMs were incubated at 2 μM and 20 μM concentrations, respectively. Lanes 1 and 2 compare the N-terminal Aryl-OSO₂F cARM 2.4 at 24 hours and time 0. Lanes 3 and 4 compare the N-terminal Aryl-SO₂F cARM 2.6 at 24 hours and time 0. Lanes 1-4 on the left show a fluorescent readout with the PMT auto calibrated to the unreacted cARM. Lanes 1-4 on the right show a fluorescent readout with the PMT auto calibrated to the reacted PSMA-cARM. A large degree of smearing was seen in lane 3, likely from intermolecular reactions occurring between fluorescein phenol groups and SO₂F groups. This ultimately reduced the effective [unreacted cARM 2.6] in lane 3 (left).

FIG. 50 is an image showing the results of an SDS-PAGE analysis of bimolecular non-specific labeling of isotype control IgG using cARM 2.5 including both Coomassie stain and fluorescein imaging of labeled IgG heavy and light chains.

FIG. 51 is a plot based on the reaction kinetics determined in FIG. 50 using quantitative standard curve methods to convert fraction antibody labeled to concentration in units of molarity. This enabled calculation of the second order rate constant employing the method of initial rates.

FIG. 52 is a plot based on the reaction kinetics determined in FIG. 50 converted to fraction of total antibody covalently labeled by cARM 2.5.

FIG. 53 is a graph showing the results of an ELISA assay to evaluate the extent of covalent labeling of LP14 mAb (100 nM) by the non-covalent ARM 2.12 (Biotin-gD), or the covalent cARM 2.7 (Biotin-FSY-gDR), cARM 2.8 (Biotin-gDR(F10FSY)), cARM 2.9 (Biotin-gDR-FSY), or cARM 2.10 (Biotin-ASF-gDR) (1 μM). Reactions involving covalent peptides were quenched with 180 μM gD peptide after 48 hours, prior to loading into wells. Each condition was performed in duplicate.

FIG. 54 is a graph showing the results of an ELISA assay using Biotin-gD peptides to probe endogenous anti-HSV antibodies in sera from mice (n=5) boosted with OV. Sera from mice not exposed to OV (n=3) were used as a control for basal anti-HSV antibody levels. A PBS blank was used in place of Biotin-gD to detect non-specific binding of serum IgG, leading to background signal. Data was quantified from two replicate measurements and summarized as the mean and standard error of the mean (*p=0.0332, ****p<0.0001, two-way analysis of variance (ANOVA)).

FIG. 55 is a graph showing the results of an ELISA assay comparing covalent versus non-covalent binding to natural anti-HSV antibodies present in mouse serum from OV boosted mice as in FIG. 54. Controls for proteolysis in mouse serum included addition of protease inhibitor cocktail. As a control for specific anti-HSV antibody binding, control serum from mice not exposed to OV was used as a serum source. “ARM”=ARM 2.12, “cARM”=cARM 2.7.

FIG. 56 is a graph showing PSMA expression on HEK+/− cells detected by flow cytometry using an anti-PSMA Alexa647-labeled antibody. Only HEK+ cells were found to express PSMA. HEK− cells demonstrated very little fluorescence, likely from non-specific binding. Without the anti-PSMA A647 antibody, no fluorescence was detected.

FIG. 57 is a set of flow cytometry scatter plots showing gating protocols for selecting single cells when evaluating double positives, reflecting ADCP events.

FIG. 58A is a flow cytometry scatter plot showing ADCP of PSMA-expressing HEK+ cells by u937 monocytes in the presence of 3.13 nM LP14 mAb and 6.26 nM cARM 2.4 (GU-FSY-gDR). Quadrant 1 (top left) indicates target cells, quadrant 2 (top right) indicates phagocytosed cells, quadrant 3 (bottom left) indicates cellular debris, and quadrant 4 (bottom right) indicates monocytes.

FIG. 58B is a flow cytometry scatter plot showing ADCP of PSMA-expressing HEK+ cells by u937 monocytes in the presence of 3.13 nM LP14 mAb and 6.26 nM ARM 2.17 (GU-gD). Quadrant 1 (top left) indicates target cells, quadrant 2 (top right) indicates phagocytosed cells, quadrant 3 (bottom left) indicates cellular debris, and quadrant 4 (bottom right) indicates monocytes.

FIG. 59A is a flow cytometry scatter plot showing ADCP of PSMA-expressing HEK+ cells by u937 monocytes in the presence of LP14 mAb and cARM 2.4 (GU-FSY-gDR).

FIG. 59B is a flow cytometry scatter plot showing ADCP of PSMA-expressing HEK+ cells by u937 monocytes in the presence of LP14 mAb and ARM 2.17 (GU-gD).

FIG. 59C is a flow cytometry scatter plot showing ADCP of PSMA-expressing HEK+ cells by u937 monocytes in the presence of LP14 mAb and cARM 2.4 (GU-FSY-gDR). A quench was performed by adding 100 μM gD peptide (ARM 2.11) after an overnight incubation of the LP14 antibody with cARM 2.4 to demonstrate the presence of covalent linkage.

FIG. 59D is a flow cytometry scatter plot showing ADCP of PSMA-expressing HEK+ cells by u937 monocytes in the presence of LP14 mAb and ARM 2.17 (GU-gD). A quench was performed by adding 100 μM gD peptide (ARM 2.11) after an overnight incubation of the LP14 antibody with ARM 2.17 to rule out the presence of covalent linkage.

FIG. 60 is a graph showing PSMA expression on HEK+/− cells detected by flow cytometry using an anti-PSMA Alexa647-labeled monoclonal antibody. The experiment was performed as in FIG. 56.

FIG. 61 is a graph of the mean fluorescence intensity (MFI) measured by flow cytometry of PSMA-expressing HEK+ cells using cARM 2.4 (GU-FSY-gDR) and non-covalent control ARM 2.17 (GU-gD). A PE-conjugated secondary anti-mouse (H+L chain) antibody was used to detect LP14 mAb recruited to HEK+ cells. MFI of each cell population was used as a measure of antibody recruitment. A quench using 25× excess of free competitor gD peptide was used to distinguish covalent from non-covalent binding, based on the retention or loss of MFI signal proportional to antibody recruitment, respectively.

FIG. 62 is 700 MHz ¹H NMR spectrum of intermediate 1 in DMSO.

FIG. 63 is 700 MHz ¹H NMR spectrum of intermediate 2 in CDCl₃.

FIG. 64 is 700 MHz ¹H NMR spectrum of intermediate 3 in CDCl₃.

FIG. 65 is 700 MHz ¹H NMR spectrum of intermediate 4 in CDCl₃.

FIG. 66 is 700 MHz ¹H NMR spectrum of intermediate 5 in D₂O.

FIG. 67 is the downfield portion of 700 MHz ¹H NMR spectrum of intermediate 6 in CDCl₃.

FIG. 68 is the upfield portion of 700 MHz ¹H NMR spectrum of intermediate 6 in CDCl₃.

FIG. 69 is 700 MHz ¹H NMR spectrum of intermediate 8 in CDCl₃.

FIG. 70 is 700 MHz ¹³C NMR spectrum of intermediate 9 in CDCl₃.

FIG. 71 is 700 MHz ¹⁹F NMR spectrum of C-terminal ArylOSO₂F peptide containing a TFA internal standard and 90% 1× PBS, 10% D₂O.

FIG. 72 is 700 MHz ¹⁹F NMR spectrum of ArylSO₂F peptide containing a TFA internal standard and 90% 1× PBS, 10% D₂O.

FIG. 73 is an illustration of a general strategy to convert viral immunogenic peptide epitopes into covalent “proximity-inducing” bi-functional antibody recruitment molecules. A peptide recognized by natural anti-viral antibodies in human blood is engineered to bind with infinite affinity using SuFEx covalent chemistry and incorporated into bi-functional molecules. Resultant bi-functional molecules are designed to enforce antibody-cancer cell proximity, leading to targeted cancer cell elimination by the host immune system.

FIG. 74 is an illustration of the chemical structures of fluorescence kinetic gD covalent peptide probes and analogous covalent immune proximity inducing molecules (cARMs 2.1-2.6).

FIG. 75A is an image showing the validation of covalent antibody binding by cARMs 2.1-2.3 in fluorescence SDS-PAGE assays. cARMs 2.1-2.3 (20 μM) were incubated with anti-gD IgG “LP14” (1 μM) in the presence or absence of free competitor gD peptide or incubated with isotype control IgG antibody. After 24 h, aliquots of the reaction solution were run on reducing SDS-PAGE and imaged by both Coomassie stain and fluorescein fluorescence. HC=antibody heavy chain, LC=antibody light chain.

FIG. 75B is a graph showing the covalent binding/labeling kinetics determined as in FIG. 75A, except that the reaction solution was assayed at 0, 0.5, 1, 2, 4, 6, 8, 10, 12, and 24 hour time points. Plots of the fraction of antibody light chain covalent labelling over time, were fit to an integrated first order rate law to extract k_{inact} according to a saturation kinetics mechanism. Relative band intensities (indicated by boxes) were quantified by densitometry analysis using Image J software.

FIG. 76A is a graph of the results of BLI ternary complex assays involving cARM 2.4, anti-gD IgG (LP14 mAb) and soluble PSMA. Solutions containing antibody and cARM 2.4 incubated for different periods of time (h) were exposed to antibody “Fc” capture probe and washed to remove non-covalent antibody bound complex. Resultant probes now immobilized with antibody covalently bound to cARM 2.4 were submerged in a solution of fixed amount of soluble PSMA.

FIG. 76B is an image showing the results of a fluorescence SDS-PAGE selectivity experiment performed identically as described in FIG. 75A but using cARM 2.5.

FIG. 76C is a graph of a covalent binding/labeling kinetics experiment performed identically as described in FIG. 75B but using cARM 2.5.

FIG. 76D is a graph showing the results of an ELISA assay to detect endogenous anti-HSV antibodies specific for the gD epitope in mouse serum. Serum of mice boosted with HSV-1d810 (oncolytic virus) (10× dilution) was incubated with cARM 2.7 (Biotin-FSY-gDR=“NT cARM”), cARM 2.8 (Biotin-gDR(F10FSY)=“Int cARM”), cARM 2.9 (Biotin-gDR-FSY=“CT cARM”), or ARM 2.12 (Biotin-gD=“ARM”) for 24 hours before ELISA-based detection. The same gD peptide competition controls were used to explore covalency and selectivity. Serum from a mouse not boosted with the oncolytic virus was used to further investigate selectivity. Detection of antibody complexes was done using anti-mouse HRP secondary antibody conjugates. Data was quantified from two replicate measurements and summarized as the mean and standard error of the mean (**p=0.0021, ****p<0.0001, two-way ANOVA).

FIG. 77 is 700 MHz ¹H NMR of GU-PEG7-NBoc in CD₃CN.

FIG. 78A is a TIC trace from a LCMS analysis of peptide 8.

FIG. 78B is a UV chromatogram from the LCMS analysis of peptide 8.

FIG. 78C is a mass spectrum of ions detected at 5.58 minutes in the LCMS analysis of peptide 8, using a 15-minute LCMS method.

FIG. 79A is a TIC trace from a LCMS analysis of cARM 2.4.

FIG. 79B is a UV chromatogram from the LCMS analysis of cARM 2.4. A UV peak at 0.72 minutes is from DMSO used to dissolve intermediate 10.

FIG. 79C is a mass spectrum of ions detected at 2.49 minutes in the LCMS analysis of cARM 2.4.

FIG. 80A is a TIC trace from a LCMS analysis of cARM 2.6.

FIG. 80B is a UV chromatogram from the LCMS analysis of cARM 2.6. A sharp peak at 0.76 minutes corresponds to DMSO used to dissolve intermediate 10.

FIG. 80C is a mass spectrum of ions detected at 2.48 minutes in the LCMS analysis of cARM 2.6.

FIG. 81A is a TIC trace from a LCMS analysis of ARM 2.18.

FIG. 81B is a UV chromatogram from the LCMS analysis of ARM 2.18. A sharp peak at 0.72 minutes corresponds to DMSO used to dissolve intermediate 10.

FIG. 81C is a mass spectrum of ions detected at 2.46 minutes in the LCMS analysis of ARM 2.18.

FIG. 82A is a TIC trace from a LCMS analysis of ARM 2.19.

FIG. 82B is a UV chromatogram from the LCMS analysis of ARM 2.19. A large UV peak at 0.75 minutes corresponds to DMSO used to dissolve biotin-PEG4-DBCO.

FIG. 82C is a mass spectrum of ions detected at 2.46 minutes in the LCMS analysis of ARM 2.19.

FIG. 83 is a set of graphs showing the mass spectra of peptide 5 (gDR(F10FSY)) at 0, 24, 48 and 72 hours of incubation with 1×PBS at room temperature, assessed by LC-HRMS.

FIG. 84 is a set of graphs showing the mass spectra of peptide 7 (ASF-gDR) at 0, 24, 48 and 72 hours of incubation with 1×PBS at room temperature, assessed by LC-HRMS.

FIG. 85 is a graph showing hydrolysis of sulfonyl fluoride. Peptide 7 (ASF-gDR) was dissolved in 90% PBS/10% D₂O and generation of F⁻ (aq) was monitored by ¹⁹F NMR as a function of time.

FIG. 86 is a set of graphs showing the ¹⁹F NMR spectra of peptide 7 (ASF-gDR) dissolved in 90% PBS/10% D₂O taken over a period of 72 hours. The peak at −75.45 ppm corresponds to TFA, which served as an internal standard. The peak at −119.8 ppm corresponds to F⁻(aq).

FIG. 87 is a graph of LP14 mAb binding to hydrolyzed aryl-sulfonyl fluoride-modified peptide Biotin-SO₃H-gDR (ARM 2.19). BLI analysis was performed as in FIGS. 42-46. A human IgG isotype control antibody was included with peptide Biotin-SO₃H-gD (ARM 2.19) as a control for selective binding.

FIG. 88 is a graph showing proximity-induced covalent labeling of H170 mAb with cARM 2.7 (Biotin-FSY-gDR), cARM 2.8 (Biotin-gDR(F10FSY)), cARM 2.9 (Biotin-gDR-FSY) or cARM 2.10 (Biotin-ASF-gDR), analyzed by BLI. cARMs 2.7-2.10 (200 nM) immobilized to streptavidin-coated biosensors were used to measure specific binding to the H170 mAb (100 nM) in 1× kinetics buffer. Dissociation was monitored by submerging the biosensor:peptide:Ab complex in free gD peptide (200 μM).

FIG. 89 is an image of a fluorescent SDS-PAGE experiment evaluating the selective binding of high concentration cARM 2.5 (Fluorescein-ASF-gDR) to LP14 mAb. cARM 2.5 (20 μM) was incubated with LP14 mAb (1 μM) alone or LP14 mAb pre-incubated with competitor gD peptide (100 μM). A human IgG isotype control antibody (1 μM) was used as a control for non-specific binding.

FIG. 90 is an image of a fluorescent SDS-PAGE experiment evaluating the selective binding of the following SuFEx group-containing antibody recruitment molecules to H170 mAb: cARM 2.1 (Fluorescein-FSY-gDR), cARM 2.2 (Fluorescein-gDR(F10FSY)), cARM 2.3 (Fluorescein-gDR-FSY), and cARM 2.5 (Fluorescein-ASF-gDR)). Each one of cARMs 2.1, 2.2, 2.3 and 2.5 (1 μM) was incubated with H170 mAb (0.5 μM) alone, or with H170 mAb pre-incubated with competitor gD peptide (500 μM). LP14 mAb (0.5 μM) was used as a positive control for covalent labeling.

FIG. 91 is an image of a fluorescent SDS-PAGE time-course experiment of the labeling kinetics between FSY-equipped peptides and anti-gD LP14 mAb. Peptides (20 μM) containing a covalently reactive FSY group at the N-terminus (cARM 2.1=“Peptide 1B”), internal F10 position (cARM 2.2=“Peptide 2B”) or C-terminus (cARM 2.3=“Peptide 3B”) were incubated with LP14 (1 μM) for 0, 3, 6, 12, 24 or 48 hours before SDS-PAGE and fluorescent detection.

FIG. 92 is an image of a fluorescent SDS-PAGE time-course experiment of the labeling kinetics between excess cARM 2.5 (Fluorescein-ASF-gDR) and anti-gD LP14 mAb. cARM 2.5 (20 μM) was incubated with LP14 mAb (1 μM) for 0, 0.25, 0.5, 1, 2, 4, 8, 12 and 24 hours before SDS-PAGE and fluorescent detection. SDS-PAGE gels were placed adjacent to one another for fluorescent imaging.

FIG. 93 is an image of a fluorescent SDS-PAGE time-course experiment of the labeling kinetics between cARM 2.1 (Fluorescein-FSY-gDR) or cARM 2.5 ((Fluorescein-ASF-gDR) and anti-gD H170 antibody. Each of cARM 2.1 (10 μM) and cARM 2.5 (10 μM) was incubated with H170 mAb (0.5 μM) for 0, 0.5, 1, 2, 4, 8, 12, 24 and 48 hours before SDS-PAGE and fluorescent detection.

FIG. 94 is an image of a fluorescent SDS-PAGE time-course experiment to evaluate the non-specific, bimolecular reaction between fluorosulfate-substituted cARM 2.1 (fluorescein-FSY-gDR) and a human IgG isotype antibody.

FIG. 95 is an image of a fluorescent SDS-PAGE time-course experiment to evaluate the non-specific, bimolecular reaction between sulfonyl fluoride-substituted cARM 2.5 (fluorescein-ASF-gDR) and a human IgG isotype antibody. SDS-PAGE gels were placed adjacent to one another for fluorescent imaging.

FIG. 96A is a graph showing the bimolecular reaction rate between cARM 2.1 (Fluorescein-FSY-gDR) and a non-binding human IgG isotype antibody, determined using fluorescent SDS-PAGE. Aliquots were flash frozen at −80° C. to generate timepoints for SDS-PAGE and subsequent fluorescent detection of labeled protein bands. The linear stage of the bimolecular reaction is depicted. HC=IgG heavy chain; LC=IgG light chain.

FIG. 96B is a graph showing the combined bimolecular reaction rate between cARM 2.1 (Fluorescein-FSY-gDR) and both the heavy and light chain of non-binding human IgG isotype antibody from the same experiment as in FIG. 96A.

FIG. 97A is a graph showing the results of an ELISA assay to detect endogenous anti-HSV antibodies specific for the gD epitope in human serum. Pooled human IgG (5 μM) isolated from serum was incubated with covalent cARM 2.7 (Biotin-FSY-gDR), cARM 2.8 (Biotin-gDR(F10FSY)), cARM 2.9 (Biotin-gDR-FSY) or cARM 2.10 (Biotin-ASF-gDR) or non-covalent ARM 2.12 (Biotin-gD) (1 μM) for 24 hours or 60 seconds (t=0). Competitor gD peptide (100 μM) was added after a 24-hour incubation time to serve as a control for covalent labeling (post-rxn). Reaction selectivity was assessed by adding competitor gD peptide to antibody source before the respective covalent peptide (pre-rxn). Data was quantified from two replicate measurements and summarized as the mean and standard error of the mean.

FIG. 97B is an image of a fluorescent SDS-PAGE experiment assessing covalent labeling of polyclonal, endogenous natural anti-HSV antibodies from human serum. cARM 2.1 (Fluorescein-FSY-gDR), cARM 2.2 (Fluorescein-gDR(F10FSY)), cARM 2.3 (Fluorescein-gDR-FSY), a combination of the cARMs 2.1-2.3 (10 μM), or cARM 2.5 (Fluorescein-ASF-gDR)) were spiked into pooled human IgG (2 μM) isolated from serum.

FIG. 97C is a graph of the fluorescence intensities obtained from the SDS-PAGE experiment shown in FIG. 97B. Selectivity was assessed using the pre-rxn control described above in FIG. 97A. Data was quantified from two replicate measurements and summarized as the mean and standard error of the mean (**p=0.0021, ****p<0.0001, two-way ANOVA).

FIG. 98 is a graph of the results of a BLI experiment to assess the binding of cARM 2.7 (Biotin-FSY-gDR) to enriched anti-gD IgG isolated from pooled human serum IgG. cARM 2.7 (200 nM) was loaded onto streptavidin-coated probes before association with 100 nM enriched human anti-gD IgG. Dissociation was performed in 200 μM competitor gD peptide.

FIG. 99A is a graph of the results of two-colour flow cytometry ADCP assays conducted using FL-4 (DID) dye stained u937 human monocyte cells and FL-1 (DIO) dye stained HEK cells engineered to express PSMA. Double positive cell events corresponding to target cell phagocytosis were recorded in the presence of LP14 mAb or isotype control antibody at the indicated concentration and 2 equivalents of cARM 2.4 (GU-FSY-gDR) or non-covalently reactive analog ARM 2.17 (GU-gD) at 37° C. Antibody and cARM 2.4 (GU-FSY-gDR) were pre-incubated with or without excess free competitor gD peptide (pre-rxn) for 24 hours prior to dilution to the indicated concentrations into solutions of target and immune cells for all conditions. Control experiments were all conducted with 100 nM antibody and 2 equivalents of cARM 2.4 (GU-FSY-gDR) or ARM 2.17 (GU-gD). Additional controls for covalency involved addition of excess free competitor gD peptide following the 24 hour incubation (post-rxn). Data was quantified from two replicate measurements and summarized as the mean and standard error of the mean (*p=0.0332, ****p<0.0001, two-way ANOVA).

FIG. 99B is a graph of the results of two-colour flow cytometry ADCP assays conducted under identical concentrations and conditions as in FIG. 99A, except that lower PSMA level-expressing Lymph Node Carcinoma of the Prostate (LNCaP) cells were used in place of the high PSMA level-expressing HEK cells. Data was quantified from two replicate measurements and summarized as the mean and standard error of the mean (****p<0.0001, two-way ANOVA).

FIG. 100A is a set of graphs (top panels) and images (bottom panels) from a fluorescent SDS-PAGE analysis of selective covalent labeling between cARM 2.5 (Fluorescein-ASF-gDR) (1 μM) and enriched (0.5 μM), depleted (0.5 μM), or pooled (0.5 μM) IgG. Pooled IgG refers to a polyclonal IgG mixture from either 5 mice boosted with HSV oncolytic virus (“OV Mouse IgG”, left panels), or a commercial polyclonal human IgG product (“Pan Human IgG”, right panels). Enriched IgG was made using a pull-down column with gD immobilized onto streptavidin agarose. Depleted IgG was obtained as the flow through from the pull-down column after 3 pull down cycles. Selectivity was demonstrated using either a non-covalent gD competitor peptide (10 μM), or cARM 2.6 (GU-ASF-gDR) (10 μM) as a covalent competitor.

FIG. 100B is an image (left panel) and a graph (right panel) from a reaction time-course between cARM 2.5 (Fluorescein-ASF-gDR) (10 μM) and pan human IgG (1 μM) over 24 hours. A plot of heavy chain+light chain labeling over time was fitted to a first order rate law under the assumption of saturating conditions.

FIG. 1000 is a graph of the results of two-colour flow cytometry ADCP assays using FL-4 (DID) dye stained u937 human monocyte cells and FL-1 (DIO) dye stained HEK cells engineered to express PSMA. Double-positive events were recorded in the presence of enriched human IgG with 2 equivalents of cARM 2.6 (GU-ASF-gDR) or ARM 2.17 (GU-gD) at 37° C. Labeling was performed for 24 hours, and selectivity was probed through competition, where 200 μM free competitor gD peptide was pre-equilibrated with enriched IgG prior to spiking with cARM 2.6 or ARM 2.17. Covalency was demonstrated through a quench, where 200 μM free competitor gD peptide was spiked into the reaction after 24 hours with either cARM 2.6 or ARM 2.17.

Other features and advantages of the present disclosure will become more apparent from the following detailed description and from the exemplary embodiments.

### DETAILED DESCRIPTION OF THE DISCLOSURE

### Definitions

The terminology used herein is for the purpose of describing particular embodiments only and is not intended to be limiting of example embodiments of the invention. Unless defined otherwise, all technical and scientific terms used herein generally have the same meaning as commonly understood by those of ordinary skill in the art. Generally, nomenclatures used in connection with synthetic chemistry, organic chemistry, biochemistry, molecular biology, cell and tissue culture, immunology, genetics, etc. described herein are those well-known and commonly used in the art. Nevertheless, definitions of selected terms are provided for clarity and consistency.

The following list includes abbreviations used repeatedly throughout the present disclosure:
ABT: antibody-binding terminus (of antibody recruitment molecule)
ACN: acetonitrile
ADCC: antibody-dependent cellular cytotoxicity
ADCP: antibody-dependent cellular phagocytosis
ANOVA: analysis of variance
ARM: antibody recruitment molecule (also referred to as antibody engager (AE)
molecule or immune proximity inducing molecule)
ASF: aryl-sulfonyl fluoride (SO₂F, a reactive group for covalent binding)
BLI: bio-layer interferometry
Boc: tert-butyloxycarbonyl protecting group
BSA: bovine serum albumin
cARM: covalent antibody recruitment molecule, a type of ARM (also referred to as covalent antibody engager (cAE) molecule, or covalent immune recruiter (CIR))
CuAAC: copper(I)—catalyzed azide-alkyne cycloaddition (a type of “click” chemistry reaction)
DBCO: dibenzocyclooctyne
DCM: dichloromethane
DIC: N,N′Diisopropylcarbodiimide
DMF: dimethylformamide
DMSO: dimethylsulfoxide
DNP: dinitrophenyl
ELISA: enzyme-linked immunosorbent assay
Fab: antigen-binding fragment region of an antibody
Fc: fragment crystallizable region of an antibody
Fmoc: fluorenylmethoxycarbonyl
FSY: fluorosulfate-I-tyrosine (OSO₂F, a reactive group for covalent binding)
gD: glycoprotein D (of herpes simplex virus)
GUL: glutamate urea lysine (also abbreviated as GU)
¹H NMR: proton nuclear magnetic resonance
HEK: human embryonic kidney (cell line)
HER2: human epidermal growth factor receptor 2 (also known as HER2/neu, ERBB2, CD340)
HRMS: high resolution mass spectrometry
HSV: herpes simplex virus
IgG: immunoglobulin G
IgM: immunoglobulin M
IEDDA: inverse electron demand Diels-Alder (a type of “click” chemistry reaction)
IV: intravenous (route of administration)
L: linker (optional component of antibody recruitment molecule)
LCMS: liquid chromatography-mass spectrometry
LNCaP: Lymph Node Carcinoma of the Prostate (cell line)
mAb: monoclonal antibody
MFI: mean fluorescence intensity
MW: molecular weight
NHS: N-hydroxy succinimide
NK: natural killer cells
OtBu: oxygen protecting tert-butyl group
OV: oncolytic virus
pAb: polyclonal antibody
PEG: polyethylene glycol
PSMA: prostate-specific membrane antigen (also known as PSM, GCP2, FOLH1, NAALAD1)
RPM: rounds per minute
SDS-PAGE: sodium dodecyl sulfate-polyacrylamide gel electrophoresis
SPAAC: strain-promoted azide-alkyne cycloaddition (a type of “click” chemistry reaction)
SPPS: solid phase peptide synthesis
SuFEx: sulfur (VI) fluoride exchange (a type of “click” chemistry reaction)
TBT: target-binding terminus (of antibody recruitment molecule)
TEA: triethylamine
TFA: trifluoroacetic acid
THPTA: Tris(3-hydroxypropyltriazolylmethyl)amine
TIC: total ion current
TIPS: triisopropyl silane
T-VEC: Talimogene Laherparepvec (marketed under the trade name IMLYGIC™ and also known as AMG-678, OncoVEX, OncoVEX^{GM-CSF})
pAU: micro-absorbance unit
uPAR: urokinase-type plasminogen activator receptor (also known as CD87)

As used herein, the singular forms “a,” “an,” and “the,” are intended to include the plural forms as well, unless the context clearly indicates otherwise.

The phrase “and/or” should be understood to mean “either or both” of the elements so conjoined, i.e., elements that are conjunctively present in some cases and disjunctively present in other cases. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein, the phrase “one or more,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “one or more” refers, whether related or unrelated to those elements specifically identified. Thus, as a non-limiting example, “one or more of A and B” (or, equivalently, “one or more of A or B,” or, equivalently “one or more of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the term “about” is used in conjunction with a numerical range, it modifies that range by extending the boundaries above and below those numerical values. In general, the term “about” is used herein to modify a numerical value above and below the stated value by a variance of 20%, 10%, 5%, or 1%. In certain embodiments, the term “about” is used to modify a numerical value above and below the stated value by a variance of 10%. In certain embodiments, the term “about” is used to modify a numerical value above and below the stated value by a variance of 5%. In certain embodiments, the term “about” is used to modify a numerical value above and below the stated value by a variance of 1%.

When a range of values is listed herein, it is intended to encompass each value and sub-range within that range. For example, “1-5 ng” is intended to encompass 1 ng, 2 ng, 3 ng, 4 ng, 5 ng, 1-2 ng, 1-3 ng, 1-4 ng, 1-5 ng, 2-3 ng, 2-4 ng, 2-5 ng, 3-4 ng, 3-5 ng, and 4- 5 ng.

It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

The term “consisting of” and its derivatives, as used herein, are intended to be closed terms that specify the presence of stated features, integers, steps, operations, elements, and/or components, and exclude the presence or addition of one or more other features, integers, steps, operations, elements and/or components.

The term “isolated molecule” (where the molecule is, for example, a small molecule, a polypeptide, a polynucleotide, or an antibody or fragment thereof) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

As used herein, an “antibody” is an immunoglobulin molecule capable of specific binding to a target, such as an antibody binding terminus of an ARM, carbohydrate, polynucleotide, lipid, polypeptide, etc., through at least one antigen recognition site, located in the variable region of the immunoglobulin molecule. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. In one embodiment, the antibody is a human antibody.

The term “linker” or “linker group” as used herein refers to any molecular structure that joins two or more other molecular structures together and that is compatible with a biological/physiological environment. The presence of a linker is optional in the ARM compounds of the present disclosure. Where present, the linker may comprise, for example, aliphatic chains, aromatic rings, PEG molecules and/or peptides with appropriate functionality for linkage to an ABT or a TBT of an ARM compound. In an embodiment, the linker is a portion of the ABT, for example, a moiety within the ABT that does not bind to an epitope or epitope mimetic of a Herpes Simplex Virus (HSV) surface protein. In an embodiment, the linker is a portion of the TBT, for example, a moiety within the TBT that does not bind to a target protein. The linker may function as a spacer of appropriate length between the ABT and the TBT of an ARM compound, for example, to minimize intramolecular and intermolecular steric effects, and optimize binding of the ARM compound to the antibody, the target, or both. Linkers suitable for use with the ARM compounds of the disclosure may be determined by a person of skill in the art.

As used herein, “substantially pure” means an object species, for example, an ARM compound or a component of an ARM compound of the disclosure, is the predominant species present (i.e., on a molar basis it is more abundant than any other individual species in the composition), and preferably a substantially purified fraction is a composition wherein the object species (e.g., a protein or a polypeptide) comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The term “treatment,” “treat,” “treating” or “amelioration” as used herein is an approach for obtaining beneficial or desired clinical results. For purposes of this disclosure, beneficial or desired clinical results include, but are not limited to, one or more of the following: decreased extent of damage from a disease, condition, or disorder, decreased duration of a disease, condition, or disorder, reduction in the number, extent, or duration of symptoms related to a disease, condition, or disorder, an increase in the period of time prior to a relapse of a disease, condition, or disorder in a subject, and/or an increase in the disease-free or overall survival rate of a subject having a disease, condition, or disorder. The term includes the administration of the compounds, agents, drugs or pharmaceutical compositions of the present disclosure to prevent or delay the onset of one or more symptoms, complications, or biochemical indicia of a disease or condition; lessening or improving one or more symptoms; shortening or reduction in duration of a symptom; arresting or inhibiting further development of a disease, condition, or disorder; or decreasing the toxicity of a therapy. Treatment may be prophylactic (to prevent or delay the onset of a disease, condition, or disorder, or to prevent the manifestation of clinical or subclinical symptoms thereof) or therapeutic suppression or alleviation of symptoms after the manifestation of the disease, condition, or disorder. Treatment may also be maintenance therapy to decrease the chances that a disease, condition, or disorder will reoccur or to delay recurrence of a disease, condition, or disorder. The beneficial result may be an increase or decrease (as appropriate) of 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100% relative to an appropriate control, for example, a subject that did not receive the therapy.

In an embodiment of the present disclosure, the disease, disorder or condition treatable by the compounds, pharmaceutical compositions, kits and methods provided herein is cancer. In an embodiment, the cancer is one that is impacted or treatable by immunotherapy, either alone or in combination with one or more other therapies, such as chemotherapy. In an embodiment, the cancer is one that is impacted or treatable by activation of endogenous immune cells. In an embodiment, the cancer is one that is impacted or treatable by stimulating an immune response to tumor cells. In an embodiment, the cancer is one that is impacted or treatable by provoking phagocytosis of tumor cells.

In an embodiment, the cancer is impacted or treatable by OV therapy, either alone or in combination with one or more other therapies, such as chemotherapy. In an embodiment, the cancer is impacted or treatable by T-VEC.

In certain embodiments, the cancer is selected from, but not limited to: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Triple Negative Breast Cancer; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioblastoma Multiforme; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor. Metastases of the aforementioned cancers can also be treated in accordance with the methods described herein.

In an embodiment, the cancer is prostate cancer, breast cancer, ovarian cancer or glioblastoma. In an embodiment, the cancer is prostate cancer. In an embodiment, the cancer is breast cancer. In an embodiment, the cancer is triple negative breast cancer. In an embodiment, the cancer is ovarian cancer. In an embodiment, the cancer is glioblastoma multiforme.

A “subject” is a vertebrate, preferably a mammal (e.g., a non-human mammal), and still more preferably a human. In an embodiment, the subject may be any human patient. In an embodiment, the subject may be limited to one or more patient subpopulations, such as, but not limited to, a female patient, a male patient, a geriatric patient, a pediatric patient, a patient with specific comorbidities and/or a patient with one or more genetic predispositions to hereditary cancer(s). In an embodiment, the subject is undergoing OV therapy or is a candidate for treatment with OV.

The term “administering” or “administration” as used herein refers to the placement of an agent, a drug, a compound, or a pharmaceutical composition as disclosed herein into a subject by a method or route which results in at least partial delivery of the composition to a desired site. The compounds and pharmaceutical compositions disclosed herein can be administered by any appropriate route which results in an effective treatment in the subject. Possible routes of administration of the compounds and pharmaceutical compositions disclosed herein include, but are not limited to, intravenous, intraperitoneal, intramuscular, subcutaneous, transdermal, oral, buccal, sublingual, intranasal, or rectal routes of administration, or a combination thereof.

The term “effective amount” or “therapeutically effective amount” as used herein is an amount sufficient to affect any one or more beneficial or desired results. In more specific aspects, an effective amount may alleviate or ameliorate one or more symptoms of cancer, decrease the duration of time that one or more symptoms of cancer are present in a subject, reduce the size of a tumor in a subject, eliminate all detectable levels of a tumor in a subject, increase the period of time prior to a relapse of cancer in a subject, and/or increase the disease-free or overall survival rate of a subject having cancer. For prophylactic use, beneficial or desired results may include eliminating or reducing the risk, lessening the severity, or delaying the onset of cancer or a particular stage/grade of the cancer, including biochemical and/or histological symptoms of the cancer, its complications and intermediate pathological phenotypes presenting during development of the cancer. For therapeutic use, beneficial or desired results may include clinical results such as reducing one or more symptoms of cancer; decreasing the dose or length of administration of other medications required to treat the cancer; enhancing the effect and/or reducing the toxicity of another medication; delaying the progression of the cancer a subject, decreasing the duration of time that one or more symptoms of cancer are present in a subject, increasing the period of time prior to a relapse of cancer in a subject, and/or increasing the disease-free or overall survival rate of a subject having cancer. An effective amount can be administered in one or more than one dose, round of administration, or course of treatment.

For purposes of this disclosure, an effective dosage of a compound or a pharmaceutical composition is an amount sufficient to accomplish prophylactic or therapeutic treatment either directly or indirectly. As is understood in the clinical context, an effective dosage of a compound, or a pharmaceutical composition may or may not be achieved in conjunction with another agent, drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved. The amount may vary from one subject to another and may depend upon one or more factors, such as, for example, subject gender, age, body weight, subject's health history, and/or the underlying cause of the disease, condition, or disorder to be prevented, inhibited and/or treated.

The term “pharmaceutically acceptable carrier, diluent, or excipient” as used herein includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. In some embodiments, diluents for aerosol or parenteral administration are phosphate buffered saline (PBS) or normal (0.9%) saline. Compositions comprising such carriers are formulated by well-known conventional methods (see, for example, Remington's Pharmaceutical Sciences, 18th edition, A. Gennaro, ed., Mack Publishing Co., Easton, P A, 1990; and Remington, The Science and Practice of Pharmacy 20th Ed. Mack Publishing, 2000).

The term “epitope” refers to the area or region of an antigen to which an antibody specifically binds, e.g., an area or region comprising a contact residue that interacts with the antibody. Thus, the term “epitope” refers to that portion of a molecule (e.g., an ABT of an ARM) capable of being recognized by and bound by an antibody at one or more of the antibody's antigen-binding regions. Typically, an epitope is defined in the context of a molecular interaction between an antibody, or antigen-binding fragment thereof, and its corresponding antigen. Epitopes often consist of a surface grouping of molecules such as amino acids or sugar side chains and have specific three-dimensional structural characteristics as well as specific charge characteristics.

The term “epitope mimetic” as used herein refers to a synthetic molecule that mimics the structure and/or function of epitopes found on the surface of a given protein or peptide. For example, an epitope mimetic can mediate the same intermolecular interactions (such as protein-protein, protein-DNA/RNA or protein-small molecule interactions) as a natural epitope found on the surface of a protein of interest.

The terms “polypeptide”, “oligopeptide”, “peptide” and “protein” are used interchangeably herein to refer to chains of amino acids of any length. The chain may be linear or branched, it may comprise modified amino acids, and/or may be interrupted by non-amino acids, The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Also included within the definition are polypeptides, oligopeptides, peptides and proteins having amino acid sequence identity to a given polypeptide, oligopeptide, peptide or protein. The percent identity can be, for example, at least about 50%, 55%, 60%, 65%, 70%, 75%, 80%, 85%, 90% 91%, 92%, 93%, 94%, 95%, 96%, 97%, 98%, 99%, or 100% amino acid identity to the given polypeptide, oligopeptide, peptide or protein. Also included within the definition are polypeptides, oligopeptides, peptides and proteins that have one or more conservative amino acid substitutions as compared to a given polypeptide, oligopeptide, peptide or protein. It is understood that the polypeptides can occur as single chains or associated chains. Methods for making polypeptides, oligopeptides, peptides and proteins are known in the art.

By the term “HSV surface protein” is meant any HSV surface protein occurring in a HSV. In one embodiment, the HSV surface protein is a surface envelope glycoprotein (gD). The term “HSV surface protein” also encompasses fragments and variants of such “HSV surface protein” molecules.

As used herein, “HSV-specific antibodies” means an antibody that binds to a target (e.g., HSV or an HSV surface protein) with greater affinity, avidity, more readily, and/or with greater duration than it binds to other substances. Also, an HSV-specific antibody “specifically binds” to a target (e.g., HSV or an HSV surface protein) if it binds with greater affinity, avidity, more readily, and/or with greater duration to that target in a sample than it binds to other substances present in the sample. For example, an antibody that specifically or preferentially binds to an HSV surface protein is an antibody that binds this protein with greater affinity, avidity, more readily, and/or with greater duration than it binds to other proteins.

The terms “anti-HSV” and “HSV-specific” may be used interchangeably to describe antibodies, including endogenous serum antibodies, that react with HSV-1 and/or HSV-2 surface antigens. Similarly, the terms “anti-DNP” and “DNP-specific” may be used interchangeably to describe antibodies, including endogenous serum antibodies, that react with dinitrophenyl.

The term “bind”, in the context of, for example, a target binding terminus (TBT) comprising at least one moiety that binds to a target protein, means an amino acid residue of the TBT that participates in an electrostatic interaction with the target protein, participates in a hydrogen bond with the target protein, or participates in a water-mediated hydrogen bond with the target protein, or participates in a salt bridge with the target protein, or it has a non-zero change in buried surface area due to interaction with the target protein, and/or a heavy atom of the TBT is located within 4A of a heavy atom of a residue of the target protein.

The term “bind”, in the context of, for example, one or more epitopes or epitope mimetics of a Herpes Simplex Virus (HSV) surface protein, means an amino acid residue of the ABT that participates in an electrostatic interaction with an HSV specific antibody, participates in a hydrogen bond with an HSV specific antibody, or participates in a water-mediated hydrogen bond with an HSV specific antibody, or participates in a salt bridge with an HSV specific antibody, or it has a non-zero change in buried surface area due to interaction with an HSV specific antibody, and/or a heavy atom of the ABT is located within 4A of a heavy atom of a residue of an HSV specific antibody.

The following symbol:

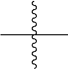

is used in chemical structures herein to represent a point of covalent attachment of a group to another group.

### General Techniques

The practice of the present disclosure will employ, unless otherwise indicated, conventional techniques of synthetic chemistry, organic chemistry, biochemistry, molecular biology, cell and tissue culture, immunology, genetics, etc. which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Sambrook and Russell, Molecular Cloning: A Laboratory Manual, 3rd. ed., Cold Spring Harbor Laboratory Press, Cold Spring Harbor, N Y (2001); Ausubel et al., Current Protocols in Molecular Biology, John Wiley & Sons, N Y (2002); Harlow and Lane Using Antibodies: A Laboratory Manual, Cold Spring Harbor Laboratory Press, Cold Spring Harbor, N Y (1998); Coligan et al., Short Protocols in Protein Science, John Wiley & Sons, N Y (2003); Short Protocols in Molecular Biology (Wiley and Sons, 1999); and Immunobiology (C. A. Janeway and P. Travers, 1997).

### Antibody Recruitment Molecules

Provided herein are low molecular weight, highly scalable and easily manufactured synthetic bi-functional ARM compounds that can simultaneously bind to both serum anti-HSV antibodies and tumor cell surface antigens. In one embodiment, the ARM compound has the structure of formula (I) or a pharmaceutically acceptable salt or solvate thereof:

(TBT)ₙ-(L)ₘ-(ABT)ₚ (I)

where: TBT is a target binding terminus comprising at least one moiety that binds to at least one target protein; L is an optional linker; ABT is an antibody binding terminus comprising at least one epitope or epitope mimetic of a Herpes Simplex Virus (HSV) surface protein; and each of n, m and p is independently 1 or any integer greater than 1.

The present disclosure provides ARM compounds, wherein the ABT comprises at least one epitope or epitope mimetic of an HSV surface protein. In certain embodiments, an epitope mimetic of an HSV surface protein is an in vitro synthesized derivative or mimic of a naturally-occurring epitope on the HSV surface protein. In certain embodiments, the ARM compounds provided herein comprise an epitope or epitope mimetic of an HSV surface glycoprotein. In certain embodiments, the ARM compounds comprise an epitope or epitope mimetic of HSV glycoprotein D (gD). In certain embodiments, the ARM compounds comprise an epitope or epitope mimetic of HSV glycoprotein D1 (gD1). The epitope or epitope mimetic of an HSV surface protein may react/interact with endogenous anti-HSV antibodies in the serum of a subject. The endogenous anti-HSV antibodies may be pre-existing in a subject, or the endogenous anti-HSV antibodies may be induced by treating a subject with an HSV-derived OV therapy, such as T-VEC.

In another embodiment, the ARM compound comprises at least one target binding terminus (TBT) comprising one or more moieties that bind to one or more target proteins; at least one antibody binding terminus (ABT) comprising one or more epitopes or epitope mimetics of a Herpes Simplex Virus (HSV) surface protein; and, optionally, at least one linker connecting the at least one TBT with the at least one ABT, or a pharmaceutically acceptable salt or solvate thereof.

Without being limited to any theory, these ARM compounds may improve the efficacy and/or reduce the toxicity of OV immunotherapy by: (i) sequestering endogenous anti-HSV antibodies that would normally be available to neutralize the OV, and/or (ii) redirecting endogenous anti-HSV antibodies to the tumor surface to elicit an anti-tumor immune response. The anti-tumor immune response may involve antibody-dependent cellular cytotoxicity (ADCC) and/or antibody-dependent cellular phagocytosis (ADCP) mechanisms.

In addition to cancer, the ARM compounds provided herein may also be useful for treating infectious diseases, by recruiting anti-HSV antibodies to a pathogen or a host cell that is infected with a pathogen, thereby enhancing the ability of the host's natural immune defenses to neutralize and clear the virus and/or the infected cells. In certain embodiments, the ARM compounds provided herein may also be useful as research reagents, diagnostic reagents, or as intermediates in the manufacture of other compounds.

In certain embodiments, the TBT comprises one or more moieties that bind to one or more target proteins. The target protein may be expressed on the surface of a cancer cell. The target may be, but is not limited to PSMA on prostate cancer cells, uPAR on glioblastoma cells, HER2 on breast cancer cells or ovarian cancer cells, or folate receptor on ovarian cancer cells.

In certain embodiments, the TBT comprises one or more of the following:
(i) glutamate urea ligand to target PSMA:

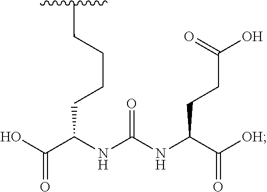 [SMILES: *CCCC[C@H](NC(=O)N[C@@H](CCC(=O)O)C(=O)O)C(=O)O]

(ii) cyclic peptides to target uPAR:

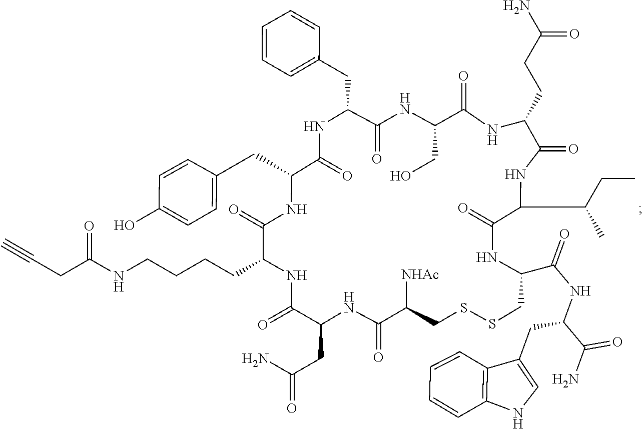 [SMILES: C#CCC(=O)NCCCC[C@H]1NC(=O)[C@H](CC(N)=O)NC(=O)[C@@H](NC(C)=O)CSSC[C@@H](C(=O)N[C@@H](Cc2c[nH]c3ccccc23)C(N)=O)NC(=O)C([C@@H](C)CC)NC(=O)[C@@H](CCC(N)=O)NC(=O)[C@H](CO)NC(=O)[C@@H](Cc2ccccc2)NC(=O)[C@@H](Cc2ccc(O)cc2)NC1=O]

(iii) trastuzumab (HERCEPTIN™) derived 9 amino acid peptide to target HER2:

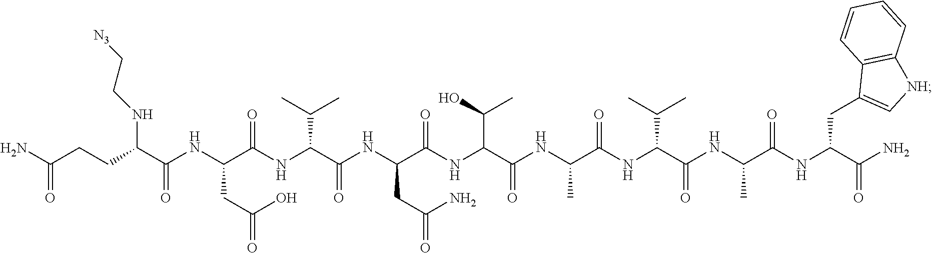 [SMILES: CC(C)[C@@H](NC(=O)[C@H](C)NC(=O)C(NC(=O)[C@@H](CC(N)=O)NC(=O)[C@H](NC(=O)[C@H](CC(=O)O)NC(=O)[C@H](CCC(N)=O)NCCN=[N+]=[N-])C(C)C)[C@H](C)O)C(=O)N[C@@H](C)C(=O)N[C@H](Cc1c[nH]c2ccccc12)C(N)=O]

(iv) methotrexate or folate to target the folate receptor;
(v) biotin or a derivative thereof to target avidin, streptavidin, neutravidin, or analogs thereof, wherein the biotin or the derivative thereof has the following structure:

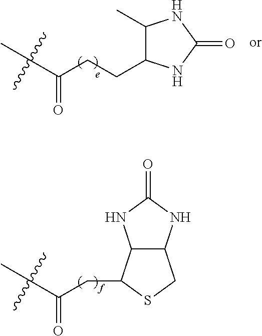

wherein e and f are, independently, an integer from 0 to 15; or
(vi) fluorescent reporter, such as fluorescein having the following structure:

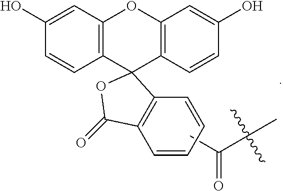

In an embodiment of the present disclosure, the ABT comprises one or more epitopes or epitope mimetics of an HSV surface protein. In an embodiment, the HSV surface protein is a glycoprotein on the viral cell surface. In an embodiment, the glycoprotein is HSV gD. In an embodiment, the glycoprotein is HSV gD1. In an embodiment, the ABT comprises the amino acid sequence set forth in any one of SEQ ID NOs: 1-9, or a variant thereof (see Table 1 below). In an embodiment, the ABT consists of the amino acid sequence set forth in any one of SEQ ID NOs: 1-9, or a variant thereof.

TABLE 1
ABT Peptide Sequences.

| SEQ ID NO | Sequence | ABT Module |
|---|---|---|
| 1 | LKMADPNRFRGKDL | |
| 2 | Ac-(N₃-K)-LKMADPNRFRGKDL | Peptide 1 (gD) |
| 3 | Ac-(N₃-K)-LRMADPNRFRGRDL | Peptide 2 (gDR) |
| 4 | Ac-(N₃-K)-LRMADPNRFRGRDLY | Peptide 3 (gDR-Y) |
| 5 | Ac-(N₃-K)-LRMADPNRFRGRDL-(Y-OSO₂F) | Peptide 4 (gDR-FSY) |
| 6 | Ac-(Y-OSO₂F)-(N₃-K)-LRMADPNRFRGRDL | Peptide 6 (FSY-gDR) |
| 7 | Ac-(N₃-K)-LRMADPNR-(Y-OSO₂F)-RGRDL | Peptide 5 (gDR(F10FSY)) |
| 8 | (Aryl-SO₂F)-(N₃-K)-LRMADPNRFRGRDL | Peptide 7 (ASF-gDR) |
| 9 | (Aryl-SO₃H)-(N₃-K)-LRMADPNRFRGRDL | Peptide 8 (SO₃H-gDR) |

In an embodiment, the linker is one or more polyethylene glycol (PEG) molecules linked together. For example, the linker may comprise 1, 2, 3, 4, 5, 6, 7, 8, 9, 10 or more PEG molecules linked together. In an embodiment, the linker comprises 4-10 PEG molecules linked together. In an embodiment, the linker comprises 4 PEG molecules linked together. In an embodiment, the linker comprises 5 PEG molecules linked together. In an embodiment, the linker comprises 6 PEG molecules linked together. In an embodiment, the linker comprises 7 PEG molecules linked together. In an embodiment, the linker comprises 8 PEG molecules linked together.

In an embodiment, the linker is a peptide or oligopeptide comprising 1 or more amino acid residues. For example, the linker may comprise 1, 2, 3, 4, 5, 6, 7, 8, 9, 10 or more amino acid residues linked together. In an embodiment, the linker is absent.

In an embodiment, the target protein is expressed on the surface of a pathogen or a cell infected with a pathogen. The pathogen maybe, for example, a virus, bacterium, fungus or parasite.

In an embodiment of the present disclosure, the ARM compound comprises:
one or more TBTs comprising a PSMA-targeting glutamate urea ligand, a uPAR-targeting cyclic peptide, a HER2-targeting trastuzumab-derived peptide, a folate receptor-targeting methotrexate or folate, an avidin/streptavidin/neutravidin-targeting biotin or derivative thereof, or a fluorescent reporter; and
one or more ABTs comprising one or more of the amino acid sequences set forth in any one of SEQ ID NOs: 1-9, or a variant thereof.

In an embodiment, the ARM compound comprises a TBT comprising PSMA-targeting glutamate urea ligand, a linker comprising 4-8 PEG molecules, and an ABT comprising the amino acid sequence set forth in SEQ ID NO: 1.

In an embodiment, the ARM compound comprises a TBT comprising PSMA-targeting glutamate urea ligand, a linker comprising 4-8 PEG molecules, and an ABT comprising the amino acid sequence set forth in SEQ ID NO: 2.

In an embodiment, the ARM compound comprises a TBT comprising PSMA-targeting glutamate urea ligand, a linker comprising 4-8 PEG molecules, and an ABT comprising the amino acid sequence set forth in SEQ ID NO: 3.

In an embodiment, the ARM compound comprises a TBT comprising PSMA-targeting glutamate urea ligand, a linker comprising 4-8 PEG molecules, and an ABT comprising the amino acid sequence set forth in SEQ ID NO: 6.

In an embodiment, the ARM compound comprises a TBT comprising PSMA-targeting glutamate urea ligand, a linker comprising 4-8 PEG molecules, and an ABT comprising the amino acid sequence set forth in SEQ ID NO: 8.

In an embodiment, the ARM compound comprises a TBT comprising desthiobiotin, a linker comprising 4-8 PEG molecules, and an ABT comprising the amino acid sequence set forth in SEQ ID NO: 1.

In an embodiment, the ARM compound comprises a TBT comprising desthiobiotin, a linker comprising 4-8 PEG molecules, and an ABT comprising the amino acid sequence set forth in SEQ ID NO: 2.

In an embodiment, the ARM compound comprises a TBT comprising desthiobiotin, a linker comprising 4-8 PEG molecules, and an ABT comprising the amino acid sequence set forth in SEQ ID NO: 3.

In an embodiment, the ARM compound comprises a TBT comprising biotin, a linker comprising 4-8 PEG molecules, and an ABT comprising the amino acid sequence set forth in SEQ ID NO: 1.

In an embodiment, the ARM compound comprises a TBT comprising biotin, a linker comprising 4-8 PEG molecules, and an ABT comprising the amino acid sequence set forth in SEQ ID NO: 2.

In an embodiment, the ARM compound comprises a TBT comprising biotin, a linker comprising 4-8 PEG molecules, and an ABT comprising the amino acid sequence set forth in SEQ ID NO: 3.

In an embodiment, the ARM compound comprises a TBT comprising biotin, a linker comprising 4-8 PEG molecules, and an ABT comprising the amino acid sequence set forth in SEQ ID NO: 4.

In an embodiment, the ARM compound comprises a TBT comprising biotin, a linker comprising 4-8 PEG molecules, and an ABT comprising the amino acid sequence set forth in SEQ ID NO: 5.

In an embodiment, the ARM compound comprises a TBT comprising biotin, a linker comprising 4-8 PEG molecules, and an ABT comprising the amino acid sequence set forth in SEQ ID NO: 6.

In an embodiment, the ARM compound comprises a TBT comprising biotin, a linker comprising 4-8 PEG molecules, and an ABT comprising the amino acid sequence set forth in SEQ ID NO: 7.

In an embodiment, the ARM compound comprises a TBT comprising biotin, a linker comprising 4-8 PEG molecules, and an ABT comprising the amino acid sequence set forth in SEQ ID NO: 8.

In an embodiment, the ARM compound comprises a TBT comprising fluorescein, a linker comprising 4-8 PEG molecules, and an ABT comprising the amino acid sequence set forth in SEQ ID NO: 3.

In an embodiment, the ARM compound comprises a TBT comprising fluorescein, a linker comprising 4-8 PEG molecules, and an ABT comprising the amino acid sequence set forth in SEQ ID NO: 5.

In an embodiment, the ARM compound comprises a TBT comprising fluorescein, a linker comprising 4-8 PEG molecules, and an ABT comprising the amino acid sequence set forth in SEQ ID NO: 6.

In an embodiment, the ARM compound comprises a TBT comprising fluorescein, a linker comprising 4-8 PEG molecules, and an ABT comprising the amino acid sequence set forth in SEQ ID NO: 7.

In an embodiment, the ARM compound comprises a TBT comprising fluorescein, a linker comprising 4-8 PEG molecules, and an ABT comprising the amino acid sequence set forth in SEQ ID NO: 8.

The TBT, linker and ABT may be conjugated via orthogonal “click” chemistry protocols known in the art, including, but not limited to strain-promoted azide-alkyne cycloaddition (SPAAC) reaction, copper(I)—catalyzed azide-alkyne cycloaddition (CuAAC) reaction, inverse electron demand Diels-Alder (IEDDA) reaction, or sulfur (VI) fluoride exchange reaction (SuFEx), to facilitate rapid, efficient and versatile conjugation of the various fragments forming the ARM compound. These conjugation methods are versatile and adaptable towards generating multivalent viral targeting ARM scaffolds that contain a plurality of targeting modules (TBTs) and, optionally, a plurality of antibody-recruiting modules (ABTs).

The ARM compounds may be further modified using covalent immune recruiting (CIR) technology to bind endogenous antibodies with “infinite” affinity, if necessary to increase the potency and efficacy of antibody recruitment by the ARM compounds. CIR technology involves the ARM-mediated formation of selective covalent linkages to the serum HSV-specific antibodies directly in vivo. The result is that the antibodies can no longer dissociate from the bi-functional molecule which can have significant pharmacokinetic and functional consequences. CIR technology is known in the art and is described in detail, for example, by Lake et al, “Covalent Immune Recruiters: Tools to Gain Chemical Control Over Immune Recognition” (2020) 15 ACS Chem Biol 1089-1095, which is incorporated herein by reference as if set forth in its entirety.

In certain embodiments of the ARM compounds provided herein, covalent antibody recruitment can be achieved by modifying the compound to include one or more reactive groups that mediate covalent conjugation of the compound with an HSV-specific antibody and/or the target protein. In certain embodiments, the HSV-specific antibody is a serum antibody. In certain embodiments, the reactive group comprises an electrophilic functional group that reacts with an amino acid nucleophile in a nucleophilic substitution reaction.

Any part of the ARM compound may be modified to contain a covalently reactive group. In certain embodiments of the disclosure, the reactive group is present on the ABT, the linker, or the TBT. In an embodiment, the reactive group is present on the ABT. In an embodiment, the reactive group is present on the linker. In an embodiment, the reactive group is present on the TBT.

In certain embodiments, the reactive group comprises an acyl imidazole group having the following structure:

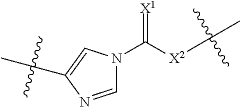

where: X¹ is S, O or NR¹; X² is O or NR²; and R¹ and R² are independently H or C₁₋₄ alkyl.

In certain embodiments, the reactive group comprises a fluorosulfate-I-tyrosine (FSY) group (OSO₂F), or an aryl-sulfonyl fluoride (ASF) group (SO₂F).

In certain embodiments, the cancer is prostate cancer, the target protein is PSMA, the TBT comprises a glutamate urea ligand, the ABT comprises the amino acid sequence set forth in any one of SEQ ID NOs: 1-9, or a variant thereof, and the compound has the following structure:

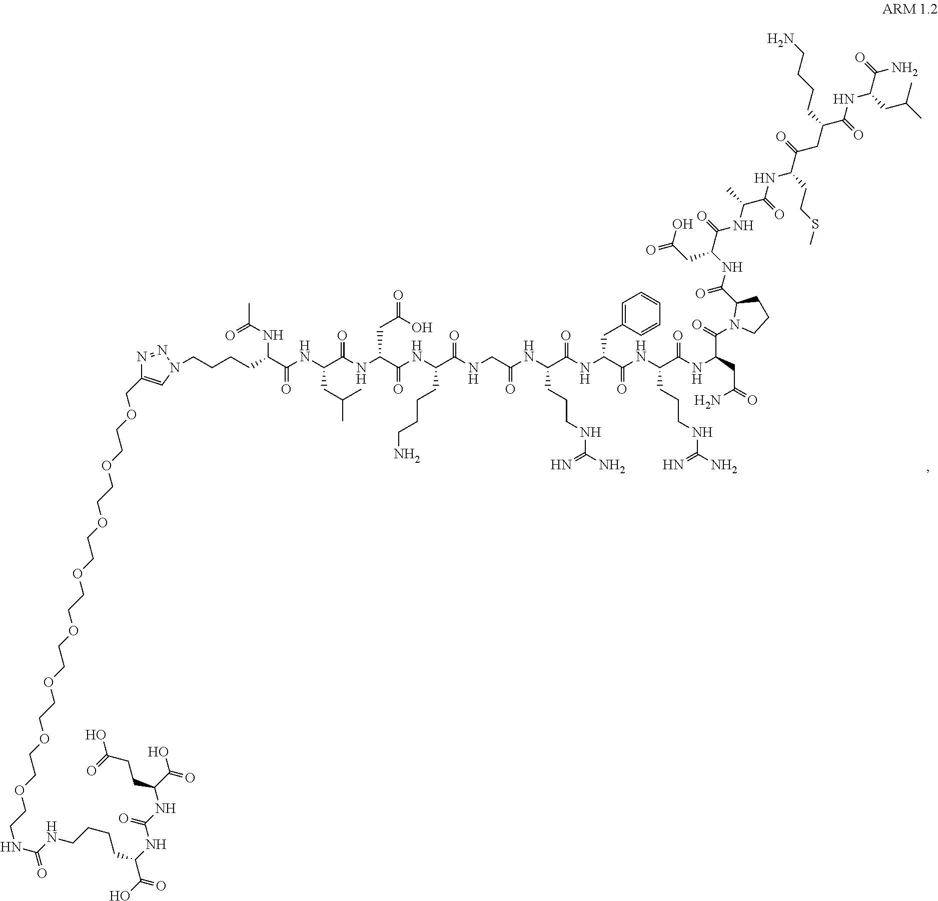 [SMILES: CSCC[C@H](NC(=O)[C@@H](C)NC(=O)[C@@H](CC(=O)O)NC(=O)[C@H]1CCCN1C(=O)[C@@H](CC(N)=O)NC(=O)[C@H](CCCNC(=N)N)NC(=O)[C@@H](Cc1ccccc1)NC(=O)[C@H](CCCNC(=N)N)NC(=O)CNC(=O)[C@H](CCCCN)NC(=O)[C@@H](CC(=O)O)NC(=O)[C@H](CC(C)C)NC(=O)[C@H](CCCCn1cc(COCCOCCOCCOCCOCCOCCOCCOCCNC(=O)NCCCC[C@H](NC(=O)N[C@@H](CCC(=O)O)C(=O)O)C(=O)O)nn1)NC(C)=O)C(=O)C[C@H](CCCCN)C(=O)N[C@@H](CC(C)C)C(N)=O]

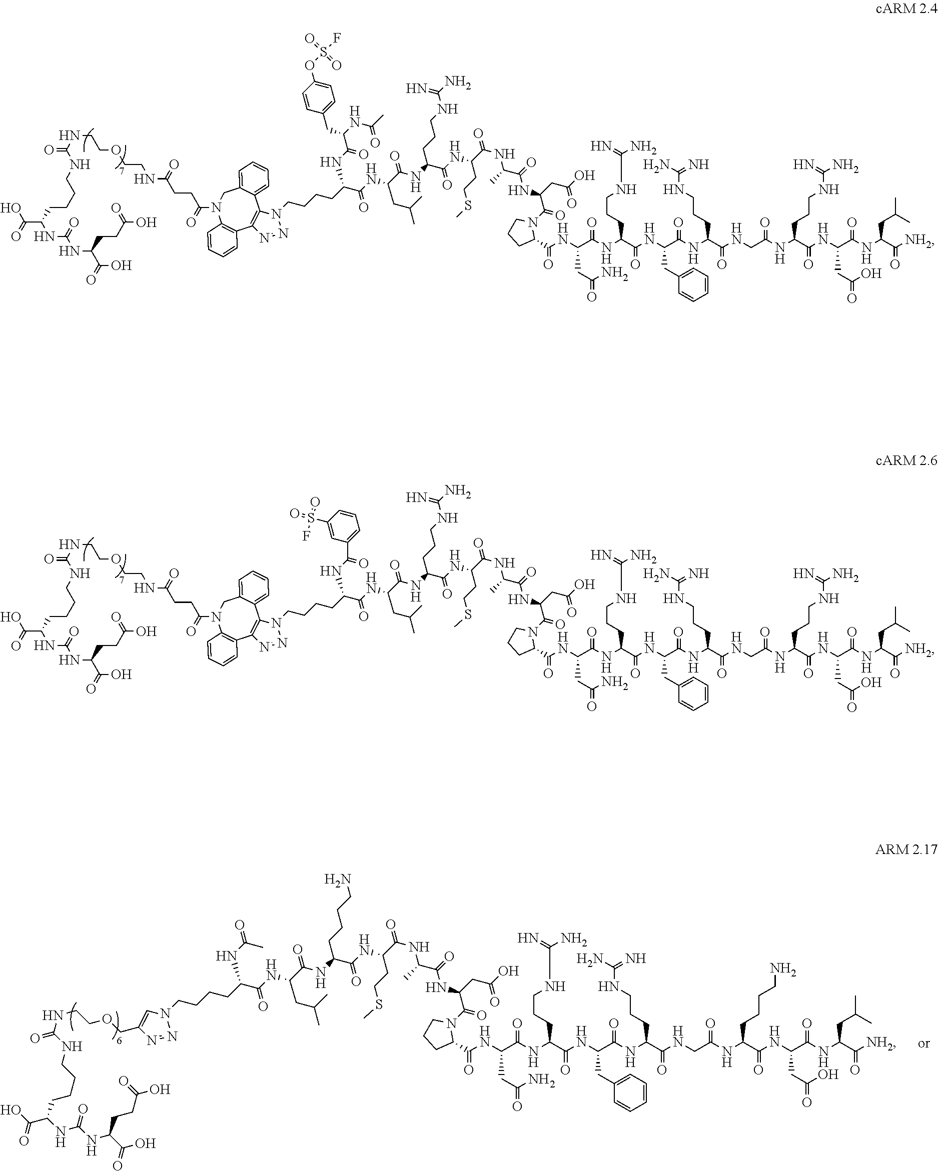

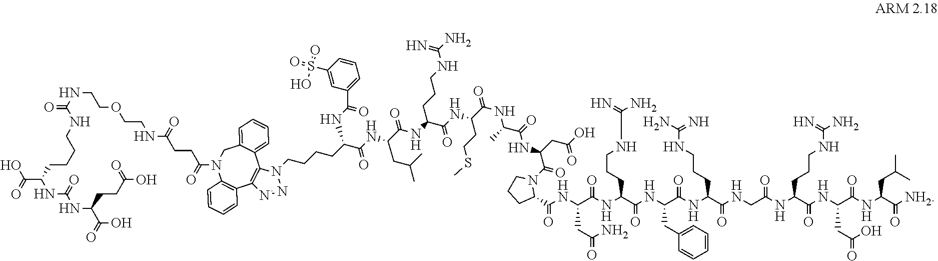 [SMILES: CSCC[C@H](NC(=O)[C@H](CCCNC(=N)N)NC(=O)[C@H](CC(C)C)NC(=O)[C@H](CCCCn1nnc2c1-c1ccccc1CN(C(=O)CCC(=O)NCCOCCNC(=O)NCCCC[C@H](NC(=O)N[C@@H](CCC(=O)O)C(=O)O)C(=O)O)c1ccccc1-2)NC(=O)c1cccc(S(=O)(=O)O)c1)C(=O)N[C@@H](C)C(=O)N[C@@H](CC(=O)O)C(=O)N1CCC[C@H]1C(=O)N[C@@H](CC(N)=O)C(=O)N[C@@H](CCCNC(=N)N)C(=O)N[C@@H](Cc1ccccc1)C(=O)N[C@@H](CCCNC(=N)N)C(=O)NCC(=O)N[C@@H](CCCNC(=N)N)C(=O)N[C@@H](CC(=O)O)C(=O)N[C@@H](CC(C)C)C(N)=O]

In certain embodiments, such as diagnostic embodiments, the target protein is avidin, streptavidin, neutravidin or an analog thereof, the TBT comprises biotin or a derivative thereof, the ABT comprises the amino acid sequence set forth in any one of SEQ ID NOs: 1-9, or a variant thereof, and the compound has the following structure:

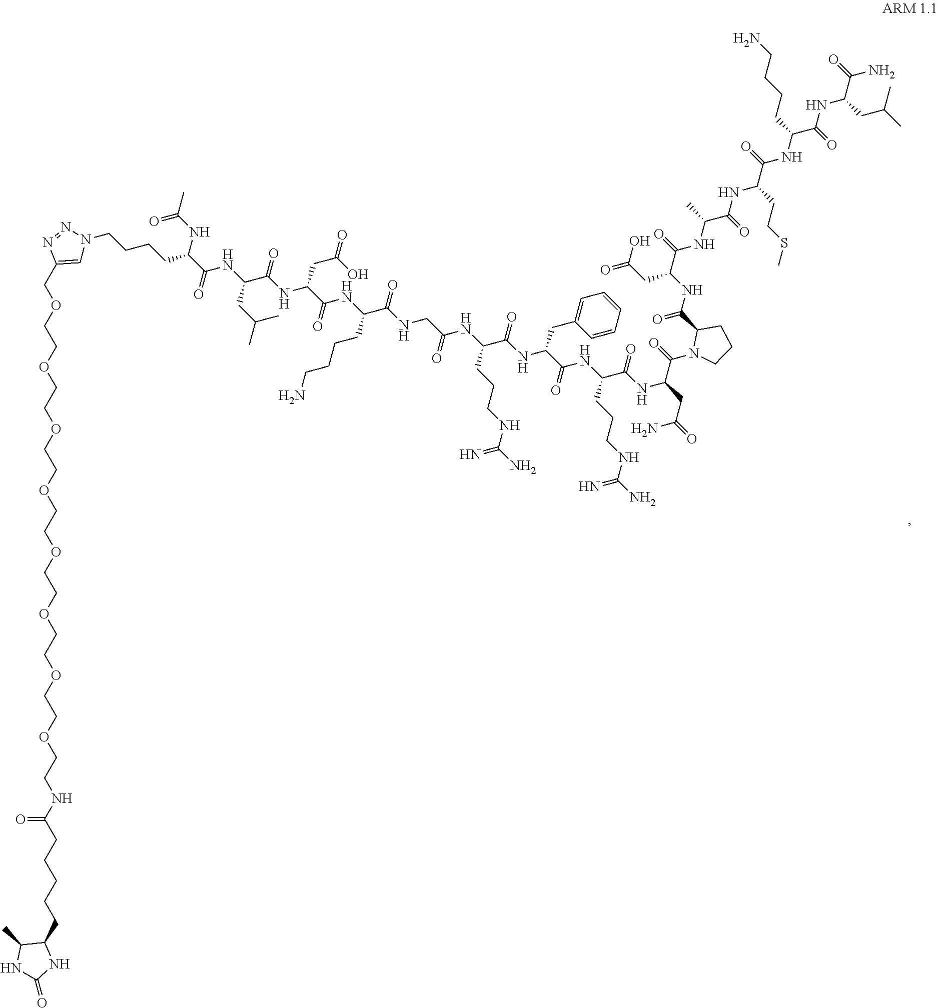 [SMILES: CSCC[C@H](NC(=O)[C@@H](C)NC(=O)[C@@H](CC(=O)O)NC(=O)[C@H]1CCCN1C(=O)[C@@H](CC(N)=O)NC(=O)[C@H](CCCNC(=N)N)NC(=O)[C@@H](Cc1ccccc1)NC(=O)[C@H](CCCNC(=N)N)NC(=O)CNC(=O)[C@H](CCCCN)NC(=O)[C@@H](CC(=O)O)NC(=O)[C@H](CC(C)C)NC(=O)[C@H](CCCCn1cc(COCCOCCOCCOCCOCCOCCOCCOCCNC(=O)CCCCC[C@H]2NC(=O)N[C@H]2C)nn1)NC(C)=O)C(=O)N[C@H](CCCCN)C(=O)N[C@@H](CC(C)C)C(N)=O]

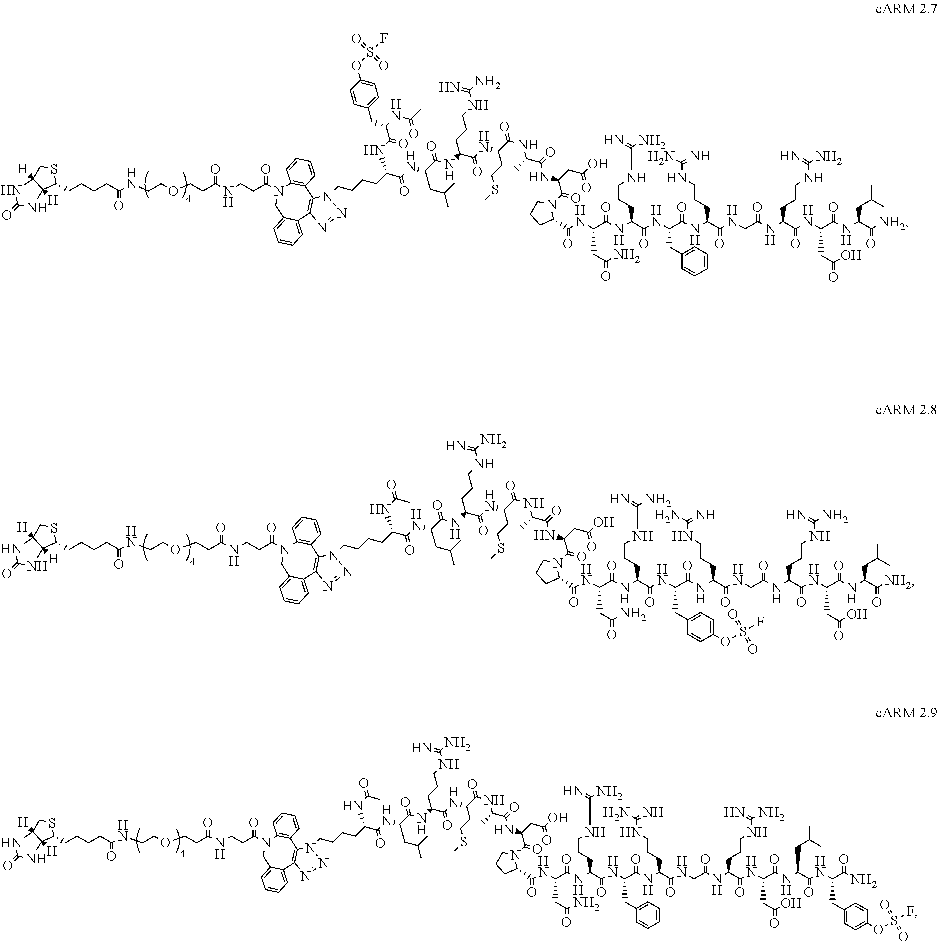

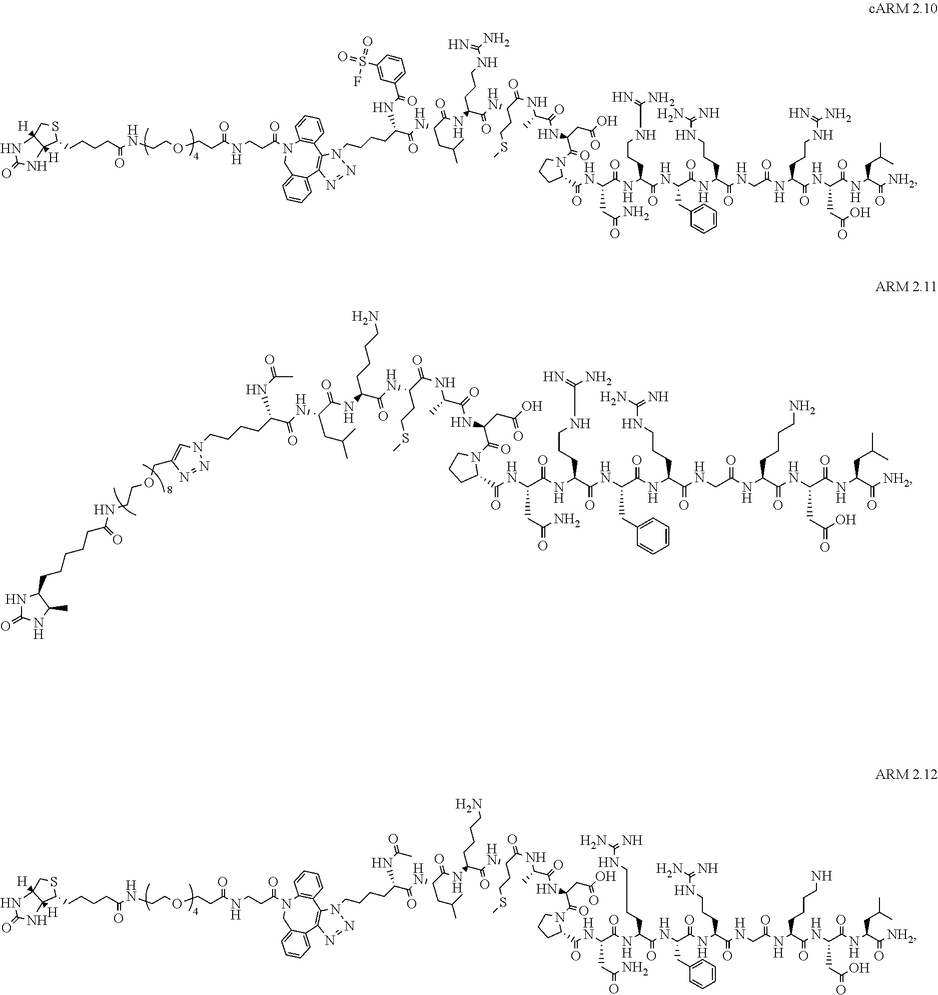

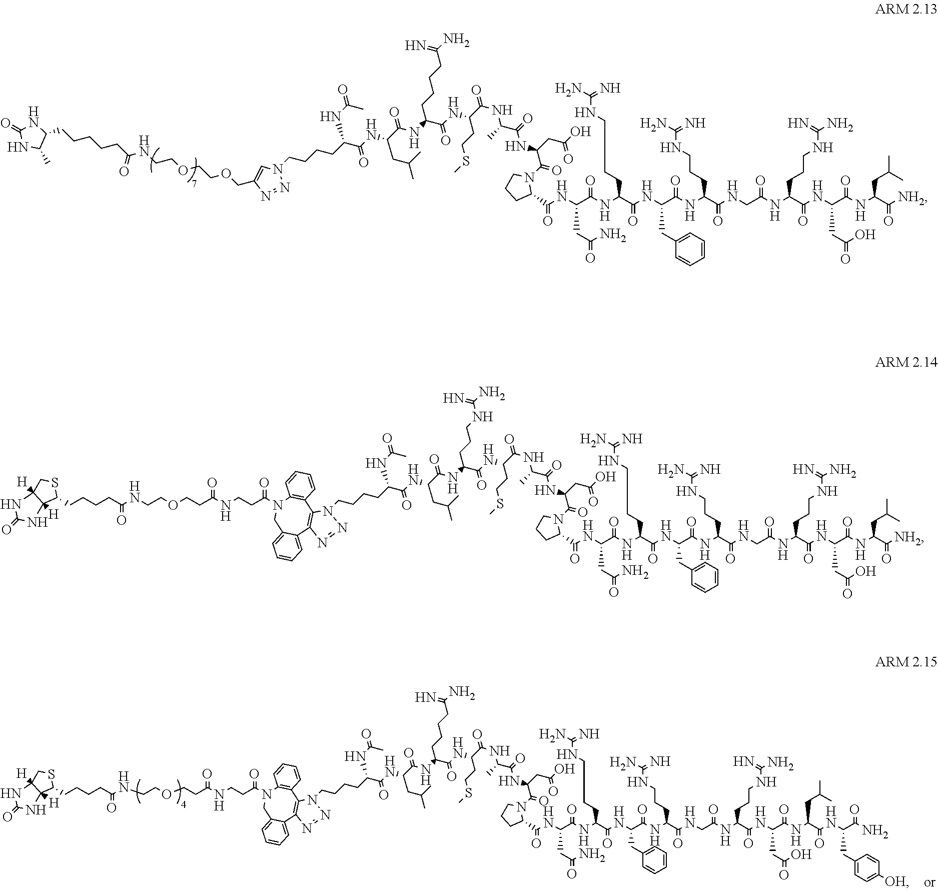

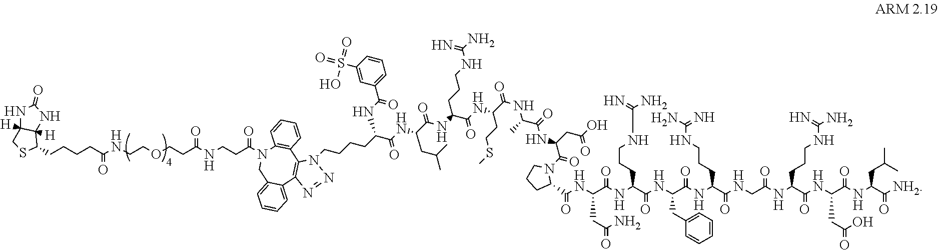

In certain embodiments, such as diagnostic embodiments, the TBT comprises a fluorescent reporter and the ABT comprises the amino acid sequence set forth in any one of SEQ ID NOs: 1-9. The fluorescent reporter may be any chemical group or molecule (synthetic, recombinant or natural in origin) that emits light upon excitation and is suitable for detection by, for example, fluorescence imaging, fluorescence microscopy, flow cytometry, fluorescence spectroscopy, etc. Numerous fluorescent reporters with a variety of excitation and emission spectra are known in the art. The selection of a particular fluorescent reporter for a given task (such as a diagnostic assay or an analytical assay) is within the purview of a person of ordinary skill in the art. In certain embodiments, the fluorescent reporter is fluorescein.

In certain embodiments, the TBT comprises fluorescein, the ABT comprises the amino acid sequence set forth in any one of SEQ ID NOs: 1-9, and the compound has the following structure:

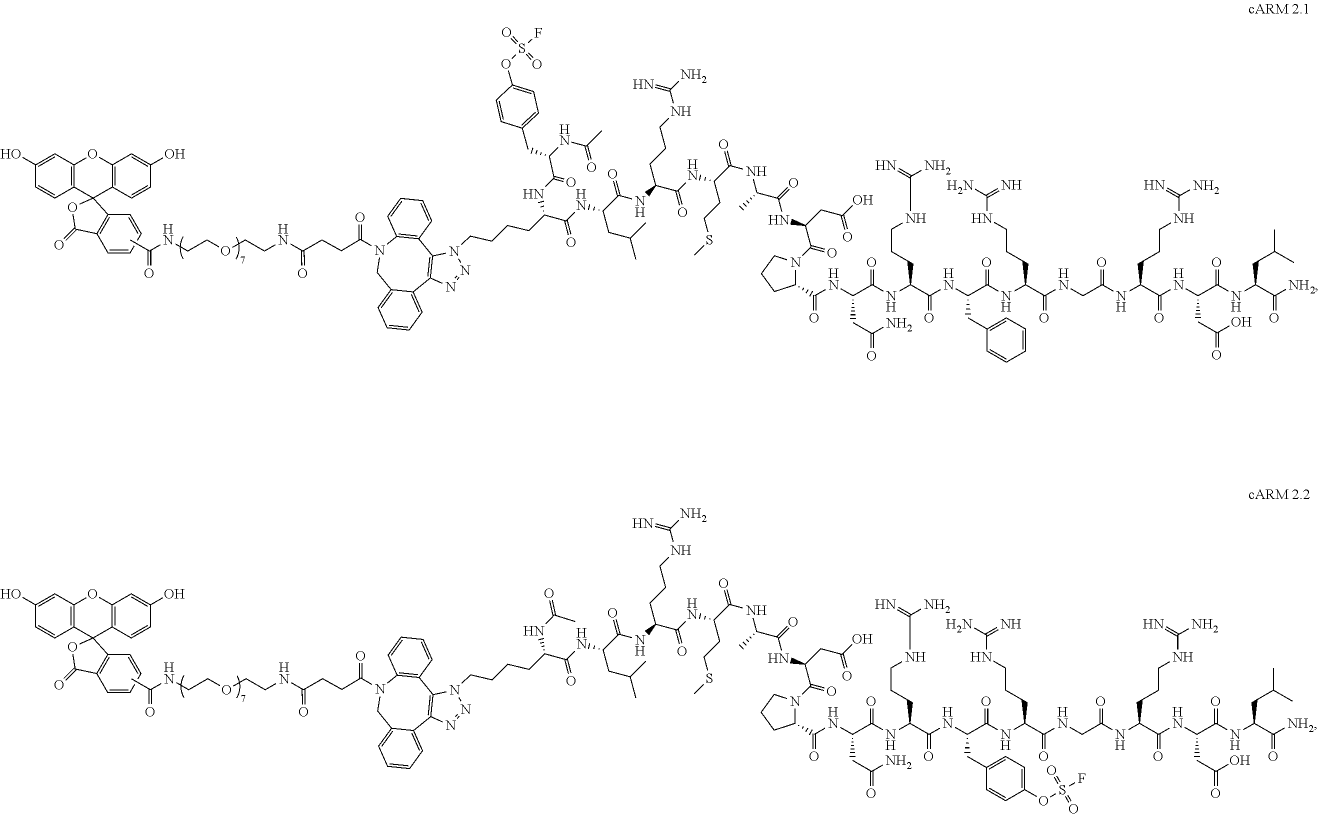

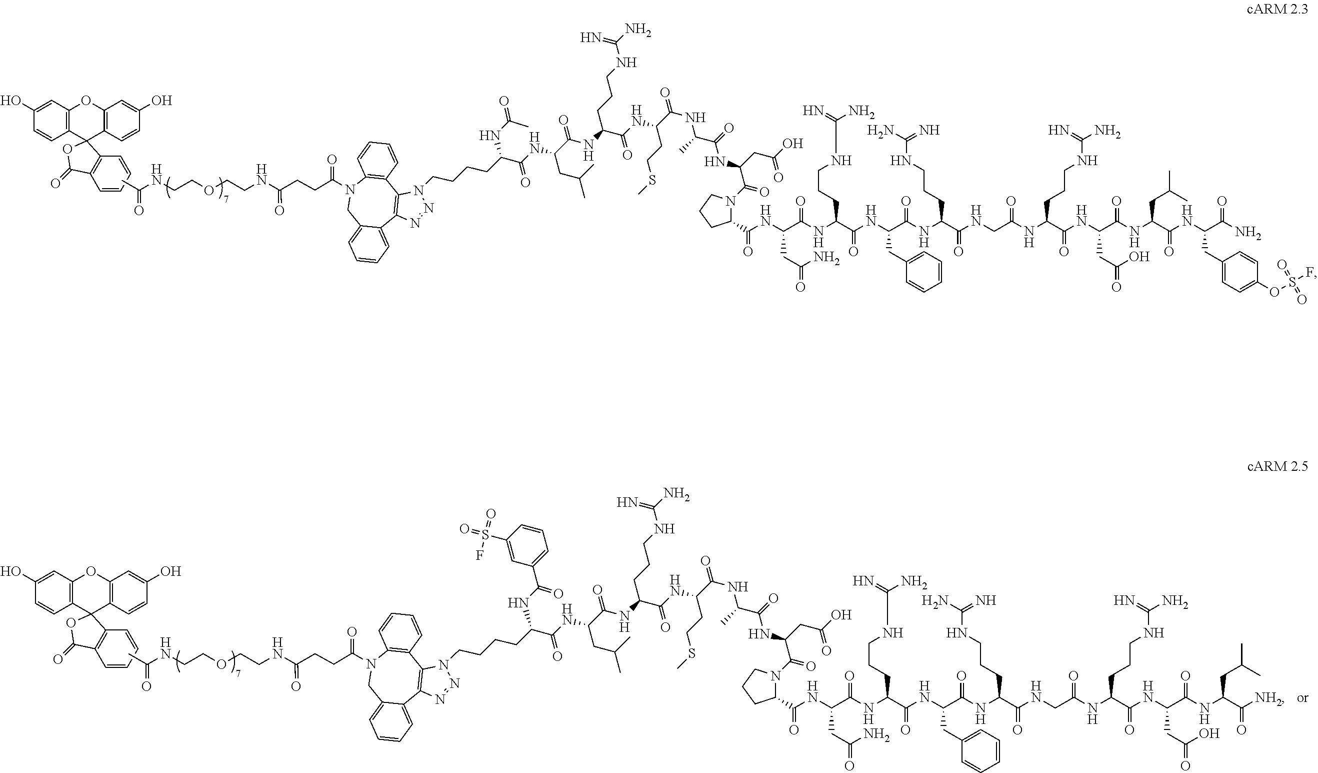

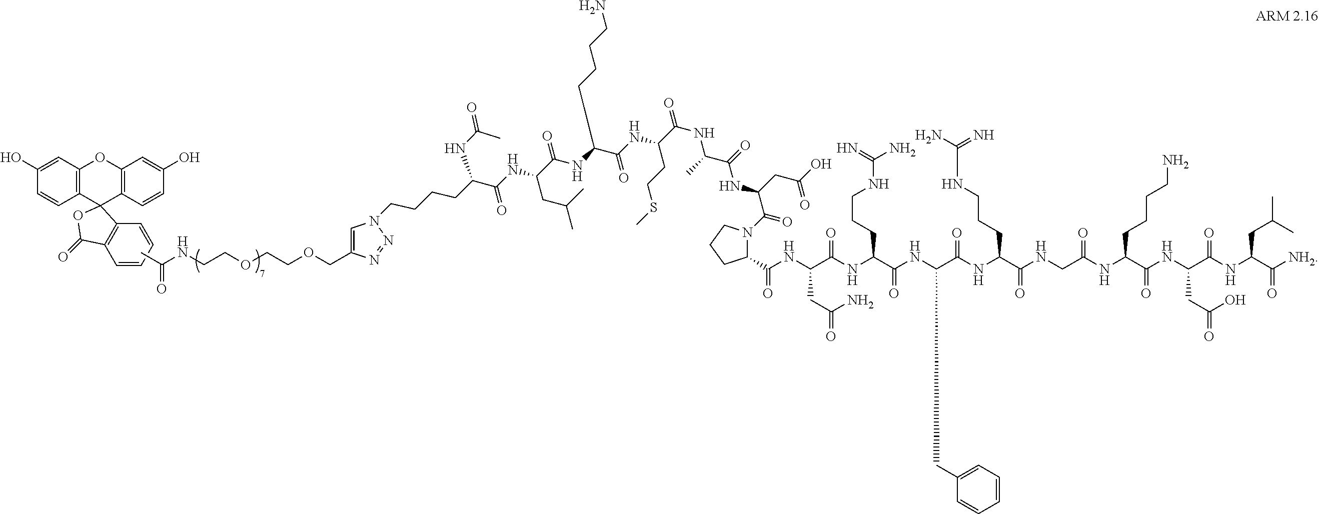

### Methods of Preparing Antibody Recruitment Molecules

The ARM compounds disclosed herein can be prepared by various synthetic processes. The choice of particular structural features and/or substituents may influence the selection of one process over another. The selection of a particular process to prepare an ARM compound is within the purview of the person of skill in the art. Some starting materials for preparing compounds of the present disclosure are available from commercial chemical sources. Other starting materials, for example as described below, are readily prepared from available precursors using straightforward transformations that are well known in the art.

The compounds of the invention may be synthesized according to the methods and protocols disclosed herein. In an embodiment, the ARM compounds may be synthesized using standard chemical connectivity between the linker(s), the TBT(s) and the ABT(s), along with appropriate protecting groups when necessary. In an embodiment, the approach uses standard functional group chemistry in order to link the TBT to the ABT through a linker to obtain ARM compounds.

In an embodiment, the ABT comprises a peptide which may be synthesized using, for example, solid phase peptide synthesis (SPPS). In an embodiment, the ABT comprises a peptide which may be expressed/overexpressed in cells using standard molecular biology techniques, and then purified using standard peptide purification protocols known in the art.

Standard functional group chemistries, that may be used in the preparation of compounds of the application, include, for example, coupling a carboxylic acid to either an amine or an alcohol to generate esters or amides through standard carbodiimide conditions (e.g., DCC, EDCI, DIC) along with base and catalytic amine (e.g., DMAP, imidazole), or by conversion to the acid chloride through oxalyl chloride or thionyl chloride, etc., followed by addition of amine/alcohol.

Additionally, for example, an amine or an alcohol may be coupled to an isocyanate or an isothiocyanate to generate ureas, thioureas, or the corresponding carbonates or thiocarbonates.

Still, in a further approach, for example, a heterolinker can be made through treating a nucleophile with the appropriate leaving group. Some leaving groups could be halogens, such as bromine, or sulfonates, such as triflates or tosylates.

The ARM compounds of the disclosure may include one or more pharmaceutically acceptable salts. The formation of a desired compound salt is achieved using standard techniques. For example, the neutral compound is treated with an acid or base in a suitable solvent and the formed salt is isolated by filtration, extraction or any other suitable method. Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxyalkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N, 1\r-dibenzylethylenediamine, N-methylglucamine, chloroprocaine, choline, diethanolamine, ethylenediamine, procaine and the like.

The ARM compounds of the disclosure may be solvates of the ARM compounds. The term “solvate” as used herein means a compound, or a salt of a compound, wherein molecules of a suitable solvent are incorporated in the crystal lattice. The formation of solvates of the compounds provided herein will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art. Examples of suitable solvents are ethanol, water and the like. When water is the solvent, the molecule is referred to as a “hydrate”.

As used herein “pharmaceutically acceptable” means compatible with the treatment, diagnosis or analysis of subjects.

Throughout the processes described herein it is to be understood that, where appropriate, suitable protecting groups will be added to, and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include, for example, straight and reversed phase chromatography on column or rotating plate, recrystallisation, distillation and liquid-liquid or solid-liquid extraction, which will be readily understood by those skilled in the art.

### Compositions Comprising Antibody Recruitment Molecules

The ARM compounds of the present invention may be in the form of a pharmaceutical composition comprising the ARM compound and at least one pharmaceutically acceptable carrier, diluent, excipient or stabilizer (Remington: The Science and practice of Pharmacy 20th Ed., 2000, Lippincott Williams and Wilkins, Ed. K. E. Hoover), in the form of lyophilized formulations or aqueous solutions. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Pharmaceutically acceptable carriers, diluents, excipients and stabilizer are further described herein.

The ARM compounds and compositions thereof can also be used in conjunction with other agents that serve to enhance and/or complement the effectiveness of the agents.

A pharmaceutical composition of the disclosure also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and non-aqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutical compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be suitable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

### Methods of Treating Cancer

The ARM compounds and pharmaceutical compositions provided herein may be administered to a subject in order to recruit an HSV-specific antibody to a cancer cell in the subject or to recruit an HSV-specific antibody to a pathogen or a cell infected with a pathogen in a subject.

Provided herein are chemical strategies to selectively inhibit endogenous serum antibodies from neutralizing an administered OV and/or redirect the endogenous serum antibodies to target the tumor itself, in order to both enhance current OV immunotherapy and add another arm of anti-tumor mode of action.

The recruitment of serum HSV-specific antibodies to the tumor surface can activate host NK cells and/or macrophages to elicit antibody dependent cellular phagocytosis (ADCP) and/or antibody dependent cellular cytotoxicity (ADCC) tumor-killing mechanisms. These mechanisms can also help stimulate the adaptive anti-tumor immune response.

The present disclosure provides: (i) methods of treating cancer in a subject and (ii) methods for enhancing the efficacy and/or reducing the toxicity of an OV therapy in a subject with cancer. The methods provided herein generally comprise administering an effective amount of a compound comprising: at least one target binding terminus (TBT) comprising one or more moieties that bind to one or more target proteins on the cancer; at least one antibody binding terminus (ABT) comprising one or more epitopes or epitope mimetics of a Herpes Simplex Virus (HSV) surface protein; and, optionally, at least one linker connecting the at least one TBT with the at least one ABT or a pharmaceutically acceptable salt or solvate thereof, and an OV therapy to the subject.

The ARM compounds, oncolytic viruses and pharmaceutical compositions provided herein may be administered to a subject in an effective amount or a therapeutically effective amount. A person of ordinary skill in the art would be able to determine such amounts based on such factors as the subject's size (e.g., weight), age and/or sex; the nature of the disease to be treated (e.g., the type of cancer); the severity of the subject's symptoms (e.g., the grade or clinical stage of the cancer); and the particular composition or route of administration selected. A person skilled the art would also know how to select the proper route of administration and to administer the compounds and compositions provided herein.

Selected routes of administration for antibodies of the disclosure include intravenous, intramuscular, intradermal, intraperitoneal, subcutaneous, spinal or other parenteral routes of administration, for example, by injection or infusion. Parenteral administration may represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a composition of the disclosure can be administered via a non-parenteral route, such as a topical, epidermal or mucosal route of administration, for example, intranasally, orally, vaginally, rectally, sublingually or topically.

If the ARM compounds and compositions, of the disclosure are administered in a controlled release or sustained release system, a pump may be used to achieve controlled or sustained release (see Langer, supra; Sefton, 1987, CRC Crit. Ref. Biomed. Eng. 14:20; Buchwald et al., 1980, Surgery 88:501; Saudek et al., 1989, N. Engl. J. Med. 321:514).

In certain embodiments, the ARM compounds or pharmaceutical compositions comprising the ARM compounds may be administered to the subject before, concurrently with, and/or after administration of the OV therapy.

The dosage of an ARM compound and oncolytic virus of the disclosure varies depending on many factors, such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. In some embodiments, an ARM compound is administered initially in a suitable dosage that is adjusted as required, depending on the clinical response. Dosages may generally be selected to maintain a serum level of the compound of the ARM compound from about 0.01 μg/cc to about 1000 μg/cc, about 0.1 μg/cc to about 100 μg/cc, or about 0.1 μM to about 10 μM. As a representative example, oral dosages of one or more compounds provided herein may range between about 1 mg per day to about 1000 mg per day for an adult. For parenteral administration (e.g., IV administration), a representative amount is from about 0.001 mg/kg to about 10 mg/kg, about 0.01 mg/kg to about 10 mg/kg, about 0.01 mg/kg to about 1 mg/kg or about 0.1 mg/kg to about 1 mg/kg can be administered. For oral administration, a representative amount is from about 0.001 mg/kg to about 10 mg/kg, about 0.1 mg/kg to about 10 mg/kg, about 0.01 mg/kg to about 1 mg/kg or about 0.1 mg/kg to about 1 mg/kg. For administration in suppository form, a representative amount is from about 0.1 mg/kg to about 10 mg/kg or about 0.1 mg/kg to about 1 mg/kg.

Additional therapies (e.g., prophylactic or therapeutic agents), which can be administered in combination with the ARM compounds and compositions of the disclosure, may be administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours, 1 week, 2, weeks, 3 weeks, 4 weeks, 5 weeks, 6 weeks, 7 weeks or 8 or more weeks apart from the ARM compounds and compositions of the disclosure, and administration can be in either order. The two or more therapies may be administered within the same patient visit or on different patient visits.

The ARM compounds and compositions of the disclosure and the other therapies may be cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time, optionally, followed by the administration of a third therapy (e.g., prophylactic or therapeutic agent) for a period of time and so forth, and repeating this sequential administration, i.e., the cycle in order to reduce the development of resistance to one of the therapies, to avoid or reduce the side effects of one of the therapies, and/or to improve the efficacy of the therapies.

The prophylactic or therapeutic agents of the combination therapies can be administered to a subject in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration.

### Kits

The invention also provides kits comprising any or all of the ARM compounds described herein. Kits of the invention include one or more containers comprising an ARM compound described herein and instructions for use in accordance with any of the methods of the invention described herein. Generally, these instructions comprise a description of administration of the ARM compound for the above described therapeutic treatments. In some embodiments, kits are provided for producing a single-dose administration unit. In certain embodiments, the kit can contain both a first container having a dried ARM compound and a second container having an aqueous formulation. In certain embodiments, kits containing an applicator, e.g., single and multi-chambered pre-filled syringes (e.g., liquid syringes and lyosyringes), are included.

The instructions relating to the use of an ARM compound generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an ARM compound of the invention. The container may further comprise a second pharmaceutically active agent.

In certain embodiments, the kits of the invention further comprise an oncolytic virus, for example, an oncolytic HSV (T-VEC) in addition to the ARM compound(s). In an embodiment of the kits provided herein, the ARM compound(s) and the OV are formulated together and provided in a single container. In an embodiment of the kits provided herein, the ARM compound(s) and the OV are formulated separately and provided in two or more containers.

Kits may optionally provide additional components such as buffers and interpretive information. Normally, the kit comprises a container and a label or package insert(s) on or associated with the container.

### EXAMPLES

The disclosure is further described by reference to the following examples. These examples are provided for purposes of illustration only and are not intended to be limiting unless otherwise specified. Thus, the disclosure should in no way be construed as being limited to the following examples, but rather, should be construed to encompass any and all variations which become evident as a result of the teaching provided herein.

### Example 1: Model of Antibody Recruitment Activity In Vivo

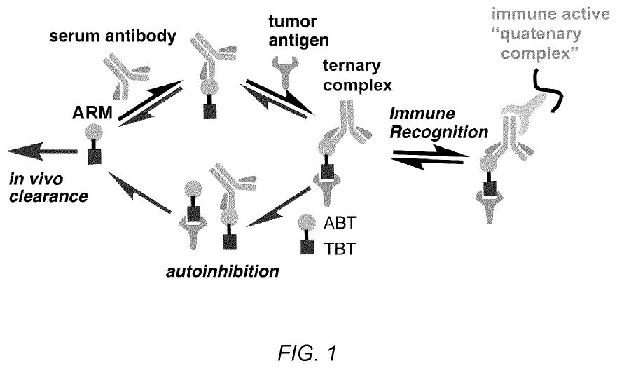

FIG. 1 is a schematic model of the in vivo mechanisms of action of an ARM compound. Simultaneous binding of the ABT to a serum antibody and the TBT to a tumor antigen results in the formation of a ternary complex at the site of the tumor. This ternary complex may in turn react with an immune cell, such as an NK cell, via serum antibody Fc-mediated interactions, to form a quaternary complex and elicit anti-tumor immune responses.

However, quaternary complex formation cannot be increased by administering excess ARM compound, as this tends to lead to the formation of autoinhibitory complexes where both the tumor cell surface and immune cell surface are saturated with the ARM compound.

### Example 2: Synthesis of Antibody Recruitment Molecules 1.1 and 1.2

Desthiobiotin-PEG8-Propargyl:

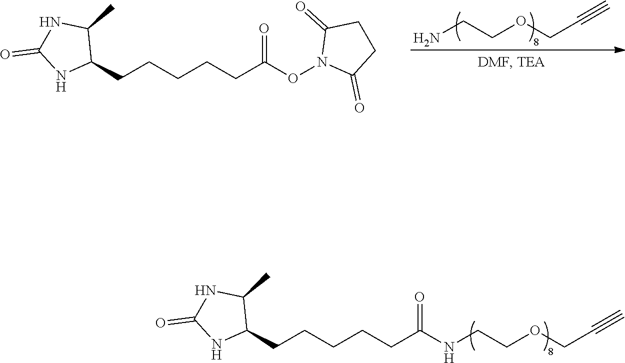

Desthiobiotin-NHS (0.138 mmol, 1 eq) was transferred to a vial with a stir bar and evacuated with argon. Propargyl-PEG8-NH2 (0.152 mmol, 1.1 eq) was added to this, followed by anhydrous DMF (3 mL), and then TEA (0.414 mmol, 3 eq). The reaction was allowed to proceed over 24 hours. The reaction was purified on a normal phase 12 g Buchi EcoFlex silica column using a shallow gradient of 5% to 20% methanol in dichloromethane. The final product was obtained in 71.17% yield. ¹H NMR (700 MHz, CDCl₃) δ 6.55 (s, 1H), 5.18 (s, 1H), 4.66 (s, 1H), 4.20 (d, J=2.5 Hz, 2H), 3.84 (m, 1H), 3.68 (m, 4H), 3.65-3.64 (m, 24H), 3.62 (m, 4H), 3.55 (t, J=5.0 Hz, 2H), 3.44 (q, J=5.2 Hz, 2H), 2.70 (s, 1H), 2.43 (t, J=2.4 Hz, 1H), 2.19 (t, J=7.4 Hz, 2H), 1.65 (p, J=7.3 Hz, 2H), 1.38 (m, 6H), 1.12 (d, J=6.5 Hz, 3H). LCMS confirmed correct expected mass of product. MS-ESI [M+H]⁺ m/z calc for [C₂₉H₅₃N₃O₁₀]604.37, found 604.4766.

qD1 Peptide:

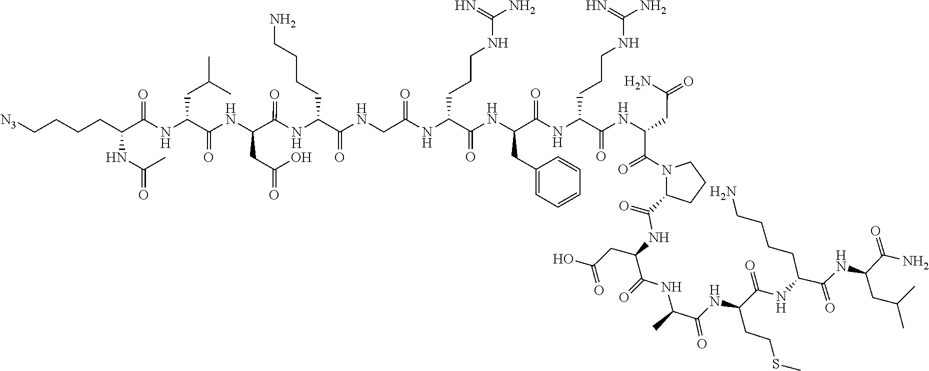

The synthesized peptide was left crude and eluted at 2.98 minutes using a 10-minute LCMS run with an acetonitrile gradient ranging from 5% for the first minute, increasing to 95% by 7.5 minutes, and then returning to 5% by 10 minutes. LCMS confirmed correct expected mass of product. MS-ESI [M+4H]⁴⁺ m/z calc for [C₈₀H₁₃₄N₂₈O₂₁S] 465.05, found 465.1765.

Desthiobiotin-PEG8-qD1 (ARM 1.1):

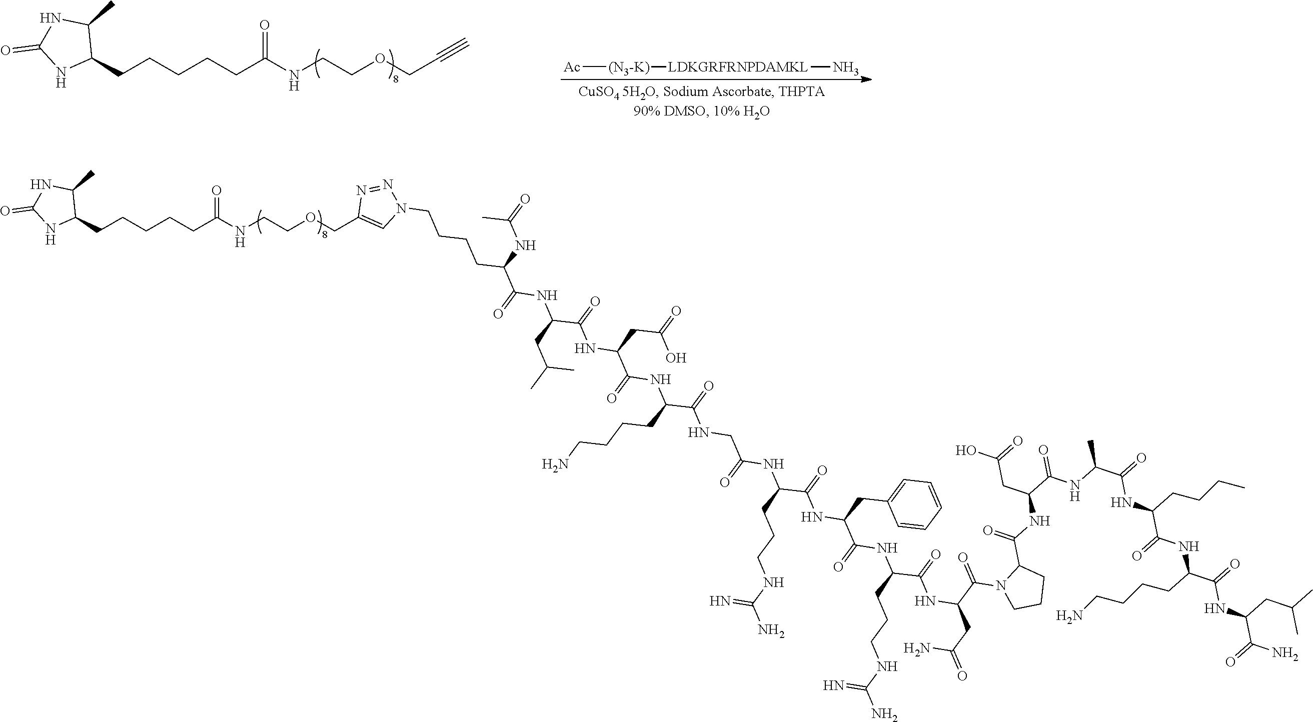

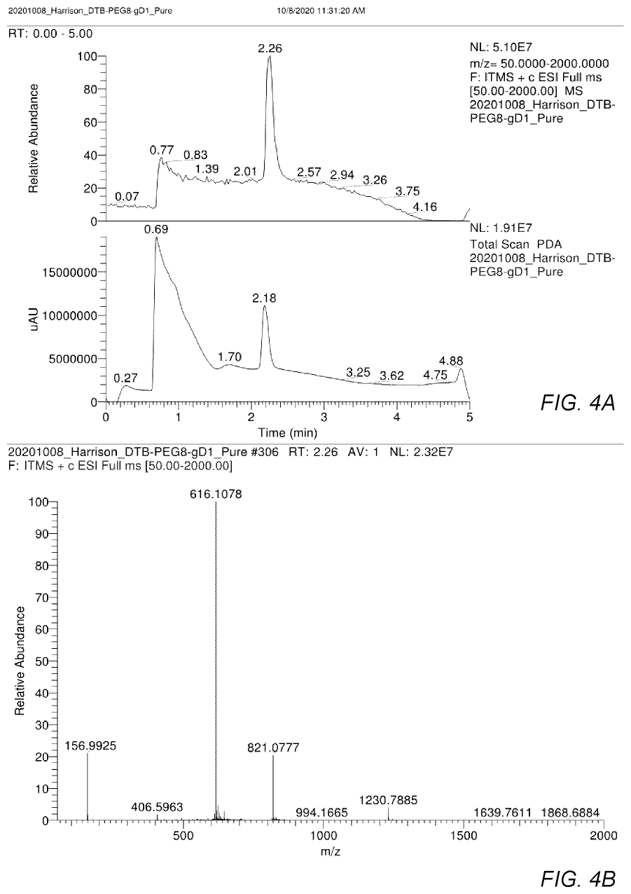

Both desthiobiotin-PEG8-propargyl and the crude gD1 peptide were transferred to a vial with a stir bar. The vial was evacuated with argon and the DMSO was added. The CuSO₄ pentahydrate, sodium ascorbate, and THPTA were dissolved in water and transferred to the click reagents to begin the reaction. The reaction proceeded over 24 hours. This was purified by first centrifuging the reaction at 14000 rpm for 5 minutes, and then injecting the supernatant on high performance liquid chromatography using a gradient of 10%-90% acetonitrile in water with 0.1% formic acid. Peaks corresponding to product were pooled and lyophilized. The final product was obtained in 14.51% yield. LCMS confirmed correct expected mass of product. MS-ESI [M+4H]4+m/z calc for [C₁₀₉H₁₈₇N₃₁O₃₁S] 615.845, found 615.8500 (FIG. 4).

Norbornene-PEG8-Propargyl:

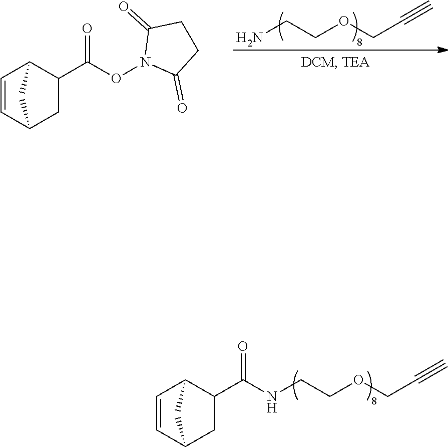

Norbornene-NHS (0.2 mmol, 1 eq) was transferred to a vial with a stir bar and evacuated with argon. Propargyl-PEG8-NH2 (0.24 mmol, 1.2 eq) was added to this, followed by anhydrous DCM (3 mL) and then TEA (0.4 mmol, 2 eq). The reaction was allowed to proceed over 24 hours. The reaction was purified on a reverse phase 20 g Buchi EcoFlex C18 column using a shallow gradient of 5% to 95% acetonitrile in water. Tubes containing only product were pooled and diluted 2× with saturated sodium bicarbonate in water. This was extracted three times with 10 mL ethyl acetate. The ethyl acetate was washed with brine, dried over magnesium sulfate, and then was rotary evaporated to provide the final product in 89.55% yield. 1H NMR (700 MHz, CDCl3) δ 6.20 (s, 1H), 6.11 (m, 2H), 4.20 (d, J=2.4 Hz, 2H), 3.68 (m, 4H), 3.65-3.64 (m, 20H), 3.62 (m, 4H), 3.56 (t, J=4.9 Hz, 2H), 3.46 (q, J=4.7 Hz, 2H), 2.92 (m, 1H), 2.90 (s, 1H), 2.43 (t, J=2.4 Hz, 1H), 2.02 (qd, J=1.9 Hz, 1H), 1.91 (dt, J=3.9 Hz, 1H), 1.73 (d, J=8.3 Hz, 1H), 1.32 (m, 2H). LCMS confirmed correct expected mass of product. MS-ESI [M+H]⁺ m/z calc for [C₂₇H₄₅NO₉] 528.31, found 528.3486.

Fluorescein-PEG8-Propargyl:

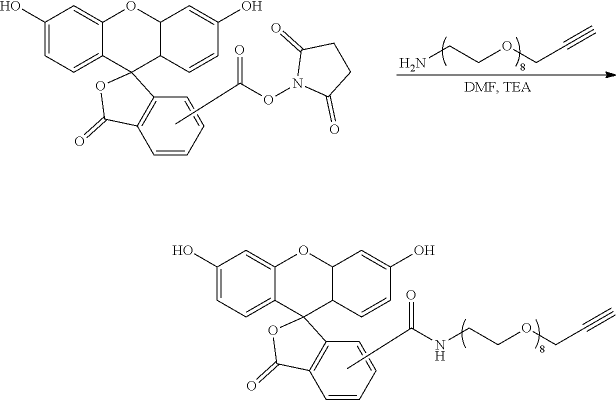

5/6-Fluorescein-NHS (0.1 mmol, 1 eq) was transferred to a vial with a stir bar and evacuated with argon. Propargyl-PEG8-NH2 (0.12 mmol, 1.2 eq) was added to this, followed by 4 mL of anhydrous DMF (4 mL) and then TEA (0.2 mmol, 2 eq). The reaction was covered in foil and allowed to react over 24 hours. The reaction was purified on a reverse phase 12 g Buchi EcoFlex C18 column using a shallow gradient of 10% to 90% acetonitrile in water. The product was lyophilized to give the final product in 62.27% yield. 1H NMR (700 MHz, CDCl₃) b 8.45 (s, 1H), 8.14 (d, J=8.0 Hz, 1H), 8.03 (d, J=8.1 Hz, 1H), 7.75 (s, 1H), 7.62 (s, 1H), 7.58 (s, 1H), 7.18 (d, J=7.8 Hz, 1H), 6.69 (d, J=24.5 Hz, 2H), 6.57 (d, J=8.5 Hz, 1H), 6.52 (d, J=8.1 Hz, 1H), 6.49 (d, J=8.5 Hz, 1H), 6.45 (d, J=8.3 Hz, 1H), 4.14 (d, J=2.3 Hz, 2H), 3.59 (m, 30H), 3.42 (s, 2H), 2.42 (m, 1H). LCMS confirmed correct expected mass of product. MS-ESI [M+H]⁺ m/z calc for [C₄₀H₄₉NO₁₄] 727.32, found 766.4681.

GUL-NHS:

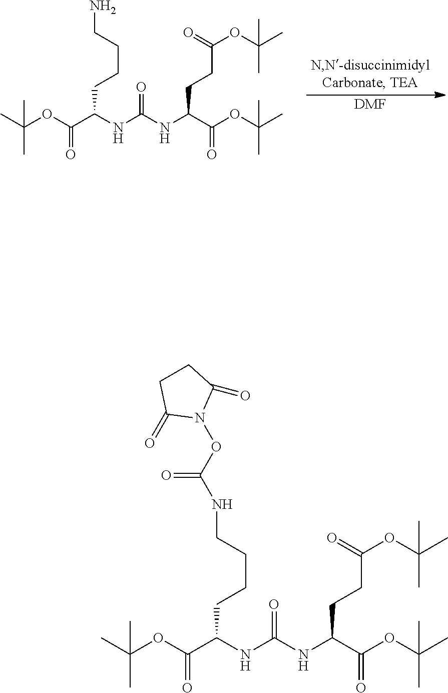

GUL (154.5 mg, 0.317 mmol) was dissolved in 2 mL of toluene and dried under vacuum, backfilled with nitrogen, and re-dissolved in 14.3 mL anhydrous DMF. Next, N,N′-Disuccinimidyl Carbonate (90 mg, 0.349 mmol) was added followed by TEA (55 uL, 0.317 mmol) and the solution was left stirring overnight. The reaction solution was then diluted with 50 mL EtOAc and washed with a 10% citric acid solution followed by brine. The organic layer was dried over anhydrous Mg2SO4 and concentrated under vacuum. The crude product was purified via flash silica gel column chromatography (3:1 EtOAc/Hexanes) to yield 16 (53.2 mg, 26.7% yield) as a clear oil. 1H NMR (700 MHz, CDCl₃) δ 6.62 (s, 1H), 5.55 (d, J=8.0 Hz, 2H), 5.46 (d, J=8.3 Hz, 2H), 4.31 (m, J=4.1 Hz, 2H), 3.24 (m, J=7.6 Hz, 2H), 2.83 (s, 4H), 2.28 (m, J=5.4 Hz, 2H), 2.02 (s, 1H), 1.80 (m, J=4.6 Hz, 1H), 1.74 (m, J=4.8 Hz, 1H), 1.60 (m, J=4.6 Hz, 1H), 1.54 (m, J=7.0 Hz, 1H), 1.43 (s, 9H), 1.43 (s, 9H), 1.41 (s, 9H), 1.35 (m, J=6.1 Hz, 1H). LCMS confirmed correct expected mass of product. MS-ESI [M+H]⁺ m/z calc for [C₂₉H₄₈N₄O₁₁] 629.33, found 629.3394.

GUL-PEG8-Propargyl:

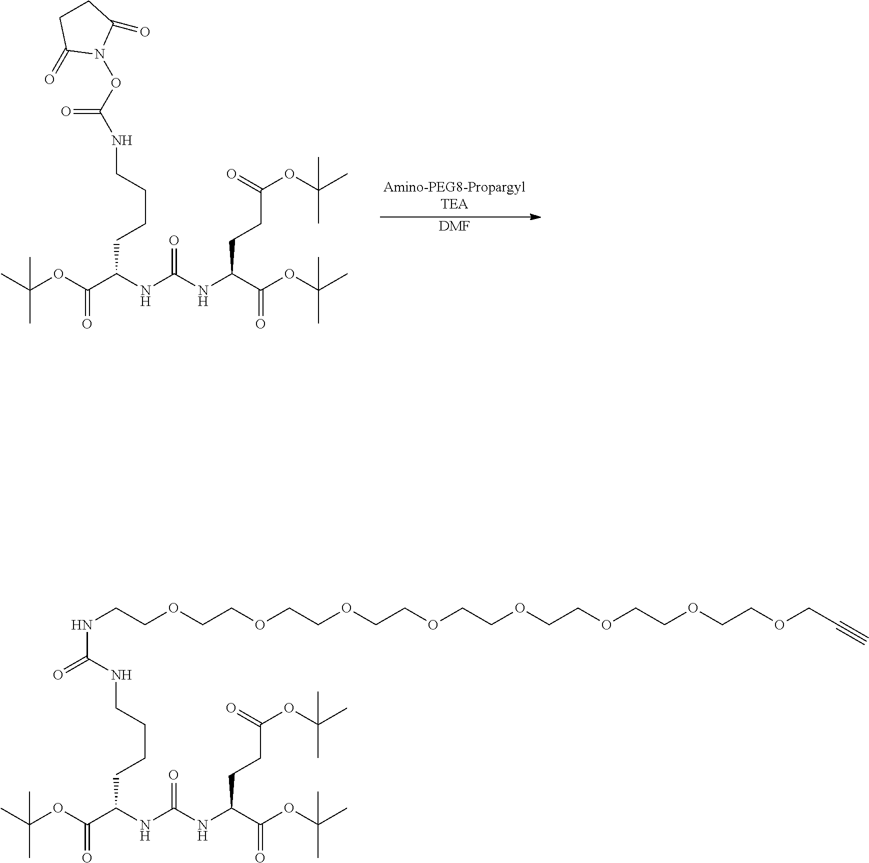

GUL-NHS (169.4 mg, 0.269 mmol) was dissolved in 1 mL of toluene and dried under vacuum, backfilled with argon, and re-dissolved in 3 mL anhydrous ACN. Amino-PEG8-propargyl (135.2 mg, 0.332 mmol) was resuspended in 1 mL toluene and dried under vacuum, backfilled with argon, and GUL-NHS solution was added. To this mix, TEA (50 uL, 0.359 mmol) was added. This mixture was stirred overnight. The solution was diluted in 75 mL EtOAc and washed once with sat. sodium bicarbonate followed two washes with sat. brine. The organic layer was dried over anhydrous Mg₂SO₄ and concentrated under vacuum. The crude product was purified via flash silica gel column chromatography (consecutive EtOAc:Hex followed by DCM:MeOH gradients) to yield semi-pure product (67.9 mg, 27.4% yield) as a clear oil. ¹H NMR (700 MHz, CDCl₃) δ 5.68 (d, J=7.6 Hz, 1H), 5.56 (d, J=8.1 Hz, 1H), 4.31 (m, 1H), 4.23 (m, 1H), 4.19 (d, J=2.4 Hz, 2H), 3.65 (m, 28H), 3.55 (m, 2H), 3.34 (m, 2H), 3.14 (m, 2H), 2.44 (t, J=2.4 Hz, 1H), 2.31 (m, 2H), 1.94 (m, 2H), 1.69 (m, 2H), 1.47 (m, 2H), 1.44 (m, 18H), 1.42 (s, 9H), 1.35 (m, 2H). LCMS confirmed correct expected mass of product. MS-ESI [M+H]*m/z calc for [C₄₄H₈₀N₄O₁₆] 921.56, found 921.4949.

GUL-PEG8-HSVqD1 (ARM 1.2):

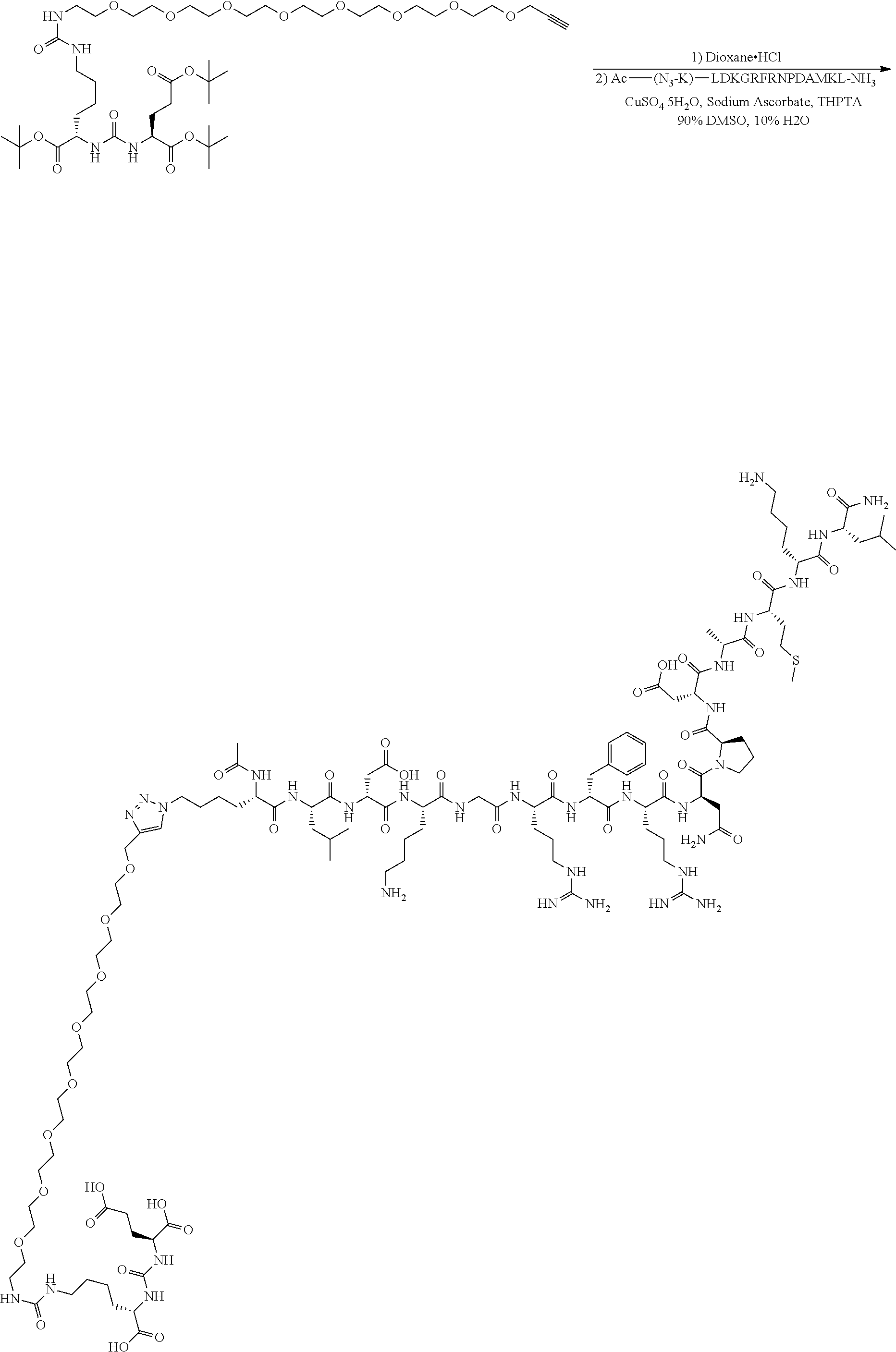

GUL-PEG8-propargyl (25.7 mg, 0.028 mmol) was dissolved in 1 mL of toluene and dried under vacuum, backfilled with argon, and re-dissolved in dioxane.HCl (1 mL, 4M). This was mixed for 3 hours and dried under vacuum. This was resuspended in 707 uL DMSO and added to crude HSV-N3 peptide (47 mg, 0.025). To this mix, 132.2 uL of an aqueous stock solution was added containing copper sulfate pentahydrate (3.16 mg, 0.013 mmol), sodium ascorbate (12.69 mg, 0.064 mmol), and THPTA (78 mg, 0.024 mmol). After 4 hours, this reaction mixture was centrifuged and supernatant purified via HPLC to yield pure product (2.6 mg, 3.94% yield) as a clear oil. LCMS confirmed correct expected mass of product. MS-ESI [M+4H]⁴⁺ m/z calc for [C₁₁₃H₁₉₁N₃₁O₃₇S] 652.59, found 653.0820 (FIG. 9).

### Example 3: Methods

Desthiobiotin-PEG8-qD1 Bio-Layer Interferometry Assay:

Streptavidin coated biosensor probes from Forte Bio were placed in 200 μL solutions of Kinetics Buffer (1× PBS, 0.01% BSA, 0.002% Tween20) spiked with 1% (v/v) DMSO for 10 minutes in an Octet Red96 for wetting. The probes were then baselined in Kinetics Buffer for 180 seconds at an RPM of 1000 and temperature of 30° C. using an acquisition rate of 5 Hz. After baselining the signal, the probes were placed in a 200 nM solution of the biotinylated peptides of interest in Kinetics Buffer (1×) spiked with 1% (v/v) DMSO for 180 seconds to load the peptide onto the probe. This was followed by placing the streptavidin probes in a 5% (w/v) milk quench solution in Kinetics Buffer (1×) spiked with 1% (v/v) DMSO for 150 seconds to block non-specific binding. To re-establish baseline, the streptavidin probes were placed back in the baseline (kinetics buffer) solution for 180 seconds. Next the streptavidin probes (loaded with desthiobiotin-PEG8-gD1) were placed in a solution of Kinetics Buffer (1×) spiked with 1% (v/v) and protein of interest (anti-gD1 monoclonal antibody LP14, Cat. No. MABF1975). Data was processed on Graph Pad Prism 8 by inputting association and dissociation nm shifts retrieved from the begging of the association phase to the end of the dissociation phase. Salt effects, such as large increases in nm shift between association and dissociation wells, were removed by omitting the data. Data was baselined by subtracting the baseline value from all proceeding nm shifts. Dissociation constants were calculated through the ratio of koff/kon.

Anti-qD1 Antibody Purification:

Pierce™ High-Capacity Streptavidin Agarose resin (Cat. No. P120357) was used to isolate anti-HSV from pooled human serum IgG (Cedarlane Labs Cat. No. IHUIGGAP1000MG). The resin was transferred to a column (1 mL total volume, 0.5 mL resin). 1 mL of 66.67 μM desthiobiotin-PEG8-gD1 was added to the resin and incubated for 20 minutes at room temperature. The column was washed with 10 column volumes of 1×PBS. 2 column volumes of pooled human IgG were added and incubated at 4° C. for 24 hours. The column was washed with 10 column volumes of 1×PBS. The anti-HSV isolate was eluted using three consecutive 2 column volume elutions containing free 2 mM gD1 peptide in 1× Kinetics Buffer. The eluate was concentrated 5× to 600 μL in an Amicon® Ultra 30 centrifugal spin filter (Ultra-4, 30 kDa cut off).

Anti-qD1 SDS-PAGE Analysis:

The enriched anti-HSV concentrate was analyzed by SDS-PAGE. The samples were diluted 1:1 in Laemmli 2× concentrate sample buffer (Sigma-Aldrich, Cat. No. S3401-10VL, 15 μL each) and heated at 95° C. for 5 minutes. These were allowed to cool for 2 minutes before loading on a Novex™ WedgeWell™ 10% Tris-Glycine Gel (Invitrogen, Cat. No. XP00100BOX). 1× running buffer used with a 90V band stacking phase and a 120V band separation phase. The gel was washed for 15 minutes in deionized water and then stained in 30 mL EZBlue™ Gel Staining Reagent (Sigma-Aldrich, Cat. No. G1041-500ML) for 2 hours. The gel was destained in DI water for 2 hours. An image was taken using an Odyssey CLx gel imager.

### Example 4: Antibody Recruitment Molecule 1.1

ARM 1.1 was designed for use as, for example, a research tool or diagnostic tool to determine the titers and binding affinities of HSV-specific antibodies from mouse and human sera.

To facilitate detection and isolation of serum antibodies, and to enable immobilization of ARM 1.1 on a solid substrate, the biotin derivative, desthiobiotin, was used as the TBT due to its high-affinity, reversible binding to avidin/streptavidin molecules and derivatives thereof. First, an intermediate of ARM 1.1 was synthesized, called targeting fragment 1A. Targeting fragment 1A consists of desthiobiotin (as the TBT) conjugated to a PEG8 linker according to the following formula:

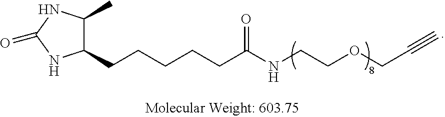

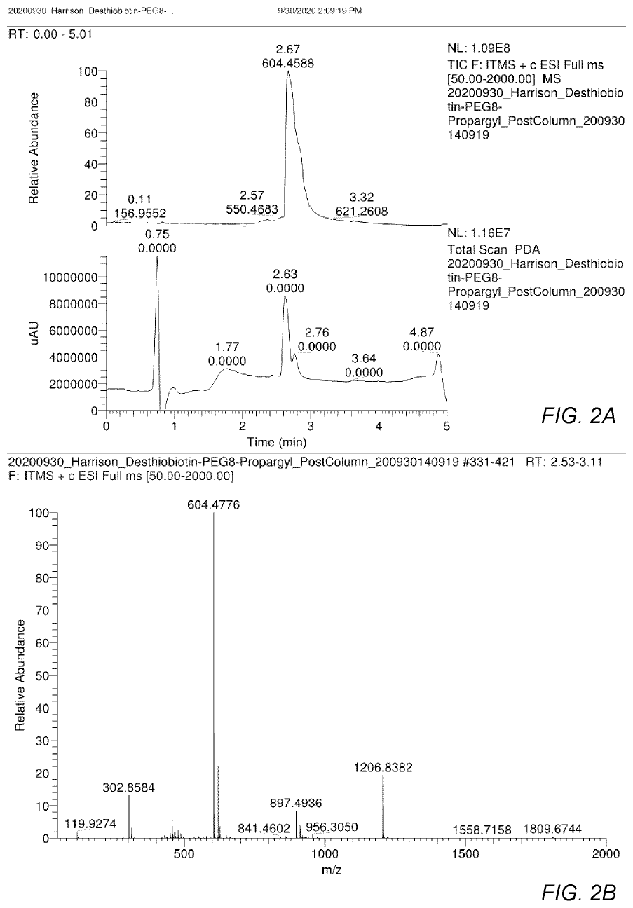

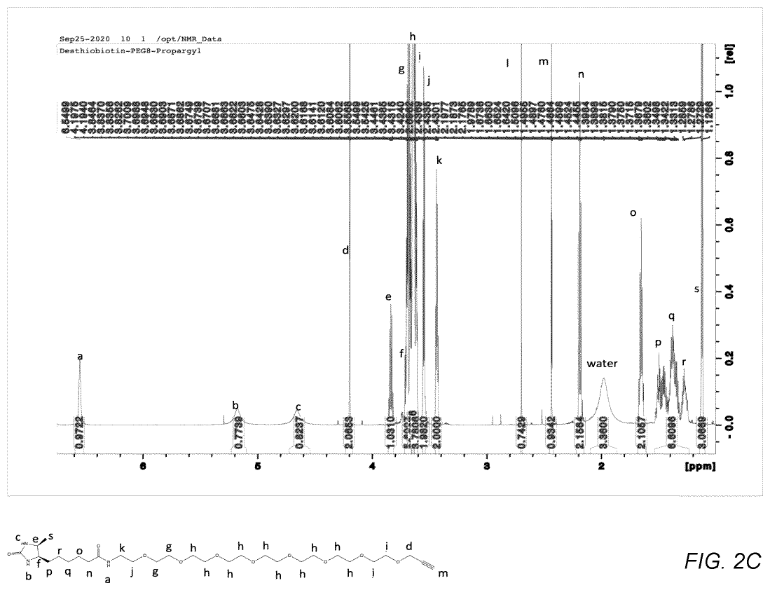

The distal end (relative to the position of desthiobiotin) of the linker of targeting fragment 1A includes a propargyl functional group that is capable of reacting with an azide group on another molecule in a click chemistry reaction to form a triazole linkage between the molecules. This mode of conjugation enables modular assembly of ARMs with various TBTs and ABTs for versatile tumor targeting and antibody recruitment. Targeting fragment 1A was successfully synthesized and characterized in vitro using LCMS (FIGS. 2A and 2B) and ¹H NMR (FIG. 2C).

To enable recruitment of HSV-specific antibodies by ARM 1.1, a peptide fragment containing the N-terminal domain of HSV glycoprotein D1 (gD1) was synthesized and used as the ABT. This synthetic peptide intermediate is called antibody-binding fragment 1B and comprises the amino acid sequence set forth in SEQ ID NO: 1 (LKMADPNRFRGKDL). Antibody-binding fragment 1B is represented by the following formula:

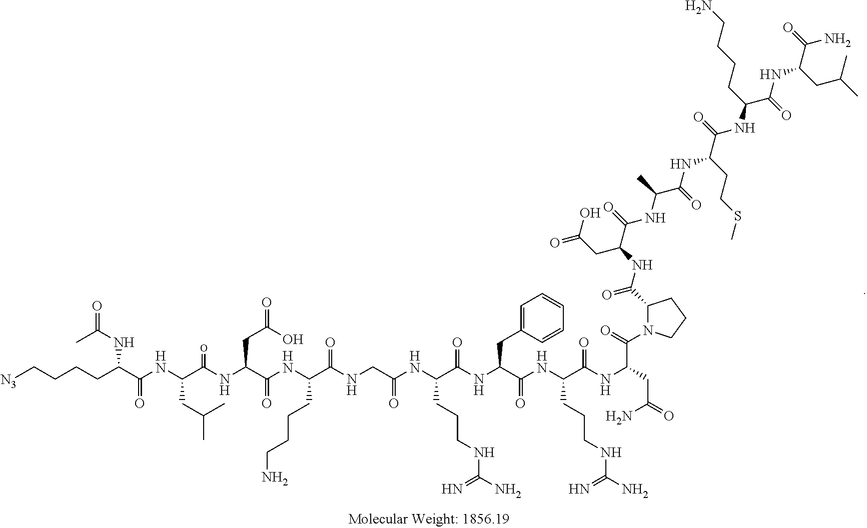 [SMILES: CSCC[C@H](NC(=O)[C@H](C)NC(=O)[C@H](CC(=O)O)NC(=O)[C@@H]1CCCN1C(=O)[C@H](CC(N)=O)NC(=O)[C@H](CCCNC(=N)N)NC(=O)[C@H](Cc1ccccc1)NC(=O)[C@H](CCCNC(=N)N)NC(=O)CNC(=O)[C@H](CCCCN)NC(=O)[C@H](CC(=O)O)NC(=O)[C@H](CC(C)C)NC(=O)[C@H](CCCCN=[N+]=[N-])NC(C)=O)C(=O)N[C@@H](CCCCN)C(=O)N[C@@H](CC(C)C)C(N)=O]

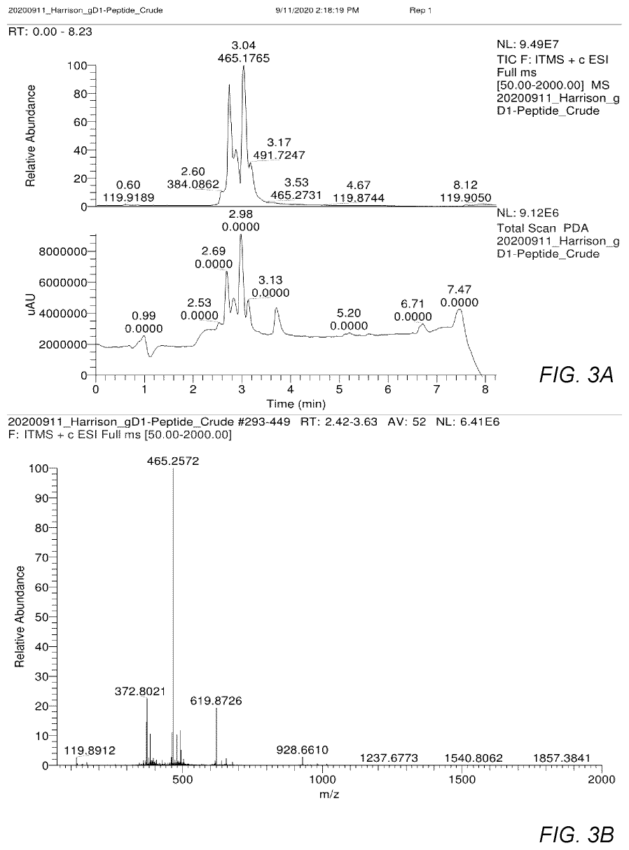

Antibody-binding fragment 1B was successfully synthesized and characterized in vitro using LCMS (FIGS. 3A and 3B).

The intermediates, targeting fragment 1A and antibody-binding fragment 1B, were conjugated using CuAAC click chemistry to obtain ARM 1.1. ARM 1.1 was characterized in vitro using LCMS (FIGS. 4A and 4B). ARM 1.1 is represented by the following formula:

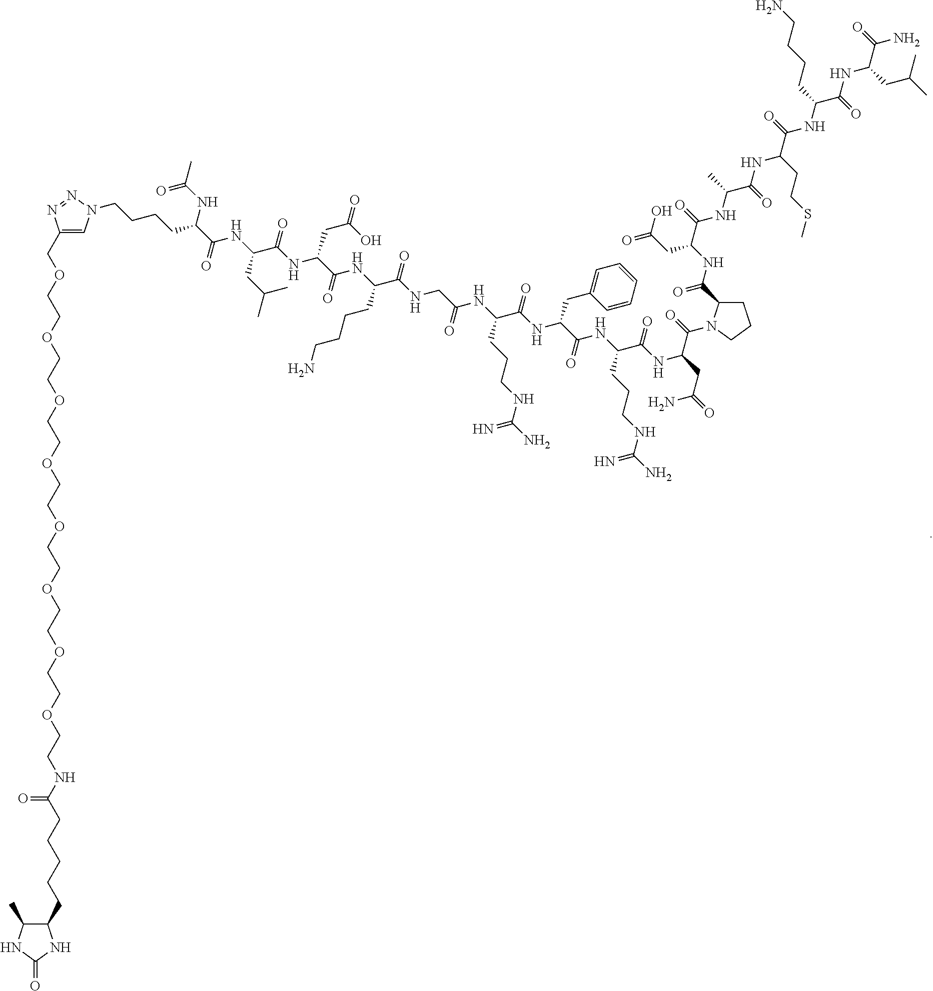 [SMILES: CSCCC(NC(=O)[C@@H](C)NC(=O)[C@@H](CC(=O)O)NC(=O)[C@H]1CCCN1C(=O)[C@@H](CC(N)=O)NC(=O)[C@H](CCCNC(=N)N)NC(=O)[C@@H](Cc1ccccc1)NC(=O)[C@H](CCCNC(=N)N)NC(=O)CNC(=O)[C@H](CCCCN)NC(=O)[C@@H](CC(=O)O)NC(=O)[C@H](CC(C)C)NC(=O)[C@H](CCCCn1cc(COCCOCCOCCOCCOCCOCCOCCOCCNC(=O)CCCCC[C@H]2NC(=O)N[C@H]2C)nn1)NC(C)=O)C(=O)N[C@H](CCCCN)C(=O)N[C@@H](CC(C)C)C(N)=O]

### Example 5: Bifunctional Click Chemistry Linkers

To facilitate rapid assembly of various TBT and ABT modules into ARMs for different uses, a bifunctional linker fragment 1C was designed. Linker fragment 1C comprises two distinct reactive groups capable of mediating conjugation via distinct click chemistry reactions, in particular IEDDA and CuAAC. Linker fragment 1C is represented by the following formula:

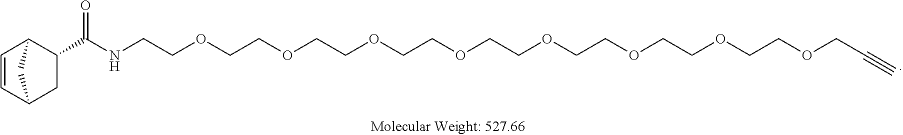 [SMILES: C#CCOCCOCCOCCOCCOCCOCCOCCOCCNC(=O)[C@@H]1C[C@H]2C=C[C@@H]1C2]

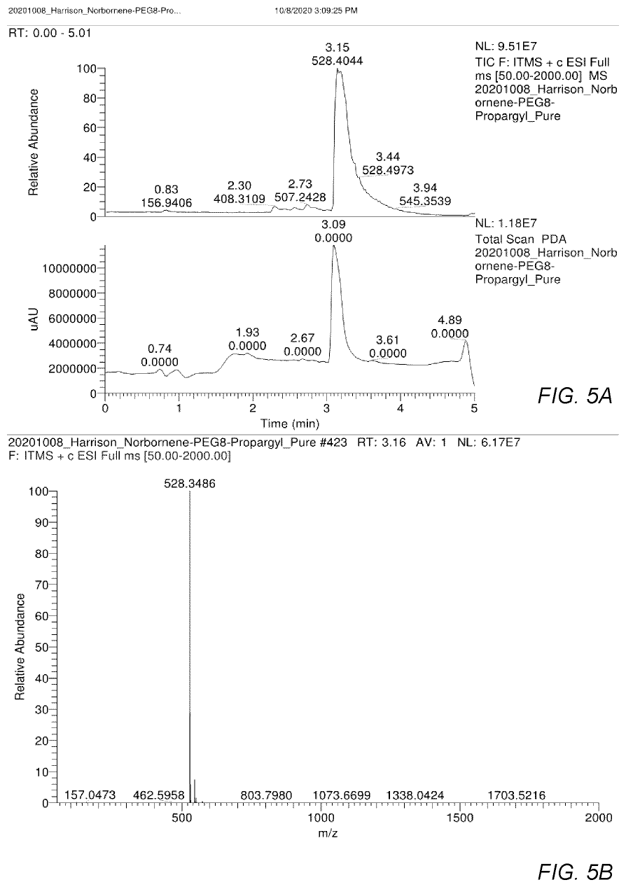

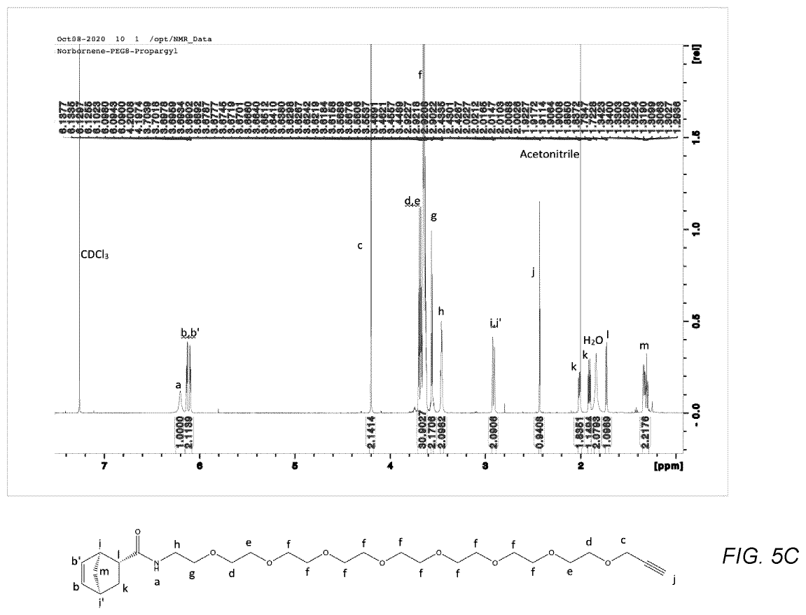

Linker fragment 1C can be used, for example, to mediate versatile attachment of the HSV-specific antibody-binding fragment 1B (ABT module) with various tumor antigen-binding TBT modules, including but not limited to: (i) glutamate urea ligand to target PSMA in prostate cancer; (ii) cyclic peptides to target uPAR in glioblastoma; (iii) trastuzumab (HERCEPTIN™) derived 9 amino acid peptide to target HER2 in breast and ovarian tumors; (iv) methotrexate or folate to target folate receptor in ovarian cancer. Linker fragment 1C was successfully synthesized and characterized in vitro using LCMS (FIGS. 5A and 5B) and ¹H NMR (FIG. 5C).

Additionally, to enable modular attachment of a fluorescent reporter with a given ABT module, a bifunctional linker fragment 1D was designed. Linker fragment 1D comprises fluorescein on one end of the linker and a reactive propargyl group on the other end of the linker. Linker fragment 1D is represented by the following formula:

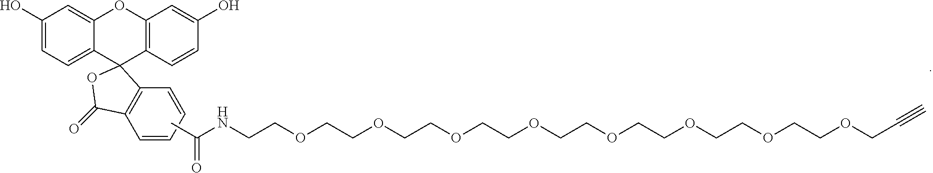

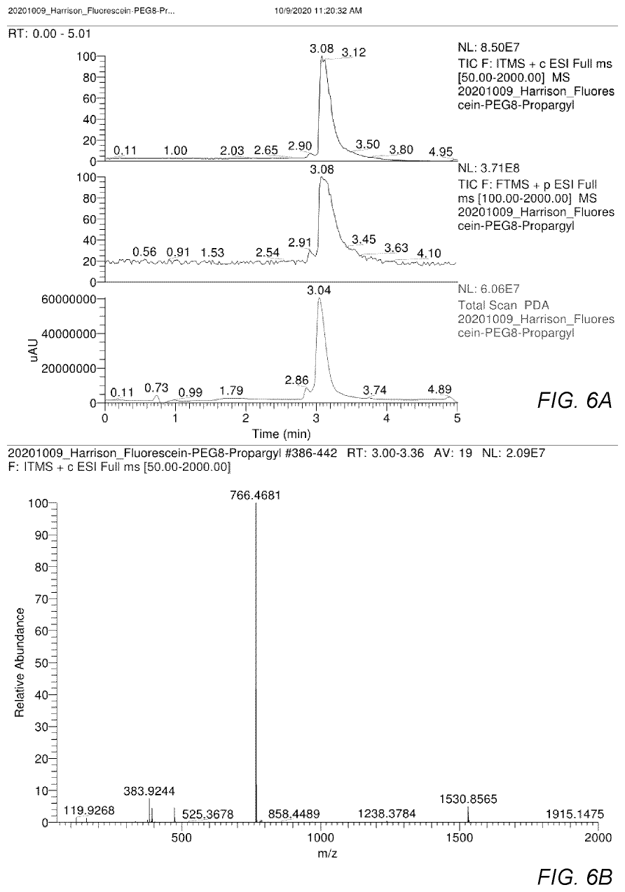

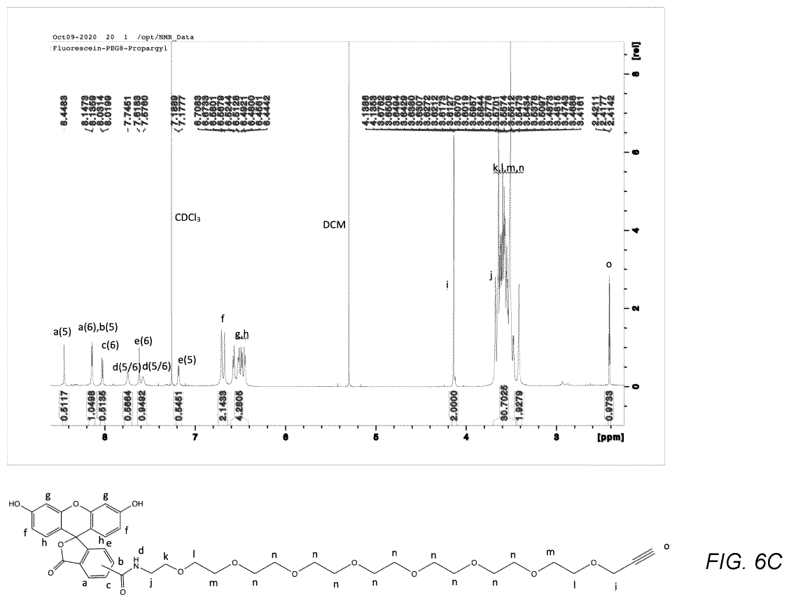

Linker fragment 1 D can be used, for example, to mediate versatile attachment of fluorescent reported with the HSV-specific antibody-binding fragment 1B (ABT module) using CuAAC chemistry. The resulting ARM derivative can be used to validate covalent labeling of HSV-specific antibodies directly in human and mouse serum via fluorescence SIDS-PAGE, or to validate antibody-binding affinity using fluorescence polarization assays. Linker fragment 1 D was successfully synthesized and characterized in vitro using LCMS (FIGS. 6A and 6B) and ¹H NMR (FIG. 6C).

### Example 6: Antibody Recruitment Molecule 1.2

ARM 1.2 was designed to recruit HSV-specific serum antibodies and target them to PSMA on prostate cancer cells. Targeting fragment 2A was used as the TBT module. Targeting fragment 2A comprises a PSMA-targeting urea glutamate ligand, represented by the following formula:

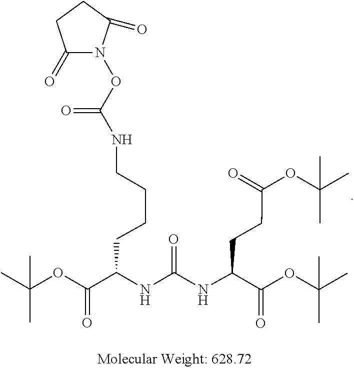 [SMILES: CC(C)(C)OC(=O)CC[C@H](NC(=O)N[C@@H](CCCCNC(=O)ON1C(=O)CCC1=O)C(=O)OC(C)(C)C)C(=O)OC(C)(C)C]

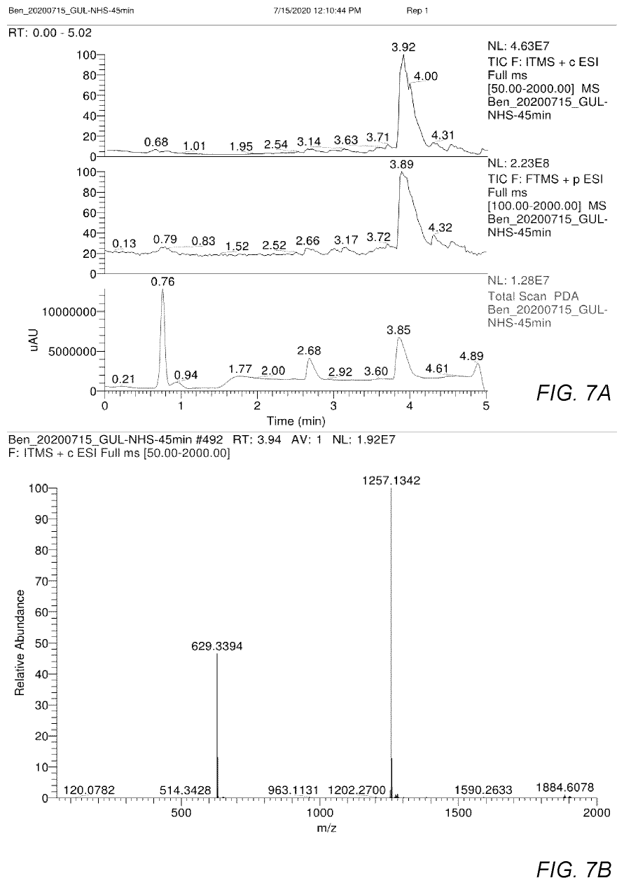

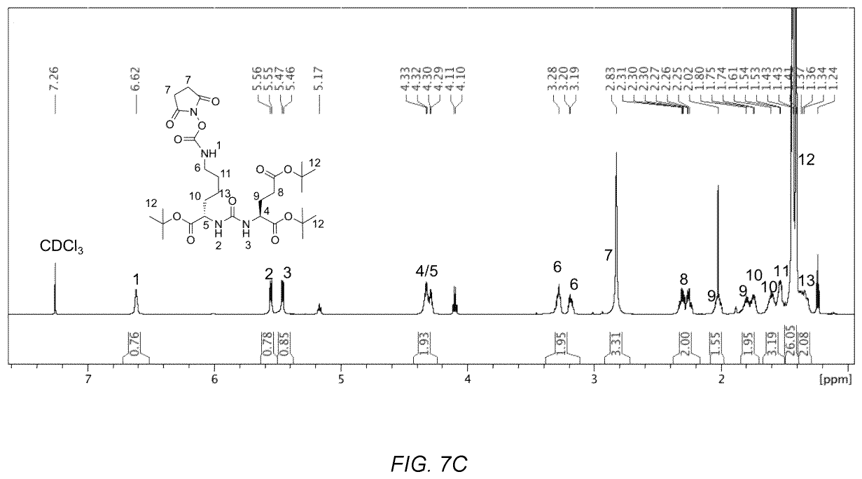

Targeting fragment 2A was successfully synthesized and characterized in vitro using LCMS (FIGS. 7A and 7B) and ¹H NMR (FIG. 7C).

Next, targeting fragment 2A was conjugated with linker fragment 1C via IEDDA click chemistry to obtain a TBT+linker fragment 2B. TBT+linker fragment 2B is represented by the following formula:

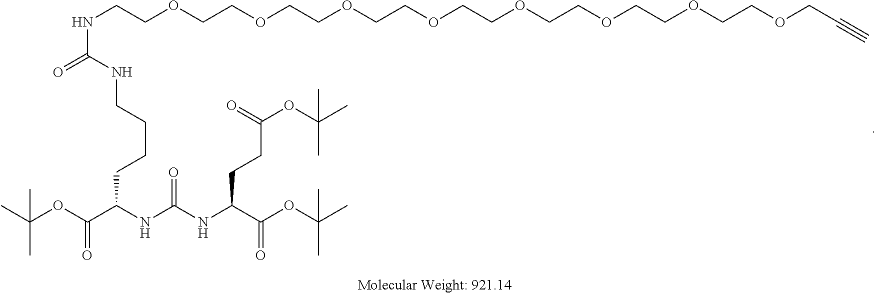 [SMILES: C#CCOCCOCCOCCOCCOCCOCCOCCOCCNC(=O)NCCCC[C@H](NC(=O)N[C@@H](CCC(=O)OC(C)(C)C)C(=O)OC(C)(C)C)C(=O)OC(C)(C)C]

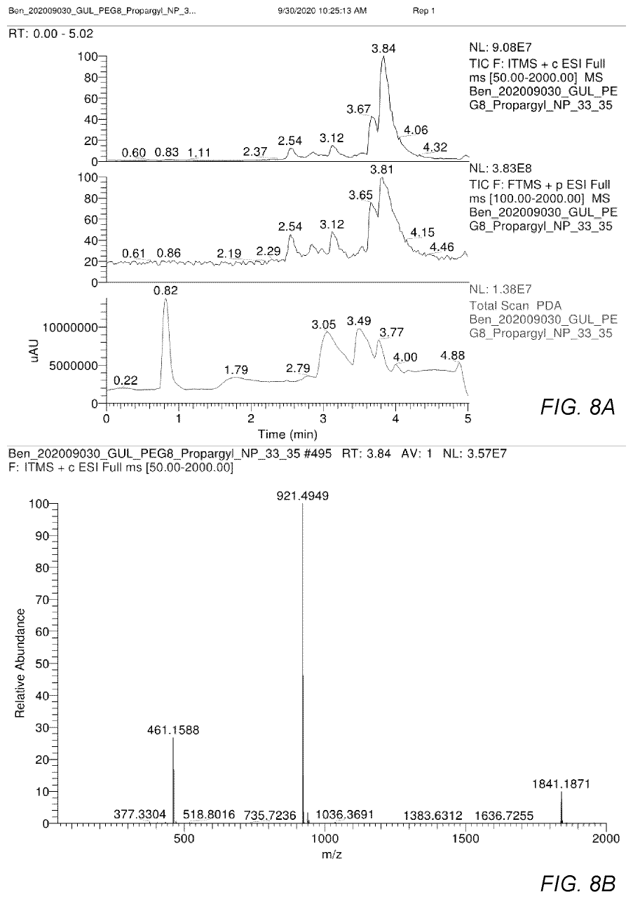

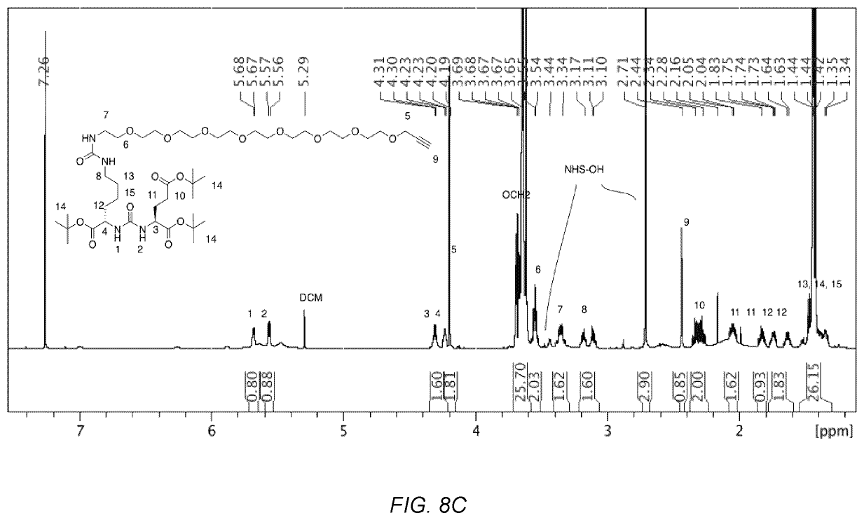

TBT+linker fragment 2B was successfully synthesized and characterized in vitro using LCMS (FIGS. 8A and 8B) and ¹H NMR (FIG. 8C).

Finally, TBT+linker fragment 2B was conjugated with antibody-binding fragment 1B (the ABT module) using CuAAC click chemistry to obtain ARM 1.2. ARM 1.2 was characterized in vitro using LCMS (FIGS. 9A and 9B). ARM 1.2 is represented by the following formula:

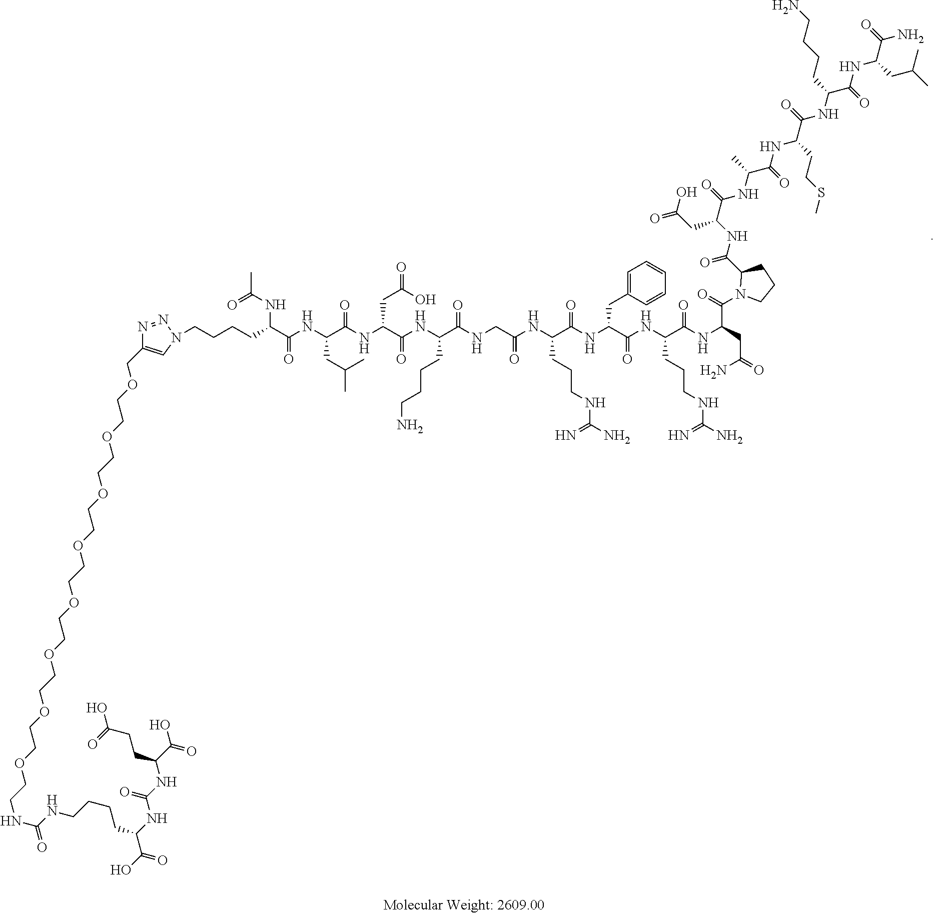 [SMILES: CSCC[C@H](NC(=O)[C@@H](C)NC(=O)[C@@H](CC(=O)O)NC(=O)[C@H]1CCCN1C(=O)[C@@H](CC(N)=O)NC(=O)[C@H](CCCNC(=N)N)NC(=O)[C@@H](Cc1ccccc1)NC(=O)[C@H](CCCNC(=N)N)NC(=O)CNC(=O)[C@H](CCCCN)NC(=O)[C@@H](CC(=O)O)NC(=O)[C@H](CC(C)C)NC(=O)[C@H](CCCCn1cc(COCCOCCOCCOCCOCCOCCOCCOCCNC(=O)NCCCC[C@H](NC(=O)N[C@@H](CCC(=O)O)C(=O)O)C(=O)O)nn1)NC(C)=O)C(=O)N[C@H](CCCCN)C(=O)N[C@@H](CC(C)C)C(N)=O]

### Example 7: Validation of HSV-Specific Antibody Recruitment to a Model Target Protein by Antibody Recruitment Molecule 1.1

FIG. 10A shows the results of a BLI assay to test ARM 1.1 (see Example 4) mediated recruitment of a model anti-HSV gD1 monoclonal antibody to a model target protein (streptavidin). Buffer alone served as a negative control. “Flipped” gD1 peptide, which served as a control for selective binding of the anti-HSV antibody to the ARM, contains the same amino acids as the “correct” gD1 peptide, but has an incorrect amino acid sequence. Specific binding of the anti-HSV antibody to an immobilized ARM-streptavidin conjugate was evidenced by the BLI wavelength shift of the gD1 sample.

FIG. 10B shows the results of a BLI assay of competitive dissociation of the anti-HSV gD1 monoclonal antibody used in FIG. 10A from the immobilized ARM 1.1-streptavidin conjugate used in FIG. 10A. Competition was performed using free gD1 peptide during the dissociation phase, and the results support selective anti-HSV antibody recruitment by ARM 1.1, with a dissociation constant (K_{D}) in the nanomolar range. The following parameters were determined using the competitive dissociation assay:
kₒₙ=31937 min⁻¹M⁻¹
k_{off}=0.006114 min⁻¹
K_{D}=191.4 nM

FIG. 11 shows the results of an antibody purification, where substantial amounts anti-HSV polyclonal antibodies were successfully isolated from pooled human serum IgG. Lanes 1 and 2 of the SDS-page gel contain serum IgG that does not retain on an affinity resin loaded with ARM 1.1 during buffer wash steps and is either non-specific IgG or weak affinity anti-HSV gD1 IgG. Lane 3 contains eluate from an additional wash step, demonstrating substantially less non-specific IgG is present on beads coated with ARM 1.1. Lane 4 contains specific anti-HSV gD1 IgG antibody that was displaced by elution with free gD1 peptide competitor from beads coated with ARM 1.1.

### Example 8: Antibody Recruitment Molecule 1.2 Targets PSMA-Expressing Human Cells for Antibody-Dependent Cellular Phagocytosis

ADCP of PSMA-expressing human cells by human monocytes was assessed in the presence of ARM 1.2 and anti-gD1 mouse IgG2a antibody (FIG. 12). ADCP was determined by 2-color flow cytometry assays, where target cell phagocytosis was measured by double positive cell events consisting of FL-1 dye-labeled U937 human monocytes and FL-4 dye-labeled engineered human embryonic kidney (HEK) cells expressing PSMA.

FIG. 12 shows selective phagocytosis of human target cells by ARM 1.2 (see Example 6), which contains a glutamate urea ligand (GUL) that binds PSMA and a peptide recognized by anti-HSV gD1 antibody. Robust ADCP was only observed when cell surface PSMA and anti-HSV antibody are both present (HEK+_EXP). Control experiments using IgG that does not bind the HSV gD1 peptide on ARM 1.2 but will activate human monocyte cells if recruited to the target cell surface (HEK+_EXP_Iso), or using ARM 1.2 in the absence of anti-HSV antibody (HEK+_ARMOnly), or in the presence of free competitor molecule that prevents binding of the anti-HSV antibody to ARM 1.2 (HEK+_HSVcomp), or in the presence of an isogenic control HEK cells that do not express PSMA (HEK−_EXP) all demonstrated a complete lack of ADCP.

### Example 9: Synthesis of TBT Modules for Antibody Recruitment Molecules 2.1-2.19

Organic Synthesis:

All chemical reagents and solvents were obtained from commercial suppliers (Sigma Aldrich, Broadpharm) and used without further purification. Thin layer chromatography (TLC) was performed on silica gel precoated aluminium sheets (Silicycle) and visualized by fluorescence quenching, ninhydrin, and potassium permanganate staining. All column chromatography purification was conducted using a Buchi Pure C-810 Flash purification system using normal phase silica gel (Buchi) or reverse phase C18 columns (Buchi). ¹H, ¹³C, ¹⁹F, and ¹H¹H COSY NMR spectra were all recorded in deuterated dimethyl sulfoxide ((CD₃)₂SO), deuterated chloroform (CDCl₃), or deuterated water (D₂O) on a Bruker 700 MHz spectrometer. Electrospray ionization quadrupole fourier transform mass spectroscopy (ESI-MS) data was obtained using a Waters QUATTRO mass spectrometer, while LCMS data was obtained on a Agilent-Sciex QTRAP system or a LTQ Orbitrap XL system using a gradient of 95:5 to 5:95 water (0.1% formic acid):ACN (0.1% formic acid). HRMS-ESI was obtained with a BRUKER MicroTOF II mass spectrometer. Where indicated, a ThermoFisher DIONEX UltiMate 3000 UHPLC+, with a Hypersil GOLD, 150×10 mm, 5μ, C18 column purchased from Sigma Aldrich (Cat. No. 25005-159070) was used for HPLC purification with a gradient of 95:5 to 5:95 water (0.1% formic acid):ACN (0.1% formic acid).

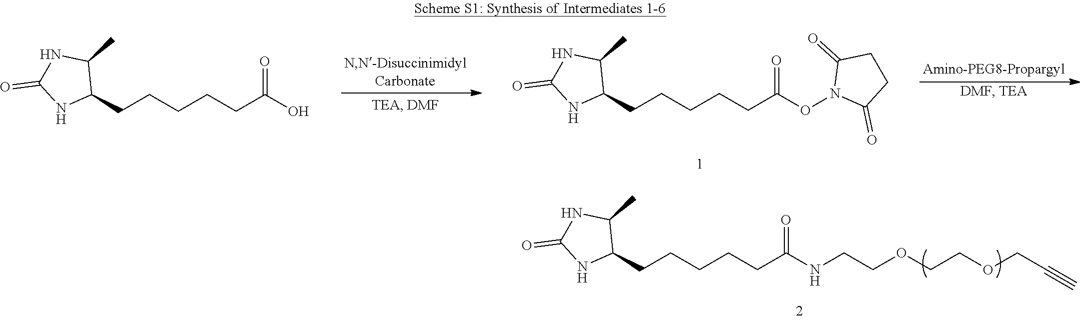

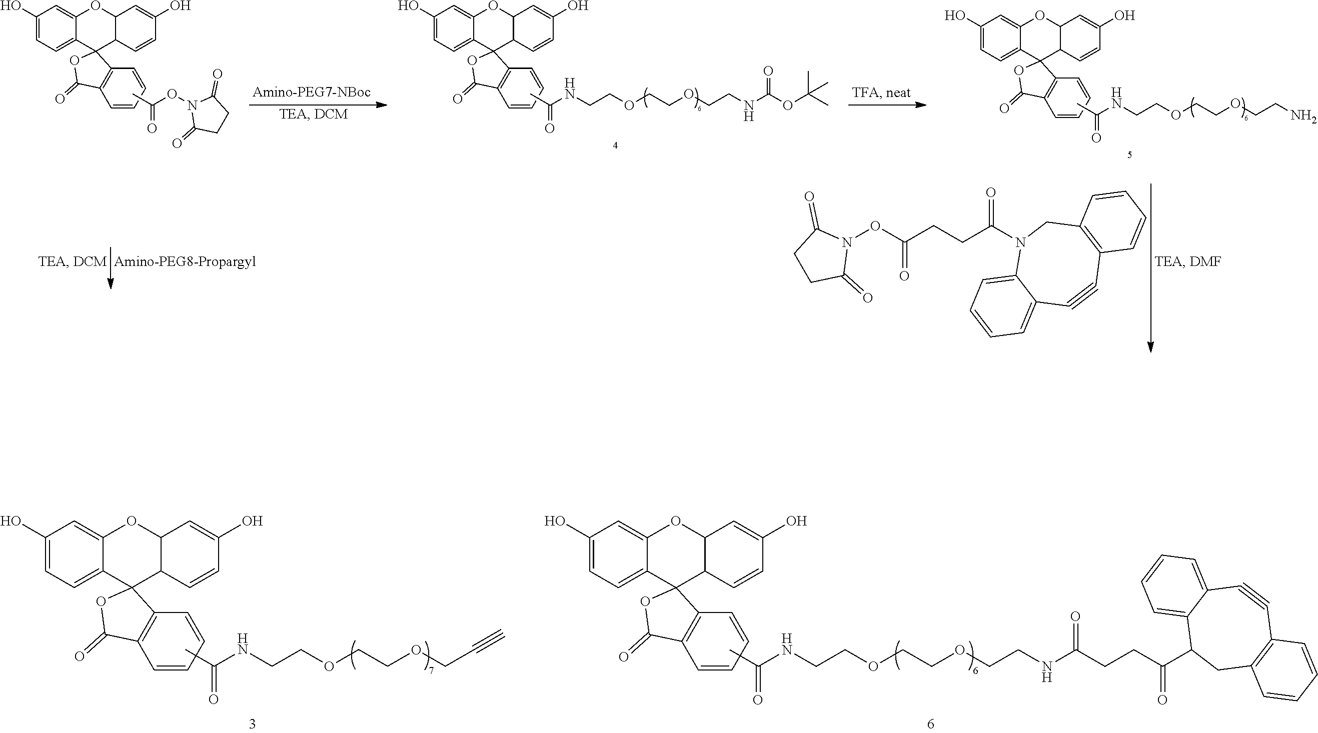

Intermediate 1:

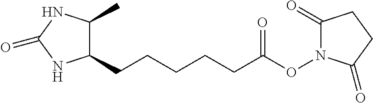 [SMILES: C[C@@H]1NC(=O)N[C@@H]1CCCCCC(=O)ON1C(=O)CCC1=O]

Desthiobiotin-COOH (0.23 mmol, eq) was added to a vial with a stir bar and was evacuated with argon. Anhydrous DMF (3 mL), TEA (0.69 mmol, eq), and N,N′-disuccinimidyl carbonate (0.253 mmol, eq) were added. After 24 hours, the reaction was transferred to 10× excess −20° C. diethyl ether to precipitate the crude product. This was centrifuged at 2000 rpm for 15 minutes. The diethyl ether was decanted, and the pellet was triturated with 2 mL EtOAc and then 2 mL diethyl ether. The product was isolated in 45.39% yield (0.104 mmol). ¹H NMR (700 MHz, DMSO) δ 6.31 (s, 1H), 6.12 (s, 1H), 3.61 (q, J=6.8 Hz, 1H), 3.49 (q, J=6.8 Hz, 1H), 2.82 (s, 4H), 2.67 (t, J=7.3 Hz, 2H), 1.63 (m, 2H), 1.36 (m, 5H), 1.22 (m, 1H), 0.97 (d, J=6.4 Hz, 3H).

LCMS data for intermediate 1 is shown in FIGS. 13A and 13B. ¹H NMR spectrum of intermediate 1 is shown in FIG. 62.

Intermediate 2:

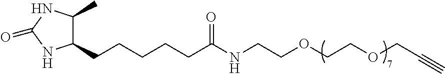

Intermediate 1 (0.138 mmol, 1 eq) was transferred to a vial with a stir bar and evacuated with argon. Propargyl-PEG8-NH₂ (0.152 mmol, 1.1 eq) was added to this, followed by anhydrous DMF (3 mL), and then TEA (0.414 mmol, 3 eq). The reaction proceeded over 24 hours. Purification was performed using a normal phase 12 g Buchi EcoFlex silica column with a 5% to 20% gradient of methanol in dichloromethane. The final product was obtained in 71.17% yield. ¹H NMR (700 MHz, CDCl₃) δ 6.55 (s, 1H), 5.18 (s, 1H), 4.66 (s, 1H), 4.20 (d, J=2.5 Hz, 2H), 3.84 (m, 1H), 3.68 (m, 4H), 3.65-3.64 (m, 24H), 3.62 (m, 4H), 3.55 (t, J=5.0 Hz, 2H), 3.44 (q, J=5.2 Hz, 2H), 2.70 (s, 1H), 2.43 (t, J=2.4 Hz, 1H), 2.19 (t, J=7.4 Hz, 2H), 1.65 (p, J=7.3 Hz, 2H), 1.38 (m, 6H), 1.12 (d, J=6.5 Hz, 3H).

LCMS data for intermediate 2 is shown in FIGS. 14A and 14B. ¹H NMR spectrum of intermediate 2 is shown in FIG. 63.

Intermediate 3:

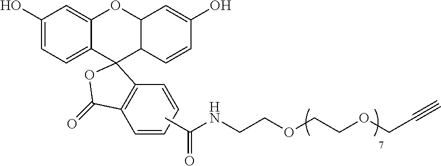

5/6-Carboxyfluorescein-NHS (0.1 mmol, 1 eq) was transferred to a vial with a stir bar and evacuated with argon. Amino-PEG8-propargyl (0.12 mmol, 1.2 eq) was transferred to a separate vial and was evacuated with argon. Anhydrous DMF (4 mL) was added to the PEG linker, followed by TEA (0.2 mmol, 2 eq). This was anhydrously transferred to the NHS ester while stirring. The reaction was covered in foil and allowed to react over 24 hours. The reaction was purified using a reverse phase 12 g Buchi EcoFlex C18 column using a gradient of 10% to 90% acetonitrile in water. The product was lyophilized to give the final product in 62.27% yield. ¹H NMR (700 MHz, CDCl₃) δ 8.45 (s, 1H), 8.14 (d, J=8.0 Hz, 1H), 8.03 (d, J=8.1 Hz, 1H), 7.75 (s, 1H), 7.62 (s, 1H), 7.58 (s, 1H), 7.18 (d, J=7.8 Hz, 1H), 6.69 (d, J=24.5 Hz, 2H), 6.57 (d, J=8.5 Hz, 1H), 6.52 (d, J=8.1 Hz, 1H), 6.49 (d, J=8.5 Hz, 1H), 6.45 (d, J=8.3 Hz, 1H), 4.14 (d, J=2.3 Hz, 2H), 3.59 (m, 30H), 3.42 (s, 2H), 2.42 (m, 1H).

LCMS data for intermediate 3 is shown in FIGS. 15A and 15B. ¹H NMR spectrum of intermediate 3 is shown in FIG. 64.

Intermediate 4:

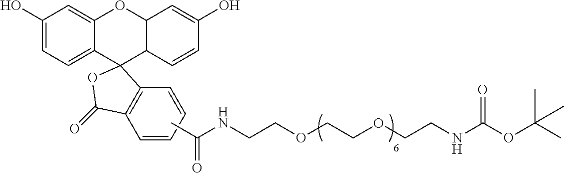

Amino-PEG7-NBoc (0.254 mmol, 1.2 eq), TEA (0.432 mmol, 2 eq), and DMF (4 mL) were added to a vial with a stir bar. Intermediate 2 (0.211 mmol, 1 eq) was added and the reaction was vigorously stirred for 24 hours. A reverse phase 12 g Buchi EcoFlex C18 column with a gradient of 5% to 95% ACN in water was used to isolate the final product in 28.13% yield. ¹H NMR (700 MHz, CDCl₃) δ 8.43 (s, 0.5H), 8.15 (m, J=3.4 Hz, 1H), 8.04 (d, J=8.1 Hz, 0.5H), 7.58 (s, 0.5H), 7.54 (s, 0.5H), 7.37 (s, 0.5H), 7.20 (d, J=7.9 Hz, 0.5H), 6.73 (d, J=2.0 Hz, 1H), 6.70 (d, J=2.3 Hz, 1H), 6.59 (s, 0.5H), 6.58 (s, 0.5H), 6.49 (m, 3H), 5.22 (m, 0.5H), 3.59 (m, 29H), 1.42 (s, 9H).

LCMS data for intermediate 4 is shown in FIG. 16. ¹H NMR spectrum of intermediate 4 is shown in FIG. 65.

Intermediate 5:

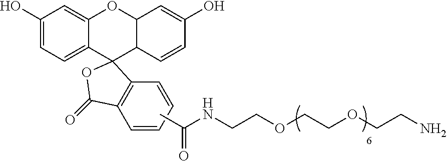

TFA (1 mL) was added to a vial with a stir bar and intermediate 3 (0.0595 mmol). After 3 hours of stirring, the TFA was blown off. DCM was transferred to the product and blown off several times to help remove the TFA. The product was obtained in quantitative yields and carried over to the next reaction. ¹H NMR (700 MHz, D₂O) δ 8.62 (s, 0.5H), 8.35 (d, J=8.5 Hz, 0.5H), 8.19 (d, J=7.7 Hz, 0.5H), 8.15 (d, J=8.7 Hz, 0.5H), 7.65 (s, 0.5H), 7.44 (d, J=7.5 Hz, 0.5H), 7.27 (d, J=9.0 Hz, 1H), 7.24 (d, J=9.1 Hz, 1H), 7.12 (s, 1H), 7.09 (s, 1H), 6.98 (d, J=9.4 Hz, 1H), 6.95 (q, J=3.4 Hz, 1H), 3.64 (m, 30H), 3.19 (t, J=4.8 Hz, 1H). ¹H NMR spectrum of intermediate 5 is shown in FIG. 66.

Intermediate 6 (Fluorescein-PEG7-DBCO):

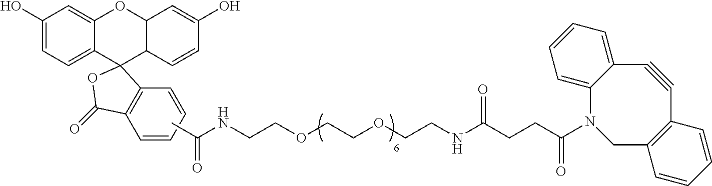

Intermediate 4 (0.0241 mmol, 1 eq), TEA (0.0241 mmol, 1 eq), and DCM (1 mL) were added to a vial with a stir bar. DBCO-NHS (0.0289 mmol, 1.2 eq) was added while stirring. After 3 hours, the reaction was washed twice with water and once with brine. The crude product was purified by normal phase flash chromatography to isolate the final product in 18% yield. ¹H NMR (700 MHz, CDCl₃) δ 8.47 (s, 0.5H), 8.17 (d, J=8.0 Hz, 0.5H), 8.14 (d, J=8.0 Hz, 0.5H), 8.05 (d, J=8.1 Hz, 0.5H), 7.64 (t, J=6.4 Hz, 1H), 7.57 (s, 0.5H) 7.56 (s, 0.5H), 7.50 (m, 2H), 7.36 (m, 3H), 7.30 (m, 2H), 7.20 (d, J=7.9 Hz, 0.5H), 7.17 (m, 1H), 6.71 (s, 1H), 6.69 (s, 1H) 6.60 (m, 2H), 6.49 (m, 2H), 6.39 (m, 1H), 5.13 (dd, J=6.6, 14.1 Hz, 1H), 3.65 (m, 7H), 3.52 (m, 20H), 3.40 (m, 2H), 3.36 (s, 1H), 3.29 (m, 2H), 2.80 (m, 1H), 2.43 (m, 1H), 2.20 (m, 1H), 1.97 (m, 1H).

LCMS data for intermediate 6 is shown in FIG. 17. The downfield and upfield regions of the ¹H NMR spectrum of intermediate 6 are shown in FIGS. 67 and 68, respectively.

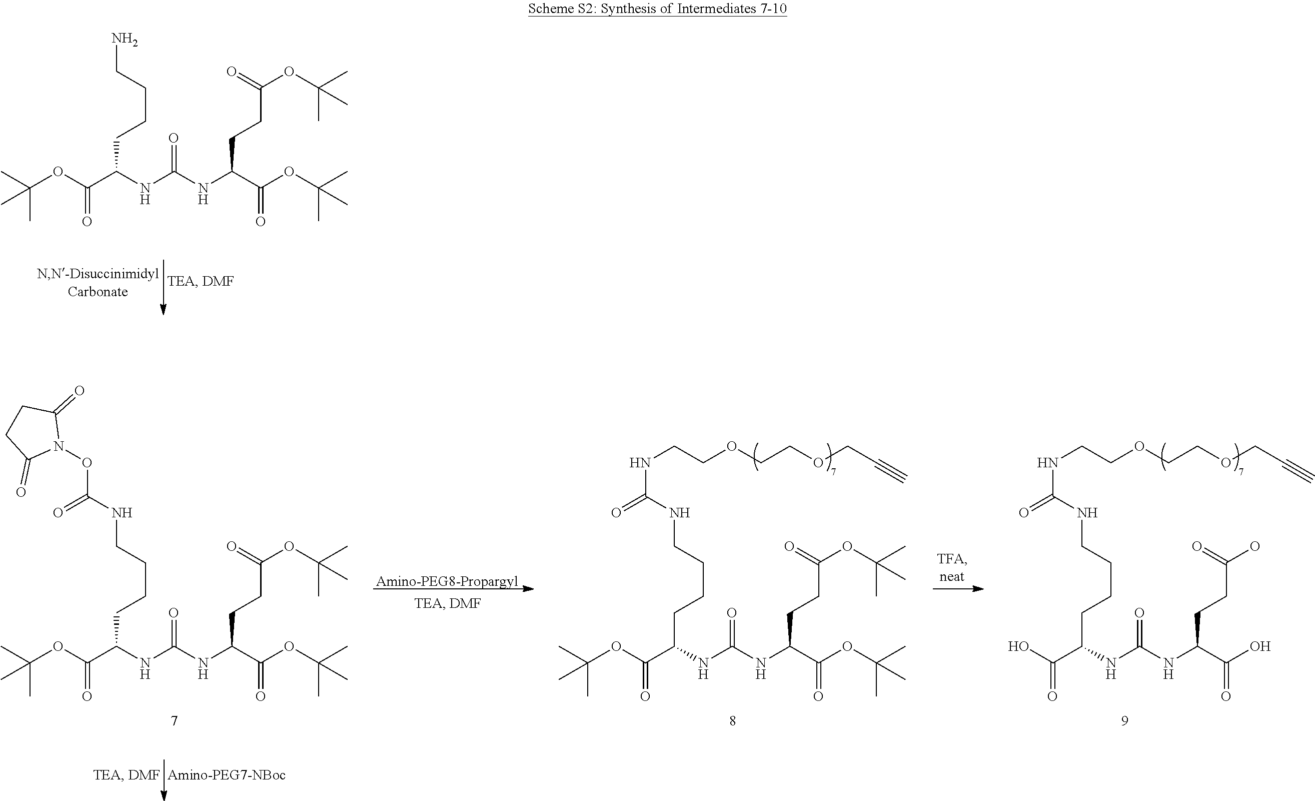

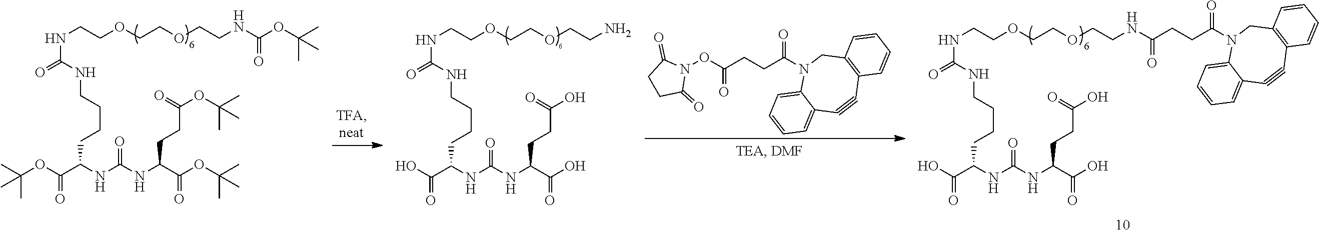

Intermediate 7 (GU-NHS):

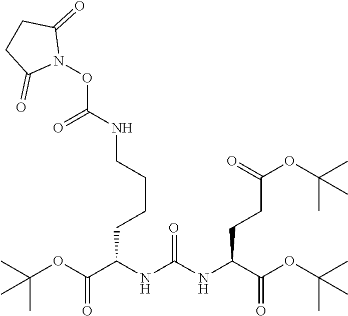 [SMILES: CC(C)(C)OC(=O)CC[C@H](NC(=O)N[C@@H](CCCCNC(=O)ON1C(=O)CCC1=O)C(=O)OC(C)(C)C)C(=O)OC(C)(C)C]

OtBu-GU-lysine (0.317 mmol, 1 eq) was dissolved in toluene (2 mL) and evaporated under vacuum to remove residual water. The flask was evacuated with nitrogen and starting material was dissolved in anhydrous DMF (14.3 mL). Under anhydrous conditions, N,N′-disuccinimidyl carbonate (0.349 mmol, 1.1 eq) was added, followed by TEA (0.317 mmol, 1 eq), and the solution was left to stir overnight. The reaction solution was then diluted with EtOAc (50 mL) and washed with 10% citric acid three times, followed by three brine washes. The organic layer was dried over anhydrous Mg₂SO₄ and concentrated under vacuum. The crude product was purified via flash silica gel column chromatography (3:1 EtOAc/Hexanes) to yield intermediate 7 in 26.7% yield (0.0846 mmol), as a clear oil. ¹H NMR (700 MHz, CDCl₃) δ 6.62 (s, 1H), 5.55 (d, J=8.0 Hz, 2H), 5.46 (d, J=8.3 Hz, 2H), 4.31 (m, J=4.1 Hz, 2H), 3.24 (m, J=7.6 Hz, 2H), 2.83 (s, 4H), 2.28 (m, J=5.4 Hz, 2H), 2.02 (s, 1H), 1.80 (m, J=4.6 Hz, 1H), 1.74 (m, J=4.8 Hz, 1H), 1.60 (m, J=4.6 Hz, 1H), 1.54 (m, J=7.0 Hz, 1H), 1.43 (s, 9H), 1.43 (s, 9H), 1.41 (s, 9H), 1.35 (m, J=6.1 Hz, 1H).

Intermediate 8:

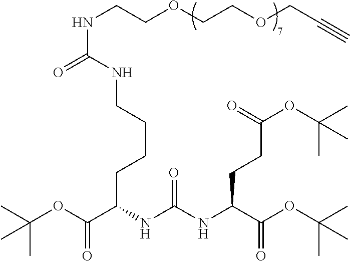

Intermediate 7 (169.4 mg, 0.269 mmol) was dissolved in 1 mL of toluene and dried under vacuum, backfilled with argon, and re-dissolved in 3 mL anhydrous ACN. Amino-PEG8-propargyl (135.2 mg, 0.332 mmol) was resuspended in 1 mL toluene and dried under vacuum, backfilled with argon, and GUL-NHS solution was added. To this mix, TEA (50 uL, 0.359 mmol) was added. This mixture was stirred overnight. The solution was diluted in 75 mL EtOAc and washed once with sat. sodium bicarbonate followed two washes with sat. brine. The organic layer was dried over anhydrous Mg₂SO₄ and concentrated under vacuum. The crude product was purified via flash silica gel column chromatography (consecutive EtOAc:Hex followed by DCM:MeOH gradients) to yield semi-pure product (0.737 mmol, 27.4% yield) as a clear oil. ¹H NMR (700 MHz, CDCl₃) δ 5.68 (d, J=7.6 Hz, 1H), 5.56 (d, J=8.1 Hz, 1H), 4.31 (m, 1H), 4.23 (m, 1H), 4.19 (d, J=2.4 Hz, 2H), 3.65 (m, 28H), 3.55 (m, 2H), 3.34 (m, 2H), 3.14 (m, 2H), 2.44 (t, J=2.4 Hz, 1H), 2.31 (m, 2H), 1.94 (m, 2H), 1.69 (m, 2H), 1.47 (m, 2H), 1.44 (m, 18H), 1.42 (s, 9H), 1.35 (m, 2H). LCMS confirmed correct expected mass of product. MS-ESI [M+H]⁺ m/z calc for [C₄₄H₈₀N₄O₁₆] 921.56, found 921.4949.

Intermediate 9:

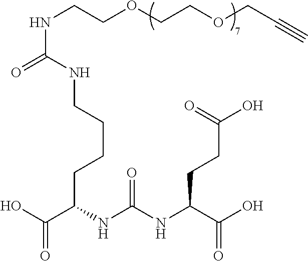

Intermediate 8 (0.737 mmol) was added to TFA (5 mL, neat) while stirring vigorously. After 24 hours, the TFA was blown off. DCM (1 mL) was added and blown off 3 times to help remove residual TFA. The final product was produced in quantitative yields and carried forward to the next reaction. The ¹H NMR and ¹³C NMR spectra of intermediate 9 are shown in FIGS. 69 and 70, respectively.

GU-PEG7-NBoc:

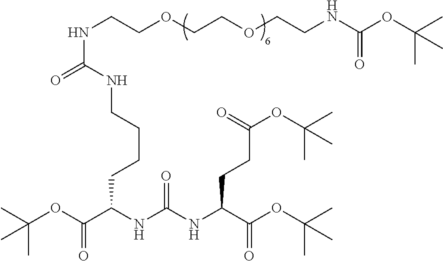

Amino-PEG7-NBoc (0.445 mmol, 1.5 eq) and TEA (0.365 mmol, 1.2 eq) were dissolved in DCM (2 mL). GU-NHS (0.297 mmol, 1 eq) was added to the vial while stirring. The reaction was left for 24 hours, before purification by normal phase flash chromatography using a 5% to 20% DCM/MeOH gradient. The final product was isolated in 43.99% yield (0.128 mmol). ¹H NMR (700 MHz, CD3CN) δ 5.66 (d, J=7.8 Hz, 1H), 5.45 (d, J=8.1 Hz, 1H), 5.40 (s, 1H), 5.17 (t, J=5.7 Hz, 1H), 5.13 (t, J=5.3 Hz, 1H), 4.13 (m, 1H), 4.03 (m, 1H), 3.56 (m 25H), 3.45 (m, 4H), 3.23 (q, J=5.4 Hz, 2H), 3.18 (q, J=5.6 Hz, 2H), 3.06 (q, J=6.5 Hz, 2H), 2.25 (m, 2H), 1.96 (s, 1H), 1.74 (m, 1H), 1.69 (m, J=3.7 Hz, 1H), 1.61 (m, J=6.2 Hz, 1H), 1.42 (q, J=7.3 Hz, 40H). The ¹H NMR spectrum of GU-PEG7-NBoc is shown in FIG. 77.

Intermediate 10 (GU-PEG7-DBCO):

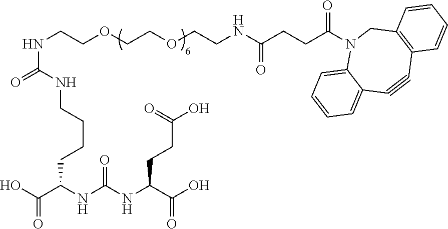

GU-PEG7-NBoc (0.031 mmol, 1 eq) was added to TFA (2 mL, neat) while stirring vigorously. After 24 hours, the TFA was evaporated, and the deprotected product dissolved in 1×PBS (pH 7.2, 0.75 mL). DBCO-NHS (0.060 mmol, 4 eq) was dissolved in acetonitrile (0.5 mL) and added to 0.25 mL of amino-PEG7-GU (0.015 mmol, 1 eq), while stirring vigorously. After 3 hours, the product was purified by HPLC for a final yield of 16.65% (0.0052 mmol) in sufficient analytical quantities to continue. LC-HRMS [M+H]⁺ m/z calc for C₄₈H₆₈N₆O₁₇ 1000.4641, found 1000.4964 (FIG. 18).

### Example 10: Synthesis of ABT Modules for Antibody Recruitment Molecules 2.1-2.19

Peptide Synthesis:

Each peptide was synthesized on a Liberty Blue peptide synthesizer using Fmoc protecting group chemistry. Rink amide resin was used on a 0.1 mmol scale. Each Fmoc deprotection was performed using 20% piperidine in DMF at 90° C. for 1 minute. Each coupling was performed using 1 mL of DIC (1.0 M) in DMF and 0.5 mL of OxymaPure (1.0 M) in DMF, and 2.5 mL of each respective amino acid (0.2 M) in DMF was added at 90° C. for 2 minutes. Double couplings were performed for two consecutive polar amino acids (amino acids A, P, V, L, I, M, F, W), as well as arginine. Once aspartic acid was added to a peptide, all subsequent couplings were performed at 40° C. for 10 minutes. Acetic anhydride was used to cap the N-terminus on bead using a 10% acetic anhydride solution in DMF. Deprotections were performed at room temperature over 3 hours using a 5 mL solution of 9.25:0.25:0.25:0.25 TFA, water, phenol, and TIPS, respectively. Cleaved peptide was crashed out in 10× the volume of −20° C. diethyl ether, pelleted at 2000 rpm over a 15-minute period, and resuspended in 1% acetic acid in water. This was lyophilized off to yield the final capped peptides.

To equip peptides with fluorosulfate-I-tyrosine (FSY) handles, an OtBu-protected tyrosine was installed in the desired sequential location using a peptide synthesizer. After N-terminal acetylated, the tBu protecting group was removed with Pd(PPh₃)₄ (0.025 mmol, 0.25 eq) and PhSiH₃ (0.5 mmol, 5 eq) in DCM (1 mL). This was done for 30 minutes and repeated once. The beads were washed 5× with DCM. AISF (0.267 mmol, 8 eq) was then added with DBU (0.267 mmol, 8 eq) in DCM (4 mL) for 0.5 hours while agitating. This was washed 5× with DCM and air dried. Cleavage was performed with a 5 mL solution of 9.5:0.25:0.25 TFA, water, and TIPS, respectively. This was crashed out in −20° C. diethyl ether, pelleted at 2000 rpm over a 15-minute period, and directly HPLC purified.

Aryl-sulfonyl fluoride (ASF) handles were installed onto peptides as a replacement to N-terminal acetylation. This was done as a standard amino acid coupling, using 1 mL DIC (1.0 M) and 0.5 mL OxymaPure (1.0 M). Cleavage was performed identically to peptides containing OSO₂F handles.

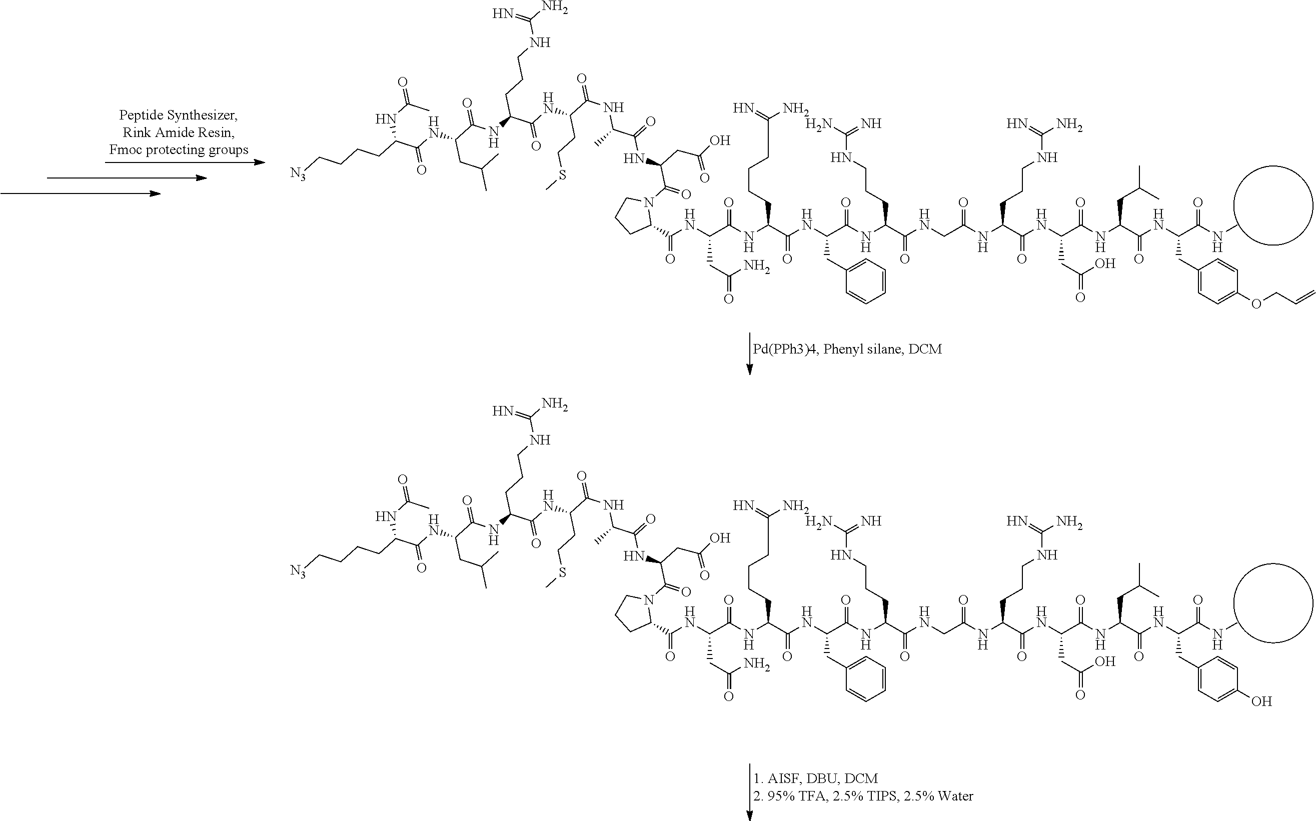

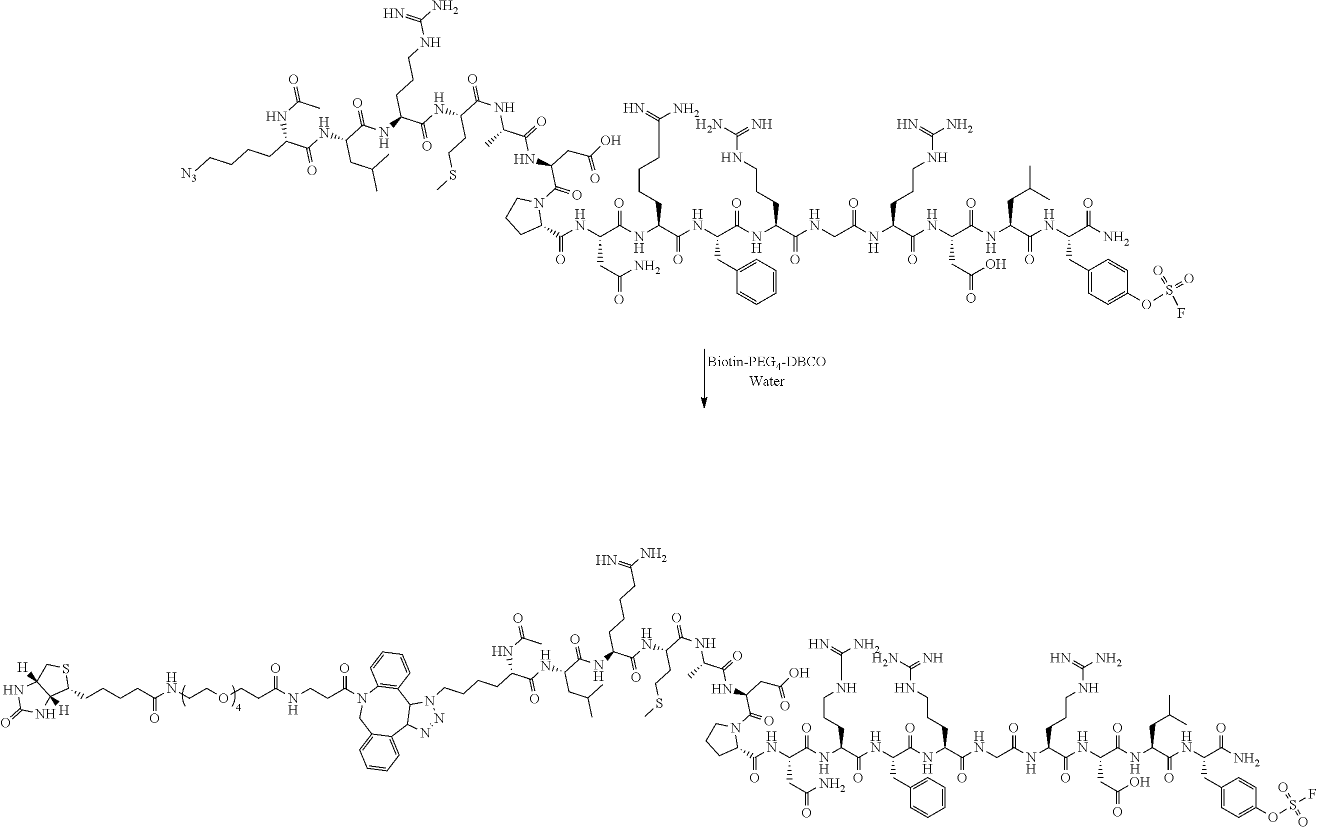

Provided below is an example of the synthesis of cARM 2.9.

Peptide 1 (qD):

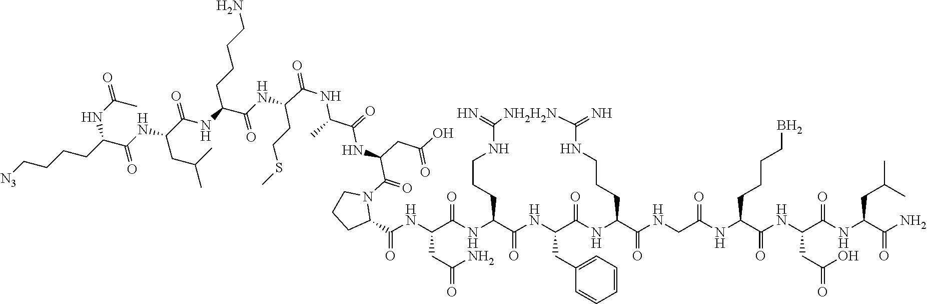

Sequence H—Ac—(N₃—K)-LKMADPNRFRGKDL-NH₂ (SEQ ID NO: 2) was synthesized and HPLC purified. The purity of peptide 1 (gD) was verified by LCMS (FIGS. 19A and 19B).

Peptide 2 (qDR):

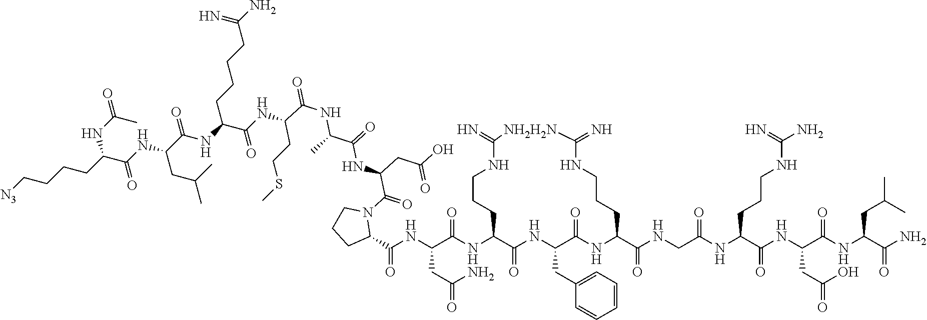

Sequence H—Ac—(N₃—K)-LRMADPNRFRGRDL-NH₂ (SEQ ID NO: 3) was synthesized and HPLC purified. The purity of peptide 2 (gDR) was verified by LCMS. MS-ESI [M+4H]⁴⁺ m/z calc for [C₈₀H₁₃₄N₂₈O₂₁S] 465.05, found 465.1765 (FIGS. 20A and 20B).

Peptide 3 (qDR-Y):

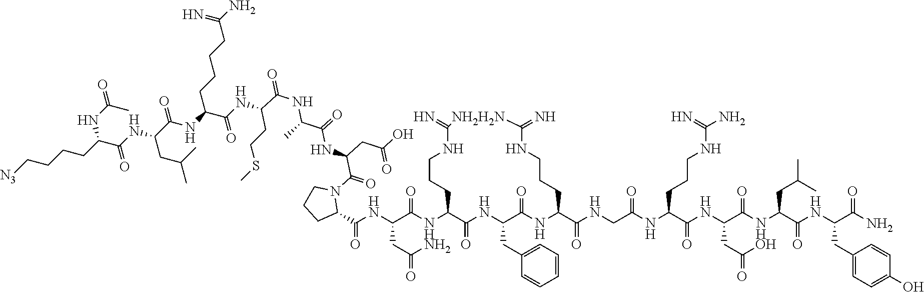

Sequence H—Ac—(N₃—K)-LRMADPNRFRGRDLY-NH₂ (SEQ ID NO: 4) was synthesized and HPLC purified. The purity of peptide 3 (gDR-Y) was verified by LCMS (FIGS. 21A and 21B). The final yield of a 20 mg purification was 22.5%.

Peptide 4 (qDR-FSY):

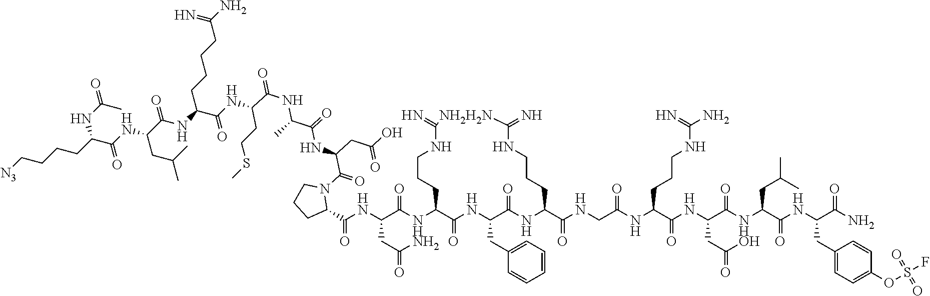

Sequence H—Ac—(N₃—K)-LRMADPNRFRGRDL-(Y—OSO₂F)—NH₂ (SEQ ID NO: 5) was synthesized and HPLC purified. The purity of peptide 4 (gDR-FSY) was verified by LCMS (FIGS. 22A and 22B). The final peptide was isolated in 44% yield. The ¹⁹F NMR spectrum of peptide 4 is shown in FIG. 71.

Peptide 5 (qDR(F10FSY)):

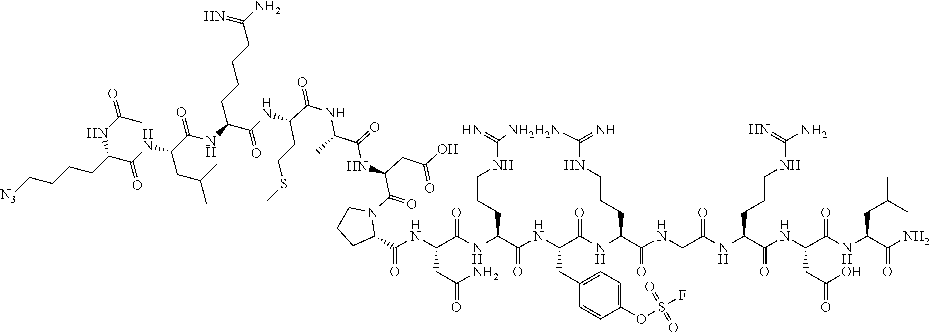

Sequence H—Ac—(N₃—K)-LRMADPNR-(Y—OSO₂F)-RGRDL-NH_{z}(SEQ ID NO: 7) was synthesized and HPLC purified. The purity of peptide 5 (gDR(F10FSY)) was verified by LCMS (FIGS. 23A and 23B). The final yield of peptide was 20.9%.

Peptide 6 (FSY-qDR):

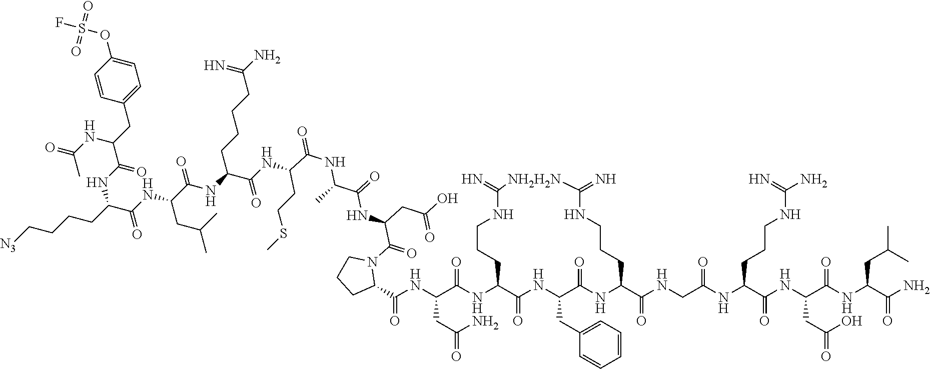

Sequence H—Ac—(Y—OSO₂F)—(N₃—K)-LRMADPNRFRGRDL-NH₂ (SEQ ID NO: 6) was synthesized and HPLC purified. The purity of peptide 6 (FSY-gDR) was verified by LCMS (FIGS. 24A and 24B). The final yield of pure peptide was 34.0%.

Peptide 7 (ASF-qDR):

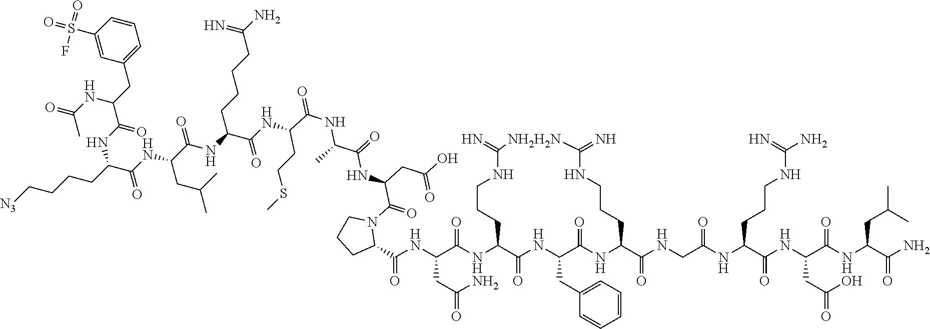

Sequence H-(Aryl-SO₂F)—(N₃—K)-LRMADPNRFRGRDL-NH₂ (SEQ ID NO: 8) was synthesized. Crude ASF-gDR (20 mg) was HPLC-purified, providing 2.6 mg of pure peptide, with an estimated yield of 13%. The purity of peptide 7 (ASF-gDR) was verified by LCMS (FIG. 25). The ¹⁹F NMR spectrum of peptide 7 is shown in FIG. 72.

Peptide 8 (SO₃H-qDR):

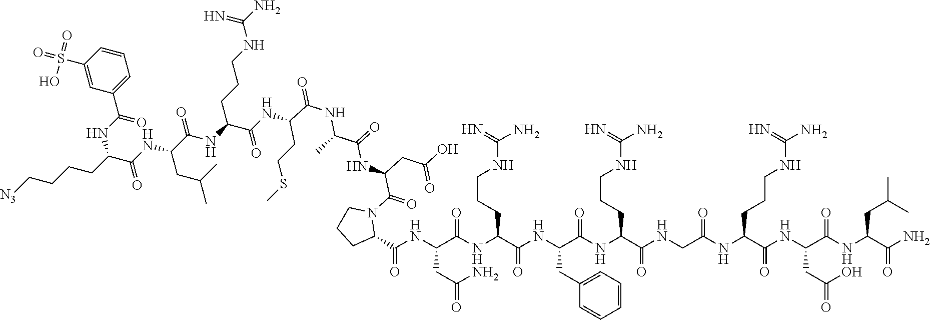 [SMILES: CSCC[C@H](NC(=O)[C@H](CCCNC(=N)N)NC(=O)[C@H](CC(C)C)NC(=O)[C@H](CCCCN=[N+]=[N-])NC(=O)c1cccc(S(=O)(=O)O)c1)C(=O)N[C@@H](C)C(=O)N[C@@H](CC(=O)O)C(=O)N1CCC[C@H]1C(=O)N[C@H]1CC(=O)NN([C@@H](CCCNC(=N)N)C(=O)N[C@@H](Cc2ccccc2)C(=O)N[C@@H](CCCNC(=N)N)C(=O)NCC(=O)N[C@@H](CCCNC(=N)N)C(=O)N[C@H]2CC(=O)ON([C@@H](CC(C)C)C(N)=O)C2=O)C1=O]

Sequence H-(Aryl-SO₃H)—(N₃—K)-LRMADPNRFRGRDL-NH₂ (SEQ ID NO: 9) was made by incubating peptide 7 (ASF-gDR) in 1×PBS for 7 days at room temperature. The purity of peptide 8 (SO₃H-gDR) was verified by LCMS (FIG. 78).

### Example 11: Synthesis of Covalent Antibody Recruitment Molecules 2.1-2.10

Final compounds were synthesized using the strain-promoted azide-alkyne cycloaddition (SPAAC) click reaction to orthogonally link peptides (antibody binding terminus, ABT) to “tumor binding domains” (target binding terminus, TBT). SPAAC click reactions were performed under aqueous conditions at room temperature. Both N₃-peptide and DBCO-biotin/fluorescein/GUL were combined at final concentrations of 200 μM in water. Reactions were complete after 3 hours and were verified by LCMS. Final stocks were stored at −20° C. until used for their respective experiment.

cARMs 2.1-2.10 were synthesized by substituting TBTs or reactive groups in four different locations on the peptide, R₁-R₄, as shown in the below formula and in Table 2. The “R” group in variables R₁-R₄ in the below formula indicates the point of covalent attachment of each variable to the peptide.

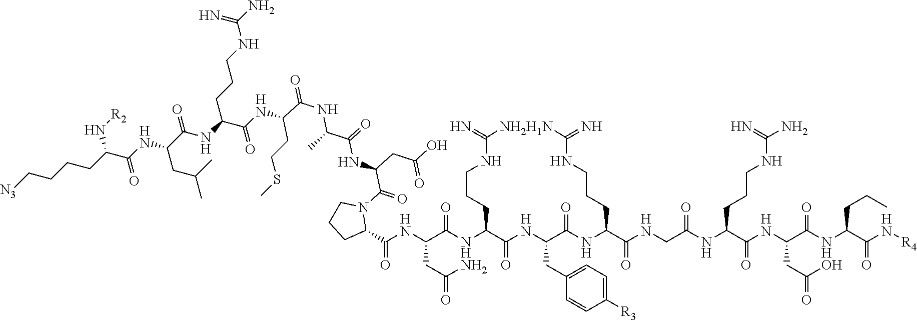

R1:

| | |
|---|---|
| A: | 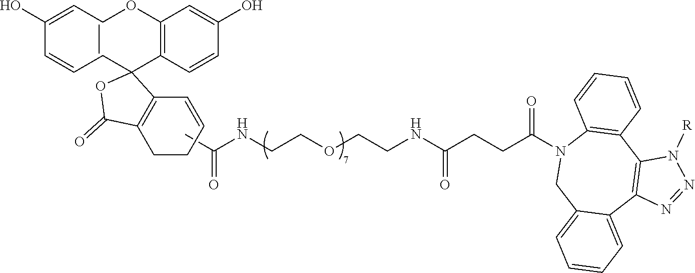 |
| B: | 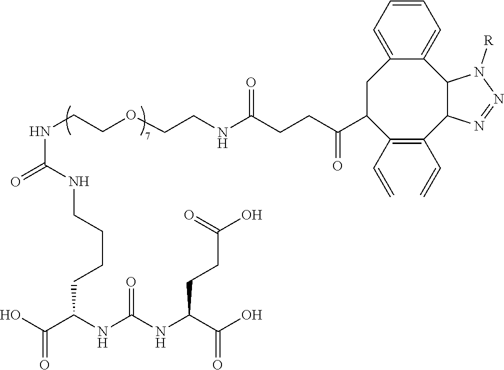 |
| C: | 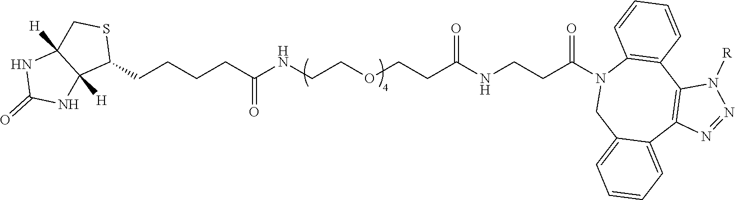 |

R2:

| | |
|---|---|
| D: | 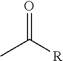 |
| E: | 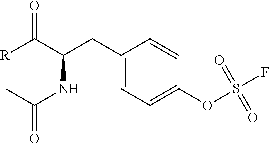 |
| F: | 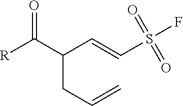 |

R3:

| | |
|---|---|
| G: | H |
| H: | 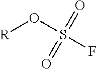 |

R4:

| | |
|---|---|
| G: | H |
| I: | 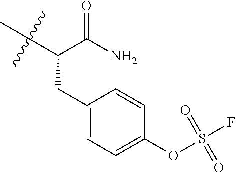 [SMILES: *[C@@H](CC/C=C\C(=C/C)OS(=O)(=O)F)C(N)=O] |

TABLE 2
Covalent Antibody Recruitment Molecules 2.1-2.10

Variable Group

| Compound | R₁ | R₂ | R₃ | R₄ |
|---|---|---|---|---|
| cARM 2.1 | A | E | G | G |
| cARM 2.2 | A | D | H | G |
| cARM 2.3 | A | D | G | I |
| cARM 2.4 | B | E | G | G |
| cARM 2.5 | A | F | G | G |
| cARM 2.6 | B | F | G | G |
| cARM 2.7 | C | E | G | G |
| cARM 2.8 | C | D | H | G |
| cARM 2.9 | C | D | G | I |
| cARM 2.10 | C | F | G | G |

cARM 2.1 (Fluorescein-FSY-qDR):

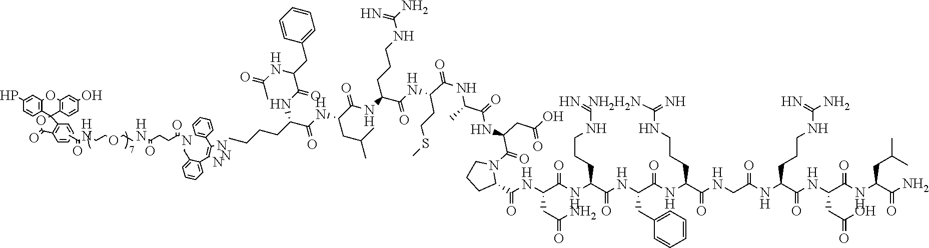

Peptide 6 (FSY-gDR) and intermediate 6 (fluorescein-PEG7-DBCO) were incubated together (150 μM each, 400 μL water) for 3 hours at room temperature. HRMS-ESI [M+5H]⁵⁺ m/z calc for [C₁₄₅H₂₀₁FN₃₆O₄₀S_{2] 634.8845}, found 635.3055 (FIGS. 26A and 26B).

cARM 2.2 (Fluorescein-gDR(F10FSY)):

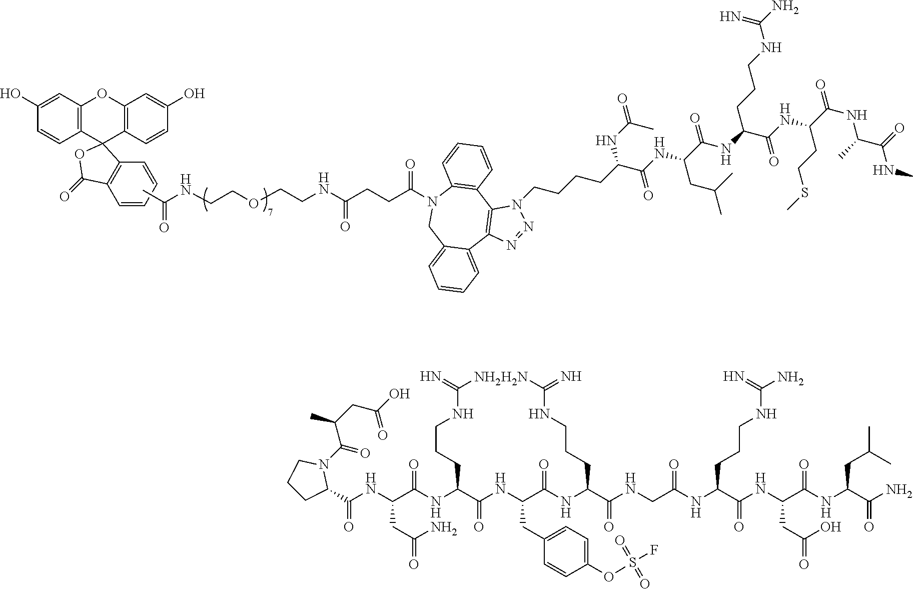

Peptide 5 (gDR(F10FSY)) and intermediate 6 (fluorescein-PEG7-DBCO) were incubated together (150 μM each, 400 μL water) for 3 hours at room temperature. HRMS-ESI [M+5H]⁵⁺ m/z calc for [C₁₃₆H₁₉₂FN₃₅O₃₉S_{2] 605.4708}, found 605.6925 (FIGS. 27A and 27B).

cARM 2.3 (Fluorescein-qDR-FSY):

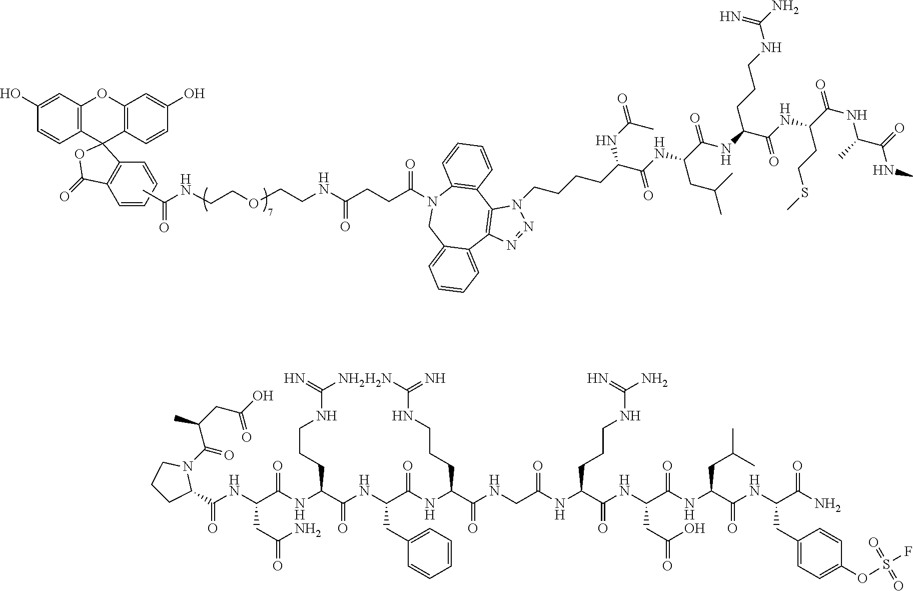

Peptide 4 (gDR-FSY) and intermediate 6 (fluorescein-PEG7-DBCO) were clicked together in a 200 μM, 400 μL reaction. This was left for 3 hours before verifying reaction completion by LCMS (FIGS. 28A and 28B).

cARM 2.4 (GU-FSY-qDR):

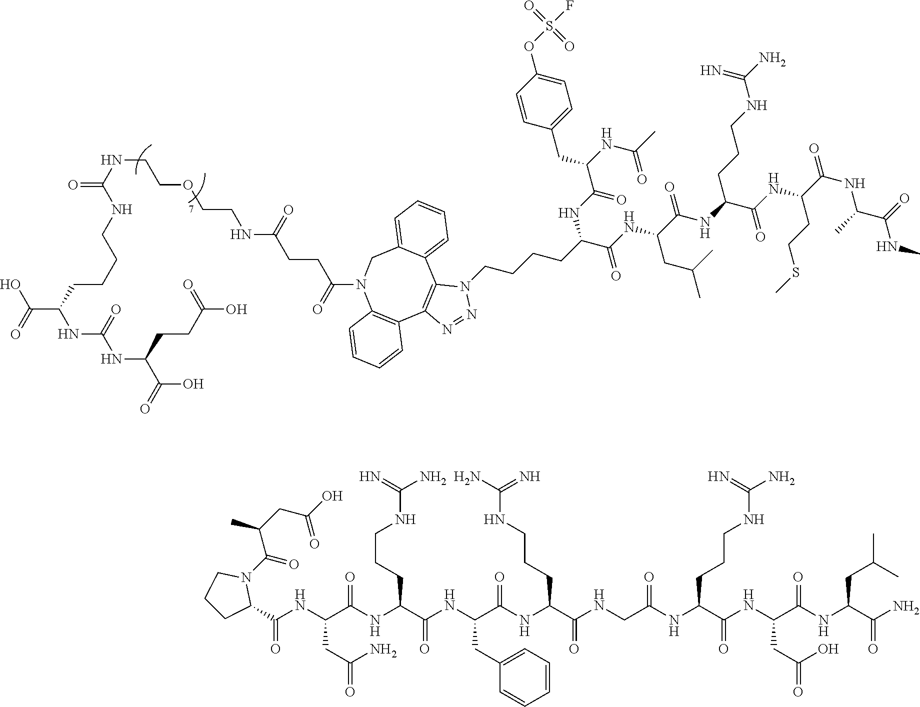

Peptide 6 (FSY-gDR) and intermediate 10 (GU-PEG7-DBCO) were incubated together (100 μM each, 200 μL water) for 3 hours at room temperature. HRMS-ESI [M+4H]⁴⁺ m/z calc for [C₁₃₇H₂₁₀FN₃₉O₄₂S₂] 790.1230, found 790.4005 (FIG. 79).

cARM 2.5 (Fluorescein-ASF-qDR):

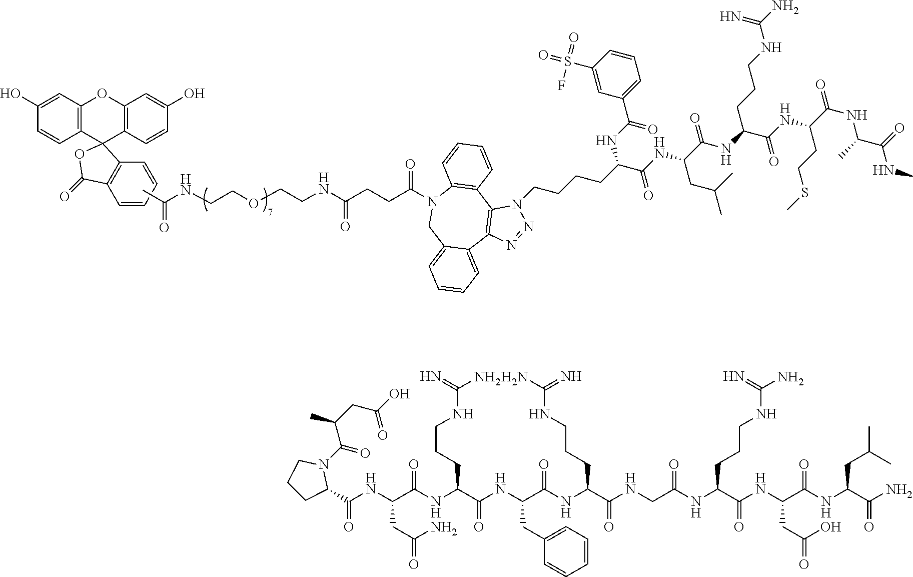

Peptide 7 (ASF-gDR) and intermediate 6 (fluorescein-PEG7-DBCO) were incubated together (150 μM each, 200 μL water) for 3 hours at room temperature. HRMS-ESI [M+5H]⁵⁺ m/z calc for [C₁₄₁H₁₉₄FN₃₅O₃₈S₂] 614.6750, found 614.9042 (FIGS. 29A and 29B).

cARM 2.6 (GU-ASF-qDR):

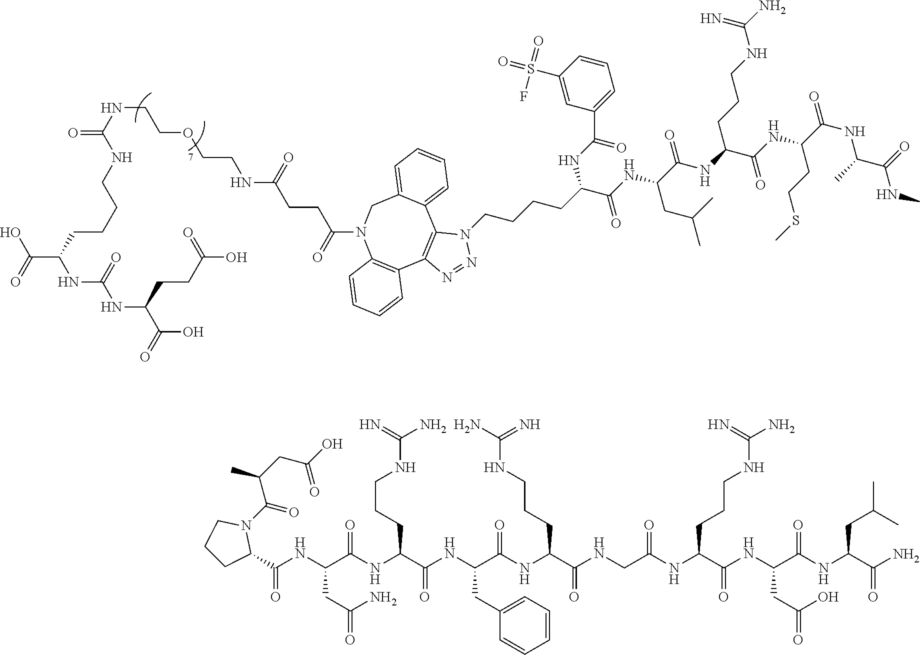

Peptide 7 (ASF-gDR) and intermediate 10 (GU-PEG7-DBCO) were incubated together (100 μM each, 200 μL water) for 3 hours at room temperature. HRMS-ESI [M+4H]⁴⁺ m/z calc for [C₁₃₃H₂₀₃FN₃₈O₄₀S₂] 764.8611, found 765.1390 (FIG. 80).

cARM 2.7 (Biotin-FSY-qDR):

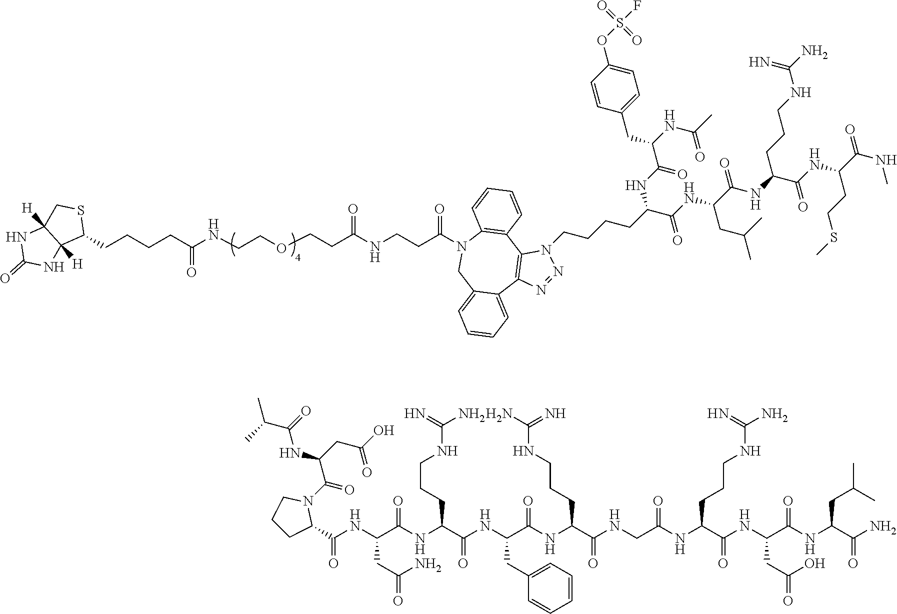

Peptide 6 (FSY-gDR) and biotin-PEG4-DBCO were incubated together (200 μM each, 400 μL water) for 3 hours, and observed to convert stoichiometrically to the desired product. Crude products of sufficiently high purity were used directly in subsequent assays. HRMS-ESI [M+4H]⁴⁺ m/z calc for [C₁₂₈H₁₉₃FN₃₈O₃₃S₃] 727.3535, found 727.6269 (FIGS. 30A and 30B).

cARM 2.8 (Biotin-gDR(F10FSY)):

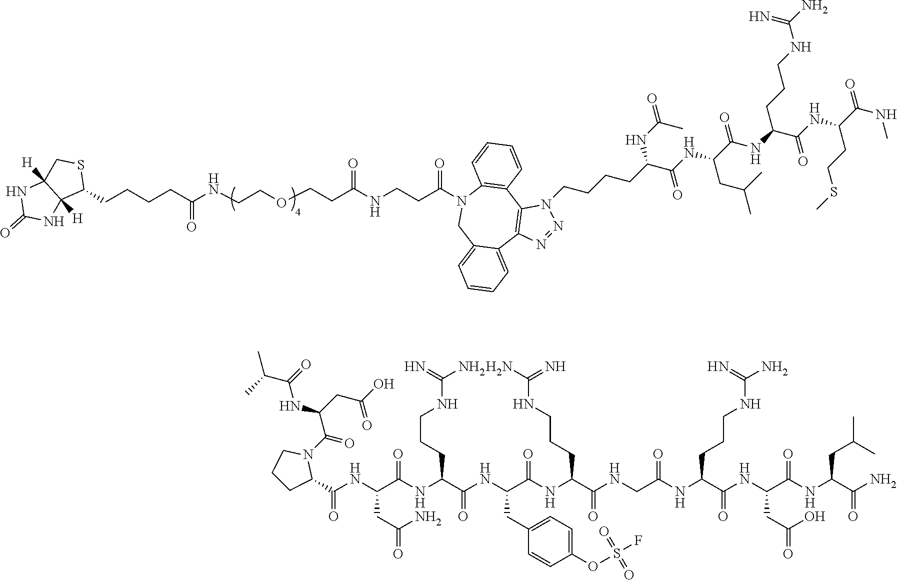

Peptide 5 (gDR(F10FSY)) and biotin-PEG4-DBCO were incubated together (200 μM each, 400 μL water) for 3 hours at room temperature. HRMS-ESI [M+4H]⁴⁺ m/z calc for [C₁₁₉H₁₈₄FN₃₇O₃₂S₃] 690.5764, found 690.8548 (FIGS. 31A and 31B).

cARM 2.9 (Biotin-gDR-FSY):

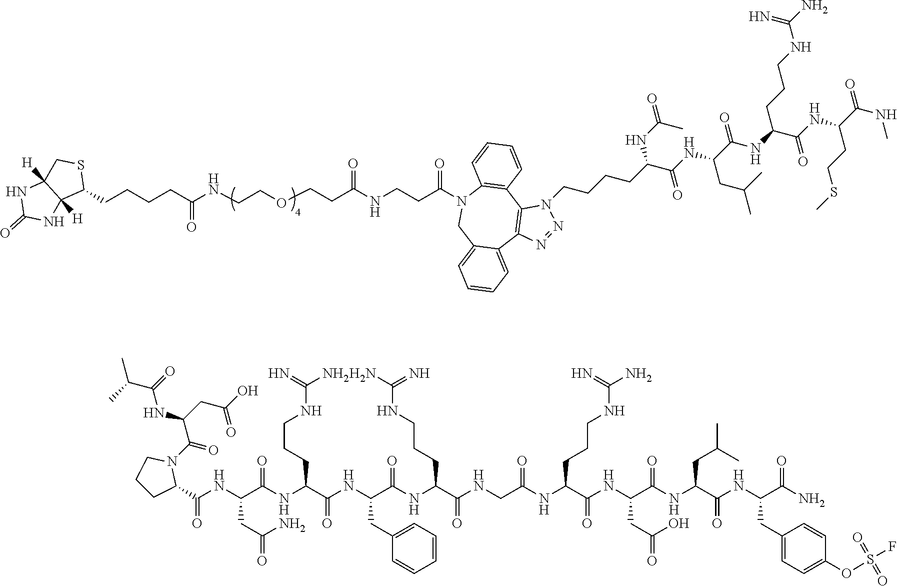

Peptide 4 (gDR-FSY) and a DBCO-biotin linker were clicked together in a 200 μM, 400 μL reaction. This was left for 3 hours before verifying reaction completion by LCMS (FIGS. 32A and 32B).

cARM 2.10 (Biotin-ASF-qDR):

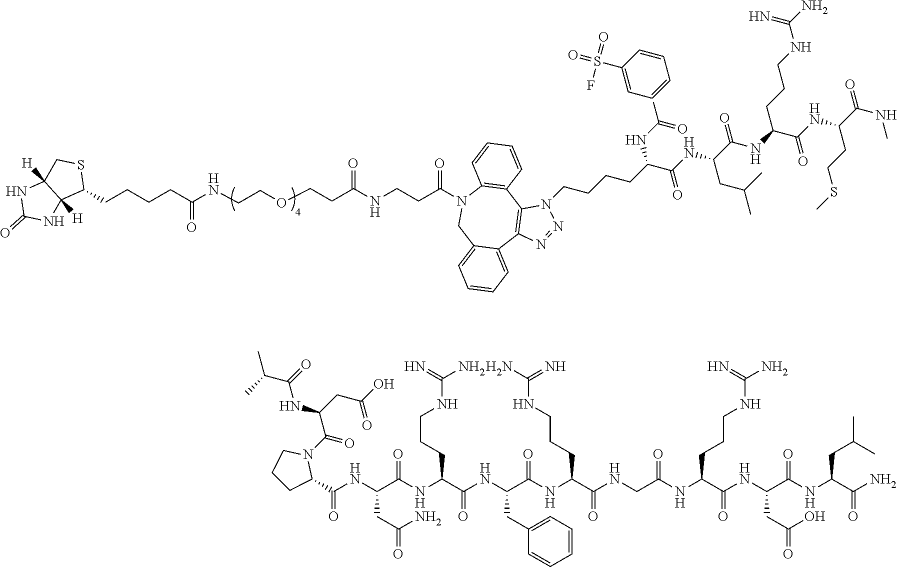

Peptide 7 (ASF-gDR) and biotin-PEG4-DBCO were incubated together (200 μM each, 400 μL water) for 3 hours at room temperature. HRMS-ESI [M+4H]⁴⁺ m/z calc for [C₁₂₄H₁₈₆FN₃₇O₃₁S₃] 702.0816, found 702.3633 (FIGS. 33A and 33B).

### Example 12: Synthesis of Non-Covalent Antibody Recruitment Molecules 2.11-2.19

ARM 2.11 (DTB-PEG8-qD):

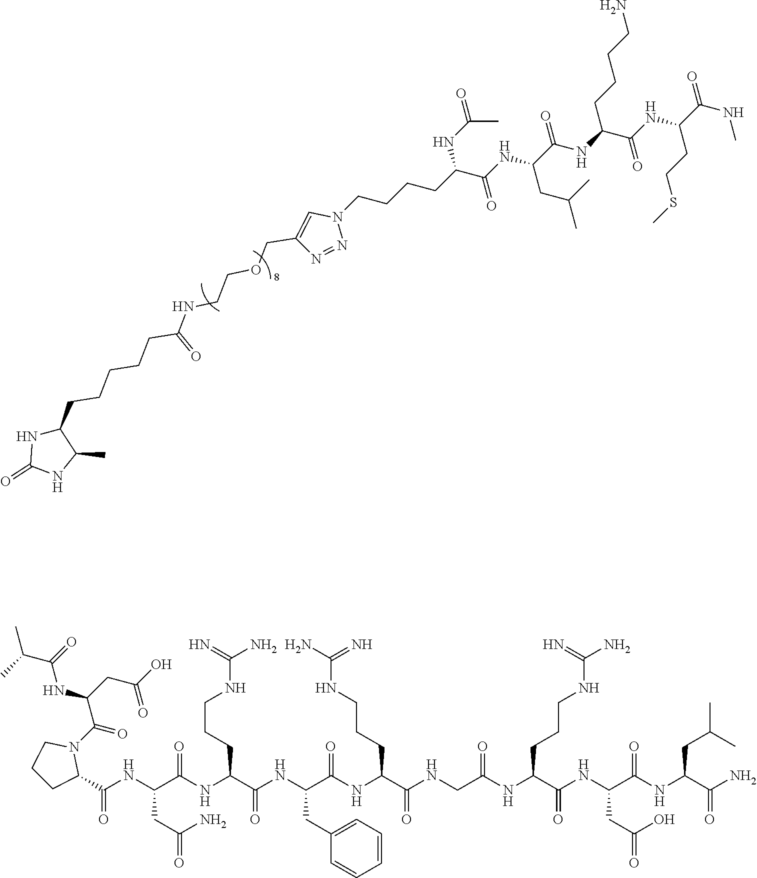

Both peptide 1 (gD) and intermediate 2 were transferred to a vial with a stir bar. The vial was evacuated with argon and the DMSO was added. The CuSO₄ pentahydrate, sodium ascorbate, and THPTA were dissolved in water and transferred to the click reagents to begin the reaction. The reaction proceeded over 24 hours. This was purified by first centrifuging the reaction at 14000 rpm for 5 minutes, and then injecting the supernatant on high performance liquid chromatography using a gradient of 10%-90% acetonitrile in water with 0.1% formic acid. Peaks corresponding to product were pooled and lyophilized. The final product was obtained in 14.51% yield. MS-ESI [M+4H]⁴⁺ m/z calc for [C₁₀₉H₁₈₇N₃₁O₃₁S]615.845, found 615.8500 (FIGS. 34A and 34B).

ARM 2.12 (Biotin-PEG4-qD):

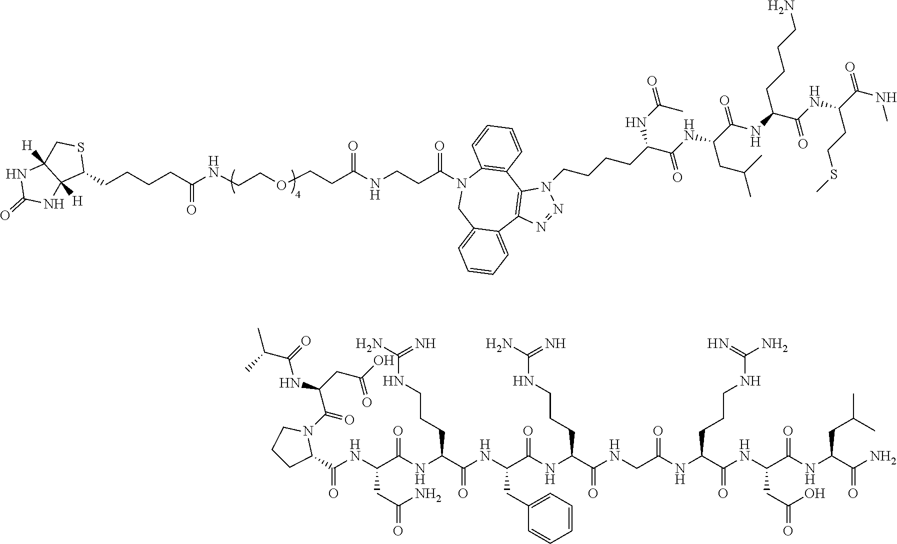

Peptide 1 (gD) and biotin-PEG4-DBCO were clicked together in a 150 μM, 400 μL reaction. This was left for 3 hours before verifying reaction completion by LCMS (FIGS. 35A and 35B).

ARM 2.13 (DTB-PEG8-qDR):

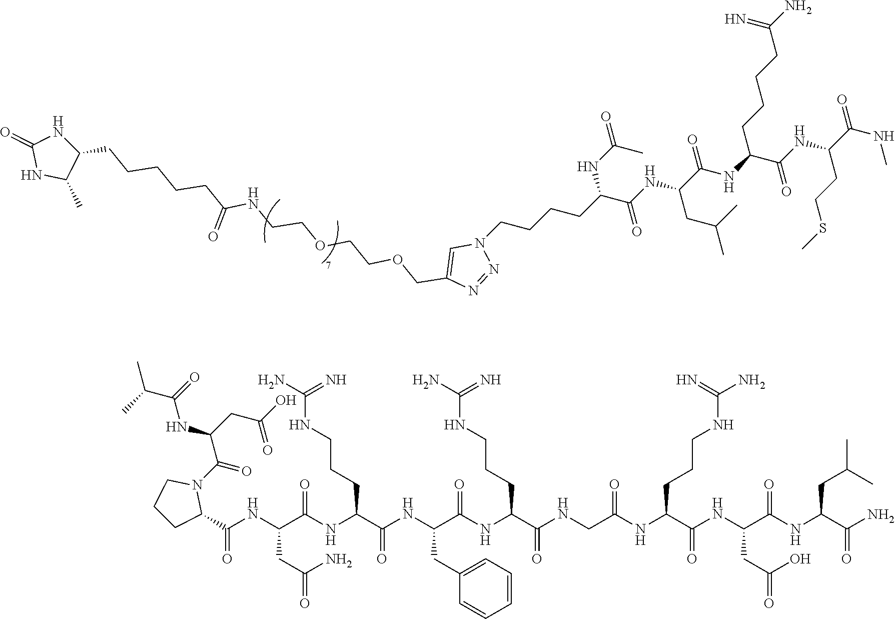

Peptide 2 (gDR, 0.0165 mmol, 1 eq) and intermediate 2 (0.0165 mmol, 1 eq) were transferred to a vial and dissolved in 1 mL of 10% DMSO, 90% water. On the side, copper (II) sulfate (0.00165 mmol, 0.1 eq), sodium ascorbate (0.0033 mmol, 0.2 eq), and THPTA (0.00297 mmol, 0.18 eq) were dissolved in 100 μL water and transferred to the vial to begin the CuAAc reaction. After the reaction turned blue, indicating excess copper (II), additional sodium ascorbate was added (0.0033 mmol, 0.2 eq). This was left for 24 hours before verifying reaction completion by LCMS (FIGS. 36A and 36B).

A RM 2.14 (Biotin-PEG4-qDR):

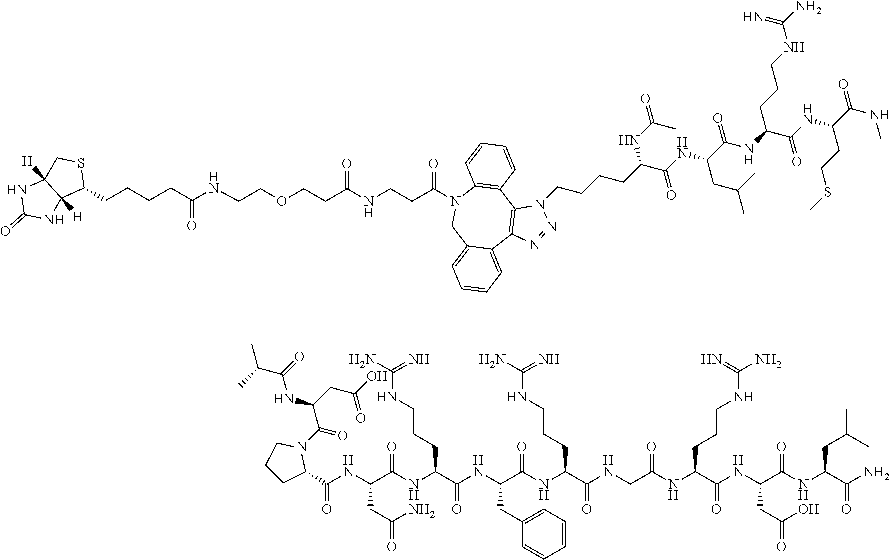

Peptide 2 (gDR) and biotin-PEG4-DBCO were clicked together in a 150 μM, 400 μL reaction. This was left for 3 hours before verifying reaction completion by LCMS (FIGS. 37A and 37B).

ARM 2.15 (Biotin-qDR-Y):

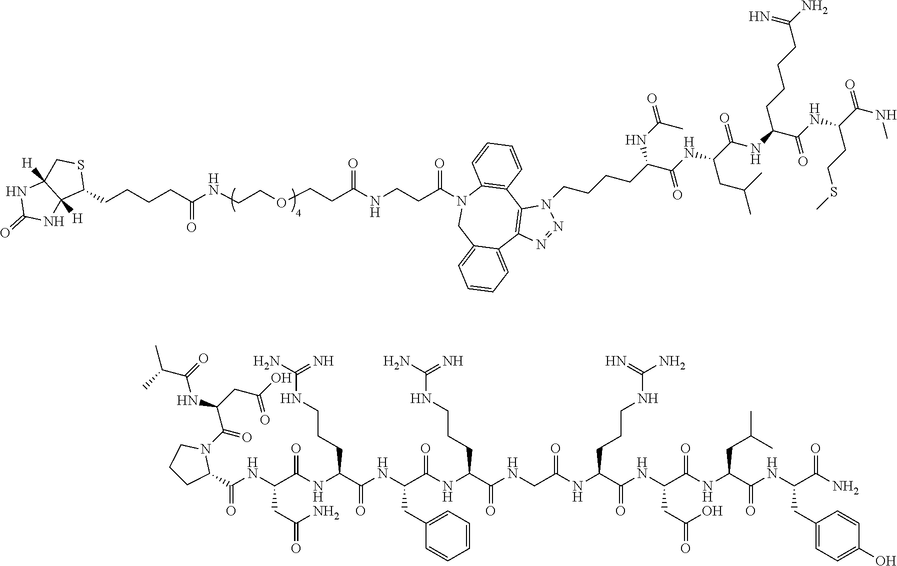

Peptide 3 (gDR-Y) and biotin-PEG4-DBCO were clicked together in a 150 μM, 400 μL reaction. This was left for 3 hours before verifying reaction completion by LCMS (FIGS. 38A and 38B).

ARM 2.16 (Fluorescein-QD):

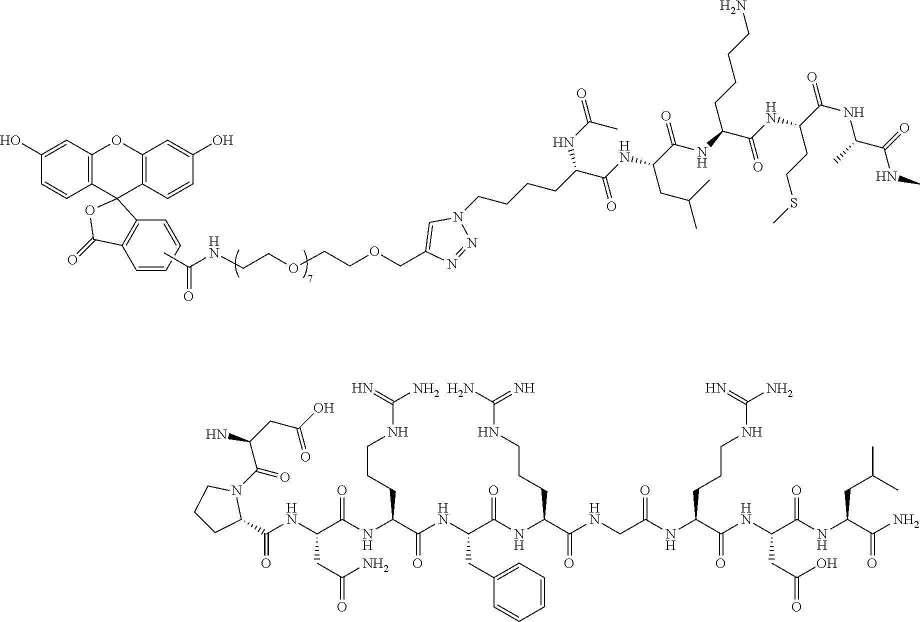

Both the crude peptide 1 (gD) and intermediate 3 were transferred to a vial with a stir bar. The vial was evacuated with argon and the DMSO was added. The CuSO₄ pentahydrate, sodium ascorbate, and THPTA were dissolved in water and transferred to the click reagents to begin the reaction. The reaction proceeded over 24 hours. This was purified by first centrifuging the reaction at 14000 rpm for 5 minutes, and then injecting the supernatant on high performance liquid chromatography using a gradient of 10%-90% acetonitrile in water with 0.1% formic acid. Peaks corresponding to product were pooled and lyophilized (FIG. 39). The final product was obtained in 14.51% yield.

ARM 2.17 (GU-qD):

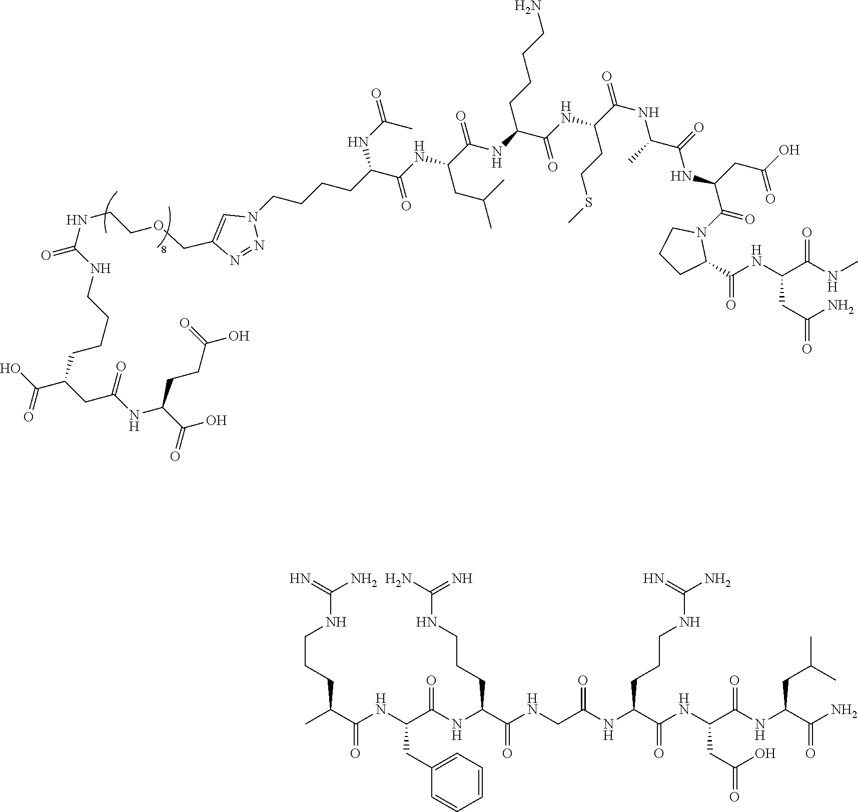

Both the crude peptide 1 (gD, 0.012 mmol, 1 eq) and intermediate 9 (0.0159 mmol, 1 eq) were transferred to a vial with a stir bar. The vial was evacuated with argon and the DMSO was added. The CuSO₄ pentahydrate, sodium ascorbate, and THPTA were dissolved in water and transferred to the click reagents to begin the reaction. The reaction proceeded over 24 hours. This was purified by first centrifuging the reaction at 14000 rpm for 5 minutes, and then injecting the supernatant on high performance liquid chromatography using a gradient of 10%-90% acetonitrile in water with 0.1% formic acid. Peaks corresponding to product were pooled and lyophilized. The final product was obtained in 14.51% yield. MS-ESI [M+4H]⁺ m/z calc for [C₁₁₂H₁₉₀N₃₂O₃₇S] 653.25, found 653.1 (FIGS. 40A and 40B).

ARM 2.18 (GU-SO₃H-qDR):

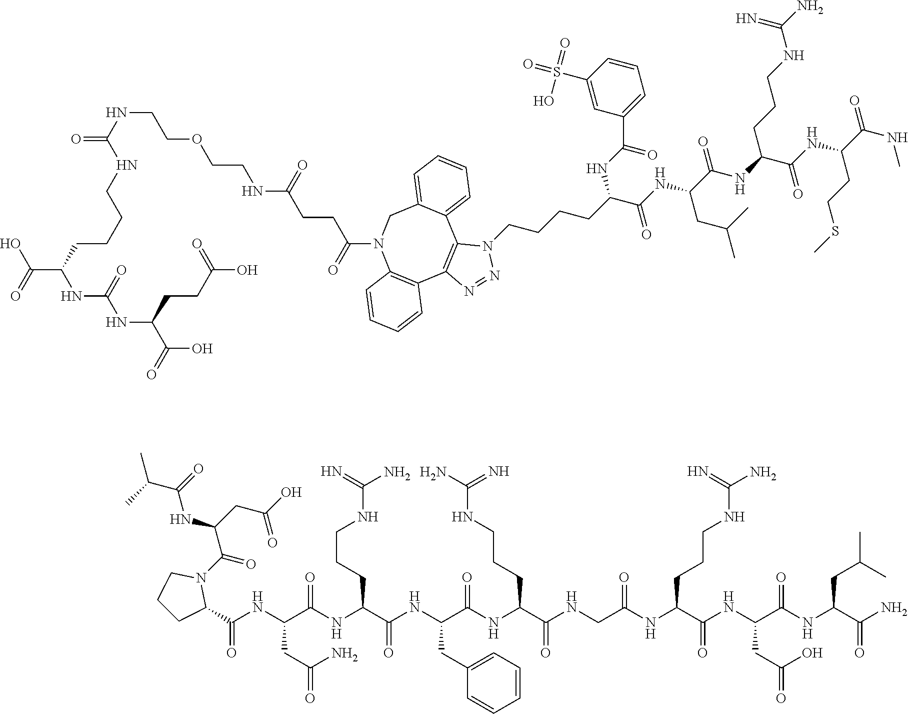

Peptide 8 (SO₃H-gDR) and intermediate 10 (GU-PEG7-DBCO) were incubated together (100 μM each, 200 μL water) for 3 hours, and observed to convert stoichiometrically to the desired product. The crude product was of sufficiently high purity to be used directly in subsequent assays. The purity of ARM 2.18 (GU-SO₃H-gD) was verified by LCMS (FIG. 81).

ARM 2.19 (Biotin-SO₃H-gDR):

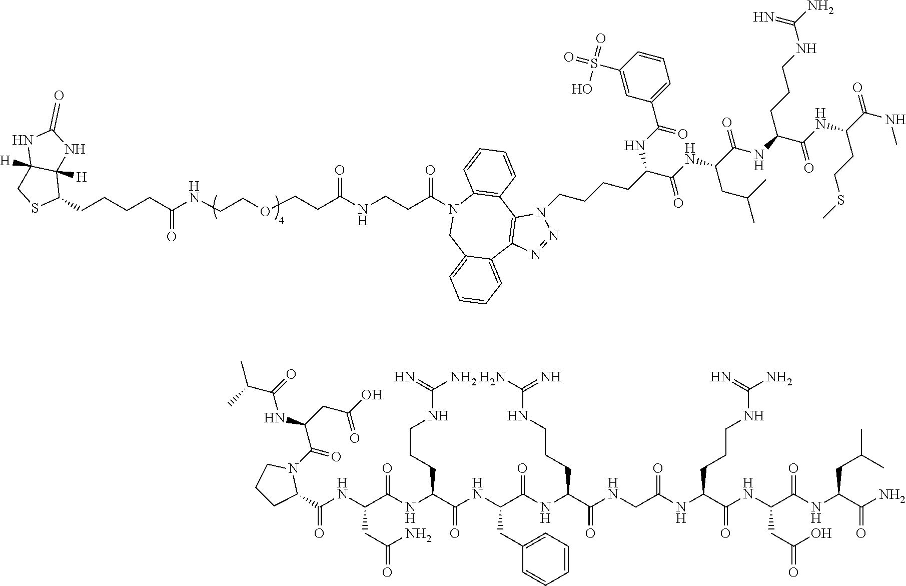

Peptide 8 (SO₃H-gDR) and biotin-PEG4-DBCO were incubated together (150 μM each, 200 μL water) for 3 hours, and observed to convert stoichiometrically to the desired product. The purity of ARM 2.19 (Biotin-SO₃H-gD) was verified by LCMS (FIG. 82).

### Example 13: Assessment of the Stability of Covalent Antibody-Binding Peptides by LC-HRMS

LC-HRMS General Procedure:

Liquid chromatography-high resolution mass spectrometry (LC-HRMS) data was obtained on a BRUKER MicroTOF II mass spectrometer. Samples were dissolved in 1×PBS at 500 μM concentrations and were monitored for stability at room temperature over indicated periods of time. Specific m/z −20 amu (loss of H⁺ and F⁻) and m/z −2 amu (F/OH exchange) masses were monitored to probe off-pathway cyclization and hydrolysis reactions, respectively.

LC-HRMS Results:

To evaluate the stability of peptide 5 (gDR(F10FSY)), the peptide (500 μM) was incubated in 1×PBS at room temperature and analyzed by LC-HRMS at 0, 24, 48 and 72 hours (FIG. 83). Observed in each chromatogram of FIG. 83 is a sharp peak attributed to the target mass of peptide 5 (indicating a stable compound), and a small broad peak of the same mass. Hydrolyzed peptide appeared to elute just before intact peptide after 3 days of incubation.

To evaluate the stability of peptide 7 (ASF-gDR), the peptide (500 μM) was incubated in 1×PBS at room temperature and analyzed by LC-HRMS at 0, 24, 48 and 72 hours. As can be seen in FIG. 84, SuFEx hydrolysis reached nearly 50% after 24 hours of incubation, appearing as an [(M-2)+3H]³⁺ peak (F/OH substitution).

### Example 14: Assessment of the Stability of Covalent Antibody-Binding Peptides by ¹⁹F NMR

¹⁹F NMR General Procedure:

A 700 MHz NMR was used to measure ¹⁹F NMR spectra. Bruker TopSpin 4.0.9 was used to process and analyze spectra. GraphPad Prism 8 software was used to visualize and perform non-linear regression of acquired data.

¹⁹F NMR Results:

Peptide 7 (ASF-gDR) was used to investigate the stability of the meta-aryl sulfonyl fluoride (ASF) group in 1×PBS at room temperature. The sample was prepared at a 0.5 mM concentration in 90% PBS, 10% D₂O. A 700 MHz NMR was used to monitor sulfonyl fluoride disappearance and fluoride (F⁻ (aq)) appearance overtime, relative to a TFA internal standard. To quantify fluoride loss and gain, peaks were positively phased and integrated relative to the TFA internal standard, which was held constant at three fluoride atoms. A plot of fluoride leaving group appearance over time was fitted with the first order decay equation. Due to the low sensitivity at decreasing concentrations of ASF, the appearance of aqueous fluoride leaving group was monitored, as this signal was found to be more sensitive. The stability curve for Aryl-SO₂F is shown in FIG. 41.

Half-life was calculated from a plot of integrated F⁻ over time using Equation 1. Under these conditions, the half-life was calculated to be 8.710 hours.

Equation 1: First Order Decay

$$- \frac{d [ {SO}_{2} F ]}{dt} = λ [ {SO}_{2} F ] \\ \frac{d [ {F}^{-} ]}{dt} = λ [ {F}^{-} ] \\ ln ( \frac{{[ {F}^{-} ]}_{t}}{{[ {F}^{-} ]}_{0}} ) = λ t$$

To further evaluate the stability of peptide 7 (ASF-gDR), hydrolysis of sulfonyl fluoride was measured by ¹⁹F NMR in order. Peptide 7 (ASF-gDR) was dissolved in 90% PBS/10% D₂O and monitored by ¹⁹F NMR for F⁻ (aq) generation as a function of time over the course of 72 hours (FIGS. 85 and 86).

### Example 15: Bio-Layer Interferometry Experiments

General Procedure:

BLI experiments were all performed using an Octet Red96. 1× kinetics buffer (1× KB; 1× PBS, 0.01% BSA, 0.002% Tween20) spiked with 1% (v/v) DMSO was used as a buffer for each solution. Soluble PSMA was generously provided by Dr. Cyril Barinka (Institute of Biotechnology CAS, Czech Republic). Streptavidin and protein G-coated biosensors were purchased from Sartorius (Part No. 18-5019). Prior to experiments, probes were wetted in 1× KB for 10 minutes. Measurements were taken at an RPM of 1000 and temperature of 30° C., with an acquisition rate of 5 Hz. An initial baseline was always performed for 60-180 seconds, or until signal was stable. Quenching was done in 5% (w/v) skim milk dissolved in 1× KB for 120 seconds. All ligand and protein conditions were diluted in 1× KB to achieve a final volume of 200 μL in each well. A polyclonal human IgG isotype control was purchased from Jackson ImmunoResearch (Cat. No. 009-000-003). Mouse IgG2a LP14 anti-HSV gD antibody was purchased from Sigma Aldrich (Cat. No. MABF1975). Mouse IgG2a Anti-HSV-½ gD Antibody (H₁₇₀) was purchased from Santa Cruz Biotechnology (Cat. No. sc-69802).

Assessing Binding Kinetics in Bio-Layer Interferometry:

Binding curves for each biotin-peptide conjugate and LP14 mAb were constructed using BLI. Streptavidin coated biosensor probes were loaded with 200 nM of biotinylated peptide for 180 seconds. A quench was then performed for 150 seconds to reduce non-specific binding. To re-establish baseline, the probes were placed in kinetics buffer for 60 seconds. An association phase followed, where probes were placed in a solution of 100 nM LP14 mAb. A dissociation phase utilized 100 μM peptide 1 in 1× KB to offset the effects of avidity; an artifact arising from the proximity of immobilized peptide, such that no k_{off} is observed in the absence of competitor. Data was processed on Graph Pad Prism 8 using association and dissociation nm shifts. A baseline was performed to the nm shift at the beginning of association. Salt effects, such as large increases in nm shift between association and dissociation wells, were removed. On and off rate constants were derived through a two-phase association, non-linear regression (Equation 2c), and first order decay (Equation 3), respectively. K_{D} values were calculated from the ratio of kₒₙ/k_{off}.

Equation 2: Two Phase Association
a) SpanFast=(Plateau−Y₀)PercentFast×0.01
b) SpanSlow=(Plateau−Y₀)(100−PercentFast)×0.01
c) Y=Y₀+SpanFast(1−exp(−K_{Fast}X))+SpanSlow(1−exp(−K_{slow}X))
Y₀=Y value when (X=0)
Plateau=Y value after infinite X values
K_{Fast}=Fast rate constant, calculated as a reciprocal of the rate constant
K_{Slow}=Slow rate constant, calculated as a reciprocal of the rate constant
PercentFast=Fraction from Y₀ to plateau accounted for by K_{Fast}

Equation 3: One Phase Exponential Decay

Y=(Y₀−NS)(exp(−KX))+NS
X: Time
Y: nm Shift
Y₀: Y at time 0
NS: Binding at very long time points
K: Rate constant (s⁻¹)

Differences in LP14 mAb binding affinity between peptides having PEG linkers of different lengths and a double Lys to Arg mutation were evaluated by BLI. Both peptide 1 (gD) and peptide 2 (gDR) were modified with biotin-PEG4-DBCO or intermediate 2 (DTB-PEG8-DBCO). Analysis was performed using biotinylated non-covalent ARMs (200 μM) which were immobilized onto streptavidin-coated probes before association with anti-HSV gD LP14 mAb (100 μM). Dissociation took place in a solution of 100 μM gD peptide. As can be seen in FIG. 42, no binding difference was observed between linker lengths of PEG8 (ARM 2.11: DTB-PEG8-gD and ARM 2.13: DTB-PEG8-gDR) and PEG4 (ARM 12: Biotin-PEG4-gD and ARM 14: Biotin-PEG4-gDR) in this experiment. When lysine was replaced with arginine residues (ARM 2.13: DTB-PEG8-gDR and ARM 2.14: Biotin-PEG4-gDR), a modest decrease in koff was observed relative to the lysine-containing peptides (ARM 2.11: DTB-PEG8-gD and ARM 2.12: Biotin-PEG4-gD).

Differences in LP14 mAb binding affinity between various fluorosulfate-I-tyrosine (FSY) insertion sites were evaluated by BLI analysis, performed as above. Each of the N-terminal (cARM 2.7: Biotin-FSY-gDR), Internal (cARM 2.8: Biotin-gDR(F10FSY)), and C-terminal (cARM 2.9: Biotin-gDR-FSY) SuFEx variants were compared with the original peptide (ARM 2.11: DTB-gD) as a positive control for differences in binding to anti-HSV gD LP14 mAb. The fast k_{off} observed in the cARM 2.8 (Biotin-gDR(F10FSY)) sample is consistent with internal fluorosulfate installation representing a deleterious substitution, due the role of the phenylalanine at position 10 (F10) in antibody binding (FIG. 44).

Differences in LP14 mAb binding affinity between C-terminal tyrosine- and SuFEx-modified ARMs were evaluated by BLI analysis, performed as above. The C-terminal tyrosine mutant (ARM 2.15: Biotin-gDR-Y) demonstrated a similar kₒₙ and k_{off} to the C-terminal SuFEx mutant (cARM 2.9: Biotin-gDR-FSY). Differences in binding amplitude were likely a result of loading differences between peptides, where slight excesses of unreacted biotin-DBCO compete for sites on the streptavidin biosensors. This data demonstrates that FSY insertion at the C-terminal position is well tolerated by the LP14 antibody (FIG. 43).

Next, the binding of a hydrolyzed aryl-sulfonyl fluoride-modified peptide Biotin-SO₃H-gDR (ARM 2.19) to LP14 mAb was analyzed by BLI. As can be seen in FIG. 87, the hydrolyzed peptide Biotin-SO₃H-gDR (ARM 2.19) was found to have a lower k_{off} (i.e., higher binding affinity) relative to Biotin-gD, while maintaining selectivity in the presence of a non-binding human IgG isotype control antibody.

TABLE 3
Binding constants for immobilized gD
peptide mutants and LP14 mAb in BLI.

| Compound | kₒₙ (M⁻¹s⁻¹) | k_{off} (s⁻¹) | K_{D} (nM) |
|---|---|---|---|
| cARM 2.7 | 67150 ± 800 | 5.69 × 10⁻³ ± | 84.74 ± 0.82 |
| | | 4.2 × 10⁻⁵ | |
| cARM 2.8 | 68800 ± 600 | 2.21 × 10⁻² ± | 321.22 ± 2.60 |
| | | 1.7 × 10⁻⁴ | |
| cARM 2.9 | 119400 ± 750 | 0.006374 ± 0.000014 | 53.39 ± 0.30 |
| cARM 2.10 | 70600 ± 400 | 0.004784 ± 0.000023 | 67.76 ± 0.71 |
| ARM 2.11 | 81450 ± 350 | 0.01199 ± 0.00003 | 147.21 ± 0.88 |
| ARM 2.12 | 60250 ± 400 | 0.0113 ± 0.00003 | 187.55 ± 1.74 |
| ARM 2.13 | 87800 ± 350 | 0.007244 ± 0.00001 | 82.51 ± 0.44 |
| ARM 2.14 | 77400 ± 450 | 0.006015 ± 0.000014 | 77.71 ± 0.63 |
| ARM 2.15 | 86350 ± 400 | 0.005763 ± 0.00001 | 66.74 ± 0.42 |

Assessing Covalent Labelling Using Bio-Layer Interferometry:

An initial assessment of covalent labelling was performed by incubating antibody and cARM for 24 hours and monitoring final nm shift amplitudes post-dissociation. Incubations were made 24 hours prior using 1 μM antibody and 2 μM cARM, which was diluted 10× in 1× KB prior to loading. A control for cARM and ARM was performed by loading only 200 nM of either compound. A selectivity control was performed by pre-incubating cARM compound with a polyclonal Human isotype IgG at the same 1:2 μM concentration for 24 hours, before diluting 10× in 1× KB. BLI was performed by placing streptavidin coated biosensor probes in 100 nM solutions of antibody with 200 nM cARM. After 10 minutes, probes were moved to a competitor well with 100 μM peptide 1 to dissociate non-covalently bound antibody for another 10 minutes.

LP14 mAb pre-incubated with cARM demonstrated a covalent reaction (FIG. 45). 2 μM ARM 2.15 or cARM 2.9 was incubated with 1 μM antibody over a 24-hour period before diluting 10-fold. This was then directly loaded onto a probe until near saturation, where dissociation was monitored in the presence of free competitor peptide. LP14 mAb+cARM 2.9 demonstrated a very small nm decrease in the competitor dissociation phase, indicating covalency. ARM 2.15 in the presence of LP14 mAb returned to a baseline nm shift in the presence of competitor. cARM 2.9 alone, and with Human IgG isotype antibody associated to a nm shift comparable to the post-dissociation nm shift of cARM 2.9+LP14 mAb. A greater loading amplitude was seen for cARM 2.9 alone.

BLI was used to assess proximity-induced covalent labeling of LP14 mAb with cARM 2.7 (Biotin-FSY-gDR) or cARM 2.8 (Biotin-gDR(F10FSY)), which contain an FSY group at an N-terminal or an internal location, respectively. Both cARMs 2.7 and 2.8 pre-incubated with LP14 mAb demonstrated a large increase in wavelength shift (nm) after loading onto streptavidin-coated probes, relative to the covalent peptides alone, indicating antibody binding to the probe (FIG. 46). After dissociation using competitor peptide (100 μM), signal amplitudes of both cARMs 2.7 and 2.8 pre-incubated with LP14 mAb plateaued well above baseline levels (i.e., a very small decrease in wavelength shift (nm) was observed during the dissociation phase), indicating covalent labeling of the LP14 mAb (FIG. 46).

BLI was used to assess proximity-induced covalent labeling of H170 mAb with cARM 2.7 (Biotin-FSY-gDR), cARM 2.8 (Biotin-gDR(F10FSY)), cARM 2.9 (Biotin-gDR-FSY) or cARM 2.10 (Biotin-ASF-gDR). cARMs 2.7-2.10 (200 nM) immobilized to streptavidin-coated biosensors were used to measure specific binding to H170 mAb (100 nM) in 1× kinetics buffer. Dissociation was monitored by submerging the biosensor:peptide:Ab complex in free gD peptide (200 μM). As shown in FIG. 88 and Table 4, the internal phenylalanine (F10) residue was found to be essential for H₁₇₀ binding, as the F10FSY substitution inhibited the binding ˜100-fold.

TABLE 4
Binding constants for immobilized gD
peptide mutants and H170 mAb in BLI.

| Peptide | kₒₙ (M⁻¹s⁻¹) | k_{off} (s⁻¹) | K_{D} (nM) |
|---|---|---|---|
| Biotin-FSY-gDR | 89600 ± 400 | 1.272 × 10⁻³ ± | 14.2 ± 0.1 |
| | | 4 × 10⁻⁶ | |
| Biotin-gDR(F10FSY) | 4800 ± 100 | 4.9 × 10⁻² ± | 1.02 × 10⁵ ± |
| | | 1 × 10⁻³ | 5 × 10³ |
| Biotin-gDR-FSY | 85300 ± 300 | 1.595 × 10⁻³ ± | 18.7 ± 0.1 |
| | | 4 × 10⁻⁶ | |
| Biotin-ASF-gDR | 84000 ± 300 | 2.331 × 10⁻³ ± | 27.8 ± 0.2 |
| | | 8 × 10⁻⁶ | |

### Example 16: Kinetic Analysis of Proximity-Induced Covalent Labelling Using Bio-Layer Interferometry

The human IgG isotype control used was purchased from Jackson ImmunoResearch (009-000-003). The mouse IgG2a monoclonal anti-HSV antibody was purchased from Sigma-Aldrich (MABF1975). Soluble PSMA was generously given by Dr. Cyril Barinka (Institute of Biotechnology CAS, Czech Republic). Protein G biosensor probes and kinetics buffer were purchased from Sartorius.

To validate in-solution proximity labelling reaction kinetics, a pre-established reverse format BLI assay was performed (FIG. 47). Timepoints for the covalent reaction were created by pre-incubating a solution of 750 nm antibody with 15 μM cARM 2.4, before diluting 10× with 1× KB prior to BLI. A 75 nM LP14 mAb only well was used to control for antibody dissociation from protein G over time. A 1.5 μM cARM only control was used to ensure no non-specific binding to protein G probes. A 60 second baseline was first performed, before beginning a loading phase, where Protein G probes were placed in each Ab-cARM solution for 60 seconds. Probes were then placed into 100 μM competitor peptide 1 for 20 minutes, followed by a baseline in 1× KB for 60 seconds. To measure the relative amount of covalently labelled antibody, probes were placed in 500 nM PSMA, and nm shift amplitude was compared across each timepoint. The maximum nm shift amplitude was plotted against reaction time (FIG. 48). A linear regression was performed using a first-order decay equation (Equation 3) to extract a k_{obs} and half-life. Here it was assumed that antibody:cARM was under pseudo-first order conditions.

### Example 17: Evaluation of Covalent Antibody Labelling Selectivity and Kinetics Using Fluorescence SDS-PAGE

General:

SDS-PAGE was performed to visualize covalent antibody labeling through the appearance of fluorescent protein bands under reducing/denaturing conditions. Samples were worked up prior to SDS-PAGE by diluting with 2× Laemmli sample buffer (Sigma Aldrich, Cat. No. S3401-10VL) and heating at 95° C. for 5 minutes. 14-20 μL of reduced, denatured protein sample was loaded into an Invitrogen™ Novex™ WedgeWell™ 14% acrylamide, Tris-Glycine Mini Protein Gel (Thermo Scientific, Cat. No. XP00140BOX). An Invitrogen Mini Gel Tank (Cat. No. NW2000) and 1× tris glycine running buffer (24.76 mM tris, 1.73 mM SDS, 95.91 mM glycine, Milli-Q water) were used for each separation. Bands were stacked by applying 90V for 15 minutes, followed by a 50-minute separation at 120V. Fluorescent bands were imaged using a Typhoon™ laser-scanner platform with a Cy3 laser and the auto-PMT setting. Mean fluorescence intensity was quantified using ImageJ. Gels were stained with EZBlue™ Gel Staining Reagent (Sigma Aldrich, Cat. No. G1041) for 1 hour. Gels were destained in deionized water for several hours, or until bands were prominent and background changed from blue to clear. Stained gels were imaged using a 700 nM laser on an Odyssey CLX imager. The human IgG isotype control used was purchased from Jackson ImmunoResearch (Cat. No. 009-000-003). Amicon Ultra-0.5 mL 10 kDa Centrifugal Filters were purchased from Sigma Aldrich (Cat. No. C82301). Pooled Human AB Serum Plasma Derived was purchased from Innovative Research (Cat. No. ISERAB100ML), found to have 3.5 g/dL serum albumin and 5.2 g/dL total protein content. Mouse IgG2a Anti-HSV-½ gD Antibody (H170) was purchased from Santa Cruz Biotechnology (Cat. No. sc-69802).

SDS-PAGE Selectivity Assays:

The selectivity of SuFEx-mediated covalent labelling of non-specific proteins was assessed by SDS-PAGE with a fluorescent readout. Incubations were carried out in the dark, at room temperature, with 1 μM LP14 mAb and 2 μM cARMs 2.1-2.3 for 24 hours in 1×PBS. A Human Isotype IgG control, and LP14 mAb pre-equilibrated with 100 μM ARM 2.11 was included to assess selectivity. ARM 2.16 was included to test for potential non-specific binding. SDS-PAGE was performed to visualize labelled protein bands. The selectivity of cARM 2.5 was assessed in the same way, but with 1 μM LP14 mAb and 20 μM cARM.

A similar experiment was performed in the presence of PSMA (FIG. 49). Here, 2 μM PSMA was incubated with 20 μM of cARMs 2.1 and 2.5 over a course of 24 hours in 1×PBS. SDS-PAGE was performed to visualize labelled protein bands. Time 0 controls were included to account for non-specific labelling resulting from heating.

Selectivity of cARM 2.5 (Fluorescein-ASF-gDR) was evaluated at high concentrations. cARM 2.5 (20 μM) was incubated with LP14 mAb (1 μM) alone or LP14 mAb pre-incubated with competitor gD peptide (100 μM). A human IgG isotype control antibody (1 μM) was used as a control for non-specific binding. As can be seen in FIG. 89, covalent labeling of the LP14 mAb with cARM 2.5 (Fluorescein-ASF-gDR) was blocked by pre-incubation with competitor peptide, while minimal signal was observed for human IgG isotype control.

Selectivity of the following SuFEx group-containing antibody recruitment molecules was evaluated: cARM 2.1 (Fluorescein-FSY-gDR), cARM 2.2 (Fluorescein-gDR(F10FSY)), cARM 2.3 (Fluorescein-gDR-FSY), and cARM 2.5 (Fluorescein-ASF-gDR)). Each one of cARMs 2.1, 2.2, 2.3 and 2.5 (1 μM) was incubated with H170 mAb (0.5 μM) alone, or with H170 mAb pre-incubated with competitor gD peptide (500 μM). LP14 mAb (0.5 μM) was used as a positive control for covalent labeling. As can be seen in FIG. 90, pre-incubation with competitor gD peptide was found to abolish labeling between H170 mAb and covalent ARMs. Labeling of the H170 mAb was predominantly observed at the light chain for cARM 2.1 (Fluorescein-FSY-gDR) and cARM 2.5 (Fluorescein-ASF-gDR), whereas cARM 2.2 (Fluorescein-gDR(F10FSY)) and cARM 2.3 (Fluorescein-gDR-FSY) showed no labeling (FIG. 90).

Kinetic Analysis of Proximity-Induced Covalent Labelling Using SDS-PAGE:

The mechanism of proximity enhanced, in-solution antibody labelling is described in brief. Specific labelling of an antibody Fab domain occurs after formation of a reversible Fab:cARM complex. Here, K_{I}=K_{D}, represented by the concentration to reach half-maximal binding. The k_{inact} represents the rate of covalent reaction once this reversible complex is formed. By ensuring saturating conditions, the model may be simplified into a pseudo first order reaction, where k_{obs}=k_{inact}. The rationale for this stems from Equation 4, as [cARM] was present in 10× excess and both [Fab] and [cARM] were >>K_{D}. Under these conditions, the concentration of non-covalently bound cARM to Fab remains above 99% until [Fab]<<K_{D}, found using Equation 5. By this point in the reaction, the concentration of non-covalent complex would drop dramatically, slowing the covalent reaction. However, also by this point, fluorescent changes become undetectable using these methods. Thus, the bulk of the reaction used to calculate k_{obs} holds saturating conditions, allowing for this equation to be used.

$${k}_{obs} = \frac{{K}_{I} ( {k}_{inact} + [ cARM ] )}{{K}_{I} + [ cARM ]} \qquad Equation 4$$

$$[ cARM : Fab ] = \frac{[ cARM ] [ Fab ]}{[ cARM ] + {K}_{D}} \qquad Equation 5$$

To monitor the formation of Ab-cARM over time, 20 μM of cARMs 2.1-2.3 and cARM 2.5 were incubated with 1 μM LP14 mAb at a ratio of 1:10 Fab to cARM. Each reaction time point was created by aliquoting from a primary stock, flash freezing using a −80° C. acetone bath, and then storing at −80° C. until SDS-PAGE. SDS-PAGE was performed to isolate labelled heavy and light chains. ImageJ was used to determine the mean intensity of fluorescent bands, which were plotted as a function of reaction time. To verify the assumption of pseudo-first order conditions, DynaFit was used to extract k_{inact} from each data set using the following script:

```
[task]
  data = progress discontinuous
  task = fit
  algorithm = DE
[mechanism]
  Ab + cARM <===> Ab.cARM : k1 k2
  Ab.cARM ----> P : kinact
[constants]
  k1 = 67150? (66750..67550), k2 = 0.005688? (0.005667..0.005709)
  kinact = 0.00005? (0.000005..0.005)
[concentrations]
  Ab = 2000e−9
[responses]
  P = 1? (0.95..1.05)
[data]
  directory ./kinetics/HSVcARMExp3
  extension txt
  monitor P
  file f1 | concentration cARM = 20000e−9 | label = 20000 nM cARM
[output]
  directory ./kinetics/HSVcARMExp3
[settings]
{Output}
  XAxisLabel = Time (s)
  YAxisLabel = [cARM-Ab] (M)
[end]
```

Adapted from Kapcan, E., Lake, B., Yang, Z., Zhang, A., Miller, M. S., Rullo, A. F. “Covalent Stabilization of Antibody Recruitment Enhances Immune Recognition of Cancer Targets” Biochemistry 2021, 60(19):1447-1458, this DynaFit script data was inputted as a set of discontinuous measurements (data=progress discontinuous). The ‘Differential-Evolution’ algorithm (algorithm=DE) denotes a least squares data fit where an evolutionary strategy computational approach is used to find global optima. The mechanism used to describe covalent labelling followed a 2-step approach. An initial binding event (Ab+cARM<===>Ab.cARM: k1 k2) where k, =kₒₙ and k₂=k_{off}, is followed by a covalent labelling step (Ab.cARM---->P: kinact). Due to the long hydrolysis half-life of OSO₂F, no hydrolysis function was included. Constants (k₁, k₂) calculated from previous BLI experiments were inputted as a range. The value for k_{inact} was estimated using k_{obs} and a large window for error. The [Ab] was inputted as the [Fab], equating to 2× Ab. The [Ab-cARM] estimated to have formed was inputted in the line (P=1? (0.95 . . . 1.05)), where 1=1 nM.

A time course experiment of the labeling kinetics between FSY-equipped peptides and anti-gD LP14 mAb is shown in FIG. 91. Peptides (20 μM) containing a covalently reactive FSY group at the N-terminus (cARM 2.1=“Peptide 1B”), internal F10 position (cARM 2.2=“Peptide 2B”) or C-terminus (cARM 2.3=“Peptide 3B”) were incubated with LP14 (1 μM) for 0, 3, 6, 12, 24 or 48 hours before SDS-PAGE and fluorescent detection. A second unknown low MW band appearing under the IgG light chain was consistent between LP14 samples, and likely represents an impurity from commercial antibody purification. Duplicate measurements were used to construct a curve of fraction labeled over time.

A time course experiment of the labeling kinetics between excess cARM 2.5 (Fluorescein-ASF-gDR) and anti-gD LP14 mAb is shown in FIG. 92. cARM 2.5 (20 μM) was incubated with LP14 mAb (1 μM) for 0, 0.25, 0.5, 1, 2, 4, 8, 12 and 24 hours before SDS-PAGE and fluorescent detection. Duplicate measurements were used to construct a curve of fraction labeled over time. SDS-PAGE gels were placed adjacent to one another for fluorescent imaging. DynaFit was used to extract k_{inact} values from each data set using a version of the script set forth above. The results are presented in Tables 5 and 6 below.

TABLE 5
A comparison k_{obs} to k_{inact} values calculated using DynaFit.

| | | k_{inact} | RMSD |
|---|---|---|---|
| Peptide | k_{obs} | (DynaFit) | (DynaFit) |
| Fluor-FSY-gDR | 2.41 × 10⁻⁵ ± | 2.45639 × 10⁻⁵ | 6.80 × 10⁻⁹ |
| | 3.3 × 10⁻⁶ | | |
| Fluor-gDR(F10FSY) | 2.51 × 10⁻⁵ ± | 2.32601 × 10⁻⁵ | 5.92 × 10⁻⁸ |
| | 6.8 × 10⁻⁶ | | |
| Fluor-gDR-FSY | 1.62 × 10⁻⁵ ± | 1.69376 × 10⁻⁵ | 2.02 × 10⁻⁸ |
| | 3.6 × 10⁻⁶ | | |
| Fluor-ASF-gDR | 3.31 × 10⁻⁴ ± | 3.62456 × 10⁻⁴ | 9.50 × 10⁻⁸ |
| | 8.5 × 10⁻⁵ | | |

TABLE 6
Second-order rate constant (k_{inact}/Kₗ)
calculated for covalent peptides and LP14 mAb.

| Peptide | k_{inact}/Kₗ | Half Life (h) |
|---|---|---|
| Fluor-FSY-gDR | 280 ± 40 | 8 ± 1 |
| Fluor-gDR(F10FSY) | 78 ± 22 | 8 ± 2 |
| Fluor-gDR-FSY | 300 ± 100 | 12 ± 2 |
| Fluor-ASF-gDR | 4900 ± 1300 | 0.6 ± 0.1 |

A time course experiment of the labeling kinetics between cARM 2.1 (Fluorescein-FSY-gDR) or cARM 2.5 ((Fluorescein-FSY-gDR) and anti-gD H170 antibody is shown in FIG. 93. Each of cARM 2.1 (10 μM) and cARM 2.5 (10 μM) was incubated with H170 mAb (0.5 μM) for 0, 0.5, 1, 2, 4, 8, 12, 24 and 48 hours before SDS-PAGE and fluorescent detection. Replicate measurements were used to construct a curve of fraction labeled over time and calculate an observed rate constant (k_{obs}), as shown in Table 7 below.

TABLE 7
Second-order rate constant (k_{inact}/Kₗ)
calculated for covalent peptides and H170 mAb.

| Peptide | k_{inact} | k_{inact}/Kₗ | Half Life (h) |
|---|---|---|---|
| Fluor-FSY-gDR | 4 × 10⁻⁶ ± | 3 × 10² ± | 50 ± 20 |
| | 2 × 10⁻⁶ | 1 × 10² | |
| Fluor-ASF-gDR | 2.5 × 10⁻⁴ ± | 9 × 10³ ± | 0.8 ± 0.1 |
| | 6 × 10⁻⁵ | 2 × 10³ | |

### Example 18: Assessing the Bimolecular Rate of Each SuFEx Chemistry

All fluorescence polarization measurements were done on a TECAN SPARK plate reader, using Greiner® Black 96 Well plates. The human IgG isotype control used was purchased from Jackson ImmunoResearch (009-000-003). Amicon Ultra-0.5 mL 10 kDa Centrifugal Filters were purchased from Sigma Aldrich (Cat. No. C82301).

The rate of the bimolecular reaction between the fluoro sulfonate and sulfonyl fluoride electrophiles and exposed nucleophilic residues on Human IgG was determined by SDS-PAGE with a fluorescent readout. To accomplish this, 2 μM Human isotype IgG was incubated with 100 μM cARM 2.1, or 2.4, respectively. Aliquots were flash frozen at the designated time points and stored at −80° C. until SDS-PAGE separation.

A high concentration of LP14 mAb and cARM 2.1 was used, along with a long timeframe, to allow for sufficient product conversion in the absence of proximity enhancement. The fluoro sulfonate group is thought to target predominantly Lys and Tyr residues, whereas the aryl sulfonyl fluoride group has a broader reactivity towards Lys, Tyr, His, Ser, and Thr residues (Martin-Gago, P. and Olsen, C. A. “Medicinal Chemistry Arylfluorosulfate-Based Electrophiles for Covalent Protein Labeling: A New Addition to the Arsenal” Angewandte Chemie 2019, 58(4):957-966). Due to the diverse subset of these nucleophilic residues present on the surface of IgG, difficulties arise in quantifying the bimolecular rate constant. Here, it was assumed that on average, 85 lysines are present throughout IgG and are available for transient non-specific labelling. It was also assumed that the resulting sulfonamide linkage would be the predominant product, due to the high nucleophilicity of Lys. It is important to note that although these assumptions are necessary to estimate the bimolecular rate in this format, the resulting rate is likely overestimated due to the participation of additional nucleophilic residues.

To quantify antibody labelling, the reaction was stopped by performing a spin filtration. The reaction was washed several times by diluting in 1×PBS and spinning at 14000 rpm. A control sample at time 0 was included to account for non-specific binding of fluorescein. Here, antibody was incubated with cARM 2.1 and immediately worked up with a spin column. The fluorescence of each sample was quantified using a Tecan plate reader. An excitation wavelength of 488 nm and emission wavelength of 535 nm were chosen. Readings consisted of 30 flashes with optimal gain, a Z-position of 17000 μm, and a settle time of 300 ms. A calibration curve was constructed using BSA-FITC, allowing for the [fluorescein] to be quantified for the antibody sample. The [Ab] after work up was quantified using a Bradford assay, with a BSA calibration curve and an A595 readout. By finding [fluorescein]/[antibody], the extent labelled at 72 hours was found, and used to calibrate the y-axis to [Ab-cARM] (see sample calculation below).

Sample calculation:
Calibration curve: Fluorescence Intensity=5077.8[Fluorescein]−236.69
Fluorescence (72 hours)=2903, fluorescence (0 hours)=532
[Fluorescein]=0.856 μM
Bradford curve: A595=0.7731[Protein (μg/mL)]⁺0.0239
A595=0.53801±0.00863
[Antibody]=0.815±0.013 μM
[Fluorescein]/[Antibody]=1.051±0.017
[Ab-cARM] (72 hours)=1.051±0.017 μM

FIG. 94 shows a fluorescent SDS-PAGE experiment of the non-specific, bimolecular reaction between fluorosulfate-substituted cARM 2.1 (fluorescein-FSY-gDR) and a human IgG isotype antibody.

FIG. 95 shows a fluorescent SDS-PAGE experiment of the non-specific, bimolecular reaction between sulfonyl fluoride-substituted cARM 2.5 (fluorescein-ASF-gDR) and a human IgG isotype antibody.

The bimolecular reaction rate between peptide 1B and a non-binding human IgG isotype antibody was evaluated by fluorescent SDS-PAGE. Aliquots were flash frozen at −80° C. to generate timepoints for SDS-PAGE and subsequent fluorescent detection of labeled protein bands. Depicted in FIG. 96 is the linear stage of the bimolecular reaction. As can be seen in FIG. 96A, the IgG heavy chain (HC) reacted at twice the rate as the IgG light chain (LC). The combined rate of heavy and light chain was used to quantify extent labeled of the entire pooled IgG sample (FIG. 96B and Table 8).

TABLE 8
Effective molarity (EM) enhancements between fluorosulfate-
and sulfonyl fluoride-equipped covalent peptides fluorescein-
FSY/ASF-gDR, which target anti-HSV gD LP14 mAb.

| Peptide | kᵢₙₜₑᵣ (M⁻¹s⁻¹) | Rate Enhancement | EM (mM) |
|---|---|---|---|
| Fluor-FSY-gDR | 0.030 ± 0.004 | 9000 ± 2000 | 0.8 ± 0.2 |
| Fluor-ASF-gDR | 0.038 ± 0.01 | 130000 ± 40000 | 8.6 ± 4.7 |

### Example 19: ELISA Analysis of Covalent Antibody Labeling Using Model LP14 Monoclonal Antibody and Endogenous Serum Polyclonal Antibodies

General:

All absorbance measurements were done on a TECAN SPARK plate reader. Pierce Streptavidin Coated High-Capacity ELISA plates pre-blocked with SuperBlock were purchased from Sigma Aldrich (Cat. No. 15500). The Mouse/Rat HSV-1 IgG ELISA Kit was purchased from Creative Diagnostics (Cat. No. DEIA3555). Goat anti-human IgG (H+L) secondary antibody, HRP conjugate was purchased from Thermo Scientific (Cat. No. A18805). Goat anti-mouse IgG (H+L) secondary antibody, HRP conjugate was purchased from Thermo Scientific (Cat. No. 62-6520). The Pierce™ TMB Substrate Kit was purchased from Fisher Scientific (Cat. No. 34-021). Sulfuric acid (2M) was purchased from VWR (Cat. No. 470302-860). Wash buffer was prepared with 50 mg/mL BSA and 0.005% Tween20 (v/v) in 1×PBS.

Verifying Fraction Labelled Antibody after 24 Hours:

To evaluate the degree of labelling between each cARM and LP14 mAb, an ELISA was performed. A Pierce Streptavidin Coated High-Capacity ELISA plate pre-blocked with SuperBlock was used to immobilize Ab:ARM. The reagents and protocol from the Mouse/Rat HSV-1 IgG ELISA Kit was used for anti-cARM/ARM detection. In short, an anti-mouse IgG-HRP conjugate was used to detect immobilized antibody, where addition of 100 μL 1:1 TMB/peroxide, quenched by 100 μL 2M sulfuric acid produced a blue-coloured readout. Absorbance at 450 nm with a reference filter set to 620 nm was used to compare values between ARM and cARMs. Absorbance readings consisted of 50 flashes with a 100 ms settle time. Conditions containing cARM were quenched with 180 μM ARM 2.17. Assuming the Ab:ARM non-covalent complex is held together by avidity, an A450 reading equal to the Ab:ARM condition is 100% covalently reacted; the rationale being if both Fabs are not labelled, a portion of the Ab:cARM population would be completely non-covalently bound on either Fab, and dissociate upon addition of competitor (FIG. 53).

Detection of Endogenous Anti-HSV Antibodies in Mouse Serum:

Serum from mice inoculated with oncolytic HSV was investigated for endogenous antibodies generated against this N-terminal gD1 peptide. To accomplish this, the serum of five mice with “boosted” serum was pooled and compared against the pooled serum of three control mice. 50 μL of 4 μM ARM 2.11 was incubated in the designated wells for 30 minutes, while wash buffer acted as a place holder in control wells. Wells were washed 3× with 300 μL wash buffer. 100 μL of diluted serum samples were added to their respective wells incubated for 1 hour. After this, each well was washed 3× with 300 μL wash buffer. 100 μL of an anti-mouse IgG HRP conjugate was added to each well for 20 minutes. After this, each well was washed 3× with 300 μL wash buffer. For detection, 100 μL TMB substrate was added to each well for 10 minutes, followed by 100 μL stop solution. A readout was performed using absorbance at 450 nm, with a reference filter set to 620 nm.

FIG. 54 shows the results of an ELISA assay using biotinylated peptides derived from the HSV gD domain (Biotin-gD) to probe endogenous anti-HSV antibodies in serum from mice boosted with OV (n=5). Sera from mice not exposed to OV (n=3) were used as a control for basal anti-HSV antibody levels. In this assay, Biotin-gD peptides were immobilized on streptavidin coated ELISA plates and treated with serum. Anti-HSV antibodies bound to the plate were detected using HRP-conjugated secondary antibody. A PBS blank was used in place of Biotin-gD to detect non-specific binding of serum IgG, leading to background signal. As can be seen in FIG. 54, serum from mice boosted with the OV showed substantially higher antibody binding to the plate coated with gD peptides, as compared to control serum from mice not exposed to the OV.

FIG. 55 shows the results of an ELISA assay comparing covalent (cARM 2.7) versus non-covalent (ARM 2.12) binding to natural anti-HSV antibodies present in mouse serum from OV boosted mice. Serum from mice boosted with the OV showed substantially higher antibody binding to plates pre-loaded with cARM 2.7 compared to plates pre-loaded with (ARM 2.12).

To detect natural anti-HSV antibodies specific for the gD epitope in mouse serum, mice were boosted with a 10× dilution of HSV-1d810 oncolytic virus. Serum from a mouse not boosted with the oncolytic virus was used as a control. The serum was incubated with covalent cARM 2.7 (Biotin-FSY-gDR=“NT cARM”), cARM 2.8 (Biotin-gDR(F10FSY)=“Int cARM”) or cARM 2.9 (Biotin-gDR-FSY=“CT cARM”), or non-covalent ARM 2.12 (Biotin-gD=“ARM”) for 24 hours before detection by ELISA. The same gD peptide competition controls were used to explore covalency and selectivity. The results of the ELISA analysis demonstrate that the compounds disclosed herein selectively bind to endogenous anti-HSV antibodies in mouse serum (FIG. 76D).

Detection of Endogenous Anti-HSV Antibodies in Human Serum:

To detect natural human anti-HSV antibodies specific for the gD epitope, pooled IgG antibodies (5 μM) isolated from human serum were incubated with covalent cARM 2.7 (Biotin-FSY-gDR), cARM 2.8 (Biotin-gDR(F10FSY)), cARM 2.9 (Biotin-gDR-FSY) or cARM 2.10 (Biotin-ASF-gDR) or non-covalent ARM 2.12 (Biotin-gD) for 24 hours or 60 seconds (t=0) before detection by ELISA. Competitor gD peptide (100 μM) was added after a 24-hour incubation time to serve as a control for covalent labeling (post-rxn). Reaction selectivity was assessed by adding competitor gD peptide to antibody source before the respective covalent peptide (pre-rxn). The results of the ELISA analysis demonstrate that the compounds disclosed herein selectively bind to endogenous anti-HSV antibodies in human serum (FIG. 97A).

A fluorescent SDS-PAGE experiment (FIG. 97B) was performed to assess cARM-mediated covalent labeling of polyclonal, endogenous natural anti-HSV antibodies in human serum. cARM 2.1 (Fluorescein-FSY-gDR), cARM 2.2 (Fluorescein-gDR(F10FSY)), cARM 2.3 (Fluorescein-gDR-FSY), a combination of the cARMs 2.1-2.3 (10 μM), or cARM 2.5 (Fluorescein-ASF-gDR)) were spiked into pooled IgG (2 μM) isolated from human serum. Selectivity was assessed using the pre-rxn control described above. A quantification of the fluorescence intensities from the SDS-PAGE experiment revealed that the compounds disclosed herein covalently label endogenous anti-HSV antibodies in human serum (FIG. 97C).

Selective engagement of natural anti-HSV antibodies in OV-boosted mouse IgG and pan human IgG was assessed using fluorescent SDS-PAGE (FIG. 100A). cARM 2.5 (Fluorescein-ASF-gDR) (1 μM) was used to covalently label enriched (0.5 μM), depleted (0.5 μM), or pooled (0.5 μM) IgG. Pooled IgG refers to a polyclonal IgG mixture from either 5 mice boosted with HSV oncolytic virus (FIG. 100A, left panels, “OV Mouse IgG”), or a commercial polyclonal human IgG product (FIG. 100A, right panels, “Pan Human IgG”). Enriched IgG was made using a pull-down column with gD immobilized onto streptavidin agarose. Depleted IgG was obtained as the flow through from the pull-down column after 3 pull down cycles. Selectivity was demonstrated using either a non-covalent gD competitor peptide (10 μM), or cARM 2.6 (GU-ASF-gDR) (10 μM) as a covalent competitor. The results of this experiment indicate that cARM 2.5 (Fluorescein-ASF-gDR) covalently binds to endogenous anti-HSV antibodies present in OV-boosted mouse IgG and pan human IgG (FIG. 100A). A time-course of the reaction between cARM 2.5 (Fluorescein-ASF-gDR) (10 μM) and pan human IgG (1 μM) over a period 24 hours is shown in FIG. 100B.

### Example 20: Purification and Characterization of Natural Anti-gD Antibodies from Pooled Human IgG

Natural anti-gD antibodies were affinity purified from pooled human IgG using the Biotin-gD peptide. Thermo Scientific™ Pierce™ High-Capacity Streptavidin Agarose resin (Cat. No. P120357) was used to immobilize Biotin-gD according to their protocol. Briefly, 0.5 mL resin was incubated with 1 mL of 66.67 μM Biotin-gD for 30 minutes. This was washed with 10 column volumes of 1×PBS before incubating with 280 μM pooled human IgG (Innovative Research, Cat. No. IHUIGGAP) overnight at 4° C., while agitating. Pooled human IgG was eluted, generating depleted human IgG. The column was washed with 10 column volumes of PBS before eluting with 10 column volumes of 0.1 M glycine, pH 2.7. Fractions were pooled and buffer exchanged into PBS using 10 kDa Amicon spin columns.

Binding of cARM 2.7 (Biotin-FSY-gDR) to enriched anti-gD IgG isolated from pooled human serum IgG was evaluated by BLI. cARM 2.7 (200 nM) was loaded onto streptavidin-coated probes before association with 100 nM enriched human anti-gD IgG. Dissociation was performed in 200 μM competitor gD peptide. As can be seen in FIG. 98, cARM 2.7 specifically bound to the enriched human anti-gD IgG.

TABLE 9
Comparison of binding constants between cARM 2.7
(Biotin-FSY-gDR) and LP14 mAb or enriched human
IgG polyclonal antibodies (pAb) using BLI.

| Antibody | kₒₙ (M⁻¹s⁻¹) | k_{off} (s⁻¹) | K_{D} (nM) |
|---|---|---|---|
| LP14 mAb | 67200 ± 800 | 5.69 × 10⁻³ ± | 84.7 ± 0.8 |
| | | 4 × 10⁻⁵ | |
| Enriched IgG pAb | 3960 ± 110 | 1.65 × 10⁻² ± | 4170 ± 10 |
| | | 4 × 10⁻⁴ | |

### Example 21: cARM 2.4 and cARM 2.6 Mediate Antibody Dependent Cellular Phagocytosis (ADCP) of PSMA-Expressing Human Cells

General:

All flow cytometry experiments were run on a BD LSRII Flow Cytometer. The human IgG isotype control used was purchased from Jackson ImmunoResearch (Cat. No. 009-000-003). The mouse IgG2a monoclonal anti-HSV antibody was purchased from Sigma-Aldrich (Cat. No. MABF1975). PSMA expression was confirmed with an anti-PSMA antibody alexa 647 conjugate (Novus Biologicals, Cat. No. FAB4234R). HEK−293T (PSMA+/−) cell lines were generously provided by Dr. Cyril Barinka (Institute of Biotechnology CAS, Czech Republic). U937 cells were generously provided by Dr. John Valliant (McMaster University, Canada). IFN-γ was purchased from Fischer Scientific (Cat. No. PHC4031). Ultra-low IgG FBS was purchased from Fischer Scientific (Cat. No. A3381901). The anti-mouse IgG (H+L) secondary antibody (PE conjugate) was purchased from Thermo Fisher Scientific (Cat. No. 12-4010-82). RPMI-1640 was purchased as a powder from Fischer Scientific (Cat. No. 31800089) and resuspended. DMEM was purchased as a powder from Fischer Scientific (Cat. No. 12800082) and resuspended. DiD cell dye was purchased from Fischer Scientific (Cat. No. V22887). DiO cell dye was purchased from Fischer Scientific (Cat. No. V22886). TrypLE Express was purchased from Fischer Scientific (Cat. No. 12604013). 96-Well U-bottom plates were purchased from Fischer Scientific (Cat. No. 08-772-17). Pen/Strep was purchased from Fischer Scientific (Cat. No. 15140-122). FBS was purchased from Fischer Scientific (Cat. No. 12484-028). Zeocin was purchased from Fischer Scientific (Cat. No. R25001). HEK-PSMA cells were cultured in DMEM media with 2 mM L-glut, 1% Pen/Strep, 10% FBS, and 50 ug/mL Zeocin. HEK cells were cultured in DMEM media with 2 mM L-glut, 1% Pen/Strep, and 10% FBS. U937 monocytes were cultured in RPMI media with 2 mM L-Glut, 1% Pen/Strep, and 10% FBS.

Evaluation of ADCP in Flow Cytometry Assays:

For preparation of effector monocytes, 24 hours prior to inducing phagocytosis U937 monocytes were seeded at 500,000 cells/mL and activated with IFN-γ (0.1 mg/mL). After incubation, these cells were then counted and washed twice with serum free assay media (neat RPMI). Cells were then suspended to a concentration of 1 million cells/mL and stained with 1.9 μM Vybrant DiD Cell-Labelling Solution for 30 minutes (37° C., 5% CO₂). Cells were then washed 3× with warm assay media (AM, 14% Ultra Low IgG FBS in RPMI) and resuspended to a concentration of 3.0×10⁶ cells/mL to be plated for use in assay (50 μL holds 150,000 cells).

Prior to overnight incubation, antibody-cARM/ARM experimental and control conditions were prepared. For each antibody condition, antibody-cARM/:ARM were incubated together at a ratio of 2:1 (cARM/ARM:Ab) and at a concentration of 4× the top antibody concentration listed. Where HSV competitor is used, ARM 2.11 was used at a concentration of 25× excess of cARM/ARM. Competition conditions had competitor present during incubation, while quench conditions had competitor added after overnight incubation. After overnight incubation, a dilution series was conducted using each condition stock which was then equilibrated for 90 minutes prior to addition to the assay well plate as described below.

On the day of the experiment, target cells (90% confluent in a T-150 flask) were suspended with TrypLE and quenched with complete growth media. These cells were then counted and washed twice with serum free assay media (neat RPMI). Cells were then suspended to a concentration of 1 million cells/mL and stained with 5.7 μM Vybrant DiO Cell-Labelling Solution for 30 minutes (37° C., 5% CO₂). Cells were then washed 3× with warm assay media (AM, 14% Ultra Low IgG FBS in RPMI) and resuspended to a concentration of 6.0×10⁶ cells/mL to be plated for use in assay (25 μL holds 150,000 cells).

To a U-bottom 96-well plate, 25 μL of target cells followed by 25 μL of each antibody condition were added (for PSMA expressions check shown in FIG. 56, 1.5 μL anti-PSMA A647 antibody was added to 25 μL assay media). Next, 50 μL of activated monocytes were added to these wells. The plate was centrifuged at 800 RPM for 2 minutes to pellet cells and placed in a 37° C. 5% CO₂ incubator for 1 hour. Plates were placed on ice and all conditions were then ran on flow cytometry to determine ADCP. DiO stained cells were detected in the A488 channel, DiD stained cells were detected in the APC Cy7 channel, PSMA expression was confirmed with the Alexa 647 channel. The following voltages were used: FSC: 390, SSC: 290, A488: 290, APC Cy7: 390, A647: 490. ADCP was determined by plotting monocyte stain against target cell stain and was quantified according to Equation 6. This was normalized to antibody only control.

$$\% Target Phagocytosed = ( \frac{\begin{matrix}Double Positive \\ Events\end{matrix}}{\begin{matrix}Target Only \\ Events + Double \\ Positive Events\end{matrix}} ) * 100 \qquad Equation 6$$

An example of a flow cytometry gating protocol for selecting single cells when evaluating ADCP data is set forth in FIG. 57. As can be seen in FIG. 58, a comparison of ADCP of PSMA-expressing HEK+ cells by monocytes in the presence of 3.13 nM LP14 mAb and 6.26 nM cARM 2.4: GU-FSY-gDR (FIG. 58A) or ARM 2.17: GU-gD (FIG. 58B) revealed increased phagocytosis using the covalent cARM 2.4 relative to the non-covalent ARM 2.17.

At a higher concentration of cARM 2.4 or ARM 2.17 (200 nM) and LP14 mAb (100 nM), both compounds showed maximum ADCP (compare top right quadrants of the scatter plots set forth in FIGS. 59A and 59B). A quench was performed by adding 100 μM gD peptide (ARM 2.11) after incubating the LP14 mAb with cARM 2.4 or ARM 2.17 overnight to determine whether binding is covalent. After quenching, cARM 2.4 (GU-FSY-gDR) maintained the same level of ADCP (indicating covalent linkage), whereas ARM 2.17 (GU-gD) lost all function (compare top right quadrants of the scatter plots set forth in FIGS. 59C and 59D).

Covalent proximity induction was analyzed using ADCP of two different human cell lines: human embryonic kidney (HEK) cells engineered to overexpress PSMA (FIG. 99A), and Lymph Node Carcinoma of the Prostate (LNCaP) cells that express a lower level of PSMA (FIG. 99B). Two-colour flow cytometry ADCP assays were conducted using FL-4 (DID) dye stained u937 human monocyte cells and FL-1 (DIO) dye stained PSMA-expressing HEK or LNCaP cells. Double positive cell events corresponding to target cell phagocytosis were recorded in the presence of LP14 mAb or isotype control antibody at the indicated concentration and 2 equivalents of cARM 2.4 (GU-FSY-gDR) or non-covalently reactive analog ARM 2.17 (GU-gD) at 37° C. Antibody and cARM 2.4 (GU-FSY-gDR) were pre-incubated with or without excess free competitor gD peptide (pre-rxn) for 24 hours prior to dilution to the indicated concentrations into solutions of target and immune cells for all conditions. Control experiments were all conducted with 100 nM antibody and 2 equivalents of cARM 2.4 (GU-FSY-gDR) or ARM 2.17 (GU-gD). Additional controls for covalency involved addition of excess free competitor gD peptide following the 24 hour incubation (post-rxn). As can be seen in FIG. 99, cARM 2.4 was found to selectively mediate covalent proximity induction in both high PSMA level-expressing HEK cells and lower PSMA level-expressing LNCaP cells.

A two-colour flow cytometry ADCP assay was used to assess the ability of cARM 2.6 (GU-ASF-gDR) to covalently engage and promote phagocytosis of PSMA-expressing human cells (FIG. 100C). FL-4 (DID) dye stained u937 human monocyte cells and FL-1 (DIO) dye stained HEK cells engineered to express PSMA were used in the assay. Double-positive events were recorded in the presence of enriched human IgG with 2 equivalents of cARM 2.6 (GU-ASF-gDR) or ARM 2.17 (GU-gD) at 37° C. Labeling was performed for 24 hours, and selectivity was probed through competition, where 200 μM free competitor gD peptide was pre-equilibrated with enriched IgG prior to spiking with cARM 2.6 or ARM 2.17. Covalency was demonstrated through a quench, where 200 μM free competitor gD peptide was spiked into the reaction after 24 hours with either cARM 2.6 or ARM 2.17. As can be seen in FIG. 100C, cARM 2.6 was found to selectively mediate covalent proximity induction in PSMA-expressing HEK cells.

### Example 22: Evaluation of cARM 2.4 in Total Antibody Recruitment

cARM 2.4 (GU-FSY-gDR) or GU-gD (500 nM) was incubated with LP14 mAb (250 nM) at room temperature, overnight. Where HSV competitor was used, gD peptide was used at a concentration of 25× excess of non-covalent peptide to covalent peptide. Competition conditions (“pre-rxn”) had competitor present during incubation, while quench conditions (“post-rxn”) had competitor added after overnight incubation. After the incubation, these conditions were diluted down in a 2× dilution series and 20 μL of each was plated in a 96-well plate in duplicate.

HEK293 cells transfected with PSMA (90% confluent in a T-150 flask) were suspended with TrypLE and quenched, washed 3× with 4° C. flow buffer (4% FBS, 0.5 mM EDTA/EGTA, 0.1% Sodium Azide) and resuspended to a concentration of 5×10⁶ cells/mL. Following this, 20 μL of cells were added to the 96-well plate (100,000 cells per sample) and kept on ice. Afterwards, 10 μL of appropriate 20× diluted (in flow buffer) secondary antibody was added to each well. PSMA loading/expression was confirmed with an anti-PSMA antibody-Alexa 647 conjugate, where 0.75 μL was diluted to 10 μL and added in place of secondary antibody. The plate was then allowed to incubate on ice for 20 minutes and run on a flow cytometer. Voltages used were FSC: 390, SSC: 290, Alexa 647: 490, PE: 350.

Antibody recruitment to PSMA-expressing HEK+ cells using cARM 2.4 (GU-FSY-gDR) and non-covalent control ARM 2.17 (GU-gD) was analyzed by flow cytometry. A PE-conjugated secondary anti-mouse (H+L chain) antibody was used to detect LP14 mAb recruited to HEK+ cells. Mean fluorescence intensity (MFI) of each cell population was used as a measure of antibody recruitment. A quench using 25× excess of free competitor gD peptide was used to distinguish covalent from non-covalent binding, based on the retention or loss of MFI signal proportional to antibody recruitment, respectively. As can be seen in FIG. 61, all available PSMA binding sites were saturated with peptide/antibody complexes by approximately 50 nM concentrations of the LP14 antibody. Due to high antibody binding avidity on multivalent PSMA-expressing cells, covalent binding did not exert any potency advantage in recruitment.

### Example 23: SuFEx Chemistry Enables Formation of Covalent Ternary Complexes Between ARM Compounds, Antibodies and Soluble Tumor Antigens

Synthetic bi-functional molecules (i.e., antibody engagers) have been developed to bridge immunological receptors like serum antibodies with protein antigens highly expressed on the surface of cancer cells, to form functional “ternary complexes”. The result of bringing immune receptors in close proximity with cancer cells for a sufficient duration of time and through the correct receptor contacts, enhances the probability of immune recognition and elimination of cancer cells. Previous approaches used bi-functional molecules equipped with small molecule haptens to bind specific serum antibodies which can engage and activate host immune effector cells if the antibody is of the correct isotype, e.g., IgG1. Notably, human serum naturally contains a high concentration of IgG antibodies comprising different IgG isotypes IgG1-4 specific for different small molecule and peptide epitopes, which includes immune inhibitory antibodies of the IgG2 isotype. Problematically, the binding affinity of bi-functional molecules for immune receptors like hapten specific antibodies may be insufficiently strong to mediate formation of highly stable bridging “ternary complexes” (e.g., cancer antigen:bi-functional molecule:antibody), required for a therapeutic immune response. High affinity binding interactions within ternary complexes are critical for immune function. This is largely due to the low endogenous concentrations of proteins involved which are often present naturally at concentrations below their K_{d} value, which destabilizes ternary complex formation. Additionally, the mechanical strain on the ternary complex induced by bridging cancer and immune receptors via bi-functional molecules, to template immune synapse formation and activation, may enforce the need for even higher stability ternary complexes.

Additionally problematic is the fact that a large proportion of natural hapten specific antibodies are not of the optimal isotype needed for activation of cancer killing immune effector cells like natural killer cells and macrophages/monocytes. To overcome this problem, bi-functional molecules have been developed to directly engage immune cells, and bridge them with cancer cells independent of serum antibodies. SyAMs molecules incorporate a peptide ligand that functions as an agonist of the native protein ligand for macrophage Fc receptors (e.g., IgG hinge region). To enhance ternary complex stability however, two copies of the peptide ligand within the construct were required to elicit robust immune cell function. This bi-valency was likely required to enhance peptide binding affinity for the immune Fc receptor via avidity.

An alternative approach for efficiently engaging immune effector cells against cancer exploits pathogen specific antibodies. This class of antibody-based immune receptor is prevalent in human serum resulting from host exposure to bacterial and viral infections, and vaccines containing derived immunogenic epitopes. As these antibodies largely recognize surface peptide epitopes on immunogenic protein ligands, they are more likely to be enriched in the correct IgG isotype required to engage immune effector cells. Although no ligands exist to engage these antibodies in a bi-functional molecule format, low molecular weight (MW) peptide sequences derived from native immunogenic epitopes/antibody binding sites represent one accessible and convenient source. Peptide ligands can possess high immune receptor binding specificity compared to small molecules, due to their ability to contact a large binding site interface and mimic key interactions of the native protein ligand. This makes peptide ligands strategic binding units to integrate into immune proximity-inducing bi-functional molecule formats.

The utility of low MW immune receptor targeting peptides derived from native protein ligands for “immune proximity-induction”, is contingent upon sufficiently high peptide binding affinity to stabilize resultant bi-functional molecule templated ternary complexes. The difficulties associated with rapidly discovering/developing sub nM-pM affinity binding peptides inspired efforts towards developing a strategy that incorporates covalent binding peptides into bi-functional molecules. In this covalent peptide-based proximity inducing strategy, the peptide ligand of interest incorporated into a bi-functional ARM can be modified with a strategic reactive electrophilic group. This would enable the resultant bi-functional molecule to engage protein binding immune receptors with “infinitely high” binding affinity. Furthermore, the resultant covalent linkage to the immune receptor can help reduce molecule dosing requirements in a therapeutic context by decreasing the clearance rate and increasing the in vivo stability of the bi-functional molecule.

The following key design principles/requirements were considered:
1. The reactive group needs to be hydrolytically stable in a therapeutically relevant in vivo setting.
2. The reaction kinetics need to be sufficiently fast to occur competitively with in vivo clearance of the bi-functional ARM.
3. The reactive group should react with diverse amino acids proximal to the peptide binding site on the target immune receptor.
4. The reactive group should be positioned on the peptide at a location that places it proximal to these amino acids upon receptor binding. This requirement is critical for covalent binding selectivity, where the reactive group experiences a substantial increase in reaction rate with target receptor amino acids relative to the same amino acids on off-target proteins (i.e., an effective molarity “EM” enhancement).
5. The reactive group cannot efficiently react intramolecularly with amino acids on the peptide ligand within the bi-functional ARM.
6. The reactive group can be efficiently and orthogonally installed on the peptide during or post solid phase peptide synthesis (SPPS).

Upon selective binding to the immune receptor of interest, the bi-functional molecule can now covalently link to the receptor and stably template a ternary complex with the cancer cell. This “covalent proximity induction” strategy can enable efficient recruitment of a diverse range of immune receptors via facile incorporation of diverse modest affinity peptide ligands derived from native protein epitopes, or rationally designed/discovered via evolution/enrichment approaches. Importantly, this approach also expands the generality of the covalent immune recruiting strategy beyond small molecule hapten specific serum antibodies.

As a proof of concept, herpes simplex virus (HSV)-specific antibodies naturally prevalent in human blood were chosen as a model system for protein binding immune receptors. These antibodies naturally arise due to host exposure to HSV infection and recognize immunogenic peptide sequences on glycoproteins decorating the viral surface. Notably the natural presence of these antibodies can also be deleterious to viral vector based tumor immunotherapies like the FDA approved anti-tumor oncolytic virus T-VEC. T-VEC both induces the production of, and is sequestered by, HSV glycoprotein specific antibodies. As such a chemical bi-functional molecule strategy to redirect these antibodies away from viral vectors against tumor targets can represent a powerful combination cancer treatment strategy. Additionally, virus-specific antibodies were strategically chosen as a model system due to their enrichment in immune cell activating isotypes (e.g., IgG1), coupled with the general applicability of this approach for redirecting other virus specific antibodies prevalent in human blood (e.g., induced by anti-viral vaccines), against cancer.

For these studies, a single immunogenic peptide epitope derived from HSV gD, which is recognized by a specific and commercially available monoclonal antibody (mAb LP14) was selected. The goal was to transform this peptide into a covalent proximity inducing bi-functional molecule, by a) engineering the peptide with an electrophilic handle and b) equipping the peptide with a cancer antigen binding ligand. To afford cancer targeting, an established glutamate urea ligand that binds the PSMA protein antigen with ≈10⁻⁸ M affinity was chosen. PSMA is generally found over-expressed on solid tumor neo-vasculature and on prostate tumors. The strategy involved first substituting potential electrophile self-reactive native lysine (K) residues on the peptide sequence, with arginine (R) amino acids to circumvent potential intramolecular side-reactions with the select electrophilic handle. To test whether antibody recognition of the peptide was not disrupted by arginine substitution as hypothesized, an in-house bio-layer interferometry (BLI) binding assay was used. This assay detects protein (i.e., antibody) binding to biotinylated peptides that are pre-immobilized on a biosensor probe. The results of the BLI binding assay indicated that arginine (R) mutations at lysine (K) positions modestly enhanced binding affinity for anti-gD IgG antibody through decreases in binding k_{off} (Table 10).

TABLE 10
BLI analysis of synthetic HSV gD peptide affinity for model anti-gD
IgG antibody and covalent SuFEx installation effects.

| Biotinylated | | | | |
|---|---|---|---|---|
| Peptide | Sequence | Kₒₙ (M⁻¹s⁻¹) | K_{off} (s⁻¹) | K_{D} (nM) |
| gD | AcK_{N3}LKMADPNRFRGKDL | 81450 ± | 0.01199 ± | 147.21 ± |
| | (SEQ ID NO: 2) | 350 | 0 | 0.88 |
| gDR (K → R) | AcK_{N3}LRMADPNRFRGRDL | 87800 ± | 0.007244 ± | 82.51 ± |
| | (SEQ ID NO: 3) | 350 | 0 | 0.44 |
| gDR*-SuFEx | AcK_{N3}LRMADPNRFRGRDLY(OSO₂F) | 119400 ± | 0.006374 ± | 53.39 ± |
| (+ C-term Tyr-OSO₂F) | (SEQ ID NO: 5) | 750 | 0 | 0.30 |
| *gDR-SuFEx | AcY(OSO₂F)K_{N3}LRMADPNRFRGRDL | 67150 ± | 0.00569 ± | 87.74 ± |
| (+ N-term Tyr-OSO₂F) | (SEQ ID NO: 6) | 800 | 0 | 0.82 |
| gD*R-SuFEx | AcK_{N3}LRMADPNRY(OSO₂F)RGRDL | 68800 ± | 0.0221 ± | 321.22 ± |
| (Phe10 → Tyr-OSO₂F) | (SEQ ID NO: 7) | 600 | 0 | 2.60 |

Next, sulfuryl (VI) fluoride (SuFEx) chemistry was selected as the electrophilic handle for peptide incorporation. In addition to its versatile amino acid reactivity (i.e., tyrosine, lysine, and histidine) and high hydrolytic stability, SuFEx has been successfully used previously in the context of a covalent binding therapeutic protein. It was hypothesized that the gD peptide ligand armed with SuFEx chemistry would enable the resultant bi-functional molecule to covalently bind anti-gD antibodies with enhanced kinetics compared to off-target proteins, and efficiently bridge these antibodies with cancer targets. This would be possible if peptide binding positions the SuFEx group close to amino acids on the antibody, and that these amino acids can act as potential nucleophiles. Other affinity labeling chemistries (e.g., acylimidazoles) are much more hydrolytically labile and/or selective for a single amino acid (e.g., lysine) that may not be close enough to the peptide binding site.

Next, a successful strategy to incorporate the SuFEx group site specifically within the peptide sequence during SPPS (Scheme S3) was devised. This strategy involved late stage installation of a fluorosulfate group at locations in the peptide sequence substituted with an O-allyl protected tyrosine. The O-allyl group enabled for efficient orthogonal introduction of the fluorosulfate upon treatment with AISF, following on-bead de-allylation using a homogenous Pd (0) catalyst (Scheme S3). To screen the optimal position for SuFEx incorporation given the antibody binding site sequence is unknown, the SuFEx group was inserted at three different positions within the peptide sequence: a) the N-terminus (*gDR-SuFEx, Table 10), b) an internal phenylalanine location previously implicated in antibody binding (gD*R-SuFEx, Table 10), and c) the C terminus (gDR*-SuFEx, Table 10). This was done to probe the location upon binding the anti-gD antibody, that optimally pre-organizes the SuFEx with nucleophilic residues proximal to the peptide binding site. The model monoclonal anti-gD antibody chosen for these studies thus models the therapeutically relevant scenario where polyclonal anti-viral antibodies can be engaged using covalent proximity-inducing bi-functional molecules, without prior knowledge of their binding site amino acid distribution. Following global peptide deprotection from the solid support, the covalent peptides which share an N-cap azido lysine were subjected to solution phase SPAAC coupling to TBTs comprising a cancer antigen binding ligand, fluorophore, or biotin (FIG. 74, Table 10).

The anti-gD IgG binding affinity of the three biotinylated covalent peptides differing in SuFEx location (“gDR-SuFEx”, Table 10) was validated, in the above mentioned BLI assays (FIGS. 42-46). Here, covalent binding to antibody can clearly be discerned from non-covalent binding, by the amplitude of the antibody binding dissociation phase in the presence of free peptide competitor ligand (FIGS. 45 and 46). In these experiments, SuFEx installation through tyrosine substitution was well tolerated by the antibody at N and C terminal locations within the peptide sequence (*gDR-SuFEx and gDR*-SuFEx respectively), giving rise to similar dissociation constants on the order of 10⁻⁸ M (Table 10). Interestingly, the gD*R-SuFEx peptide with the internal native phenylalanine substituted with a tyrosine containing SuFEx experienced an approximate 7-fold loss in affinity (FIG. 42). Since this position has been implicated in direct binding to anti-gD antibody, this result is consistent with a tight binding contact between phenylalanine and the antibody. As such, it was hypothesized that having the SuFEx at this position could enable for a substantial enhancement in reaction effective molarity, if the group is pre-organized close to nucleophilic amino acids around the peptide binding site on anti-gD IgG. This would effectively transform the moderate affinity binding gD peptide, into to one that binds the anti-gD antibody with “infinite affinity” through a proximity induced covalent linkage reaction.

Next the ability of these three covalent peptides to irreversibly link to anti-gD IgG was tested using fluorescence SDS-PAGE. For these assays, fluorophore substituted derivatives of *gDR-SuFEx (cARM 2.1), gD*R-SuFEx (cARM 2.2), and gDR*-SuFEx (cARM 2.3) were synthesized and incubated with anti-gD IgG antibody for 24 hr (FIGS. 74 and 75A). In this assay format, the reaction can be monitored by the emergence of fluorescence protein bands corresponding to covalently labeled antibody heavy and/or light chains. cARMs 2.1-2.3 were each incubated in 20 fold excess to anti-gD antibody for 24 hours, to rigorously assess labeling selectivity. Only selective labeling follows a pre-complexation dependent reaction mechanism, dependent on both binding to the gD peptide binding site of anti-gD IgG antibody (described by K_{D}, FIG. 75B), and a subsequent pseudo-intramolecular reaction with a proximal nucleophilic amino acid (described by k_{inact}, FIG. 75B). Off-target bimolecular reactions with other amino acids on IgG (or other proteins) on the other hand, occur independent of a selective binding step. These are more likely to occur as covalent peptide concentrations (and stoichiometry) exceed antibody. Incubation of cARMs 2.1-2.3 with only 1 μM anti-gD IgG gave rise to appreciable covalently linked antibody product after 24h. Control experiments involved incubating cARMs 2.1-2.3 with a non-binding IgG isotype control antibody, or with the selective anti-gD IgG antibody in the presence of free competitor gD peptide. Both control experiments showed baseline signal consistent with high covalent binding selectivity. As an additional control that only anti-gD IgG covalently linked to peptide was detected in these assays, a non-reactive analog of cARM 2.3, where the SuFEx covalent binding group is hydrolyzed prior to incubation with anti-gD IgG, was also incubated and gave rise to baseline signal.

cARMs 2.1 and 2.3 were observed to predominantly link to the antibody light chain, with some minor reaction with the heavy chain. As this covalent reaction is dependent on selective binding, this result supports the assertion that both heavy and light chains comprising the antibody binding site, contain proximal SuFEx reactive nucleophilic amino acids. Interestingly, cARM 2.2 which contains the fluorosulfate group at the internal phenylalanine position directly involved in antibody binding, exclusively labels the light chain. This result supports a more stringent pre-organization of the SuFEx upon cARM 2.2/antibody binding where the SuFEx is likely restricted to accessing a smaller subset of potential reactive amino acids. As a reciprocal validation of selective covalent antibody binding, biotinylated analogs of cARMs 2.1-2.3 were independently validated in parallel BLI labeling assays at reaction endpoint (FIGS. 45 and 46).

To estimate the pseudo-intramolecular reaction rate constant k_{inact}, which describes the efficiency of covalent peptide linkage to the anti-gD antibody within the bound complex, a full reaction time-course analysis was performed using the gel assays above. Next, a progress curve was generated for each of the three covalent binding peptides 2.1-2.3 under first order reaction conditions. At these concentrations, anti-gD IgG is saturated with covalent peptide in non-covalent complexes. Prior to curve fitting analysis to extract kinact, parallel ELISA assays were conducted. The ELISA assays confirmed that the reaction plateaus observed in fluorescence SDS-PAGE experiments corresponded to 100% antibody labeling (FIG. 53). Strikingly cARMs 2.1-2.3 exhibited similar antibody labeling reaction kinetics sharing a calculated k_{inact} value on the order of 10⁻⁵s⁻¹. This suggests the fluorosulfate group in each of the three different SuFEx positions experiences a similar reaction effective molarity/pre-organization upon peptide binding to antibody. A more rigorous kinetics and proteolytic analysis of covalent labeling location on the antibody is not possible, given access to limited quantities of antibody coupled with its unknown composition. However, it is reasonable, given the high reaction selectivity observed, coupled with the fact covalent labeling likely blocks the binding site from additional peptide labeling events, to estimate that the formation of anti-gD antibody-peptide covalent conjugates in a 2:1 peptide:antibody stoichiometry (i.e., one peptide per Fab domain) is being monitored.

The pseudo-intramolecular labeling kinetics observed enable moderate affinity binding peptides (≈10⁻⁷ M) to bind “irreversibly” to the target antibody at low M concentrations on the order of hours (e.g., cARM 2.2). This finding is highly significant for potential function in an in vivo therapeutic setting where low concentrations of protein binding immune receptors, can be covalently recruited to cancer targets using bi-functional molecules. Although these rates would enable some covalent binding to occur competitively with the hour time scale in vivo clearance rates associated with low MW therapeutics, excess compound would likely need to be administered to covalently modify all available anti-gD antibody. As such, the potential therapeutic utility of covalent proximity inducing molecules depends both on a) sufficiently fast reaction kinetics, and b) their ability to covalently react with the target protein faster than off-target proteins containing the same nucleophilic residues. Faster reaction with the target protein can be achieved if the reactants are sufficiently pre-organized upon binding, prior to the reaction rate limiting transition state. In the system of the disclosure, pre-organization was accomplished through compound:antibody binding, prior to concerted nucleophilic amino acid attack at the sulfur (VI) center on fluorosulfate. This occurs concomitant with fluoride departure from a trigonal bi-pyramidal transition state. Reactant pre-organization in the binding step pays for the translational and rotational entropic cost of the covalent reaction step leading to subsequent rate enhancements. The rate enhancement achievable from pre-organization effects known as kinetic “effective molarity (EM)”, is described by the ratio of first and second order reaction rate constants (i.e., kᵢₙₜᵣₐ/kᵢₙₜₑᵣ, in units of M). In the system of the disclosure, this is the ratio of the rate constants describing nucleophilic attack on SuFEx within the compound:antibody non-covalent complex, compared to the rate constant describing the analogous second order bi-molecular reaction between antibody and compound without binding. The better nucleophilic amino acids on the antibody and SuFEx on the bound peptide are pre-organized into the reaction transition state geometry, the faster the reaction rate will be compared to the analogous bimolecular reaction without pre-organization (i.e., large EM). The maximal theoretical kinetic enhancement or EM achievable has been estimated on the order of 10⁸ M, with values in the 1-55 M range achieved in practice. This estimate assumes translational and overall rotational entropic costs are paid prior to the reaction transition state, with no additional energetic penalties arising from strain, desolvation, or losses in linker conformational entropy.

The apparent independence of covalent binding kinetics on peptide positioning of the covalent SuFEx group suggests one of the following scenarios is operative: 1. each antibody recruitment molecule (cARM 2.1-2.3) reacts with the same amino acid; 2. amino acid nucleophilic attack on SuFEx is not rate limiting; or 3. the kinetic benefits of reactant pre-organization (i.e., effective molarity) are similar despite likely targeting different combinations of reactive tyrosines/lysines/histidines. To estimate the EM the apparent bimolecular antibody covalent reaction rate constant kᵢₙₜₑᵣ, was determined using non-binding IgG isotype antibody lacking the gD binding site. The apparent rate constant kᵢₙₜₑᵣ for labeling full IgG isotype control molecule was determined with kᵢₙₜᵣₐ determined above for labeling anti-gD IgG (FIGS. 50-52). This gave rise to an estimated EM enhancement on the order of; 1 mM for covalent peptide binding to anti-gD IgG. This estimation suggested that cARMs 2.1-2.3 which selectively label anti-gD antibody at low M concentrations would need to be increased to a concentration of 1M to react non-specifically with off-target proteins at the same rate. EM represents a critically important yet relatively unexplored parameter in covalent drug/peptide development. For example, covalent binding groups associated with fast reaction kinetics but lower EM values may be problematic when incorporated into a covalent drug with lower binding affinity for the target protein. In this scenario, higher covalent drug concentrations would be required to achieve the fraction of target protein binding needed to facilitate the selective covalent rxn. Unfortunately, this cannot be efficiently achieved without substantial competing off-target reactions with nucleophiles in a bi-molecular fashion.

Prior to studying covalent immune proximity induction in functional immune assays, the ability of the peptides disclosed herein to form covalent ternary complexes with antibody and soluble tumor antigen was validated in established BLI assays described previously. For these purposes, bi-functional cARM 2.4 which shares the covalent binding peptide in cARM 2.1 but substitutes fluorescein with a PSMA tumor antigen binding ligand was synthesized (FIG. 74). In these assays, only covalently labeled antibody (captured by biosensor probes) contained the PSMA binding ligand, which would lead to signal increases upon addition of soluble PSMA. Indeed the results of these experiments confirmed cARM 2.4 mediated covalent ternary complex formation (i.e., antibody-cARM2.4:PSMA, where the dash represents a covalent linkage and the colon represents non-covalent binding). This assay format was further used to validate cARM 2.4 covalent antibody binding kinetics. A plot of the reaction progress with time enabled for estimation of k_{inact} in very good agreement (≈10⁻⁵ s⁻¹) with that calculated for analogous peptide cARM 2.1 in fluorescence gel assays above (FIGS. 47, 48 and 76). PSMA binding resulted in increases in BLI signal (nm shift) on the order of seconds (FIG. 76A). Collectively, this data validates the first example of a proximity inducing bi-functional antibody recruitment molecule using a covalent binding peptide reported in the literature.

### Example 24: Covalent Antibody Recruitment Molecules Promote Antibody-Dependent Cellular Phagocytosis of Human Cells Expressing a Tumor Antigen

Covalent immune proximity induction was then studied in a previously established flow cytometry phagocytosis assay using PSMA expressing cancer targets. In these assays human immune monocytes can be recruited to phagocytose and destroy cancer cells decorated with IgG antibodies. Here, the ability of cARM 2.4 (GU-FSY-gDR) to bridge viral protein specific anti-gD IgG antibodies with cancer targets expressing the PSMA tumor antigen can be directly evaluated using cancer cell phagocytosis as a readout. This was initiated by incubating different concentrations of anti-gD antibody with stoichiometric amounts of cARM 2.4 giving rise to a dose dependent increase in phagocytosis of target PSMA+HEK cells. Repeating these experiments with a non-reactive analog of cARM 2.4 (i.e., ARM 2.17 (GU-gD)) that can only reversibly bind anti-gD antibodies, also showed dose dependent phagocytosis. Phagocytosis with this analog occurred with lower potency compared to the covalent binding cARM 2.4 illustrating the functional advantage associated with covalent proximity induction. Control experiments for covalency involved the addition of excess free gD peptide competitor, to solutions containing cARM 2.4 or ARM 2.17 and anti-gD IgG that were already incubated for 24 hr, prior to initiation of phagocytosis. This competitor “quench” abolished signal for phagocytosis mediated by non-reactive ARM 2.17 which cannot form a covalent linkage but could not disrupt phagocytosis promoted by cARM 2.4. An additional control for the covalent binding selectivity of cARM 2.4 involved incubations with isotype control IgG giving rise to near baseline signal. This IgG contains the same amino acid content as anti-gD IgG with analogous capabilities to activate immune cells, but cannot pre-complex cARM 2.4 to induce a selective covalent reaction.

Notably, the high potency of both cARM 2.4 and non-reactive ARM 2.17 is consistent with high avidity binding to surface PSMA and in the case of non-reactive ARM 2.17, high avidity binding to anti-gD IgG. In the presence of a multivalent array of cancer antigens on the cell surface, the apparent binding affinity of bi-functional compound for both antigen and antibody increases substantially. High avidity binding is supported by the observation of a sigmoidal dose response curve upon titrations with non-reactive ARM 2.17. Further support for avidity is founded in the fact that maximal efficacy is reached at such low anti-gD IgG concentrations (≈12 nM) when the calculated solution K_{d} for both PSMA and antibody binding are on the order of ≈10-8 M. Additional antibody recruitment studies on the same target cells demonstrated all available PSMA are already saturated with ternary complexes at antibody concentrations as low as 25 nM (FIG. 61).

cARM 2.1 was re-synthesized to substitute the fluorosulfate (Ar—OSO₂F) with a more electrophilic aryl-sulfonyl fluoride (Ar—SO₂F) to yield cARM 2.5 (FIG. 74). In fluorescence gel assays run at the same concentrations used to characterize cARMs 2.1-2.3, cARM 2.5 was validated as maintaining the high selectivity of cARM 2.1 but experienced an order of magnitude enhancement in the rate of covalent antibody binding (FIGS. 76B and 76C). Interestingly, the aryl-sulfonyl fluoride (ASF) group on cARM 2.5 appears to preferentially link to the anti-gD heavy chain in contrast to its fluorosulfate (FSY) substituted analog cARM 2.1. As differences in covalent group pre-organization and proximity to target amino acids on the antibody are unlikely, this difference likely reflects a propensity to react with a closer but weaker nucleophile which is not possible with the more stable fluorosulfate (e.g., tyrosine or histidine vs. lysine).

While not being bound by any specific theory, it is reasonable to hypothesize these kinetics would enable for a single low M dose of compound to largely covalently label stoichiometric concentrations of target antibody before getting cleared from systemic circulation. Although endogenous concentrations of anti-gD HSV antibodies in the latently infected patient population are likely far below M, much higher concentrations can likely be induced in cancer patients treated with HSV oncolytic virus tumor immunotherapies.

Although the disclosure has been described with reference to certain specific embodiments, various modifications thereof will be apparent to those of ordinary skill in the art. Any examples provided herein are included solely for the purpose of illustrating the disclosure and are not intended to limit the disclosure in any way. Any drawings provided herein are solely for the purpose of illustrating various aspects of the disclosure and are not intended to be drawn to scale or to limit the disclosure in any way. The scope of the claims appended hereto should not be limited by the preferred embodiments set forth in the above description, but should be given the broadest interpretation consistent with the present specification as a whole. The disclosures of all documents cited herein are incorporated herein by reference as if set forth in their entirety.

We claim:

1. A compound of Formula (I) or a pharmaceutically acceptable salt or solvate thereof
(TBT)ₙ-(L)ₘ-(ABT)ₚ (I)
wherein:
TBT is a target binding terminus comprising at least one moiety that binds to at least one target protein;
L is an optional linker;
ABT is an antibody binding terminus comprising at least one epitope or epitope mimetic of a Herpes Simplex Virus (HSV) surface protein;
each of n and p is independently 1 or any integer greater than 1; and
m is 0 or any integer greater than 0.

2. A compound comprising:
a. at least one target binding terminus (TBT) comprising one or more moieties that bind to one or more target proteins;
b. at least one antibody binding terminus (ABT) comprising one or more epitopes or epitope mimetics of a Herpes Simplex Virus (HSV) surface protein; and
c. optionally, at least one linker connecting the at least one TBT with the at least one ABT,
or a pharmaceutically acceptable salt or solvate thereof.

3. The compound of claim 1 or 2, wherein the HSV surface protein is a glycoprotein.

4. The compound of any one of claims 1-3, wherein the HSV surface protein is glycoprotein D (gD).

5. The compound of any one of claims 1-4, wherein the epitope or epitope mimetic comprises the amino acid sequence set forth in any one of SEQ ID NOs: 1-9, or a variant thereof.

6. The compound of any one of claims 1-5, wherein the epitope or epitope mimetic consists of the amino acid sequence set forth in any one of SEQ ID NOs: 1-9, or a variant thereof.

7. The compound of any one of claims 1-6, further comprising one or more reactive groups that mediate covalent conjugation of the compound with an HSV-specific antibody and/or the target protein.

8. The compound of claim 7, wherein the HSV-specific antibody is a serum antibody.

9. The compound of claim 7 or 8, wherein the reactive group comprises an electrophilic functional group that reacts with an amino acid nucleophile in a nucleophilic substitution reaction.

10. The compound of any one of claims 7-9, wherein the reactive group comprises an acyl imidazole group having the following structure: 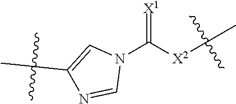
wherein:
X¹ is S, O or NR¹;
X² is O or NR²; and
R¹ and R² are independently H or C₁₋₄ alkyl.

11. The compound of any one of claims 7-9, wherein the reactive group comprises a fluorosulfate-I-tyrosine (FSY) group or an aryl-sulfonyl fluoride (ASF) group.

12. The compound of any one of claims 1-11, wherein the target protein is expressed on the surface of a cancer cell.

13. The compound of any one of claims 1-12, wherein the target protein is urokinase receptor (uPAR), prostate-specific membrane antigen (PSMA), human epidermal growth factor receptor 2 (HER2), or folate receptor.

14. The compound of any one of claims 1-13, wherein the target protein is PSMA and the TBT has the following structure:

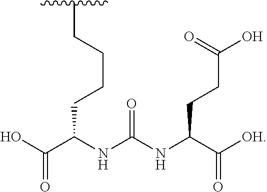 [SMILES: *CCCC[C@H](NC(=O)N[C@@H](CCC(=O)O)C(=O)O)C(=O)O]

15. The compound of any one of claims 1-14, wherein the compound is

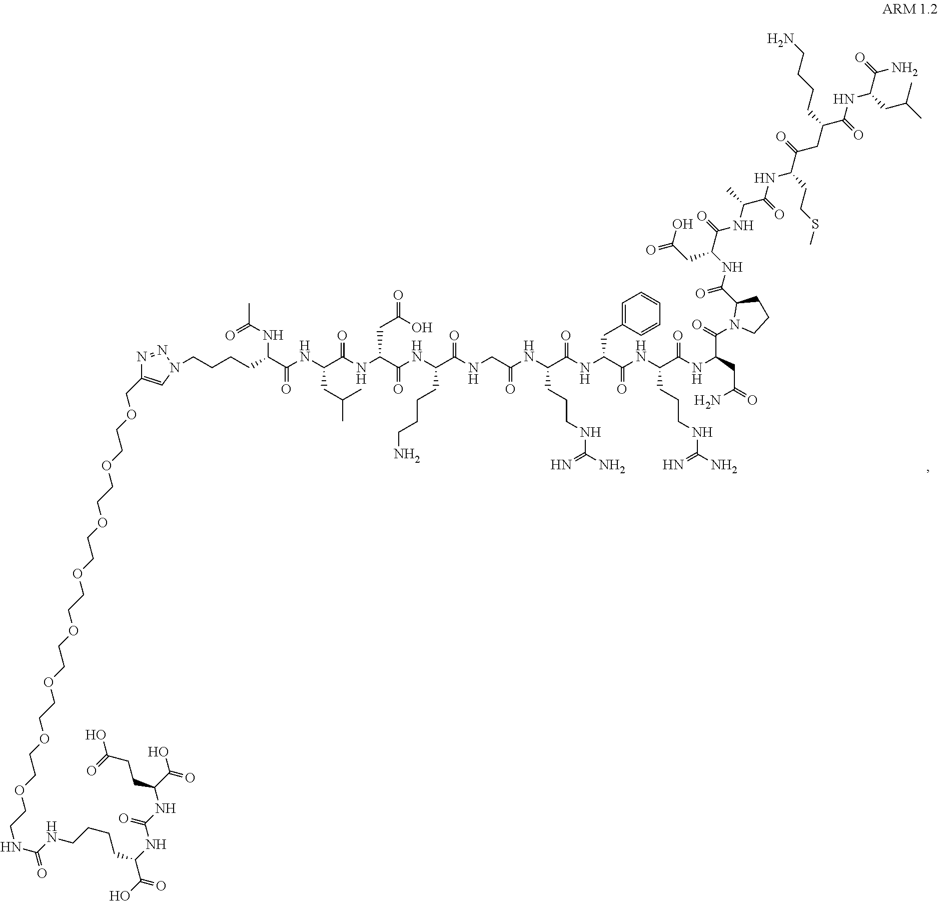 [SMILES: CSCC[C@H](NC(=O)[C@@H](C)NC(=O)[C@@H](CC(=O)O)NC(=O)[C@H]1CCCN1C(=O)[C@@H](CC(N)=O)NC(=O)[C@H](CCCNC(=N)N)NC(=O)[C@@H](Cc1ccccc1)NC(=O)[C@H](CCCNC(=N)N)NC(=O)CNC(=O)[C@H](CCCCN)NC(=O)[C@@H](CC(=O)O)NC(=O)[C@H](CC(C)C)NC(=O)[C@H](CCCCn1cc(COCCOCCOCCOCCOCCOCCOCCOCCNC(=O)NCCCC[C@H](NC(=O)N[C@@H](CCC(=O)O)C(=O)O)C(=O)O)nn1)NC(C)=O)C(=O)C[C@H](CCCCN)C(=O)N[C@@H](CC(C)C)C(N)=O]

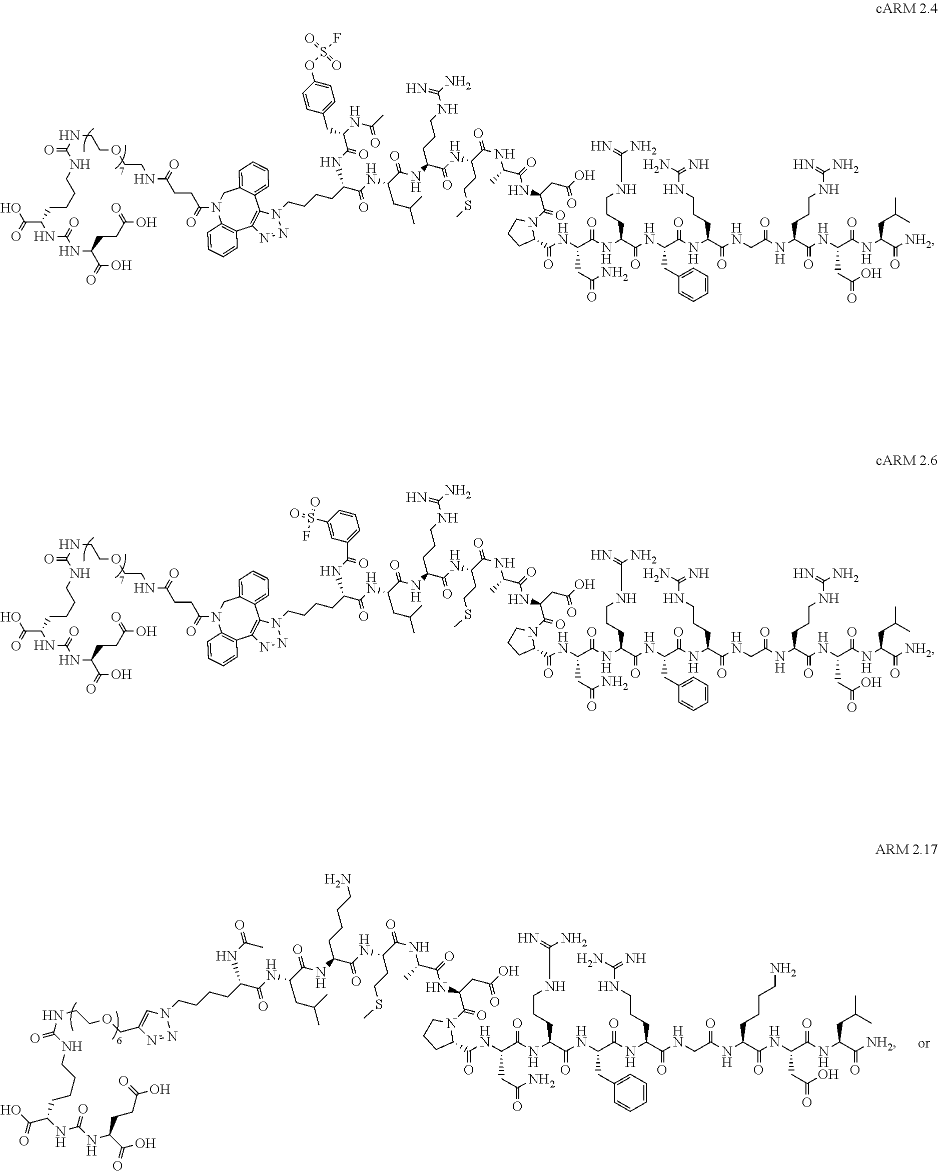

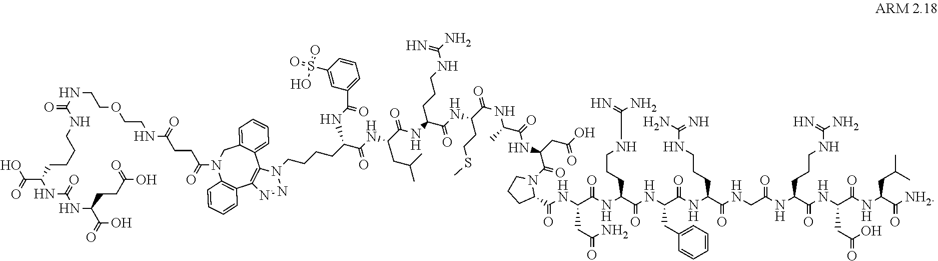 [SMILES: CSCC[C@H](NC(=O)[C@H](CCCNC(=N)N)NC(=O)[C@H](CC(C)C)NC(=O)[C@H](CCCCn1nnc2c1-c1ccccc1CN(C(=O)CCC(=O)NCCOCCNC(=O)NCCCC[C@H](NC(=O)N[C@@H](CCC(=O)O)C(=O)O)C(=O)O)c1ccccc1-2)NC(=O)c1cccc(S(=O)(=O)O)c1)C(=O)N[C@@H](C)C(=O)N[C@@H](CC(=O)O)C(=O)N1CCC[C@H]1C(=O)N[C@@H](CC(N)=O)C(=O)N[C@@H](CCCNC(=N)N)C(=O)N[C@@H](Cc1ccccc1)C(=O)N[C@@H](CCCNC(=N)N)C(=O)NCC(=O)N[C@@H](CCCNC(=N)N)C(=O)N[C@@H](CC(=O)O)C(=O)N[C@@H](CC(C)C)C(N)=O]

16. The compound of any one of claims 1-13, wherein the target protein is uPAR and the TBT has the following structure:

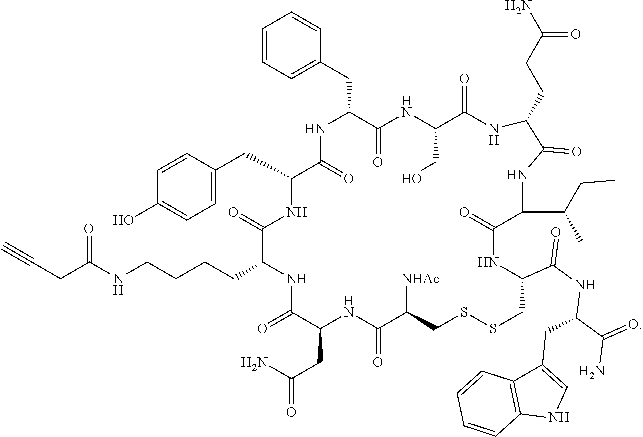 [SMILES: C#CCC(=O)NCCCC[C@H]1NC(=O)[C@H](CC(N)=O)NC(=O)[C@@H](NC(C)=O)CSSC[C@@H](C(=O)N[C@@H](Cc2c[nH]c3ccccc23)C(N)=O)NC(=O)C([C@@H](C)CC)NC(=O)[C@@H](CCC(N)=O)NC(=O)[C@H](CO)NC(=O)[C@@H](Cc2ccccc2)NC(=O)[C@@H](Cc2ccc(O)cc2)NC1=O]

17. The compound of any one of claims 1-13, wherein the target protein is HER2 and the TBT has the following structure:

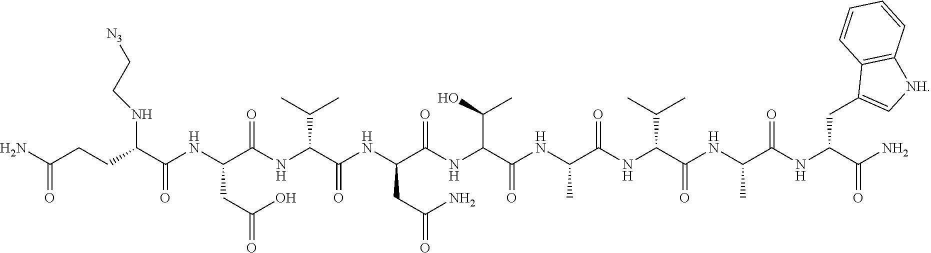 [SMILES: CC(C)[C@@H](NC(=O)[C@H](C)NC(=O)C(NC(=O)[C@@H](CC(N)=O)NC(=O)[C@H](NC(=O)[C@H](CC(=O)O)NC(=O)[C@H](CCC(N)=O)NCCN=[N+]=[N-])C(C)C)[C@H](C)O)C(=O)N[C@@H](C)C(=O)N[C@H](Cc1c[nH]c2ccccc12)C(N)=O]

18. The compound of any one of claims 1-13, wherein the target protein is folate receptor and the TBT comprises methotrexate or folate.

19. The compound of any one of claims 1-11, wherein the target protein is expressed on the surface of a pathogen or a cell infected with a pathogen.

20. The compound of claim 19, wherein the pathogen comprises a virus, bacterium, fungus or parasite.

21. The compound of any one of claims 1-11, wherein the TBT is biotin or a derivative thereof.

22. The compound of claim 21, wherein the TBT has the following structure: 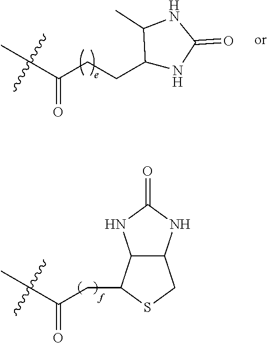
wherein e and f are, independently, an integer from 0 to 15.

23. The compound of claim 21 or 22, wherein the compound is

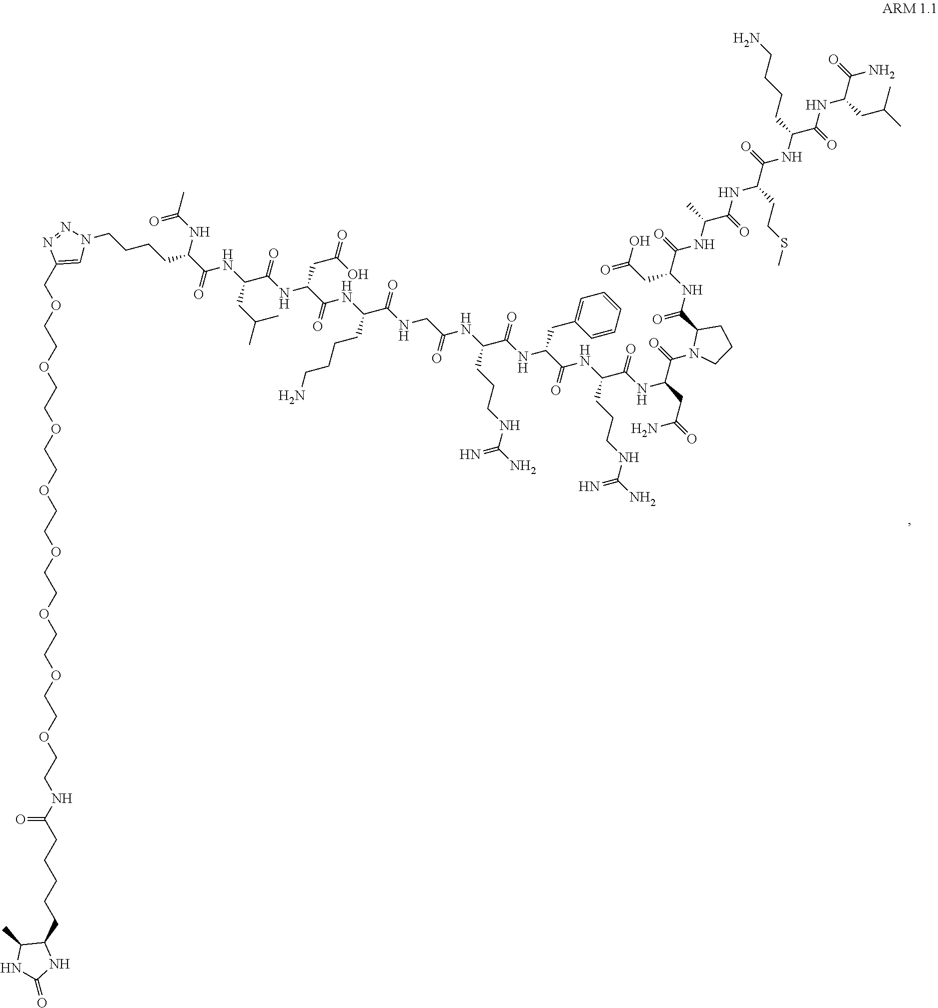 [SMILES: CSCC[C@H](NC(=O)[C@@H](C)NC(=O)[C@@H](CC(=O)O)NC(=O)[C@H]1CCCN1C(=O)[C@@H](CC(N)=O)NC(=O)[C@H](CCCNC(=N)N)NC(=O)[C@@H](Cc1ccccc1)NC(=O)[C@H](CCCNC(=N)N)NC(=O)CNC(=O)[C@H](CCCCN)NC(=O)[C@@H](CC(=O)O)NC(=O)[C@H](CC(C)C)NC(=O)[C@H](CCCCn1cc(COCCOCCOCCOCCOCCOCCOCCOCCNC(=O)CCCCC[C@H]2NC(=O)N[C@H]2C)nn1)NC(C)=O)C(=O)N[C@H](CCCCN)C(=O)N[C@@H](CC(C)C)C(N)=O]

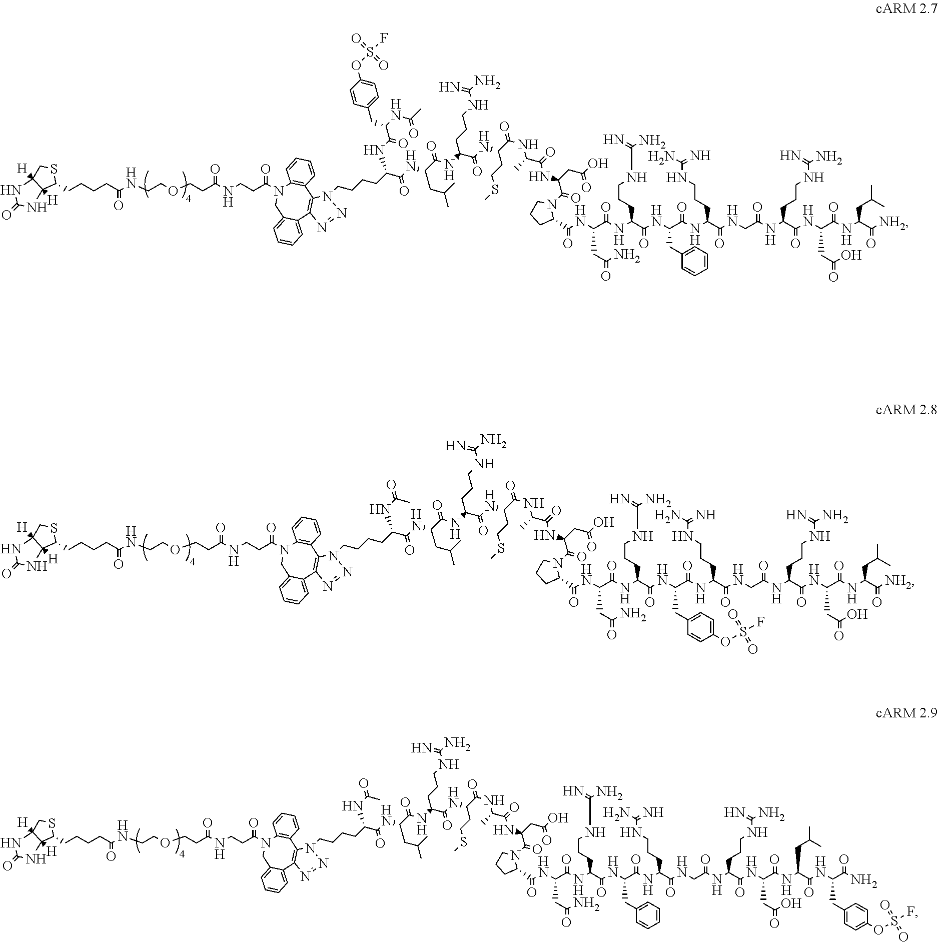

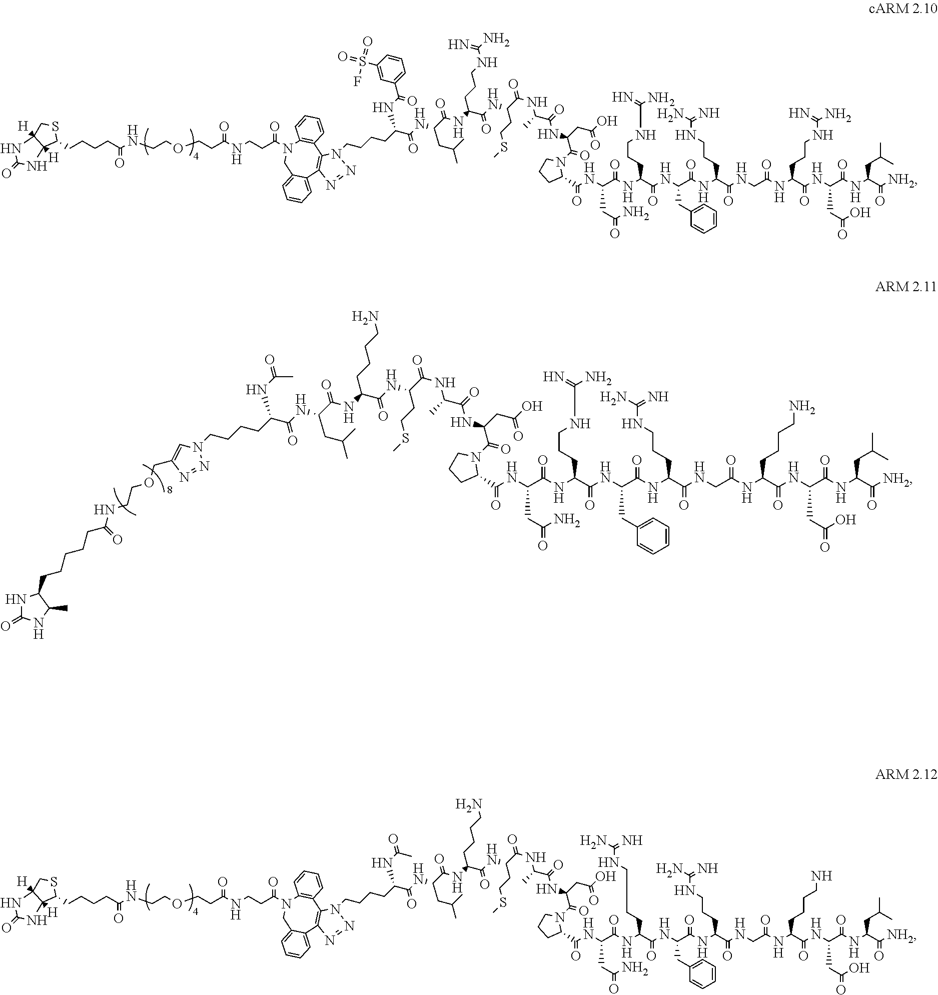

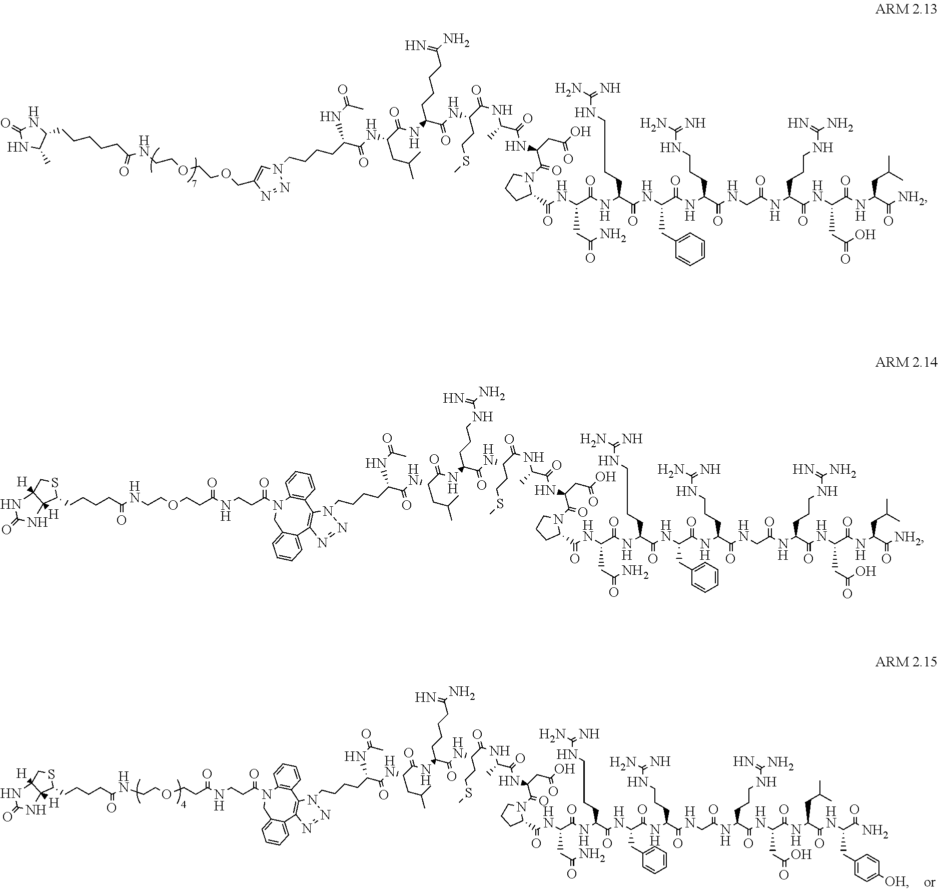

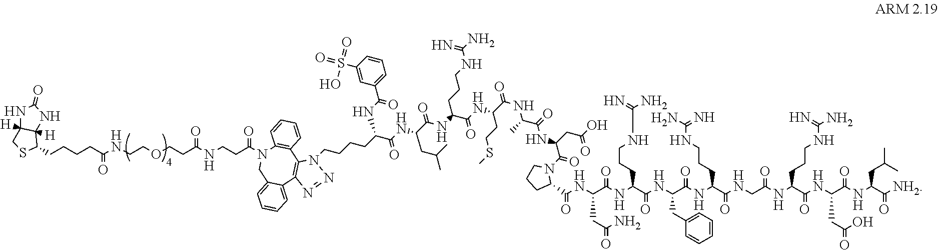

24. The compound of any one of claims 1-11, wherein the TBT comprises a fluorescent reporter.

25. The compound of claim 24, wherein the TBT has the following structure:

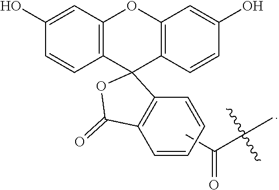

26. The compound of claim 24 or 25, wherein the compound is

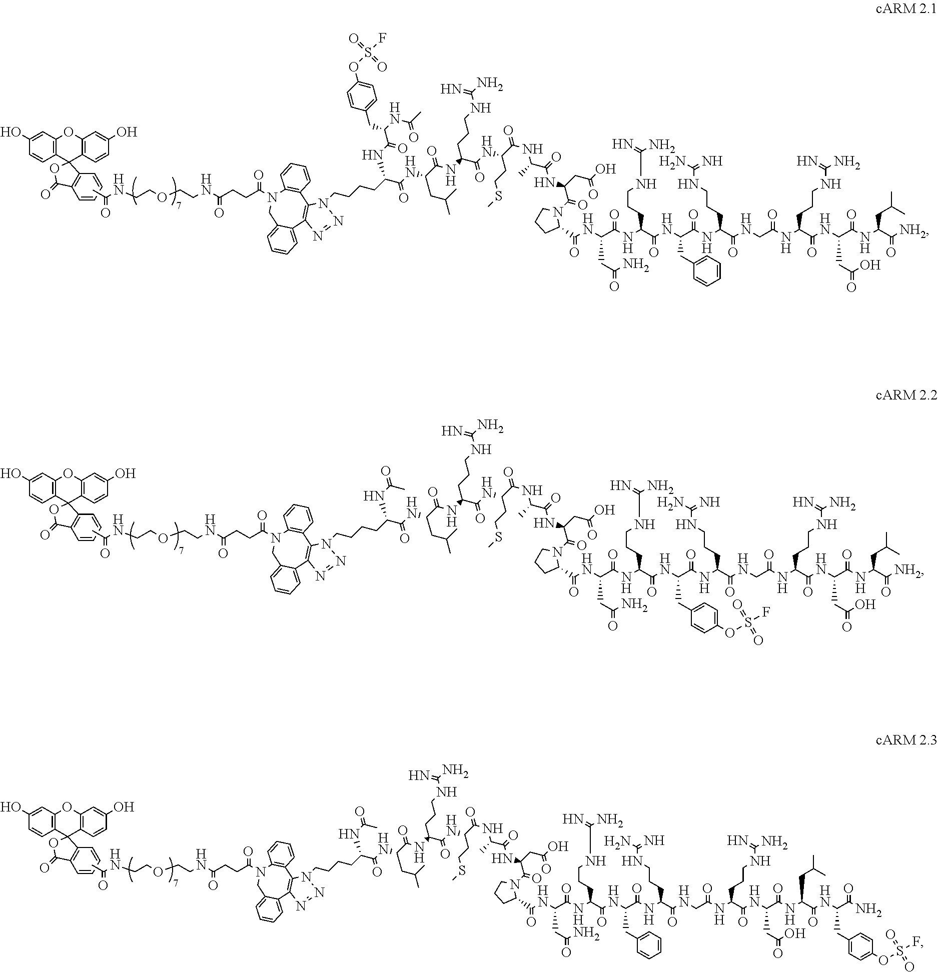

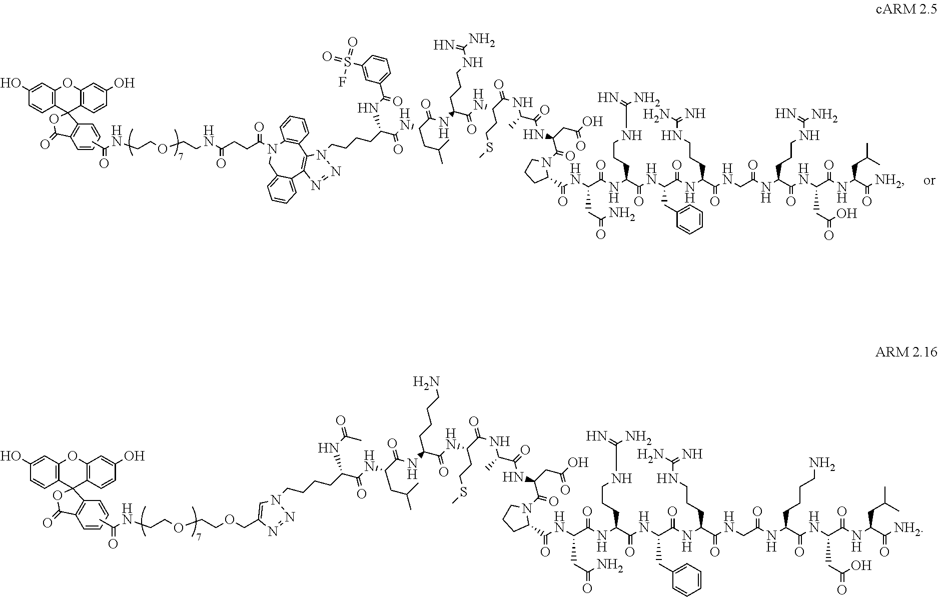

27. A composition comprising the compound of any one of claims 1-26 or a pharmaceutically acceptable salt or solvate thereof, and at least one pharmaceutically acceptable carrier, diluent or excipient.

28. A kit comprising the compound of any one of claims 1-26 or a pharmaceutically acceptable salt or solvate thereof, and an oncolytic virus (OV).

29. The kit of claim 28, wherein the compound and the OV are formulated together.

30. The kit of claim 28, wherein the compound and the OV are formulated separately.

31. The kit of any one of claims 28-30, wherein the OV comprises an oncolytic HSV.

32. The kit of any one of claims 28-31, wherein the OV is Talimogene Laherparepvec (T-VEC).

33. A method of recruiting an HSV-specific antibody to a cancer cell in a subject, the method comprising administering the compound of any one of claims 1-18 or a pharmaceutically acceptable salt or solvate thereof, or the composition of claim 27 to the subject.

34. A method of recruiting an HSV-specific antibody to a pathogen or a cell infected with a pathogen in a subject, the method comprising administering the compound of any one of claims 1-11, 19 and 20 or a pharmaceutically acceptable salt or solvate thereof, or the composition of claim 27 to the subject.

35. The method of claim 33 or 34, wherein the HSV-specific antibody is a serum antibody.

36. A method of treating cancer in a subject, the method comprising administering an effective amount of a compound comprising
a. at least one target binding terminus (TBT) comprising one or more moieties that bind to one or more target proteins on the cancer;
b. at least one antibody binding terminus (ABT) comprising one or more epitopes or epitope mimetics of a Herpes Simplex Virus (HSV) surface protein; and
c. optionally, at least one linker connecting the at least one TBT with the at least one ABT,
or a pharmaceutically acceptable salt or solvate thereof, and an oncolytic virus (OV) therapy to the subject.

37. A method for enhancing the efficacy and/or reducing the toxicity of an oncolytic virus (OV) therapy in a subject with cancer, the method comprising administering an effective amount of a compound comprising
a. at least one target binding terminus (TBT) comprising one or more moieties that bind to one or more target proteins on the cancer;
b. at least one antibody binding terminus (ABT) comprising one or more epitopes or epitope mimetics of a Herpes Simplex Virus (HSV) surface protein; and
c. optionally, at least one linker connecting the at least one TBT with the at least one ABT,
or a pharmaceutically acceptable salt or solvate thereof to the subject.

38. The method of claim 36 or 37, wherein the OV therapy comprises an oncolytic HSV.

39. The method of any one of claims 36-38, wherein the OV therapy is Talimogene Laherparepvec (T-VEC).

40. The compound of any one of claims 36-39, wherein the HSV surface protein is a glycoprotein.

41. The method of any one of claims 36-40, wherein the HSV surface protein is glycoprotein D (gD).

42. The method of any one of claims 36-41, wherein the epitope or epitope mimetic comprises the amino acid sequence set forth in any one of SEQ ID NOs: 1-9, or a variant thereof.

43. The method of any one of claims 36-42, wherein the epitope or epitope mimetic consists of the amino acid sequence set forth in any one of SEQ ID NOs: 1-9, or a variant thereof.

44. The method of any one of claims 36-43, wherein the compound further comprises one or more reactive groups that mediate covalent conjugation of the compound with an HSV-specific antibody and/or the target protein.

45. The method of claim 44, wherein the HSV-specific antibody is a serum antibody.

46. The method of claim 44 or 45, wherein the reactive group comprises an electrophilic functional group that reacts with an amino acid nucleophile in a nucleophilic substitution reaction.

47. The method of any one of claims 44-46, wherein the reactive group comprises an acyl imidazole group having the following structure: 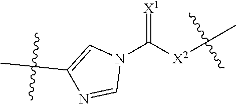
wherein:
X¹ is S, O or NR¹;
X² is O or NR²; and
R¹ and R² are independently H or C₁₋₄ alkyl.

48. The method of any one of claims 44-46, wherein the reactive group comprises a fluorosulfate-I-tyrosine (FSY) group or an aryl-sulfonyl fluoride (ASF) group.

49. The method of any one of claims 36-48, wherein the cancer is prostate cancer and the target protein is PSMA.

50. The method of claim 49, wherein the TBT has the following structure:

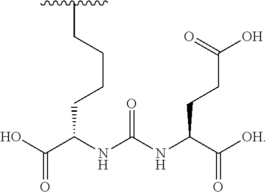 [SMILES: *CCCC[C@H](NC(=O)N[C@@H](CCC(=O)O)C(=O)O)C(=O)O]

51. The method of claim 49 or 50, wherein the compound is

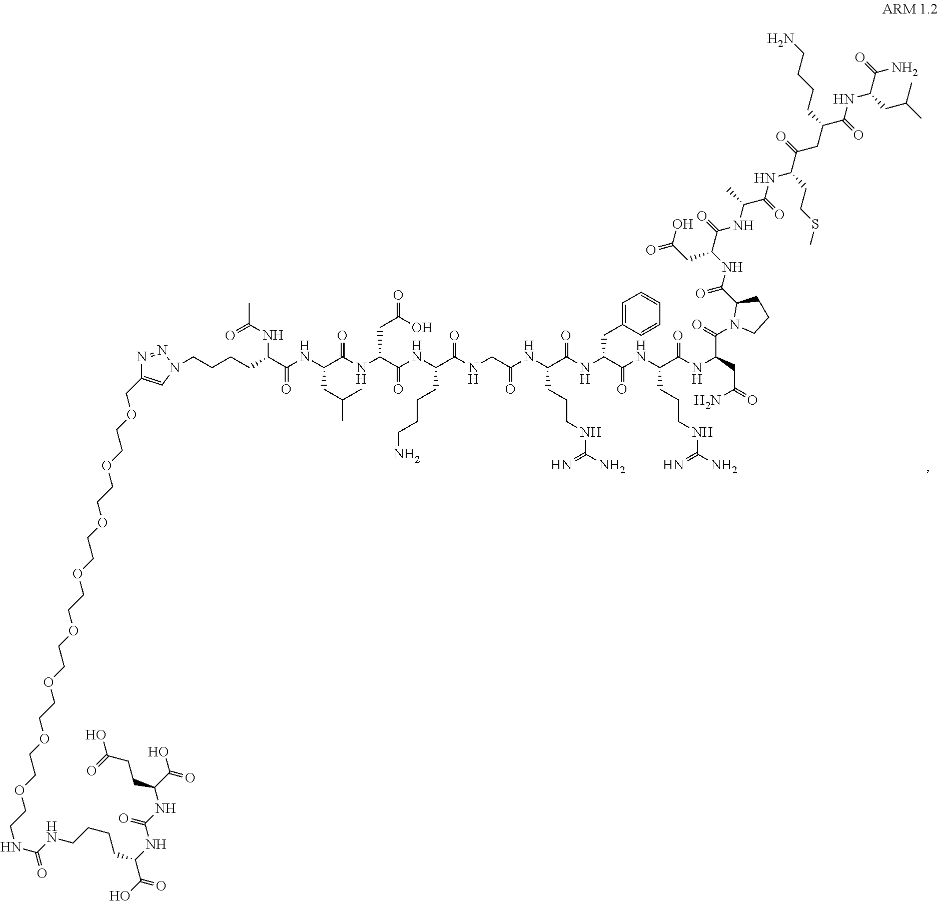 [SMILES: CSCC[C@H](NC(=O)[C@@H](C)NC(=O)[C@@H](CC(=O)O)NC(=O)[C@H]1CCCN1C(=O)[C@@H](CC(N)=O)NC(=O)[C@H](CCCNC(=N)N)NC(=O)[C@@H](Cc1ccccc1)NC(=O)[C@H](CCCNC(=N)N)NC(=O)CNC(=O)[C@H](CCCCN)NC(=O)[C@@H](CC(=O)O)NC(=O)[C@H](CC(C)C)NC(=O)[C@H](CCCCn1cc(COCCOCCOCCOCCOCCOCCOCCOCCNC(=O)NCCCC[C@H](NC(=O)N[C@@H](CCC(=O)O)C(=O)O)C(=O)O)nn1)NC(C)=O)C(=O)C[C@H](CCCCN)C(=O)N[C@@H](CC(C)C)C(N)=O]

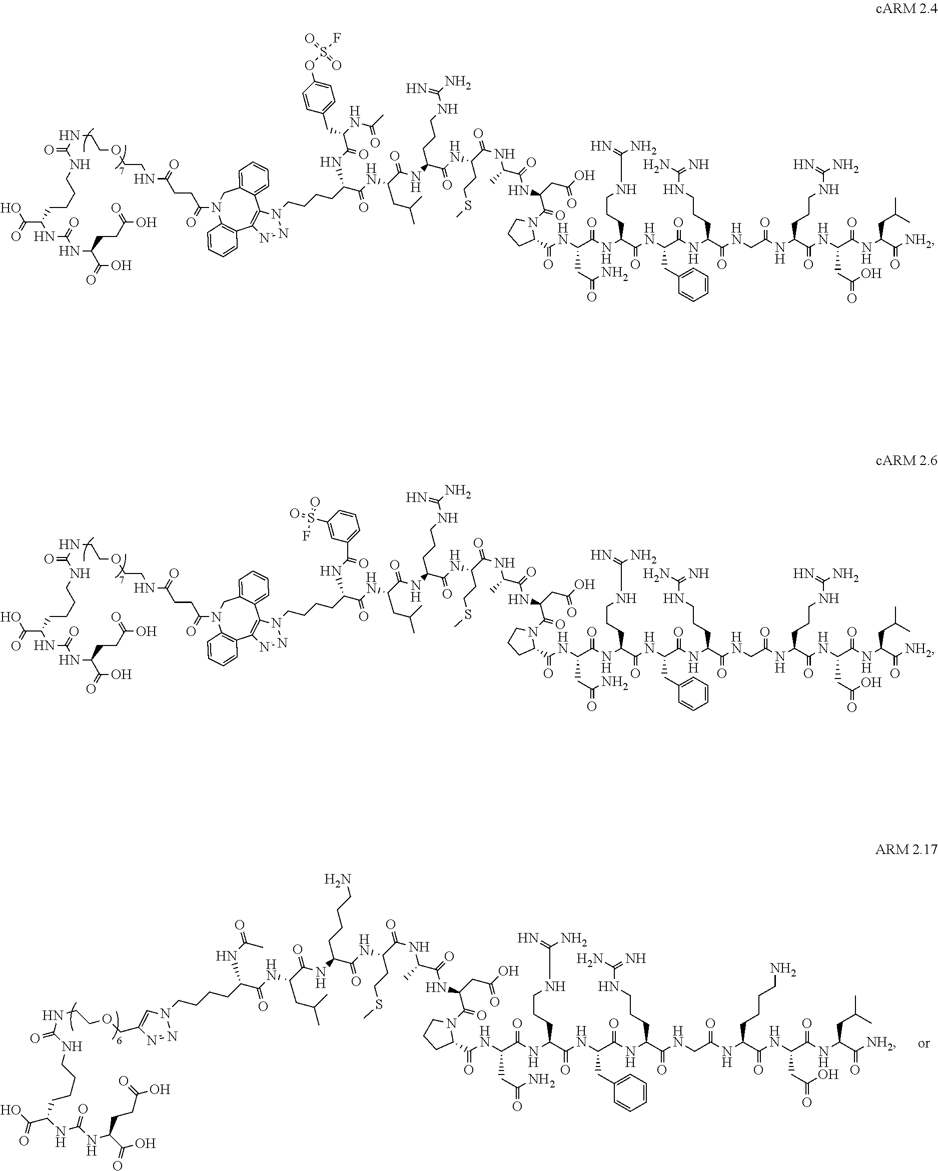

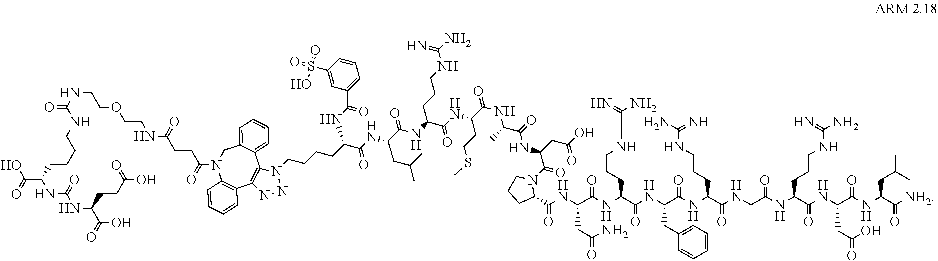 [SMILES: CSCC[C@H](NC(=O)[C@H](CCCNC(=N)N)NC(=O)[C@H](CC(C)C)NC(=O)[C@H](CCCCn1nnc2c1-c1ccccc1CN(C(=O)CCC(=O)NCCOCCNC(=O)NCCCC[C@H](NC(=O)N[C@@H](CCC(=O)O)C(=O)O)C(=O)O)c1ccccc1-2)NC(=O)c1cccc(S(=O)(=O)O)c1)C(=O)N[C@@H](C)C(=O)N[C@@H](CC(=O)O)C(=O)N1CCC[C@H]1C(=O)N[C@@H](CC(N)=O)C(=O)N[C@@H](CCCNC(=N)N)C(=O)N[C@@H](Cc1ccccc1)C(=O)N[C@@H](CCCNC(=N)N)C(=O)NCC(=O)N[C@@H](CCCNC(=N)N)C(=O)N[C@@H](CC(=O)O)C(=O)N[C@@H](CC(C)C)C(N)=O]

52. The method of any one of claims 36-48, wherein the cancer is glioblastoma and the target protein is uPAR.

53. The method of claim 52, wherein the TBT has the following structure:

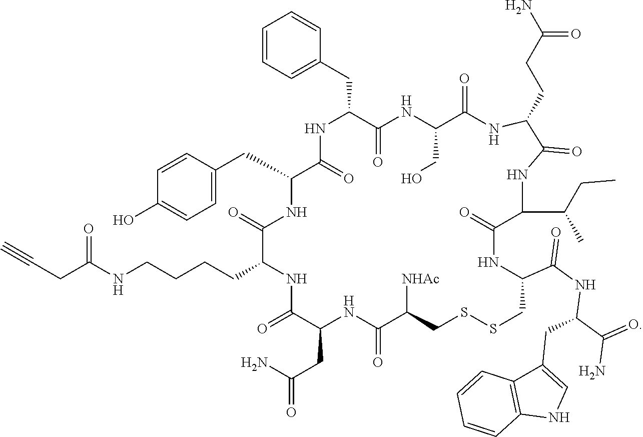 [SMILES: C#CCC(=O)NCCCC[C@H]1NC(=O)[C@H](CC(N)=O)NC(=O)[C@@H](NC(C)=O)CSSC[C@@H](C(=O)N[C@@H](Cc2c[nH]c3ccccc23)C(N)=O)NC(=O)C([C@@H](C)CC)NC(=O)[C@@H](CCC(N)=O)NC(=O)[C@H](CO)NC(=O)[C@@H](Cc2ccccc2)NC(=O)[C@@H](Cc2ccc(O)cc2)NC1=O]

54. The method of any one of claims 36-48, wherein the cancer is breast or ovarian cancer, and the target protein is HER2.

55. The method of claim 54, wherein the TBT has the following structure:

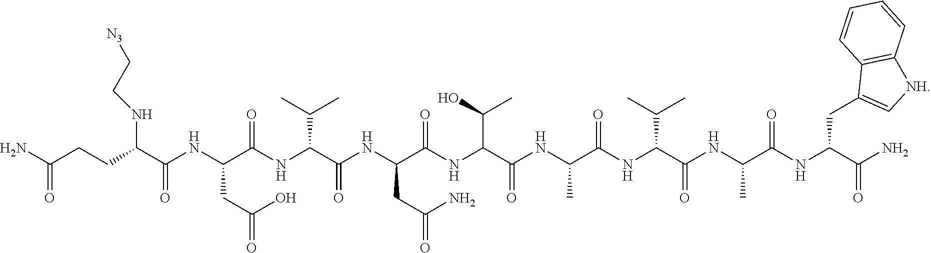 [SMILES: CC(C)[C@@H](NC(=O)[C@H](C)NC(=O)C(NC(=O)[C@@H](CC(N)=O)NC(=O)[C@H](NC(=O)[C@H](CC(=O)O)NC(=O)[C@H](CCC(N)=O)NCCN=[N+]=[N-])C(C)C)[C@H](C)O)C(=O)N[C@@H](C)C(=O)N[C@H](Cc1c[nH]c2ccccc12)C(N)=O]

56. The method of any one of claims 36-48, wherein the cancer is ovarian cancer and the target protein is folate receptor.

57. The method of claim 56, wherein the TBT comprises methotrexate or folate.

58. The method of any one of claims 36-56, wherein the compound is administered to the subject before, concurrently with, and/or after administration of the OV therapy.
